SEQUENCE LISTING (664 sequences)

SEQ ID NO: 1 - DNA, 290 bp, synthetic construct
  misc_feature 1..290: note = Synthetic: FNR Responsive Promoter
  source 1..290: mol_type = other DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 2 - DNA, 173 bp, synthetic construct
  misc_feature 1..173: note = Synthetic: FNR Responsive Promoter
  source 1..173: mol_type = other DNA
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 3 - DNA, 305 bp, synthetic construct
  misc_feature 1..305: note = Synthetic: FNR Responsive Promoter
  source 1..305: mol_type = other DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 4 - DNA, 180 bp, synthetic construct
  misc_feature 1..180: note = Synthetic: FNR Responsive Promoter
  source 1..180: mol_type = other DNA
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 5 - DNA, 199 bp, synthetic construct
  misc_feature 1..199: note = Synthetic: FNR Responsive Promoter
  source 1..199: mol_type = other DNA
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 6 - DNA, 117 bp, synthetic construct
  misc_feature 1..117: note = Synthetic: FNR-responsive regulatory region sequence
  source 1..117: mol_type = other DNA
atccccatcactcttgatggagatcaattccccaagctgctagagcgttaccttgcccttaaacattagcaatgtcgatttatcagagggccgacaggctcccacaggagaaaaccg

SEQ ID NO: 7 - DNA, 108 bp, synthetic construct
  misc_feature 1..108: note = Synthetic: FNR-responsive regulatory region sequence
  source 1..108: mol_type = other DNA
ctcttgatcgttatcaattcccacgctgtttcagagcgttaccttgcccttaaacattagcaatgtcgatttatcagagggccgacaggctcccacaggagaaaaccg

SEQ ID NO: 8 - DNA, 433 bp, synthetic construct
  misc_feature 1..433: note = Synthetic: FNR-responsive regulatory region, nirB2
  source 1..433: mol_type = other DNA
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 9 - DNA, 290 bp, synthetic construct
  misc_feature 1..290: note = Synthetic: FNR-responsive regulatory region, nirB3
  source 1..290: mol_type = other DNA
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 10 - DNA, 207 bp, synthetic construct
  misc_feature 1..207: note = Synthetic: FNR-responsive regulatory region, fnrS2
  source 1..207: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 11 - DNA, 390 bp, synthetic construct
  misc_feature 1..390: note = Synthetic: FNR-responsive regulatory region, Pfnr6 nirB+crp
  source 1..390: mol_type = other DNA
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 12 - DNA, 200 bp, synthetic construct
  misc_feature 1..200: note = Synthetic: FNR-responsive regulatory region, Pfnr7 fnrS+crp
  source 1..200: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 13 - DNA, 164 bp, synthetic construct
  misc_feature 1..164: note = Synthetic: FNR-responsive promoter
  source 1..164: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaa

SEQ ID NO: 14 - DNA, 201 bp, synthetic construct
  misc_feature 1..201: note = Synthetic: FNR-responsive promoter
  source 1..201: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgcatcccacgctaactaatataaggggggcaaga

SEQ ID NO: 15 - DNA, 1083 bp, synthetic construct
  misc_feature 1..1083: note = Synthetic: LacI in reverse orientation
  source 1..1083: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 16 - protein, 360 aa, synthetic construct
  REGION 1..360: note = Synthetic: LacI
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 17 - DNA, 20 bp, synthetic construct
  misc_feature 1..20: note = Synthetic: Lac operator
  source 1..20: mol_type = other DNA
aattgtgagcgctcacaatt

SEQ ID NO: 18 - DNA, 263 bp, synthetic construct
  misc_feature 1..263: note = Synthetic: Promoter comprising Lac operon
  source 1..263: mol_type = other DNA
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaa

SEQ ID NO: 19 - DNA, 222 bp, synthetic construct
  misc_feature 1..222: note = Synthetic: Description of Artificial Sequence: Synthetic polynucleotide
  source 1..222: mol_type = other DNA
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 20 - DNA, 714 bp, synthetic construct
  misc_feature 1..714: note = Synthetic: Description of Artificial Sequence: Synthetic polynucleotide
  source 1..714: mol_type = other DNA
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 21 - protein, 237 aa, synthetic construct
  REGION 1..237: note = Synthetic: Description of Artificial Sequence: Synthetic polypeptide
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 22 - DNA, 84 bp, synthetic construct
  misc_feature 1..84: note = Synthetic: Description of Artificial Sequence: Synthetic oligonucleotide
  source 1..84: mol_type = other DNA
acgttaaatctatcaccgcaagggataaatatctaacaccgtgcgtgttgactattttacctctggcggtgataatggttgcat

SEQ ID NO: 23 - DNA, 714 bp, synthetic construct
  misc_feature 1..714: note = Synthetic: Description of Artificial Sequence: Synthetic polynucleotide
  source 1..714: mol_type = other DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgccttatttaatggcatcaatgcattaaatgcttataacgccgcatcgcttacaagaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgctctcacctgagcttagaacctttaccaaaggtggtgcggaaaggtgggtaagcacaaccaaaaaagccagtgattctgcattctggcttgaggttgaaggtaattccatgacagcaccaacaggctccaagccaagctttcctgacggaatgttaattctcgttgaccctgagcaggctgttgagccaggtgatttctgcatagccagactcgggggtggtgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 24 - protein, 237 aa, synthetic construct
  REGION 1..237: note = Synthetic: Description of Artificial Sequence: Synthetic polypeptide
MSTKKKPLTQEQLEDARRLKAIYEKKKNELGLSQESVADKMGMGQSGVGALFNGINALNAYNAASLTRILKVSVEEFSPSIAREIYEMYEAVSMQPSLRSEYEYPVFSHVQAGMLSPELRTFTKGGAERWVSTTKKASDSAFWLEVEGNSMTAPTGSKPSFPDGMLILVDPEQAVEPGDFCIARLGGGEFTFKKLIRDSGQVFLQPLNPQYPMIPCNESCSVVGKVIASQWPEETFG

SEQ ID NO: 25 - DNA, 183 bp, synthetic construct
  misc_feature 1..183: note = Synthetic: Description of Artificial Sequence: Synthetic polynucleotide
  source 1..183: mol_type = other DNA
aaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataaggtagaaac

SEQ ID NO: 26 - DNA, 56 bp, synthetic construct
  misc_feature 1..56: note = Synthetic: ptet promoter
  source 1..56: mol_type = other DNA
gttgacactctatcattgatagagttattttaccactccctatcagtgatagagaa

SEQ ID NO: 27 - DNA, 123 bp, synthetic construct
  misc_feature 1..123: note = Synthetic: native cumate inducible promoter
  source 1..123: mol_type = other DNA
taacaccgtgcgtgttgactattttacctctggcggtgataatggttgcaacaaacagacaatctggtctgtttgtattatgttaaccagcacactggcggccgcttactagaaataattttg

SEQ ID NO: 28 - DNA, 161 bp, synthetic construct
  misc_feature 1..161: note = Synthetic: native cumate inducible promoter
  source 1..161: mol_type = other DNA
taacaccgtgcgtgttgactattttacctctggcggtgataatggttgcaacaaacagacaatctggtctgtttgtattatgttaaccagcacactggcggccgcttactagaaataattttggccgcatcccacgctaactaatataaaggaggttttaa

SEQ ID NO: 29 - DNA, 79 bp, synthetic construct
  misc_feature 1..79: note = Synthetic: native salicylate inducible promoter
  source 1..79: mol_type = other DNA
tgtatttatcaatattgtttgctccgttatcgttattaacaagtcatcaataaagccatcacgagtaccatagaggatc

SEQ ID NO: 30 - DNA, 117 bp, synthetic construct
  misc_feature 1..117: note = Synthetic: native salicylate inducible promoter
  source 1..117: mol_type = other DNA
tgtatttatcaatattgtttgctccgttatcgttattaacaagtcatcaataaagccatcacgagtaccatagaggatcgccgcatcccacgctaactaatataaaggaggttttaa

SEQ ID NO: 31 - DNA, 42 bp, synthetic construct
  misc_feature 1..42: note = Synthetic: apFAB124 promoter
  source 1..42: mol_type = other DNA
tcgacatttatcccttgcggcgaatacttacagccatagcaa

SEQ ID NO: 32 - DNA, 49 bp, synthetic construct
  misc_feature 1..49: note = Synthetic: apfab338 promtoer
  source 1..49: mol_type = other DNA
ggcgcgccttgacaattaatcatccggctcctaggatgtgtggagggac

SEQ ID NO: 33 - DNA, 50 bp, synthetic construct
  misc_feature 1..50: note = Synthetic: apFAB66 promoter
  source 1..50: mol_type = other DNA
ggcgcgccttgacatcaggaaaatttttctgtataatagattcatctcaa

SEQ ID NO: 34 - DNA, 51 bp, synthetic construct
  misc_feature 1..51: note = Synthetic: apFAB54 promoter
  source 1..51: mol_type = other DNA
ggcgcgccttgacataaagtctaacctataggatacttacagccatacaag

SEQ ID NO: 35 - DNA, 66 bp, synthetic construct
  misc_feature 1..66: note = Synthetic: Lpp promoter from E. coli
  source 1..66: mol_type = other DNA
ataagtgccttcccatcaaaaaaatattctcaacataaaaaactttgtgtaatacttgtaacgcta

SEQ ID NO: 36 - DNA, 96 bp, synthetic construct
  misc_feature 1..96: note = Synthetic: Lpp promoter from E. coli
  source 1..96: mol_type = other DNA
ataagtgccttcccatcaaaaaaatattctcaacataaaaaactttgtgtaatacttgtaacgctagtgaattatataaaagtgggaggtgcccga

SEQ ID NO: 37 - DNA, 30 bp, synthetic construct
  misc_feature 1..30: note = Synthetic: Description of Artificial Sequence: Synthetic oligonucleotide BBa_I14018
  source 1..30: mol_type = other DNA
gtttatacataggcgagtactctgttatgg

SEQ ID NO: 38 - DNA, 30 bp, synthetic construct
  misc_feature 1..30: note = Synthetic: Description of Artificial Sequence: Synthetic oligonucleotide BBa_I14033
  source 1..30: mol_type = other DNA
agaggttccaactttcaccataatgaaaca

SEQ ID NO: 39 - DNA, 30 bp, synthetic construct
  misc_feature 1..30: note = Synthetic: Description of Artificial Sequence: Synthetic oligonucleotide BBa_I14034
  source 1..30: mol_type = other DNA
taaacaactaacggacaattctacctaaca

SEQ ID NO: 40 - DNA, 30 bp, synthetic construct
  misc_feature 1..30: note = Synthetic: Description of Artificial Sequence: Synthetic oligonucleotide BBa_I732021
  source 1..30: mol_type = other DNA
acatcaagccaaattaaacaggattaacac

SEQ ID NO: 41 - DNA, 30 bp, synthetic construct
  misc_feature 1..30: note = Synthetic: Description of Artificial Sequence: Synthetic oligonucleotide BBa_I742126
  source 1..30: mol_type = other DNA
gaggtaaaatagtcaacacgcacggtgtta

SEQ ID NO: 42 - DNA, 30 bp, synthetic construct
  misc_feature 1..30: note = Synthetic: Description of Artificial Sequence: Synthetic oligonucleotide BBa_J01006
  source 1..30: mol_type = other DNA
caggccggaataactccctataatgcgcca

SEQ ID NO: 43 - DNA, 30 bp, synthetic construct
  misc_feature 1..30: note = Synthetic: Description of Artificial Sequence: Synthetic oligonucleotide BBa_J23100
  source 1..30: mol_type = other DNA
ggctagctcagtcctaggtacagtgctagc

SEQ ID NO: 44 - DNA, 30 bp, synthetic construct
  misc_feature 1..30: note = Synthetic: Description of Artificial Sequence: Synthetic oligonucleotide BBa_J23101
  source 1..30: mol_type = other DNA
agctagctcagtcctaggtattatgctagc

SEQ ID NO: 45 - DNA, 30 bp, synthetic construct
  misc_feature 1..30: note = Synthetic: Description of Artificial Sequence: Synthetic oligonucleotide BBa_J23102
  source 1..30: mol_type = other DNA
agctagctcagtcctaggtactgtgctagc

SEQ ID NO: 46 - DNA, 30 bp, synthetic construct
  misc_feature 1..30: note = Synthetic: Description of Artificial Sequence: Synthetic oligonucleotide BBa_J23103
  source 1..30: mol_type = other DNA
agctagctcagtcctagggattatgctagc

SEQ ID NO: 47 - DNA, 30 bp, synthetic construct
  misc_feature 1..30: note = Synthetic: Description of Artificial Sequence: Synthetic oligonucleotide BBa_J23104
  source 1..30: mol_type = other DNA
agctagctcagtcctaggtattgtgctagc

SEQ ID NO: 48 - DNA, 30 bp, synthetic construct
  misc_feature 1..30: note = Synthetic: Description of Artificial Sequence: Synthetic oligonucleotide BBa_J23105
  source 1..30: mol_type = other DNA
ggctagctcagtcctaggtactatgctagc

SEQ ID NO: 49 - DNA, 30 bp, synthetic construct
  misc_feature 1..30: note = Synthetic: Description of Artificial Sequence: Synthetic oligonucleotide BBa_J23106
  source 1..30: mol_type = other DNA
ggctagctcagtcctaggtatagtgctagc

SEQ ID NO: 50 - DNA, 30 bp, synthetic construct
  misc_feature 1..30: note = Synthetic: Description of Artificial Sequence: Synthetic oligonucleotide BBa_J23107
  source 1..30: mol_type = other DNA
ggctagctcagccctaggtattatgctagc

SEQ ID NO: 51 - DNA, 30 bp, synthetic construct
  misc_feature 1..30: note = Synthetic: Description of Artificial Sequence: Synthetic oligonucleotide BBa_J23108
  source 1..30: mol_type = other DNA
agctagctcagtcctaggtataatgctagc

SEQ ID NO: 52 - DNA, 30 bp, synthetic construct
  misc_feature 1..30: note = Synthetic: Description of Artificial Sequence: Synthetic oligonucleotide BBa_J23109
  source 1..30: mol_type = other DNA
agctagctcagtcctagggactgtgctagc

SEQ ID NO: 53 - DNA, 30 bp, synthetic construct
  misc_feature 1..30: note = Synthetic: Description of Artificial Sequence: Synthetic oligonucleotide BBa_J23110
  source 1..30: mol_type = other DNA
ggctagctcagtcctaggtacaatgctagc

SEQ ID NO: 54 - DNA, 30 bp, synthetic construct
  misc_feature 1..30: note = Synthetic: Description of Artificial Sequence: Synthetic oligonucleotide BBa_J23113
  source 1..30: mol_type = other DNA
ggctagctcagtcctagggattatgctagc

SEQ ID NO: 55 - DNA, 30 bp, synthetic construct
  misc_feature 1..30: note = Synthetic: Description of Artificial Sequence: Synthetic oligonucleotide BBa_J23115
  source 1..30: mol_type = other DNA
agctagctcagcccttggtacaatgctagc

SEQ ID NO: 56 - DNA, 30 bp, synthetic construct
  misc_feature 1..30: note = Synthetic: Description of Artificial Sequence: Synthetic oligonucleotide BBa_J23116
  source 1..30: mol_type = other DNA
agctagctcagtcctagggactatgctagc

SEQ ID NO: 57 - DNA, 30 bp, synthetic construct
  misc_feature 1..30: note = Synthetic: Description of Artificial Sequence: Synthetic oligonucleotide BBa_J23117
  source 1..30: mol_type = other DNA
agctagctcagtcctagggattgtgctagc

SEQ ID NO: 58 - DNA, 30 bp, synthetic construct
  misc_feature 1..30: note = Synthetic: Description of Artificial Sequence: Synthetic oligonucleotide BBa_J23118
  source 1..30: mol_type = other DNA
ggctagctcagtcctaggtattgtgctagc

SEQ ID NO: 59 - DNA, 30 bp, synthetic construct
  misc_feature 1..30: note = Synthetic: Description of Artificial Sequence: Synthetic oligonucleotide BBa_J23150
  source 1..30: mol_type = other DNA
ggctagctcagtcctaggtattatgctagc

SEQ ID NO: 60 - DNA, 30 bp, synthetic construct
  misc_feature 1..30: note = Synthetic: Description of Artificial Sequence: Synthetic oligonucleotide BBa_J44002
  source 1..30: mol_type = other DNA
aaagtgtgacgccgtgcaaataatcaatgt

SEQ ID NO: 61 - DNA, 30 bp, synthetic construct
  misc_feature 1..30: note = Synthetic: Description of Artificial Sequence: Synthetic oligonucleotide BBa_J48104
  source 1..30: mol_type = other DNA
gacgaatacttaaaatcgtcatacttattt

SEQ ID NO: 62 - DNA, 30 bp, synthetic construct
  misc_feature 1..30: note = Synthetic: Description of Artificial Sequence: Synthetic oligonucleotide BBa_J54200
  source 1..30: mol_type = other DNA
aaacctttcgcggtatggcatgatagcgcc

SEQ ID NO: 63 - DNA, 30 bp, synthetic construct
  misc_feature 1..30: note = Synthetic: Description of Artificial Sequence: Synthetic oligonucleotide BBa_J56015
  source 1..30: mol_type = other DNA
tgatagcgcccggaagagagtcaattcagg

SEQ ID NO: 64 - DNA, 30 bp, synthetic construct
  misc_feature 1..30: note = Synthetic: Description of Artificial Sequence: Synthetic oligonucleotide BBa_J64951
  source 1..30: mol_type = other DNA
ttatttaccgtgacgaactaattgctcgtg

SEQ ID NO: 65 - DNA, 30 bp, synthetic construct
  misc_feature 1..30: note = Synthetic: Description of Artificial Sequence: Synthetic oligonucleotide BBa_K088007
  source 1..30: mol_type = other DNA
catacgccgttatacgttgtttacgctttg

SEQ ID NO: 66 - DNA, 30 bp, synthetic construct
  misc_feature 1..30: note = Synthetic: Description of Artificial Sequence: Synthetic oligonucleotide BBa_K119000
  source 1..30: mol_type = other DNA
ttatgcttccggctcgtatgttgtgtggac

SEQ ID NO: 67 - DNA, 30 bp, synthetic construct
  misc_feature 1..30: note = Synthetic: Description of Artificial Sequence: Synthetic oligonucleotide BBa_K119001
  source 1..30: mol_type = other DNA
ttatgcttccggctcgtatggtgtgtggac

SEQ ID NO: 68 - DNA, 30 bp, synthetic construct
  misc_feature 1..30: note = Synthetic: Description of Artificial Sequence: Synthetic oligonucleotide BBa_K137029
  source 1..30: mol_type = other DNA
atatatatatatatataatggaagcgtttt

SEQ ID NO: 69 - DNA, 30 bp, synthetic construct
  misc_feature 1..30: note = Synthetic: Description of Artificial Sequence: Synthetic oligonucleotide BBa_K137031
  source 1..30: mol_type = other DNA
ccccgaaagcttaagaatataattgtaagc

SEQ ID NO: 70 - DNA, 30 bp, synthetic construct
  misc_feature 1..30: note = Synthetic: Description of Artificial Sequence: Synthetic oligonucleotide BBa_K137085
  source 1..30: mol_type = other DNA
tgacaatatatatatatatataatgctagc

SEQ ID NO: 71 - DNA, 30 bp, synthetic construct
  misc_feature 1..30: note = Synthetic: Description of Artificial Sequence: Synthetic oligonucleotide BBa_K137086
  source 1..30: mol_type = other DNA
acaatatatatatatatatataatgctagc

SEQ ID NO: 72 - DNA, 30 bp, synthetic construct
  misc_feature 1..30: note = Synthetic: Description of Artificial Sequence: Synthetic oligonucleotide BBa_K137087
  source 1..30: mol_type = other DNA
aatatatatatatatatatataatgctagc

SEQ ID NO: 73 - DNA, 30 bp, synthetic construct
  misc_feature 1..30: note = Synthetic: Description of Artificial Sequence: Synthetic oligonucleotide BBa_K137088
  source 1..30: mol_type = other DNA
tatatatatatatatatatataatgctagc

SEQ ID NO: 74 - DNA, 30 bp, synthetic construct
  misc_feature 1..30: note = Synthetic: Description of Artificial Sequence: Synthetic oligonucleotide BBa_K137090
  source 1..30: mol_type = other DNA
aaaaaaaaaaaaaaaaaatataatgctagc

SEQ ID NO: 75 - DNA, 30 bp, synthetic construct
  misc_feature 1..30: note = Synthetic: Description of Artificial Sequence: Synthetic oligonucleotide BBa_K1585100
  source 1..30: mol_type = other DNA
ggaattgtgagcggataacaatttcacaca

SEQ ID NO: 76 - DNA, 30 bp, synthetic construct
  misc_feature 1..30: note = Synthetic: Description of Artificial Sequence: Synthetic oligonucleotide BBa_K1824896
  source 1..30: mol_type = other DNA
gattaaagaggagaaatactagagtactag

SEQ ID NO: 77 - DNA, 30 bp, synthetic construct
  misc_feature 1..30: note = Synthetic: Description of Artificial Sequence: Synthetic oligonucleotide BBa_K256002
  source 1..30: mol_type = other DNA
caccttcgggtgggcctttctgcgtttata

SEQ ID NO: 78 - DNA, 30 bp, synthetic construct
  misc_feature 1..30: note = Synthetic: Description of Artificial Sequence: Synthetic oligonucleotide BBa_K292001
  source 1..30: mol_type = other DNA
tgctagctactagagattaaagaggagaaa

SEQ ID NO: 79 - DNA, 30 bp, synthetic construct
  misc_feature 1..30: note = Synthetic: Description of Artificial Sequence: Synthetic oligonucleotide BBa_K418000
  source 1..30: mol_type = other DNA
ttgtgagcggataacaagatactgagcaca

SEQ ID NO: 80 - DNA, 30 bp, synthetic construct
  misc_feature 1..30: note = Synthetic: Description of Artificial Sequence: Synthetic oligonucleotide BBa_M13101
  source 1..30: mol_type = other DNA
cctgtttttatgttattctctctgtaaagg

SEQ ID NO: 81 - DNA, 30 bp, synthetic construct
  misc_feature 1..30: note = Synthetic: Description of Artificial Sequence: Synthetic oligonucleotide BBa_M13102
  source 1..30: mol_type = other DNA
aaatatttgcttatacaatcttcctgtttt

SEQ ID NO: 82 - DNA, 30 bp, synthetic construct
  misc_feature 1..30: note = Synthetic: Description of Artificial Sequence: Synthetic oligonucleotide BBa_M13103
  source 1..30: mol_type = other DNA
gctgataaaccgatacaattaaaggctcct

SEQ ID NO: 83 - DNA, 30 bp, synthetic construct
  misc_feature 1..30: note = Synthetic: Description of Artificial Sequence: Synthetic oligonucleotide BBa_M13104
  source 1..30: mol_type = other DNA
ctcttctcagcgtcttaatctaagctatcg

SEQ ID NO: 84 - DNA, 30 bp, synthetic construct
  misc_feature 1..30: note = Synthetic: Description of Artificial Sequence: Synthetic oligonucleotide BBa_M13105
  source 1..30: mol_type = other DNA
atgagccagttcttaaaatcgcataaggta

SEQ ID NO: 85 - DNA, 30 bp, synthetic construct
  misc_feature 1..30: note = Synthetic: Description of Artificial Sequence: Synthetic oligonucleotide BBa_M13106
  source 1..30: mol_type = other DNA
ctattgattgtgacaaaataaacttattcc

SEQ ID NO: 86 - DNA, 30 bp, synthetic construct
  misc_feature 1..30: note = Synthetic: Description of Artificial Sequence: Synthetic oligonucleotide BBa_M13108
  source 1..30: mol_type = other DNA
gtttcgcgcttggtataatcgctgggggtc

SEQ ID NO: 87 - DNA, 30 bp, synthetic construct
  misc_feature 1..30: note = Synthetic: Description of Artificial Sequence: Synthetic oligonucleotide BBa_M13110
  source 1..30: mol_type = other DNA
ctttgcttctgactataatagtcagggtaa

SEQ ID NO: 88 - DNA, 30 bp, synthetic construct
  misc_feature 1..30: note = Synthetic: Description of Artificial Sequence: Synthetic oligonucleotide BBa_M31519
  source 1..30: mol_type = other DNA
aaaccgatacaattaaaggctcctgctagc

SEQ ID NO: 89 - DNA, 30 bp, synthetic construct
  misc_feature 1..30: note = Synthetic: Description of Artificial Sequence: Synthetic oligonucleotide BBa_R1074
  source 1..30: mol_type = other DNA
caccacactgatagtgctagtgtagatcac

SEQ ID NO: 90 - DNA, 30 bp, synthetic construct
  misc_feature 1..30: note = Synthetic: Description of Artificial Sequence: Synthetic oligonucleotide BBa_R1075
  source 1..30: mol_type = other DNA
gccggaataactccctataatgcgccacca

SEQ ID NO: 91 - DNA, 30 bp, synthetic construct
  misc_feature 1..30: note = Synthetic: Description of Artificial Sequence: Synthetic oligonucleotide BBa_S03331
  source 1..30: mol_type = other DNA
ttgacaagcttttcctcagctccgtaaact

SEQ ID NO: 92 - DNA, 30 bp, synthetic construct
  misc_feature 1..30: note = Synthetic: Description of Artificial Sequence: Synthetic oligonucleotide BBa_J45992
  source 1..30: mol_type = other DNA
ggtttcaaaattgtgatctatatttaacaa

SEQ ID NO: 93 - DNA, 30 bp, synthetic construct
  misc_feature 1..30: note = Synthetic: Description of Artificial Sequence: Synthetic oligonucleotide BBa_J45504
  source 1..30: mol_type = other DNA
tctattccaataaagaaatcttcctgcgtg

SEQ ID NO: 94 - DNA, 30 bp, synthetic construct
  misc_feature 1..30: note = Synthetic: Description of Artificial Sequence: Synthetic oligonucleotide BBa_K1895002
  source 1..30: mol_type = other DNA
gaccgaatatatagtggaaacgtttagatg

SEQ ID NO: 95 - DNA, 30 bp, synthetic construct
  misc_feature 1..30: note = Synthetic: Description of Artificial Sequence: Synthetic oligonucleotide BBa_K1895003
  source 1..30: mol_type = other DNA
ccacatcctgtttttaaccttaaaatggca

SEQ ID NO: 96 - DNA, 30 bp, synthetic construct
  misc_feature 1..30: note = Synthetic: Description of Artificial Sequence: Synthetic oligonucleotide BBa_K143012
  source 1..30: mol_type = other DNA
aaaaatgggctcgtgttgtacaataaatgt

SEQ ID NO: 97 - DNA, 30 bp, synthetic construct
  misc_feature 1..30: note = Synthetic: Description of Artificial Sequence: Synthetic oligonucleotide BBa_K143013
  source 1..30: mol_type = other DNA
aaaaaaagcgcgcgattatgtaaaatataa

SEQ ID NO: 98 - DNA, 30 bp, synthetic construct
  misc_feature 1..30: note = Synthetic: Description of Artificial Sequence: Synthetic oligonucleotide BBa_K780003
  source 1..30: mol_type = other DNA
aattgcagtaggcatgacaaaatggactca

SEQ ID NO: 99 - DNA, 30 bp, synthetic construct
  misc_feature 1..30: note = Synthetic: Description of Artificial Sequence: Synthetic oligonucleotide BBa_K823000
  source 1..30: mol_type = other DNA
caagcttttcctttataatagaatgaatga

SEQ ID NO: 100 - DNA, 30 bp, synthetic construct
  misc_feature 1..30: note = Synthetic: Description of Artificial Sequence: Synthetic oligonucleotide BBa_K823002
  source 1..30: mol_type = other DNA
tctaagctagtgtattttgcgtttaatagt

SEQ ID NO: 101 - DNA, 30 bp, synthetic construct
  misc_feature 1..30: note = Synthetic: Description of Artificial Sequence: Synthetic oligonucleotide BBa_K823003
  source 1..30: mol_type = other DNA
aatgggctcgtgttgtacaataaatgtagt

SEQ ID NO: 102 - DNA, 30 bp, synthetic construct
  misc_feature 1..30: note = Synthetic: Description of Artificial Sequence: Synthetic oligonucleotide BBa_K143010
  source 1..30: mol_type = other DNA
atccttatcgttatgggtattgtttgtaat

SEQ ID NO: 103 - DNA, 30 bp, synthetic construct
  misc_feature 1..30: note = Synthetic: Description of Artificial Sequence: Synthetic oligonucleotide BBa_K143011
  source 1..30: mol_type = other DNA
taaaagaattgtgagcgggaatacaacaac

SEQ ID NO: 104 - DNA, 30 bp, synthetic construct
  misc_feature 1..30: note = Synthetic: Description of Artificial Sequence: Synthetic oligonucleotide BBa_K112706
  source 1..30: mol_type = other DNA
tacaaaataattcccctgcaaacattatca

SEQ ID NO: 105 - DNA, 30 bp, synthetic construct
  misc_feature 1..30: note = Synthetic: Description of Artificial Sequence: Synthetic oligonucleotide BBa_K112707
  source 1..30: mol_type = other DNA
tacaaaataattcccctgcaaacattatcg

SEQ ID NO: 106 - DNA, 30 bp, synthetic construct
  misc_feature 1..30: note = Synthetic: Description of Artificial Sequence: Synthetic oligonucleotide BBa_I712074
  source 1..30: mol_type = other DNA
agggaatacaagctacttgttctttttgca

SEQ ID NO: 107 - DNA, 23 bp, synthetic construct
  misc_feature 1..23: note = Synthetic: Description of Artificial Sequence: Synthetic oligonucleotide BBa_I719005
  source 1..23: mol_type = other DNA
taatacgactcactatagggaga

SEQ ID NO: 108 - DNA, 28 bp, synthetic construct
  misc_feature 1..28: note = Synthetic: Description of Artificial Sequence: Synthetic oligonucleotide BBa_J34814
  source 1..28: mol_type = other DNA
gaatttaatacgactcactatagggaga

SEQ ID NO: 109 - DNA, 19 bp, synthetic construct
  misc_feature 1..19: note = Synthetic: Description of Artificial Sequence: Synthetic oligonucleotide BBa_J64997
  source 1..19: mol_type = other DNA
taatacgactcactatagg

SEQ ID NO: 110 - DNA, 30 bp, synthetic construct
  misc_feature 1..30: note = Synthetic: Description of Artificial Sequence: Synthetic oligonucleotide BBa_K113010
  source 1..30: mol_type = other DNA
gagtcgtattaatacgactcactatagggg

SEQ ID NO: 111 - DNA, 30 bp, synthetic construct
  misc_feature 1..30: note = Synthetic: Description of Artificial Sequence: Synthetic oligonucleotide BBa_K113011
  source 1..30: mol_type = other DNA
agtgagtcgtactacgactcactatagggg

SEQ ID NO: 112 - DNA, 30 bp, synthetic construct
  misc_feature 1..30: note = Synthetic: Description of Artificial Sequence: Synthetic oligonucleotide BBa_K113012
  source 1..30: mol_type = other DNA
gagtcgtattaatacgactctctatagggg

SEQ ID NO: 113 - DNA, 18 bp, synthetic construct
  misc_feature 1..18: note = Synthetic: Description of Artificial Sequence: Synthetic oligonucleotide BBa_K1614000
  source 1..18: mol_type = other DNA
taatacgactcactatag

SEQ ID NO: 114 - DNA, 23 bp, synthetic construct
  misc_feature 1..23: note = Synthetic: Description of Artificial Sequence: Synthetic oligonucleotide BBa_R0180
  source 1..23: mol_type = other DNA
ttatacgactcactatagggaga

SEQ ID NO: 115 - DNA, 23 bp, synthetic construct
  misc_feature 1..23: note = Synthetic: Description of Artificial Sequence: Synthetic oligonucleotide BBa_R0181
  source 1..23: mol_type = other DNA
gaatacgactcactatagggaga

SEQ ID NO: 116 - DNA, 23 bp, synthetic construct
  misc_feature 1..23: note = Synthetic: Description of Artificial Sequence: Synthetic oligonucleotide BBa_R0182
  source 1..23: mol_type = other DNA
taatacgtctcactatagggaga

SEQ ID NO: 117 - DNA, 23 bp, synthetic construct
  misc_feature 1..23: note = Synthetic: Description of Artificial Sequence: Synthetic oligonucleotide BBa_R0183
  source 1..23: mol_type = other DNA
tcatacgactcactatagggaga

SEQ ID NO: 118 - DNA, 30 bp, synthetic construct
  misc_feature 1..30: note = Synthetic: Description of Artificial Sequence: Synthetic oligonucleotide BBa_Z0251
  source 1..30: mol_type = other DNA
taatacgactcactatagggagaccacaac

SEQ ID NO: 119 - DNA, 30 bp, synthetic construct
  misc_feature 1..30: note = Synthetic: Description of Artificial Sequence: Synthetic oligonucleotide BBa_Z0252
  source 1..30: mol_type = other DNA
taattgaactcactaaagggagaccacagc

SEQ ID NO: 120 - DNA, 30 bp, synthetic construct
  misc_feature 1..30: note = Synthetic: Description of Artificial Sequence: Synthetic oligonucleotide BBa_Z0253
  source 1..30: mol_type = other DNA
cgaagtaatacgactcactattagggaaga

SEQ ID NO: 121 - DNA, 19 bp, synthetic construct
  misc_feature 1..19: note = Synthetic: Description of Artificial Sequence: Synthetic oligonucleotide BBa_J64998
  source 1..19: mol_type = other DNA
atttaggtgacactataga

SEQ ID NO: 122 - DNA, 30 bp, synthetic construct
  misc_feature 1..30: note = Synthetic: Description of Artificial Sequence: Synthetic oligonucleotide BBa_I766555
  source 1..30: mol_type = other DNA
acaaacacaaatacacacactaaattaata

SEQ ID NO: 123 - DNA, 30 bp, synthetic construct
  misc_feature 1..30: note = Synthetic: Description of Artificial Sequence: Synthetic oligonucleotide BBa_I766556
  source 1..30: mol_type = other DNA
ccaagcatacaatcaactatctcatataca

SEQ ID NO: 124 - DNA, 30 bp, synthetic construct
  misc_feature 1..30: note = Synthetic: Description of Artificial Sequence: Synthetic oligonucleotide BBa_I766557
  source 1..30: mol_type = other DNA
gatacaggatacagcggaaacaacttttaa

SEQ ID NO: 125 - DNA, 30 bp, synthetic construct
  misc_feature 1..30: note = Synthetic: Description of Artificial Sequence: Synthetic oligonucleotide BBa_J63005
  source 1..30: mol_type = other DNA
tttcaagctataccaagcatacaatcaact

SEQ ID NO: 126 - DNA, 30 bp, synthetic construct
  misc_feature 1..30: note = Synthetic: Description of Artificial Sequence: Synthetic oligonucleotide BBa_K105027
  source 1..30: mol_type = other DNA
cctttgcagcataaattactatacttctat

SEQ ID NO: 127 - DNA, 30 bp, synthetic construct
  misc_feature 1..30: note = Synthetic: BBa_K122000, pPGK1
  source 1..30: mol_type = other DNA
ttatctactttttacaacaaatataaaaca

SEQ ID NO: 128 - DNA, 30 bp, synthetic construct
  misc_feature 1..30: note = Synthetic: BBa_K124002, Yeast GPD (TDH3) Promoter
  source 1..30: mol_type = other DNA
gtttcgaataaacacacataaacaaacaaa

SEQ ID NO: 129 - DNA, 30 bp, synthetic construct
  misc_feature 1..30: note = Synthetic: BBa_M31201, Yeast CLB1 promoter region, G2/M cell cycle specific
  source 1..30: mol_type = other DNA
accatcaaaggaagctttaatcttctcata

SEQ ID NO: 130 - DNA, 30 bp, synthetic construct
  misc_feature 1..30: note = Synthetic: BBa_I712004, CMV promoter
  source 1..30: mol_type = other DNA
agaacccactgcttactggcttatcgaaat

SEQ ID NO: 131 - DNA, 30 bp, synthetic construct
  misc_feature 1..30: note = Synthetic: BBa_K076017, Ubc Promoter
  source 1..30: mol_type = other DNA
ggccgtttttggcttttttgttagacgaag

SEQ ID NO: 132 - DNA, 52 bp, synthetic construct
  misc_feature 1..52: note = Synthetic: PapFAB46
  source 1..52: mol_type = other DNA
aaaaagagtattgacttcgcatctttttgtacctataatagattcattgcta

SEQ ID NO: 133 - DNA, 59 bp, synthetic construct
  misc_feature 1..59: note = Synthetic: PJ23101+UP element
  source 1..59: mol_type = other DNA
ggaaaatttttttaaaaaaaaaactttacagctagctcagtcctaggtattatgctagc

SEQ ID NO: 134 - DNA, 59 bp, synthetic construct
  misc_feature 1..59: note = Synthetic: PJ23107+UP element
  source 1..59: mol_type = other DNA
ggaaaatttttttaaaaaaaaaactttacggctagctcagccctaggtattatgctagc

SEQ ID NO: 135 - DNA, 64 bp, synthetic construct
  misc_feature 1..64: note = Synthetic: PSYN23119
  source 1..64: mol_type = other DNA
ggaaaatttttttaaaaaaaaaacttgacagctagctcagtccttggtataatgctagcacgaa

SEQ ID NO: 136 - protein, 21 aa, synthetic construct
  REGION 1..21: note = Synthetic: PhoA secretion tag
MKQSTIALALLPLLFTPVTKA

SEQ ID NO: 137 - protein, 20 aa, synthetic construct
  REGION 1..20: note = Synthetic: PhoA secretion tag
KQSTIALALLPLLFTPVTKA

SEQ ID NO: 138 - protein, 22 aa, synthetic construct
  REGION 1..22: note = Synthetic: OmpF secretion tag
MMKRNILAVIVPALLVAGTANA

SEQ ID NO: 139 - protein, 15 aa, synthetic construct
  REGION 1..15: note = Synthetic: cvaC secretion tag
MRTLTLNELDSVSGG

SEQ ID NO: 140 - protein, 43 aa, synthetic construct
  REGION 1..43: note = Synthetic: TorA secretion tag
MNNNDLFQASRRRFLAQLGGLTVAGMLGTSLLTPRRATAAQAA

SEQ ID NO: 141 - protein, 33 aa, synthetic construct
  REGION 1..33: note = Synthetic: fdnG secretion tag
MDVSRRQFFKICAGGMAGTTVAALGFAPKQALA

SEQ ID NO: 142 - protein, 45 aa, synthetic construct
  REGION 1..45: note = Synthetic: dmsA secretion tag
MKTKIPDAVLAAEVSRRGLVKTTAIGGLAMASSALTLPFSRIAHA

SEQ ID NO: 143 - protein, 21 aa, synthetic construct
  REGION 1..21: note = Synthetic: PelB secretion tag
KYLLPTAAAGLLLLAAQPAMA

SEQ ID NO: 144 - protein, 52 aa, synthetic construct
  REGION 1..52: note = Synthetic: HlyA secretion signal
LNPLINEISKIISAAGNFDVKEERAAASLLQLSGNASDFSYGRNSITLTASA

SEQ ID NO: 145 - DNA, 159 bp, synthetic construct
  misc_feature 1..159: note = Synthetic: HlyA secretion signal
  source 1..159: mol_type = other DNA
cttaatccattaattaatgaaatcagcaaaatcatttcagctgcaggtaattttgatgttaaagaggaaagagctgcagcttctttattgcagttgtccggtaatgccagtgatttttcatatggacggaactcaataactttgacagcatcagcataa

SEQ ID NO: 146 - protein, 52 aa, synthetic construct
  REGION 1..52: note = Synthetic: ECOLIN_05715 Secretion signal
MKRHLNTSYRLVWNHITGAFVVASELARARGKRAGVAVALSLAAATSLPALA

SEQ ID NO: 147 - protein, 40 aa, synthetic construct
  REGION 1..40: note = Synthetic: ECOLIN_16495 Secretion signal
MFWRDMTLSVWRKKTTGLKTKKRLLALVLAAALCSSPVWA

SEQ ID NO: 148 - protein, 33 aa, synthetic construct
  REGION 1..33: note = Synthetic: ECOLIN_19410 Secretion signal
MGYKMNISSLRKAFIFMGAVAALSLVNAQSALA

SEQ ID NO: 149 - protein, 53 aa, synthetic construct
  REGION 1..53: note = Synthetic: ECOLIN_19880 Secretion signal
MNKIFKVIWNPATGSYTVASETAKSRGKKSGRSKLLISALVAGGLLSSFGASA

SEQ ID NO: 150 - DNA, 159 bp, synthetic construct
  misc_feature 1..159: note = Synthetic: adhesion Secretion signal
  source 1..159: mol_type = other DNA
atgaacaagattttcaaggttatctggaaccccgcgacaggcagctacactgtggcctccgaaaccgctaagtcgcgcggcaagaagtcaggacgttcaaagctgcttatttctgctttagtagccggaggattactttctagctttggagcctcggca

SEQ ID NO: 151 - protein, 19 aa, synthetic construct
  REGION 1..19: note = Synthetic: DsbA secretion tag
MKKIWLALAGLVLAFSASA

SEQ ID NO: 152 - DNA, 57 bp, synthetic construct
  misc_feature 1..57: note = Synthetic: DsbA secretion tag
  source 1..57: mol_type = other DNA
atgaaaaaaatctggctggcattagcaggcttagtcctggcctttagtgcctcggca

SEQ ID NO: 153 - protein, 22 aa, synthetic construct
  REGION 1..22: note = Synthetic: GltI secretion tag
MQLRKPATAILALALSAGLAQA

SEQ ID NO: 154 - DNA, 66 bp, synthetic construct
  misc_feature 1..66: note = Synthetic: GltI secretion tag
  source 1..66: mol_type = other DNA
atgcagttgcgcaagcctgccacggcaattttagcattagctctttctgcgggcttggcccaggca

SEQ ID NO: 155 - DNA, 120 bp, synthetic construct
  misc_feature 1..120: note = Synthetic: GspD secretion tag
  source 1..120: mol_type = other DNA
atgttctggcgcgacatgacactgagcgtttggcgtaaaaaaacaacgggactgaaaaccaaaaagcgtttgttagcccttgtcctggcggcagccctgtgctcgtccccggtatgggca

SEQ ID NO: 156 - DNA, 99 bp, synthetic construct
  misc_feature 1..99: note = Synthetic: HdeB secretion tag
  source 1..99: mol_type = other DNA
atgggatacaagatgaacatttcatcacttcgcaaagcttttatcttcatgggtgccgttgctgctctgtcactggtcaatgctcaatcggccctggca

SEQ ID NO: 157 - protein, 26 aa, synthetic construct
  REGION 1..26: note = Synthetic: MalE secretion tag
MKIKTGARILALSALTTMMFSASALA

SEQ ID NO: 158 - DNA, 78 bp, synthetic construct
  misc_feature 1..78: note = Synthetic: MalE secretion tag
  source 1..78: mol_type = other DNA
atgaagattaaaacaggcgctcgtattttggccctgtctgcgcttacgactatgatgtttagtgcctcggccctggca

SEQ ID NO: 159 - protein, 26 aa, synthetic construct
  REGION 1..26: note = Synthetic: OppA secretion tag
MTNITKRSLVAAGVLAALMAGNVALA

SEQ ID NO: 160 - DNA, 78 bp, synthetic construct
  misc_feature 1..78: note = Synthetic: OppA secretion tag
  source 1..78: mol_type = other DNA
atgacgaatatcactaaacgtagccttgtggccgcgggggtcttggctgcattgatggctggcaatgttgctctggca

SEQ ID NO: 161 - protein, 22 aa, synthetic construct
  REGION 1..22: note = Synthetic: PelB secretion tag
MKYLLPTAAAGLLLLAAQPAMA

SEQ ID NO: 162 - DNA, 66 bp, synthetic construct
  misc_feature 1..66: note = Synthetic: PelB secretion tag
  source 1..66: mol_type = other DNA
atgaaatatctgttgcccacggctgccgcgggtctgctgctgctggcagcgcaaccggctatggca

SEQ ID NO: 163 - DNA, 63 bp, synthetic construct
  misc_feature 1..63: note = Synthetic: PhoA secretion tag
  source 1..63: mol_type = other DNA
atgaagcaaagtacgatcgcccttgcgctgttgccccttcttttcacgcccgtcaccaaggca

SEQ ID NO: 164 - protein, 24 aa, synthetic construct
  REGION 1..24: note = Synthetic: PpiA secretion tag
MFKSTLAAMAAVFALSALSPAAMA

SEQ ID NO: 165 - DNA, 72 bp, synthetic construct
  misc_feature 1..72: note = Synthetic: PpiA secretion tag
  source 1..72: mol_type = other DNA
atgtttaagtctacacttgcagccatggccgcagtcttcgcactgtcagccttgagtcctgctgcaatggca

SEQ ID NO: 166 - protein, 21 aa, synthetic construct
  REGION 1..21: note = Synthetic: tolb Secretion Tag
MKQALRVAFGFLILWASVLHA

SEQ ID NO: 167 - protein, 18 aa, synthetic construct
  REGION 1..18: note = Synthetic: tort Secretion Tag
MRVLLFLLLSLFMLPAFS

SEQ ID NO: 168 - protein, 21 aa, synthetic construct
  REGION 1..21: note = Synthetic: ompA Secretion Tag
MKKTAIAIAVALAGFATVAQA

SEQ ID NO: 169 - DNA, 57 bp, synthetic construct
  misc_feature 1..57: note = Synthetic: dsba Secretion Tag
  source 1..57: mol_type = other DNA
atgaagaaaatttggctggcacttgccggtttggtattagcgttttccgcctcggca

SEQ ID NO: 170 - DNA, 63 bp, synthetic construct
  misc_feature 1..63: note = Synthetic: tolb secretion tag
  source 1..63: mol_type = other DNA
atgaagcaggcgcttcgcgttgcgtttgggttccttatcctgtgggcatccgtacttcacgcc

SEQ ID NO: 171 - DNA, 54 bp, synthetic construct
  misc_feature 1..54: note = Synthetic: tort secretion tag
  source 1..54: mol_type = other DNA
atgcgcgtactgttattccttctgttgtctctttttatgttgcccgctttcagt

SEQ ID NO: 172 - DNA, 63 bp, synthetic construct
  misc_feature 1..63: note = Synthetic: ompA secretion tag
  source 1..63: mol_type = other DNA
atgaagaagactgccatcgctattgccgtcgcccttgcgggtttcgcaaccgtggcgcaagca

SEQ ID NO: 173 - DNA, 66 bp, synthetic construct
  misc_feature 1..66: note = Synthetic: pelB secretion tag
  source 1..66: mol_type = other DNA
atgaaatatctgcttccaacggctgctgctggcctgttgcttcttgccgcccagcctgcgatggct

SEQ ID NO: 174 - DNA, 99 bp, synthetic construct
  misc_feature 1..99: note = Synthetic: 19410 secretion tag
  source 1..99: mol_type = other DNA
atggggtacaaaatgaacattagctcgcttcgcaaagcattcatttttatgggggctgttgcagctttaagccttgtcaatgcccagtcagcgcttgcc

SEQ ID NO: 175 - DNA, 57 bp, synthetic construct
  misc_feature 1..57: note = Synthetic: dsba secretion tag
  source 1..57: mol_type = other DNA
atgaaaaagatttggctggctcttgccggtttagtcctggcattcagcgcaagcgcg

SEQ ID NO: 176 - DNA, 63 bp, synthetic construct
  misc_feature 1..63: note = Synthetic: phoA secretion tag
  source 1..63: mol_type = other DNA
atgaaacagagcacaatcgctttagccttgctgcctcttcttttcacgcctgtcacgaaggca

SEQ ID NO: 177 - DNA, 63 bp, synthetic construct
  misc_feature 1..63: note = Synthetic: tolB secretion tag
  source 1..63: mol_type = other DNA
atgaagcaggctctgcgcgtggcatttgggttcctgatcttatgggcgtccgtcttacacgca

SEQ ID NO: 178 - DNA, 78 bp, synthetic construct
  misc_feature 1..78: note = Synthetic: malE secretion tag
  source 1..78: mol_type = other DNA
atgaaaatcaagacgggggcacgcattcttgccttgagcgccttgacaacgatgatgttcagtgcaagtgcattagcg

SEQ ID NO: 179 - DNA, 69 bp, synthetic construct
  misc_feature 1..69: note = Synthetic: mglB secretion tag
  source 1..69: mol_type = other DNA
atgaataagaaagtcttgacattgtcggcggtcatggcctcgatgttgttcggggcagcagcccacgca

SEQ ID NO: 180 - DNA, 66 bp, synthetic construct
  misc_feature 1..66: note = Synthetic: ompF secretion tag
  source 1..66: mol_type = other DNA
atgatgaagcgtaacatcttagccgttattgtccccgcattgcttgtggccgggacggctaacgca

SEQ ID NO: 181 - DNA, 63 bp, synthetic construct; identical to SEQ ID NO: 172
  misc_feature 1..63: note = Synthetic: ompA secretion tag
  source 1..63: mol_type = other DNA

SEQ ID NO: 182 - DNA, 54 bp, synthetic construct
  misc_feature 1..54: note = Synthetic: tort secretion tag
  source 1..54: mol_type = other DNA
atgcgcgtgttattgttcttgctgctgagcttgtttatgttaccagctttcagt

SEQ ID NO: 183 - DNA, 75 bp, synthetic construct
  misc_feature 1..75: note = Synthetic: lamB secretion tag
  source 1..75: mol_type = other DNA
atgatgattaccttacgcaagttgccccttgcggtagccgttgctgctggtgtgatgtccgcgcaagcaatggct

SEQ ID NO: 184 - DNA, 66 bp, synthetic construct
  misc_feature 1..66: note = Synthetic: pelB secretion tag
  source 1..66: mol_type = other DNA
atgaaatatcttcttccaacggctgctgctggtttattgcttcttgccgcccagcctgcgatggct

SEQ ID NO: 185 - protein, 23 aa, synthetic construct
  REGION 1..23: note = Synthetic: secretion tag
MNKKVLTLSAVMASMLFGAAAHA

SEQ ID NO: 186 - protein, 25 aa, synthetic construct
  REGION 1..25: note = Synthetic: secretion tag
MMITLRKLPLAVAVAAGVMSAQAMA

SEQ ID NO: 187 - DNA, 65 bp, synthetic construct
  misc_feature 1..65: note = Synthetic: secretion tag
  source 1..65: mol_type = other DNA
tgcagttgcgcaagcctgccacggcaattttagcattagctctttctgcgggcttggcccaggca

SEQ ID NO: 188 - DNA, 121 bp, synthetic construct
  misc_feature 1..121: note = Synthetic: secretion tag
  source 1..121: mol_type = other DNA
aatgttctggcgcgacatgacactgagcgtttggcgtaaaaaaacaacgggactgaaaaccaaaaagcgtttgttagcccttgtcctggcggcagccctgtgctcgtccccggtatgggca

SEQ ID NO: 189 - DNA, 57 bp, synthetic construct; identical to SEQ ID NO: 169
  misc_feature 1..57: note = Synthetic: dsba secretion tag
  source 1..57: mol_type = other DNA

SEQ ID NO: 190 - protein, 607 aa, synthetic construct
  REGION 1..607: note = Synthetic: Invasin display tag
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAV

SEQ ID NO: 191 - protein, 148 aa, synthetic construct
  REGION 1..148: note = Synthetic: LppOmpA display tag
KATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPG

SEQ ID NO: 192 - protein, 658 aa, synthetic construct
  REGION 1..658: note = Synthetic: IntiminN display tag
ITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYYRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 193 - DNA, 456 bp, synthetic construct
  misc_feature 1..456: note = Synthetic: pelB anchor
  source 1..456: mol_type = other DNA
tgcagtagcaataaaaatgcttcgaatgacggatcggaaggaatgttaggtgctggtactggcatggacgccaacggtggcaacggaaatatgtctagcgaagaacaagctcgtttacaaatgcagcaattacagcaaaacaacatagtctacttcgacttagacaagtacgacatccgtagcgattttgcacaaatgttggatgcccatgccaacttccttcgttctaatccaagctacaaagtcactgttgagggccacgcggatgagagaggcactcctgaatataacatatccctgggggagcgcagagcaaacgccgtaaaaatgtacctgcaaggcaagggcgtctcggccgatcaaatctcgatcgtgagctatggcaaagagaaaccggctgtattgggacatgacgaagctgcttacagcaagaatcggcgggccgtcttggtctac

SEQ ID NO: 194 - DNA, 63 bp, synthetic construct
  misc_feature 1..63: note = Synthetic: BAN anchor
  source 1..63: mol_type = other DNA
gcttttgaccccaatctggttgggcctacgttacctccaattccgcctttcactctgcctacg

SEQ ID NO: 195 - DNA, 442 bp, synthetic construct
  misc_feature 1..442: note = Synthetic: LppOmpA anchor
  source 1..442: mol_type = other DNA
atgactagttaaagcaacaaaacttgtgttaggcgcggttatacttggctccaccctgcttgcaggttgctcgtctaacgcgaagatcgaccagggtatcaatccttacgtcgggtttgaaatgggatacgattggttgggacgtatgccttataagggaagtgttgaaaacggcgcttataaggcgcagggagtacagttaacggccaagcttgggtaccccataacagacgatttagatatttatacccgtttaggaggaatggtttggagagccgacacgaagtctaatgtatatggtaagaaccacgacacgggagtatcccccgtctttgcagggggagtggaatatgctatcacaccagagatcgctacccgtttggaatatcaatggacgaataatataggcgacgcccatacgataggaacgcggcccgacaac

SEQ ID NO: 196 - DNA, 915 bp, synthetic construct
  misc_feature 1..915: note = Synthetic: NGIgAsig anchor
  source 1..915: mol_type = other DNA
ccgcgtgctgcacaaccgcgtacacaagctgcagcccaggccgacgcagtatccacgaacacaaattcagcgttgagtgatgctatggcttcaactcaatcaattcttttggacaccggcgcatatttaactcgccatatcgcccaaaagtctcgtgcggatgcagaaaaaaatagcgtgtggatgtcgaatactgggtatgggcgcgactacgcctctgcgcagtaccgccgtttcagcagcaaacgcacccagactcaaatcgggatcgatcgtagtcttagtgagaatatgcagattggcggagttctgacttattccgactctcaacatacgttcgaccaggcagggggcaagaatacgtttgtacaggccaatctttacggaaagtactacttgaatgatgcctggtatgtcgctggggacattggcgcagggagcttgcgttcccgccttcaaacgcaacaaaaggcgaatttcaatcgcaccagcattcagactggccttacacttgggaatacacttaagattaatcaatttgagattgtgcccagtgctgggattcgttatagtcgtttatccagcgcagactataagttaggcgacgactctgtgaaagttagctcaatggccgtgaagacgttgacggctggtttggatttcgcctatcgctttaaagttggaaatctgaccgtgaagccgctgctttcggccgcttattttgcaaactacggcaagggcggcgttaatgtcggtggtaagagtttcgcctataaagctgacaatcagcagcaatattcggccggcgtcgcgttgttgtatcgcaacgtgactttaaatgtaaacggctccatcacaaagggcaagcagttggagaagcaaaagtcaggccaaatcaagattcaaatccgtttc

SEQ ID NO: 197 - DNA, 603 bp, synthetic construct
  misc_feature 1..603: note = Synthetic: OsmY anchor
  source 1..603: mol_type = other DNA
atgacaatgactcgtttgaaaatttccaaaaccctgctggccgtaatgcttacgtccgcagtcgccactggttccgcatacgcagagaataatgcgcagacaactaacgagtcagcagggcaaaaagtagactccagtatgaataaggtgggcaattttatggacgactcagctatcaccgctaaggtgaaagcggcgctggtggaccacgacaacatcaagtccacggacatctcagttaaaacggaccaaaaggtagtaaccttaagcgggttcgtagaaagccaggcgcaagcagaggaagcggtaaaagtcgctaaaggcgtagaaggggtcacttcggtgtcagacaagttgcatgtgcgtgacgcaaaggaaggatcagtaaagggttatgccggagatacggcaacgacctctgagatcaaagcgaaattactggcagacgacattgttccctcgcgtcatgttaaagtcgaaacgactgatggcgtagtccagctttcgggtacagttgactcccaagcacaaagtgatcgcgcggaatctattgcaaaggcggtggacggagtgaaatccgtcaaaaacgatcttaaaacgaaa

SEQ ID NO: 198 - DNA, 1820 bp, synthetic construct
  misc_feature 1..1820: note = Synthetic: invasinN anchor
  source 1..1820: mol_type = other DNA
atggttttccaacccatcagcgaatttttgctgattcgtaacgctgggatgtccatgtattttaacaagatcatttcttttaacatcatttcacgtatcgttatttgcatttttcttatctgtggtatgttcatggccggtgcatctgaaaagtatgatgcaaacgcaccccaacaggtgcagccatactcggtttcatcatcagcgttcgagaatctgcaccccaataacgagatggagtcgagtatcaacccttttagtgcttcggacaccgagcgtaatgcagctatcatcgatcgtgctaacaaggaacaagaaacggaagcagtcaacaaaatgatctccactggcgctcgtttagctgccagcggtcgcgcgtccgatgtggcgcacagtatggtaggggatgcggtcaaccaggagattaaacaatggctgaatcgcttcggcactgctcaagtgaatttaaattttgacaagaacttctcgttaaaggagtcttcgcttgactggttggccccatggtacgattcggcgtcattccttttcttttctcagttgggcatccgtaacaaggacagtcgtaatacacttaaccttggtgttggcattcgcacattagaaaatggttggttgtatggcctgaacaccttttacgacaatgacttaacgggacacaatcaccgtatcgggctgggcgccgaggcgtggactgactacttgcagttagccgcgaatgggtacttccgtcttaatggttggcactcttcccgtgacttcagcgactacaaagaacgccctgctaccgggggagatttgcgtgcgaatgcgtacctgcccgctcttccgcaacttggcgggaagttaatgtatgagcagtatactggggaacgcgtggctctgttcggaaaggacaacctgcagcgcaacccatacgctgtcactgcgggtatcaactatacgccagttccgttgctgacggtcggcgtggatcaacgtatggggaagtcgagtaaacatgaaacgcaatggaatttacaaatgaactatcgcttaggggagagtttccaaagtcagcttagcccttcggcggtcgcagggactcgtttgcttgctgagtcccgctacaacctggttgatcgcaataacaatatcgtactggaataccagaaacaacaagtggttaagctgacgttgagccctgcgaccatcagtggattgcccggacaagtttaccaggtaaatgcccaggtccagggggcctctgcggttcgcgaaattgtctggtcagacgcagaattaatcgctgcaggaggcaccttaacgccactttccactacacaattcaatttagtccttcccccatacaaacgtaccgcccaggtatcgcgcgtaactgatgacttaactgctaatttttattcactgtcggcgttagcagttgaccatcaaggcaaccgtagtaattccttcacattatctgtaacggtgcagcagccgcaactgacgcttaccgcagcggtcattggtgatggggccccagctaatgggaaaaccgcaatcactgtcgagttcacagttgcagattttgaaggcaagccgctggcgggtcaggaggttgtgattacgactaataacggtgctcttcctaataagattactgaaaagactgacgctaacggcgttgcccgcattgcccttacgaacacaaccgatggggtcacggtagttaccgcagaggtcgaggggcaacgccaatccgttgacacgcacttcgttaagggtactatcgcggccgataaaagcacgctggccgcggt

SEQ ID NO: 199 - DNA, 696 bp, synthetic construct
  misc_feature 1..696: note = Synthetic: yiaT anchor
  source 1..696: mol_type = other DNA
atgttaatcaatagaaacattgtcgccctttttgccctgccattcatggcgagtgcgaccgcttccgagttaagcataggggccggcgctgcgtataatgagagtccttaccgtggctataatgaaaacacaaaggcaatcccccttatctcgtatgaaggcgacaccttctatgtacgtcagactacattaggtttcatcttaagccagtccgagaaaaacgagttatcgctgacagcttcatggatgccattggaattcgacccgacagataatgatgattatgcgatgcagcagctggacaaacgggattcgacagctatggccggcgtggcttggtatcaccacgagcggtggggaactgtcaaagcttccgccgccgctgacgtgttagacaacagcaatggctgggtgggggaattaagcgtgttccataagatgcagataggtagattatcccttacaccggctttgggagtcctgtattatgatgagaacttttccgactattattatgggatttctgagtctgaaagtcgtcggagcggtcttgcgtcgtactcggcgcaagacgcgtgggtgccttatgtatctttgacagcgaagtatccaataggcgagcacgtggtattgatggcaagtgctggttattccgagttgcccgaagaaattactgacagtcctatgatagaccgt

SEQ ID NO: 200 - DNA, 1977 bp, synthetic construct
  misc_feature 1..1977: note = Synthetic: intiminN anchor
  source 1..1977: mol_type = other DNA
atgattacgcatggctgttatacccgtacgcgtcataaacacaagttgaagaaaactctgatcatgttatccgctggacttggactttttttttacgtgaatcagaactctttcgctaatggggaaaattattttaaactgggatcagacagcaaattacttacgcatgactcataccagaatcgtctgttttatacgctgaaaactggtgaaaccgttgcagatttaagtaaaagtcaggacattaacctgtcaactatttggtcacttaataaacacttatattcgagcgaatcggaaatgatgaaagctgcaccggggcaacaaatcatcttgcccctgaagaaattgccctttgaatactccgctttgcccttgctgggctcggctcctctggtagccgccggaggcgttgccggtcacactaataagctgacaaaaatgtcacccgacgtgacgaagagcaacatgacggatgataaggctttaaattacgcagctcagcaagcggcctcgttgggaagtcagttacagagtcgttcgttaaatggtgattatgctaaggataccgcattgggtattgccggcaaccaagcgtcgagccaacttcaggcatggttgcaacattacggcactgctgaagtaaatctgcaatcaggtaataattttgacggtagttccctggatttccttttacctttttacgattcagaaaagatgttggctttcggacaggtgggggcgcgttacatcgattcacgttttaccgctaacttgggggccggtcaacgcttcttcttacctgccaatatgttgggctataatgtatttatcgaccaggacttcagtggtgacaatacacgtctgggaattggtggagagtattggcgcgattactttaagtcatctgtaaatggctattttcgcatgagcggttggcatgaaagttacaacaagaaagactacgatgagcgccccgcgaacgggtttgacatccgttttaatggttatttgccatcttatcccgccttgggagctaaattaatctacgagcaatactatggagataacgtagctttgtttaatagcgacaagttacagtctaatccaggagcggctacagtgggagttaattataccccaatcccactggtcacaatgggaatcgattatcgccacgggactggtaatgaaaacgatttattatactccatgcagtttcgttatcagttcgataagagttggtcgcagcagattgagcctcaatatgttaacgaattacgtaccttgtccggcagtcgctacgatctggtacaacgcaataacaatatcatccttgagtataagaaacaggacattctgtctttgaacattccacatgatattaatggtaccgagcactcaacacaaaaaattcagctgattgtgaaatcaaagtatggactggaccgtatcgtgtgggatgatagcgctctgcgcagtcagggtggacagatccagcactcgggtagccagtctgcccaagactaccaggctatcctgccagcgtatgtccaagggggaagtaacatctacaaagttacagctcgcgcctatgaccgcaacggtaattctagtaataatgtgcagttgacaattacggtgctgtccaatgggcaggtcgtcgatcaggtaggtgtgacggattttacagccgataaaacctctgcgaaggcagataacgcggataccatcacatacactgccactgtaaaaaaaaacggtgtcgcgcaggcaaacgttcctgttagcttcaacatcgtgtcgggtacagccacccttggggccaactcggcaaagactgacgcgaatggcaaggctacagtcacgttgaaatcctcgacaccaggacaggtcgttgtgtctgccaagacagcagagatgacctccgcccttaatgcatctgctgttatcttcttcgatcaaacgaaggcatct

SEQ ID NO: 201 - protein, 152 aa, synthetic construct
  REGION 1..152: note = Synthetic: pelB anchor
CSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYSKNRRAVLVY

SEQ ID NO: 202 - protein, 24 aa, synthetic construct
  REGION 1..24: note = Synthetic: BAN anchor
MTSAFDPNLVGPTLPPIPPFTLPT

SEQ ID NO: 203 - protein, 147 aa, synthetic construct
  REGION 1..147: note = Synthetic: LppOmpA anchor
MTSKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDN

SEQ ID NO: 204 - protein, 305 aa, synthetic construct
  REGION 1..305: note = Synthetic: NGIgAsig anchor
PRAAQPRTQAAAQADAVSTNTNSALSDAMASTQSILLDTGAYLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGVALLYRNVTLNVNGSITKGKQLEKQKSGQIKIQIRF

SEQ ID NO: 205 - protein, 201 aa, synthetic construct
  REGION 1..201: note = Synthetic: OsmY anchor
MTMTRLKISKTLLAVMLTSAVATGSAYAENNAQTTNESAGQKVDSSMNKVGNFMDDSAITAKVKAALVDHDNIKSTDISVKTDQKVVTLSGFVESQAQAEEAVKVAKGVEGVTSVSDKLHVRDAKEGSVKGYAGDTATTSEIKAKLLADDIVPSRHVKVETTDGVVQLSGTVDSQAQSDRAESIAKAVDGVKSVKNDLKTK

SEQ ID NO: 206 - protein, 232 aa, synthetic construct
  REGION 1..232: note = Synthetic: yiaT anchor
MLINRNIVALFALPFMASATASELSIGAGAAYNESPYRGYNENTKAIPLISYEGDTFYVRQTTLGFILSQSEKNELSLTASWMPLEFDPTDNDDYAMQQLDKRDSTAMAGVAWYHHERWGTVKASAAADVLDNSNGWVGELSVFHKMQIGRLSLTPALGVLYYDENFSDYYYGISESESRRSGLASYSAQDAWVPYVSLTAKYPIGEHVVLMASAGYSELPEEITDSPMIDR

SEQ ID NO: 207 - protein, 659 aa, synthetic construct
  REGION 1..659: note = Synthetic: intiminN anchor
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 208 - protein, 656 aa, synthetic construct
  REGION 1..656: note = Synthetic: intiminN anchor
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDGA

SEQ ID NO: 209 - protein, 5 aa, synthetic construct
  REGION 1..5: note = Synthetic: linker
GGGGS

SEQ ID NO: 210 - protein, 10 aa, synthetic construct
  REGION 1..10: note = Synthetic: linker
GGGGSGGGGS

SEQ ID NO: 211 - protein, 15 aa, synthetic construct
  REGION 1..15: note = Synthetic: linker
GGGGSGGGGSGGGGS

SEQ ID NO: 212 - protein, 20 aa, synthetic construct
  REGION 1..20: note = Synthetic: linker
GGGGSGGGGSGGGGSGGGGS

SEQ ID NO: 213 - protein, 5 aa, synthetic construct
  REGION 1..5: note = Synthetic: linker
EAAAK

SEQ ID NO: 214 - protein, 10 aa, synthetic construct
  REGION 1..10: note = Synthetic: linker
EAAAKEAAAK

SEQ ID NO: 215 - protein, 15 aa, synthetic construct
  REGION 1..15: note = Synthetic: linker
EAAAKEAAAKEAAAK

SEQ ID NO: 216 - protein, 8 aa, synthetic construct
  REGION 1..8: note = Synthetic: linker
GGGGGGGG

SEQ ID NO: 217 - protein, 6 aa, synthetic construct
  REGION 1..6: note = Synthetic: linker
GGGGGG

SEQ ID NO: 218 - protein, 46 aa, synthetic construct
  REGION 1..46: note = Synthetic: linker
AEAAAKEAAAKEAAAKEAAAKALEAEAAAKEAAAKEAAAKEAAAKA

SEQ ID NO: 219 - protein, 17 aa, synthetic construct
  REGION 1..17: note = Synthetic: linker
VSQTSKLTRAETVFPDV

SEQ ID NO: 220 - protein, 5 aa, synthetic construct
  REGION 1..5: note = Synthetic: linker
PAPAP

SEQ ID NO: 221 - protein, 12 aa, synthetic construct
  REGION 1..12: note = Synthetic: linker
AEAAAKEAAAKA

SEQ ID NO: 222 - DNA, 45 bp, synthetic construct
  misc_feature 1..45: note = Synthetic: scFvLinker
  source 1..45: mol_type = other DNA
ggtggaggcggctctggcggtggtgggagtggaggcggtggcagt

SEQ ID NO: 223 - protein, 34 aa, synthetic construct
  REGION 1..34: note = Synthetic: linker
APAPAPAPAPAPAPAPAPAPAPAPAPAPAPAPAP

SEQ ID NO: 224 - protein, 6 aa, synthetic construct
  REGION 1..6: note = Synthetic: linker
PLGLWA

SEQ ID NO: 225 - protein, 6 aa, synthetic construct
  REGION 1..6: note = Synthetic: linker
RVLAEA

SEQ ID NO: 226 - protein, 10 aa, synthetic construct
  REGION 1..10: note = Synthetic: linker
EDVVCCSMSY

SEQ ID NO: 227 - protein, 9 aa, synthetic construct
  REGION 1..9: note = Synthetic: linker
GGIEGRGSC

SEQ ID NO: 228 - protein, 10 aa, synthetic construct
  REGION 1..10: note = Synthetic: linker
TRHRQPRGWE

SEQ ID NO: 229 - protein, 10 aa, synthetic construct
  REGION 1..10: note = Synthetic: linker
AGNRVRRSVG

SEQ ID NO: 230 - protein, 9 aa, synthetic construct
  REGION 1..9: note = Synthetic: linker
RRRRRRRRR

SEQ ID NO: 231 - protein, 4 aa, synthetic construct
  REGION 1..4: note = Synthetic: linker
GFLG

SEQ ID NO: 232 - protein, 28 aa, synthetic construct
  REGION 1..28: note = Synthetic: linker
GGGSGGGGGGSGGGGGGSGGGGGGSGGG

SEQ ID NO: 233 - protein, 20 aa, synthetic construct
  REGION 1..20: note = Synthetic: linker
GGSGGGGSGGGGSGGGGSGG

SEQ ID NO: 234 - protein, 12 aa, synthetic construct
  REGION 1..12: note = Synthetic: linker
GSGGSGGSGGSG

SEQ ID NO: 235 - protein, 24 aa, synthetic construct
  REGION 1..24: note = Synthetic: linker
GGGSGGGGGSGGGGGSGGGGGSGG

SEQ ID NO: 236 - protein, 18 aa, synthetic construct
  REGION 1..18: note = Synthetic: linker
KESGSVSSEQLAQFRSLD

SEQ ID NO: 237 - protein, 14 aa, synthetic construct
  REGION 1..14: note = Synthetic: linker
EGKSSGSGSESKST

SEQ ID NO: 238 - protein, 12 aa, synthetic construct
  REGION 1..12: note = Synthetic: linker
GSAGSAAGSGEF

SEQ ID NO: 239 - DNA, 24 bp, synthetic construct
  misc_feature 1..24: note = Synthetic: UP element
  source 1..24: mol_type = other DNA
ggaaaatttttttaaaaaaaaaac

SEQ ID NO: 240 - protein, 173 aa, synthetic construct
  REGION 1..173: note = Synthetic: PAL
MQLNKVLKGLMIALPVMAIAACSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYAKNRRAVLVY

SEQ ID NO: 241 - DNA, 28 bp, synthetic construct
  misc_feature 1..28: note = Synthetic: RBS
  misc_feature 13..15: note = n is a, c, g, or t
  misc_feature 18..20: note = n is a, c, g, or t
  source 1..28: mol_type = other DNA
tctagagaaagannngannnactagatg

SEQ ID NO: 242 - DNA, 28 bp, synthetic construct
  misc_feature 1..28: note = Synthetic: RBS
  source 1..28: mol_type = other DNA
tctagagaaagaggggacaaactagatg

SEQ ID NO: 243 - DNA, 28 bp, synthetic construct
  misc_feature 1..28: note = Synthetic: RBS
  source 1..28: mol_type = other DNA
tctagagaaagacaggacccactagatg

SEQ ID NO: 244 - DNA, 28 bp, synthetic construct
  misc_feature 1..28: note = Synthetic: RBS
  source 1..28: mol_type = other DNA
tctagagaaagatccgatgtactagatg

SEQ ID NO: 245 - DNA, 28 bp, synthetic construct
  misc_feature 1..28: note = Synthetic: RBS
  source 1..28: mol_type = other DNA
tctagagaaagattagacaaactagatg

SEQ ID NO: 246 - DNA, 28 bp, synthetic construct
  misc_feature 1..28: note = Synthetic: RBS
  source 1..28: mol_type = other DNA
tctagagaaagaagggacagactagatg

SEQ ID NO: 247 - DNA, 28 bp, synthetic construct
  misc_feature 1..28: note = Synthetic: RBS
  source 1..28: mol_type = other DNA
tctagagaaagacatgacgtactagatg

SEQ ID NO: 248 - DNA, 28 bp, synthetic construct
  misc_feature 1..28: note = Synthetic: RBS
  source 1..28: mol_type = other DNA
tctagagaaagataggagacactagatg

SEQ ID NO: 249 - DNA, 28 bp, synthetic construct
  misc_feature 1..28: note = Synthetic: RBS
  source 1..28: mol_type = other DNA
tctagagaaagaagagactcactagatg

SEQ ID NO: 250 - DNA, 28 bp, synthetic construct
  misc_feature 1..28: note = Synthetic: RBS
  source 1..28: mol_type = other DNA
tctagagaaagacgagatatactagatg

SEQ ID NO: 251 - DNA, 28 bp, synthetic construct
  misc_feature 1..28: note = Synthetic: RBS
  source 1..28: mol_type = other DNA
tctagagaaagactggagacactagatg

SEQ ID NO: 252 - DNA, 28 bp, synthetic construct
  misc_feature 1..28: note = Synthetic: RBS
  source 1..28: mol_type = other DNA
tctagagaaagaggcgaattactagatg

SEQ ID NO: 253 - DNA, 28 bp, synthetic construct
  misc_feature 1..28: note = Synthetic: RBS
  source 1..28: mol_type = other DNA
tctagagaaagaggcgatacactagatg

SEQ ID NO: 254 - DNA, 28 bp, synthetic construct
  misc_feature 1..28: note = Synthetic: RBS
  source 1..28: mol_type = other DNA
tctagagaaagaggtgacatactagatg

SEQ ID NO: 255 - DNA, 28 bp, synthetic construct
  misc_feature 1..28: note = Synthetic: RBS
  source 1..28: mol_type = other DNA
tctagagaaagagtggaaaaactagatg

SEQ ID NO: 256 - DNA, 28 bp, synthetic construct
  misc_feature 1..28: note = Synthetic: RBS
  source 1..28: mol_type = other DNA
tctagagaaagatgagaagaactagatg

SEQ ID NO: 257 - DNA, 28 bp, synthetic construct
  misc_feature 1..28: note = Synthetic: RBS
  source 1..28: mol_type = other DNA
tctagagaaagaagggatacactagatg

SEQ ID NO: 258 - DNA, 28 bp, synthetic construct
  misc_feature 1..28: note = Synthetic: RBS
  source 1..28: mol_type = other DNA
tctagagaaagacatgaggcactagatg

SEQ ID NO: 259 - DNA, 28 bp, synthetic construct
  misc_feature 1..28: note = Synthetic: RBS
  source 1..28: mol_type = other DNA
tctagagaaagacatgagttactagatg

SEQ ID NO: 260 - DNA, 28 bp, synthetic construct
  misc_feature 1..28: note = Synthetic: RBS
  source 1..28: mol_type = other DNA
tctagagaaagagacgaatcactagatg

SEQ ID NO: 261 - DNA, 28 bp, synthetic construct
  misc_feature 1..28: note = Synthetic: RBS
  source 1..28: mol_type = other DNA
tctagagaaagatttgatatactagatg

SEQ ID NO: 262 - DNA, 28 bp, synthetic construct
  misc_feature 1..28: note = Synthetic: RBS
  source 1..28: mol_type = other DNA
tctagagaaagacgcgagaaactagatg

SEQ ID NO: 263 - DNA, 28 bp, synthetic construct
  misc_feature 1..28: note = Synthetic: RBS
  source 1..28: mol_type = other DNA
tctagagaaagagacgagtcactagatg

SEQ ID NO: 264 - DNA, 28 bp, synthetic construct
  misc_feature 1..28: note = Synthetic: RBS
  source 1..28: mol_type = other DNA
tctagagaaagagaggagccactagatg

SEQ ID NO: 265 - DNA, 28 bp, synthetic construct
  misc_feature 1..28: note = Synthetic: RBS
  source 1..28: mol_type = other DNA
tctagagaaagagatgactaactagatg

SEQ ID NO: 266 - DNA, 28 bp, synthetic construct
  misc_feature 1..28: note = Synthetic: RBS
  source 1..28: mol_type = other DNA
tctagagaaagagccgacatactagatg

SEQ ID NO: 267 - DNA, 28 bp, synthetic construct
  misc_feature 1..28: note = Synthetic: RBS
  source 1..28: mol_type = other DNA
tctagagaaagagccgagttactagatg

SEQ ID NO: 268 - DNA, 28 bp, synthetic construct
  misc_feature 1..28: note = Synthetic: RBS
  source 1..28: mol_type = other DNA
tctagagaaagaggtgactcactagatg

SEQ ID NO: 269 - DNA, 28 bp, synthetic construct
  misc_feature 1..28: note = Synthetic: RBS
  source 1..28: mol_type = other DNA
tctagagaaagagtggaactactagatg

SEQ ID NO: 270 - DNA, 28 bp, synthetic construct
  misc_feature 1..28: note = Synthetic: RBS
  source 1..28: mol_type = other DNA
tctagagaaagataggactcactagatg

SEQ ID NO: 271 - DNA, 28 bp, synthetic construct
  misc_feature 1..28: note = Synthetic: RBS
  source 1..28: mol_type = other DNA
tctagagaaagattggacgtactagatg

SEQ ID NO: 272 - DNA, 28 bp, synthetic construct
  misc_feature 1..28: note = Synthetic: RBS
  source 1..28: mol_type = other DNA
tctagagaaagaaacgacatactagatg

SEQ ID NO: 273 - DNA, 28 bp, synthetic construct
  misc_feature 1..28: note = Synthetic: RBS
  source 1..28: mol_type = other DNA
tctagagaaagaaccgaattactagatg

SEQ ID NO: 274 - DNA, 28 bp, synthetic construct
  misc_feature 1..28: note = Synthetic: RBS
  source 1..28: mol_type = other DNA
tctagagaaagacaggattaactagatg

SEQ ID NO: 275 - DNA, 28 bp, synthetic construct
  misc_feature 1..28: note = Synthetic: RBS
  source 1..28: mol_type = other DNA
tctagagaaagacccgagacactagatg

SEQ ID NO: 276 - DNA, 28 bp, synthetic construct
  misc_feature 1..28: note = Synthetic: RBS
  source 1..28: mol_type = other DNA
tctagagaaagaccggaaatactagatg

SEQ ID NO: 277 - DNA, 28 bp, synthetic construct
  misc_feature 1..28: note = Synthetic: RBS
  source 1..28: mol_type = other DNA
tctagagaaagaccggagacactagatg

SEQ ID NO: 278 - DNA, 28 bp, synthetic construct
  misc_feature 1..28: note = Synthetic: RBS
  source 1..28: mol_type = other DNA
tctagagaaagagctgagcaactagatg

SEQ ID NO: 279 - DNA, 28 bp, synthetic construct
  misc_feature 1..28: note = Synthetic: RBS
  source 1..28: mol_type = other DNA
tctagagaaagagtagatcaactagatg

SEQ ID NO: 280 - DNA, 28 bp, synthetic construct
  misc_feature 1..28: note = Synthetic: RBS
  source 1..28: mol_type = other DNA
tctagagaaagatatgaataactagatg

SEQ ID NO: 281 - DNA, 28 bp, synthetic construct
  misc_feature 1..28: note = Synthetic: RBS
  source 1..28: mol_type = other DNA
tctagagaaagattagagtcactagatg

SEQ ID NO: 282 - DNA, 31 bp, synthetic construct
  misc_feature 1..31: note = Synthetic: RBS
  source 1..31: mol_type = other DNA
tctagagttcacacaggaaacctactagatg

SEQ ID NO: 283 - DNA, 31 bp, synthetic construct
  misc_feature 1..31: note = Synthetic: RBS
  source 1..31: mol_type = other DNA
tctagagattaaagaggagaaatactagatg

SEQ ID NO: 284 - DNA, 30 bp, synthetic construct
  misc_feature 1..30: note = Synthetic: RBS
  source 1..30: mol_type = other DNA
tctagagtcacacaggaaacctactagatg

SEQ ID NO: 285 - DNA, 29 bp, synthetic construct
  misc_feature 1..29: note = Synthetic: RBS
  source 1..29: mol_type = other DNA
tctagagtcacacaggaaagtactagatg

SEQ ID NO: 286 - DNA, 27 bp, synthetic construct
  misc_feature 1..27: note = Synthetic: RBS
  source 1..27: mol_type = other DNA
tctagagtcacacaggactactagatg

SEQ ID NO: 287 - DNA, 28 bp, synthetic construct
  misc_feature 1..28: note = Synthetic: RBS
  source 1..28: mol_type = other DNA
tctagagaaagaggagaaatactagatg

SEQ ID NO: 288 - DNA, 30 bp, synthetic construct
  misc_feature 1..30: note = Synthetic: RBS
  source 1..30: mol_type = other DNA
tctagagattaaagaggagaatactagatg

SEQ ID NO: 289 - DNA, 28 bp, synthetic construct
  misc_feature 1..28: note = Synthetic: RBS
  source 1..28: mol_type = other DNA
tctagagaaagaggggaaatactagatg

SEQ ID NO: 290 - DNA, 25 bp, synthetic construct
  misc_feature 1..25: note = Synthetic: RBS
  source 1..25: mol_type = other DNA
ttatataaaagtgggaggtgcccga

SEQ ID NO: 291 - DNA, 72 bp, synthetic construct
  misc_feature 1..72: note = Synthetic: RBS
  source 1..72: mol_type = other DNA
gaccagaggtaaggaggtaacaaccatgcgagtgttgaagaaacatcttaatcatgctggggagggtttcta

SEQ ID NO: 292 - DNA, 9687 bp, synthetic construct
  misc_feature 1..9687: note = Synthetic: Phage 3 DNA E. coli
  source 1..9687: mol_type = other DNA
aatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtg

SEQ ID NO: 293 - DNA, 424 bp, synthetic construct
  misc_feature 1..424: note = Synthetic: clbA knockout sequence
  source 1..424: mol_type = other DNA
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 294 - DNA, 735 bp, synthetic construct
  misc_feature 1..735: note = Synthetic: clbA
  source 1..735: mol_type = other DNA
atgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattga

SEQ ID NO: 295 - DNA, 9621 bp, synthetic construct
  misc_feature 1..9621: note = Synthetic: clbB
  source 1..9621: mol_type = other DNA
atggataatacctctggagattttccatgtaataagatggacacgcgtaagcagttaccgctaacaccaagtcaacaggggtttttattccattccttaaaggataagaaaaggagtaactaccatgagcattttacatgcattttttctcagcatgtagatagcgcccacttcaagtgggcgctggaaacgttatttcgaaagcatgagtgttttcgcactgattataactgggagattgatgagcgcccttgtcaggtggtgaagaccgatgtgttgccggatatatatgtgttagactgtgagcaagaggaaatacgttttctactagctaatgatgacattatcattcctgtcccgcaggatgacggtattgatgctataattcctcaactgctacaggctgatttaaaatacccattttccttgaaaacgatcccagtccgggcctaccttattcagtcaacgaaagaaagtgcttttatactatcataccatcatattgtgatggatggctggagcttatcccttttcattaaacagttgctccaactctatggagcggctgtggtcagtggtgtgagggatgatagcgccattatcccctcatctctgaaaccccttgtagacacactgtcggcccgacgtcacacctttcagcacgactattgggctgcatatcttcgggagggaacaccaacttgtatcgtgccgctgtcacaatatcacacagatactgaagccgagaacaattcttacgttaatcaaacaaatcatgtggagatcaatttgtctccggatgtgtgtcagaaaatacagacgctatgcagcgattatcgtatcacccccgcagtaatcttctatgtggcctggggcatcctgctacaacgttggtgctatgctgacgatgtgttattcggcgcgacaatatcagggcgaaatataccaattgatggtatagaagaaacactagggctatttattaacacgttgccgctgcgtctgcgtgatgatggggcgacactgttgcaacacttacaacgtatgcaccaaacactaatagctcactacagcaatgaacatgatgccttagccagcatacaacggttggtacataaagaaggtcatgctggggatctttttaataccttagtggtgttggagaattatcccgttgatatgacattattgtcatgcgcgtcgcccgtggcaattcgccatctcagtgtacatgaacaaacgcactatccgctgaccttgaccatcactcaacagaagggattccgtttcagtattgcatatgcccttaactacctgaccaacaacatggcgcaagcgttgctgatgcacctgagttatctgcttgaacaactggtggacaatccgcagcgccccattgctgcgttggtaaacttgtccccatgccagcaggcgcaggtacttcaaccctatctggaacgcatggcatgccgggattgggatagtcaatccaacgtcatcgaacaatttcatcaagttgctgcgacttcgccagcacaggtcgcagtggttgatgaattgtgcgcgttgacctattcggagttggcagcacaagctgagcagctcgcggcttatttggtacagcagggtgttatggttggcgatactgtaggcataattagcgaacgccgggtaaacaccgtggttgccatcatcgccatcatgttgatcggggctgcttatgtgcccatcagccccgactacccagtgggtaggatgcaggaaattattgatgacagcggcttggcgctgttgctggtacatggcaaaccgctagatgcattaaacgttgcgcagagtgacctctgtgcatttcccgtcgcgccctcggtggtatttccggttatcacaccagattctcgcgcttatgtgatttattcatcggggtcgacgggtaagccaaaaggtatcgcggtggcgcaccgcggtttattgcgcctgatacaaggcgacagcccgctgaaggtggagagcggtgagacaacgctgctgacctgcccatttgagtttgatgtttcggtgtttgagatgtggtctaccttgctcaaccacggcaaactagtattactcagcaaacaggcattacttgatatcaatcacattcgccgcacgatcgctgatgaacaggtggcgcgcgcctggtttacctcatccttgtttaactcctatgtggcagaaggtgccgatttcttcggtatgttacaacacattacggtgggcggcgaagcagtatctgcgtggcatgtcaacgacgtgatgcaaaaatacccgcatctggtggtgacaaacggttatgggccgacggaaaacactatttttaccaccgcatatcgtttcaacgggttgcaacccgcccgagtcccgataggatacgcggtaccgggcacctcgctctacattaccgatctccatgggcatttgttgcctatcggtgccaccggtgaactagtggcgggtggagtgggggtcgccatcggctatcagaacaacccggcgctaagtgcgacggtttttgtccctgatccttttattcccggcggtatgatgtacaaaactggcgattatgcccggttgctggatgatggctgtgttgactgttttgggcgtaaagacggtcaaattaagatcaatggacaacgaattgaaaccggagagatcgagcagcgcctgctggagtgctccggcattatcgaggcggtagtggttccttaccgcgtacgtgaaacgctgcatattgcggcagtggtctgcgtcaatgatagctatgatgaggtggaagttcgagggcaattggctgacagattgccgccattcgctattccggaatccttggtggtggtgacggagattgcaaaaagccatagtggcaaggccgacttggcgcagttgcggtatctcctgcccgcaactcagtgcaacgccgtgtccaccacgatatcagaggtgcatagtgacatggaacatgcgctgcatgctatctggcaacgcgtacttgatagacaagacattgatagcaatgcctcctttttcgccctcggtggcacctcattggataccatcagggttaaaggggatattaagcggcaacttggcttggagattgatattaccgatctctttaagtacccaacgctcacggcgttagcacattttctcgatactgccgtatcgccggaggatgcaattccaacgcgtgctgttgtctacagcgacatgccggtggcgattgtcggtatggcgggacgtttccccggtgcggcgaatattgcagcgttgtggacgctggttgtaggaggggaatcgggattaacactgttcagtgatgaagagttgcgcgcgcatggtgtgacacctgacacgcttaaacaagcgaattatataaaaaccaaagggattgttgatgatcacgaatggtttgatgcggatttctttggttatactcccaacgaggcggaatgtatggatccgcaaattcgcttattgcatcagtgctgctggcaaactctggaacacgctgggtgcgatcctgccacctttactggtgcgattggcatttatgccggactgctgacatccccccactggcttaatgcggtaatgcaagacactaccgactctaccgccctgtacaaggccagtatcctgaatatccattccgtcacagcattgattgcccatgcgcttaacctcaccggccctgccgtgacgcttgacaccgcctgctccacttcggcagtggctatccaccaggcctgcatcgcactacgtaaccgggattgcgatgcggcattggcgggcggcgtttctatcgagatgcctgcgtaccggggctatgaatatcatgaaggcatgatcaatgcgcgagatggtgtctgtcgtcctttcgatagccaagcctctggcacagtcaccggtgatggcttaggaatgctgctgctaaaacgtctggacgacgcgctggcggatcgggactgcatctatggtgtgatcaaaggttcggcggtcaataatgatggcaataacaaaatcggctataccgcgccgagtgtgatcgggcaatcaacggtaatccgcaccagtttgcgccgtgccggttttgatagcgacagtatcggattggtggaagcgcacggcaccggtacggtgctgggtgatcctattgagttacgtgcgcttaatgaggtgtttggcccaacacctgttccgttttgtgtggtttcagcgctgaaaagtaatattggccacctcaactccgctgcgggagtggcgggcgtcatcaaaacaacgctcgccctgcatcatcaagtgttgccaccaacagcgcactttcgccaactcaatcctgctatagatctatcgcgttctgcattgtacgttaaccagcaagtccaaccgtggccgtcgacgcgtcctcgtcgtgcactggtgagctccttcggcatcggcggcaccaatgccagcatcgcactagaagcacatcaacatgaggacgacccttcagcgacgggggtacgcgacagctatctgttgttgttctccgctaaaacacccgctgcgttagagttgcgcgtggcctccacactggaatatgtcaagcatggagtaggggtgcgcctgccggatgtggcttatacattgcaaactggacgcacagcctttgaccatcggcgggcttatttggtgagtcgtgggtcgaaaatcgatctctcctgtgccacgatattgcaagcggaaatcttcaatggtcagcgcacgacagcggagatctgcttcatgtttcctggtcaaggtagccaatatcacggcatggccagcgcgctctatgctcatcaacccatgtttcgccagcacatggatcgctgctttgctgcattccaacgctattcgacggtcgatctcaaggcgttgttgtttgacgatgaggatacgcgggatattgatcaaacgcagttcacacaaccggcgttgttctgtgtcgaatacagcctagcgcgcaccttgattgatctggggattacgccggacagtatgatcgggcacagtctgggcgagtatgttgcggcctgtattgctggcgtatttactcttgaggatgcgctgcacgtcattgaggcgcgcggacgtttgatgcagtccatgcgtcccggtagcatgatggcggtctaccttagtcgcgaacagttgaccccatggctagctgcagaacggggtattgaactggcagctaataatagcgcgcatttttgcgtagtcgcgggcgagcaggcggccatttcccgtttgagcacacgcttagtcgagggcgggatacagcacaggcgcctgaaaacctctcacgcattccactcggccatgatgacgccgatgctgcacgattttgcacagttgctggggcaaatcccgatgcacgcgccgcacaagcgctttatatctaatgtaagcggtacatggattactgaggagcaagctacctcgccggattactgggtgcagcaagtgcgcaacgcggtgctgtttagcgaaggtgcggcgcaactgttggtacaacccacgctgtttatcgaatgtgggccgggtaatacgctctctacctttattcaaggacataaccaatacagcgatcagccgaccctgttgacgctacgcaaagccaacgcggcgatcgatgatgagcacatgttgcatcgtacgctggcggcgctgtgggtcaggggggagaatattgattggagacgctttaaccagacggcactcggcaagcacattccattgccggattaccccttcgaacagacttattactatcgctatggtgctgcactttccggttatcgccagtatccaaatcctctgcgccgtccgcaagatgagtggctccagcgtgtgctgtggcggatgcacgacacatccttgcgggaggcgttctatgcgccgggcgaattgatcatcattattagtgctgacggcgacaagttacagcagacgctgatgagtagtggtgtcgacagcatcacaatgccgctgccgatatcgtcagaggacgacgtgtgggataatgaccgtattctgacgcatttccacgacatctgcgcacttttagcacacaaaacctaccgacagttacattgcttgtatgctcccggtgcggaggcaggatcatcgttgacacagtcgctctcgggactttatcgtgttgctcgctggtgtatgcacagcaccacgccgctggcgtccttaacagtattgacccatggtgcgtttcgcgtacaggaagaggataatcccgaaccgacgctggcagcgttgtcgggcgcagtaaacgtgtttgcccaagagctgcacccgaccgaagtacgattgatcgatatcgatgcgcagagcagtgatgagaatctgaacttgctaacccagcgtctggccccgaaacaagaaacggtaatggcgctcaggcaggggatgctttacctgcgacgctttatcccgacacggctgttggcgcacttaccccctcaaacagggtgtataccgggcaacgtactgtggattatcggcggtgaaaaggggattggccgcatgatcggcgaagcgcttgctcagcgtgagggagtccgtgtggtactgagcagccgcacaggttatcaccatgaagcggtgcagcaggacgcattagacgttatccactgcgacgtgacgcaggcggaggcggttagagcttgtctggcaactctgctcgaacgctatggacggttggatggcgtgatttttgctgccgacgctaccaccacattgacactgcatcaattgagcgaatctgcgctacgcgacacgctaacggtgaaagaacggggtactgctaatgtgctgcatgcgttagcgcaacggaatctgctggatgagcgtctgctactgctgttctgtaactcgttggctgccgtgaatgcggagattggccagacaggctatgctaccgccagcgcctatttggatgcactggcacagcaactgcgtacccgctacaaggtgaatgcgctcagtatcgggttggatgcattgcgtgagcagggcatgttgttggatgctataaacggcagtgaatacgatgtcttgcgtggactgcgcccattgatgacgggaacgttgctacaggcttataaacaacaaggggctgacaccagctactacgcgcgattatcccccgagtccgattggctgctggatgagcaccggatatccggtatcgccacgctgccgggaaccggctatctggcgctggcgtatgaagctctgcgccattactttgtgcaagaccaaatctgcattgatgaattggtctttttggcaccgttgactgtgatggacaactgcagtgttgacgtttttgttgacatttcacctaacgggcaaggagttagtgtcgaggtgaaatcaatgacggagcgctttagtggcacgttaacaacccatgccagaggcagggcgacgcgtctgatggtagacgataatgttgtgtgcgatctcacggggctgatgcgcgagatgcacactatcactcctccaacaaaggaattatcgagcacgcacttccactatgggccgcgctggcacagcgtacaacaactgtatggcaataccgcccagactcaggttttcgcaacgctggccctgcccaccgttgccgctaatgatacgatcgcactgcaccctgcgctgttggacatcgccagcagtgttgtcgaacaactgcctggttttcatactgattcggtacctttcctttatcaggatttacgcctgtaccgcccgttgccgaacaccttacatgtggcgctgactgtcaatcggcacgatgaggagggtgacagctacgctttcacgctctacgacatggcaggcgagatggttgcccgctgtgcggcaatggtgaagcgcaaggtacagctccacatacaagatgtcgatgacgacacgcgactgcgcgtgcccagtgccgataactaccaactgcggctggccgctgagggggagggggcaggaaagctagcgttgtgccctacgccgcgcttagcgctgggggattcacaagtagagattgaggtactggccaccggactgaactttaaggatgtgctgttcaccacgggattgctccggcagcagccgggtgaggctccgctgcaattgggactggagtgcgccggacgcattactcgcgtgggtaaaaatgtcactgaatttgccccgggagaggatgttatggcggtgctaaacggtggttttgtccagtacgcacgggtagaaagcgattgcgtagtgagaaaaccagcccattgtcgcatcgaacaggcggctgcgctgcctatcgcatacctcaccgcctattacgcactggtggtgcgcgctaatttgcaacccggagaacgagtattgatccacagtgcggcggggggcgttggcttggctgcgctacatattgccaaacgctgcggagcacagattttcgccacagcaggtagcgagcagaaacgcgattacttgctttcgctaggcgtacatgccgtagctgattcacacgacgaacagttcgctgccactctgctgaccgcatcggacggacaggggatggatgtgatccttaactccctcacaggccgtctgcttgacgctagcctcgcgctgctggcaccgctgggccgttttcttgagctgggcagcaaggacattgtggaagacaaagcgctaccgatgcgtttcttcgcccaaggcggcacctttattccgattaattttcacgcggcgcatggtgcgtttagtcgctacctgcaacagattgtcgcttggatagatgataacacgctaccgctccttccatgcaaatccgtaccattgcccgaggttgcacgcgccttcgccaccctgaccacgccgcagcatattggcaaggtggtggtaacgcatcgcactgcggcaggcatggaccggctgaacgccatgatagcagaaaggcgcctcggcggctatgcgctcagcatgagcaatgccgaggtgatgcgccaattgtggccgatactgaacactcgcagtccgtgggcgcaactgctgctctcacctcgggcgatcgatagattagcgcggggcaaccgggtggatcgcggtgtaccgtctgccgctaacgatacgattactcagcagacagtgaaaaagcggccccgtccggaaattggcgtgccttacagccccgcgacacgtgaagtggaacgcgtgctctgccaaatcctggaagagtatctgggtctggatagagtaggcattgacgacaactatgccgaattaggggcaacctcactcgatatggtgcaactgagtgggcaaatggcgcgtcactatccgcaagtgagcgtggtgtcgctgtacaaccacgccaccgtgcgccagctggcgacgttttgccagcccccggagggcgagtcaaatgcgccatcaccacagcctgcagtacagacgaatacgcgcgcgaatcagatagcaaaacgtgctttgcaaattgcgaaaaatactgcgcgcagtcacacgtctttgcattaa

SEQ ID NO: 296 - DNA, 2601 bp, synthetic construct
  misc_feature 1..2601: note = Synthetic: clbC
  source 1..2601: mol_type = other DNA
atggaatacgcaagcgaaatgaacggcatggaaatcgccattattggtatggcggtccgtttcccgcagtcccggacgttacacgaattctggcataacatcgttcagggtaaagagtgcgtcaccttctttagcgaggaggagttgctggccgaaggcgtcgaacagagtactctggacaacccggcctatgtacgggccaagccctatatcgagggcatatgcgactttgatgctgcatttttcggttacagtcacaaagaggcgcagactctggatccgaaatcccgcgtattacatgaagttgcctaccatgcgctggaagatgcaggctatgcccaacgtaccagcgatctgatcaccggggtattcgtgggggcgtcagaagatgtggattggctacggcgttcgctgtcacagattggcggcgatgcgctgaatcgttttgagtctggcatctatggtcataaggatctgctcgcacatctcattgcctacagtctcaatctcaacggtccggtgtatagtctctacaccagttgttcgacttctctgagtgcaacgcatatcgcttgccgcagcttgttgtttggcgaatgtgatctggcgttggcgggtggaattactatcgatttaccgcagaagtcaggctacttctgtcaacagggcatgatccactccaccgatggccactgccgtccttttgacagtcaggcttctggcaccctgtttggcgatggcgcgggtgtggttgtgcttcggcgtttggaagatgctctggcagcgggcgatcgcatctatgcggtgatccggggtagcgcggtcaacaatgacggtaaacaaaaaatcggttttgtcgcgcctggtcatgagggacagaaggcggtcatttgtgcggcctgtcatctggcagaagtaagcccagaaagcatcggctacgttgaaacccacggtaccggcacccgtattggcgatcctatcgagttcgctgcgttgacggaggcgttcgatacttcacaccgccaatactgtgcactaggggctgtaaaggcgaatattggccacacccacgcggcggcgggcgtggctgggttaatcaagacggcgctggtacttcatcaccggaccattccgccgctcgccaactatcaaatgcctaactcgaagctggatctggcgcattcaccgttttatatcccgatacagccgcaggagtggcccgcatcgcggatgccgccccgcgctggcgtcagttcttttggcattggcggtactaatgtgcatatgattttggaagggctgaatcctgcggtgcgcgatgaccatgaccaagtgcgagcaccggtgtttatccctctctccgcgccgtctttcgagcagttggatgagctgacgcaacagcttaccccgttgctggctacccttgatgcgtcaacgctagcctacacacaacaagtggcgcgccccgtgtttgattgccgccgagtgatacaagtggaaaacgatggtacgcaagcgatgctggcatcgctggataacctaatgcccgacgctccttggggcctacactgtccagatctacgtactacgaacgattgtacttacgcacagtggctggcgcattcggcacattatcaacgcgaagcgactgctctgacggcattactcgacggcatgaatattccacccgcttattgccacgctgaaacgtgggcggcacaagcgaacagcagcctgctaatcagaggctgccagactatcgccgcgctaaaaacctggatgaacctgctaccgacattaacactgctgtcgggtgctggaacaggccttttgcctgccgctgcagccagtggcatgattgcgacgcaagacgtgttgcatttgctgtgggaaatggagcaaaaggcgcttcatctctggcttcctgagcgccatgaacctatccccggctacgtgctggcttggcaaggaaatcccatcaccgatgcacagcgtaacgacagagggttttggagtgaagcgctgttggcagatacccgagaactgggggagggcgttcacagtatcaactgggttaggctgccgccggaaataagagaagacgttgatgtattgcgctacgtggcgcaactgtggtgtgcaggtatcaatgtggattgggccgtgtggtacggcactccgctgccgcaacgcgggagcgcatcagcatatcccttcgcacacaaccactacccattgcctggacgggtaatgggtagtgtggagacccaacccgaagcaggacccgaaacgcaccacccttatcaggcacgtcccgtgttaagcgtacctttcgtagccgcacactcgcggggtatgcagtatatcacaggtctgatggaactgttgctggaaatatcgccggtcggggtggacgacgacttttttgaattaggcgggcattcgctgctggtgacgcaattgacctcccgtttagaacgcgatttcaacgtacatatcgatcttttgaccctgatggaaaaccccaatccgcgcaatatctacgcgcatatcgcggcgcaactggggggcgaagacaacctcgaaatagcctgtcagtaa

SEQ ID NO: 297 - DNA, 870 bp, synthetic construct
  misc_feature 1..870: note = Synthetic: clbD
  source 1..870: mol_type = other DNA
atgatgaacgtggcggtaataggtgcaggagtaatgggaactggcgtcgctcataacatggcgcaatacggcatatcaactaacgttgtggatatttctcaatctcagttggataaatgccgacagatgatcgaagcgaacttacgcttatataattttcacccgcagcataaaaaaaagacgcatagcacagcggagataatggaaaatatccgtttcacaactgagttggacgacattgtagaatgtgatttggtgattgagaatattactgaggatatagagaaaaaaaatgcactatacacacggatgaatacgatctgcggcgcgtcgacagtgtttggtgttaatacgtccgccatatctatcactgcgttgtcaaaattaatgcgacatccggagaatgtagtcggcgtccattttatgaacccggtaccgctgatgcacaccgtcgaattaatccgtggtgtacatactgcagagcgtacactgaacatatttcaccacttatttgctcagttgaataaaaccggcatcgtggtcaatgattctccgggctttgtcactaatcgtgccatgatgatttttgttaacgaagccatatttatggtgcaggagcagatcgcccgtgctgaggatatcgatacgttgtttaaaacctgcttcgggcacaagatggggccactacaaactgccgatttgattggcttggatacgattttgcaatcgttgcaggtgctatatgaaagttttaatgatgataagtatcgccccagctttttattaaagaaaatggtcgatgcagggtatttgggcgtgaaatcagggcaggggttttatcgctatcaacagacgtacgccgagcagtga

SEQ ID NO: 298 - DNA, 249 bp, synthetic construct
  misc_feature 1..249: note = Synthetic: clbE
  source 1..249: mol_type = other DNA
atgaaaaagcaagatatgaaagccgccattcgggaatttctttcacgctcattacgtgggcatacgttgaacgatgatgacgatattttttctctcgggcttgtccattcgttatttactgtgcaaatcatactgtttatagaaaaaaattttcaggttgagctggaagtgagtgagttgaaaacagaacagattgctaccgtcaataaaatagtggagctcattcagcgacaaacaggcctggagtaa

SEQ ID NO: 299 - DNA, 1131 bp, synthetic construct
  misc_feature 1..1131: note = Synthetic: clbF
  source 1..1131: mol_type = other DNA
atgtgcactgaaaattatgagctggctcagcaagaggcagtcctgtttgccaaacaacatctcgccctggctgcacagaatattgaacgtcagcagtttattgtgccagacattatttcttgcgtggcgcaggccgggtacttaggtgcgtcaatcccccaaaaatatggcggacgaggttacgattcttatcaactgtgcgctttgcatgaagtgatggctggggttcacggttcgttagaaaatctcataacagtgactggcatggtcagcacgctgctgcaacgcgtgggcagcgcagcgcaaaaagcccattatttgccaaagctggcaacgggggaattgatcggcgcgattgccctcacagagccgaatatcggcagtgacttagtgaacgtggaaacagaattacagcaggatggcgacggttggcggctgaacgggaaaaagaaatggatcacgctagggcagattgctgatttttttattgttttaatccactgtggtaatcaattagcgacagtgcttatcgatcgcaataccgacggctttactatcactccactcaacgacatgttggggttacgcggcaatatgttggcagagctgcattttaatgactgccgcctgaaggaggatgcattgctcggtccgctcacccctggggtaccgctggcggtgaattttgcgctcaatgaagggcggttcaccaccgcttgtggcagcctaggattatgtcgggccgctgtggatgtggcagcgcgctacatccgccagcgtaaacagttcaagcggcggttattttctcatggcattgttcagcacttgtttgccacaatgctgacccaaacgcgcagtgcgcaactaatgtgcttcagcgcggcggaataccgcgaaacgctgcacccggccatgataaaccaaatcctgatggcgaagtatgtcgcttccaaagctgcggtggacgtggccgggaaggcggtgcaactgctcggtgcgaatggttgccatgccgattatgccgtcgaacgttactaccgcgacgccaaaatcatggaaatcatcgaagggacatcgcaaattcatgaaatccaaattgcgatgaattacatgatggggagtgaagcatga

SEQ ID NO: 300 - DNA, 1269 bp, synthetic construct
  misc_feature 1..1269: note = Synthetic: clbG
  source 1..1269: mol_type = other DNA
atgacgaaggatgtcgcactgatgttccctggctccggttcgcaatatgtaggcatggcacggtggctgtatgagcgttatccgcaggtgcgtactctgtttgatgaagctagccagatcacggaacgggacatggcagcgctgtgcctgtcgggtactttggtacaacttgctgagccgacggcaatggcgctggctatctataccaccagcgtggcccactttgtcgcgtggcagcagtttttggcgcaaaccggtgtccatgtcaacctacgttatatgttgggtcatagtttgggcgaatatgctgcgctgacctgtagtggtgcgctgagcttttcccaagcactggcgctggtggcgatgcgtagccgtttagccagtgagatcgcccgcgaaatggacgcgagtaccactatcatcaagcaagggaatcaggcactggtggcagcggcgtgcgaggtggcggagcgtgaaacccgacagcaggtcggtattgcctgcttcaattcgccgcaacagtttatgttatccggacaaaactcggccattatcgccgccgagcaatacctgctggatcatgatcgccaagtcgaagtagtgccgctgattggtggtgttccttaccacagcccactattgaaaccttgcggacagcaattgcgcaaggcgctggatcgctgcgaatggcgacgtccgtgctgcccagtgatctccaatgtcaacgctcaaccctatcccgatacggtcacgccagtccagtggctagaacaacagctctctcagccagtgcagtggcagcgctctctcacctacctgacaggacacttgtcaccgatcgcgatagagatcggtccgcaaagtgtgctgaaaaacctgctgctggagaatcgctatccagcaccagtttacgcatttgacaatcgtcatgatcgtgcgcaactagcattggtgctgggggataacatggcggtgaagaccgatccggaagccgtgcgccgccaacgcatcacgctgctcactaacgccctgacagccacacgccatcaccgcgccgccgatgtggctgccagtgcgcaattgaaagaattgctcagtcgtttttttgagcgcatccagcagattgagcagcgcggcacatccagcgaagaggacattgcttttttgcatgagcttcttgaacagggatttcaactcaaaggcagtagtcaggcagaaatcgacgcctgccacgcgcggttggcttccgacaacggcggacaggcgtaa

SEQ ID NO: 301 - DNA, 4797 bp, synthetic construct
  misc_feature 1..4797: note = Synthetic: clbH
  source 1..4797: mol_type = other DNA
atggaacagcaagggattatgagacagttgcctaccgacgaccaaacgatagtcgactatttgtatcgtatcgccggagaatatggggaaaaagccgctgtattgatgggggacgcggcgctgagctatcacgatcttaatgcacgctctaaccaactggcgcattatctgcgtgggctggggatcggcgaggatcgtgtggtagctatccgcctgccgcgcggcatggcaatgctgattgccattttcgctattgtaaaagctggtggtgcctatttaccgctggcgtacaatgcaccgcgcagccgcattgaaaatatactgagcaacagcggcgctgtttgtctgatcggtactgacgatggtgatcgctggccgattcctcgcgtcgaaatcgacagcgcggcggtcagtgccatgccaacgacggatctgcgttaccgaccacatgcgcggcaactggcgtatattatttacacctccggttccaccggtgtccctaaaggggtggcgacggaacatgcagcgctgctaaaccgaattgtctggatgcagaacgcttatcctatcagttctcaggacgtgctgtttcagaagacggtgtacacctttgacgtctctgtctgggagatgttctggtgggcgatgtacggcgcatctgtagtgctgttaccgtccggactggagagcgatccgcgaaccttggctcgactgattcagcgtcaccgcgtgtcggttgtgcactttgtcccttcgatgctgaacctgtttgtcgagtatctggagatgaaacaggatcctcgtttgaccgcctcattgcgattagtgttttccagcggtgagaaactcacggtccacagtgtggctcgcttttatcaatcggtggcgcagggtgatcttataaatctctatggcccgaccgaagcagcaatcgatgtcagtcatcaccgctgcctgcgcgggtacgactacgacgatatccctatcggtcaagcgattgacggttgccgactctatgtgctggatgaccatggtaatccggtagcagacggcgaagagggcgaactgtatctcgctggtatcgggctggcacgtggctatctcaacaacgtggcgttaactgatcgctgttttactatacatccaactttgcgccatttagggaaaccggagcggctgtataaaactggagatctggtgtggcgcgacggggaaagccaacaaattcattacattggccgtaatgattttcagattaaaattagagggttgcgcgttgaattgggagaaatcgaagcccatgcgatgcgtttcccgggggtacagcaggcagtcgtggtggcggatcaggatgatcccgacaatcagttgatttacgcttttgtcgtcagtagcgtgccgctcaatttggcggccttaatggacgcactgtccaaaaacttgcctgcctacatgctgccgaaccgtttgttggcaatgtcagagttaccactctccgacaatggcaagtgctgtcgtaaaacgttgctcgacttggcgcgggcgtactcagccagtcgcgtcgatttacgtgaaactcccgccgtgcgctacctaccgttgtcatcggctcaatcgtcgatgtggtttatgcaacaattggcgccgcatactgcactatacaataaccccaccgccttgctgcttgaaggagaactggatcgcacgcggatggacggcgcgattcgtcaattgatgagtcggcatactctgttacgtgctatggcggaaacccacaatggacaaccagtattggcggtgcctcagtgcgtatcgtcgcaggcgttgctgaccatagtgccactcccctcggtgagtgatgataacgcgctacaggcgatgatcaaccagcgtgcggcacaccccatgcccttaacgtcaggcacaccgctgtgccggtttgaactgttgacgctcgatgacgatcgtagcgtattgttgattcatctgcaccacatcatcagtgatggctggtcgaaaggcgttttgttacgcgaattgcaggccgcatataacggtgaatcgctgacgccagaaccgttgttggagtatgccgactacatggagtaccaagaagagtggcgtcagagtgacgcctatcaggacgcgatgcgttactggcaaaataccctggcggggacgttgccgattcttgatatccccaccgatcaaccgcgtcagaaggtggcacgttaccaaggcgcgtttgttgccttcgcattgtctgccaacacatgcgagcgagtgttggcggcggcgcgtgcgcagcgcgtgtcgttgtataactatctgctgaccgccttcgtcctcctgctgcatcgtaatgcgcgtcaacaggaatacatcgtcggtatgccgattgctgcgcggctgacgaaagaacaggagcatatgatcgcgccgctggtcaacgtactaccgctgcgcttacctcttgacgaagcggcatcgttttccgagttagtacagacgatcagaggtattctgtttgccgctttcaggcaccagcgtctcgaatttaccgacatagtgcgcgcagtgaatgtggatcgcagcgccggacatttcccaatatatcagtgcatgttccaactcgacaatatgcctctggcgagcccgacactaaacggtgtcaacgttacaccattattgttggataccagtgcatctcaggtggatatttcgctaagtatgcagcatatcgatgggcgcatcaccggcactttcgaatacgacgctgggttatacagtgcggatcgtattcagcacctggtggcgcagtggagagaactgctagatgaggccagcagccaacccacgcagttagtccgcgatttgattcgtttcacccctcgagagcacgcttggctggcgcggcacaacgccactgaagttgctctgccgcccgtagataacctgctggcgctagtgttaccgcactgccagcagcgccctacacaggtggctttgcgccacgctgacgacgccatgacctatggcgaattgcaacaggccacgatgcagatgtgtacttggctgcgtgcgcaaggcgtaaaacgcggagaatctgtcgcactgcaactgcctttttgtttcgaattgattatcgcacaattggcgatcctctctttgggggcgagctatgtgccgctggatggtaacgcgccagcggcccgcaatgccctgatcttggcgcaggcaacgccgtgcatgctgctggtggcgcaaccgctggaatcccctcatgggctgacgataccttgggtgctagtgcctgactggcgtagcctgctgacagaaataccgaacctgccagttagtgttgcgccagatgctttagattgcgatgcggtggtgatctttacctccggtactaccgggcagcccaaaggcgtccgactcagccaacgtaatctggtcaacttaaccgcatcctttatatccagctatcaagtgacgcatcaggatgttttgctacccattacctcagtcgcgtctgctagctttgtcggggaggtactgccgctgctcgccgctggcggtaccttagtattggcgcaaaaagcgcagagtttggatagtgatgcgctcattgcgctgctggcatctcagcgggtgactatcctgagcaccaccccatcgctttctgccagcctctctgtgctggctcagtcgatgggatcgctgcgcttgtttctgtgcggtggtgaagcgttagagtatgagcagatagcgccgcttctgccgcacatggcagtggttaacggttatggcctgaccgaaagcggtatctgctcgacctactttcctgtcgcaaagcgtagggagcaagaaacgggagcattgcccattggccgcccgattcagaacacccaagcttatgtggtggatgcatataatcgtttggtaccgccaggtgcctgtggagaactctgtttctctggtctgggcatttcgcccggctaccttgatgcacgacaggatcccgagcgctttgtcgagttgccggaataccctggcgttcgggtgctgaaaactggcgatcgtgctcgttgggcaaccgacgggatgttgttctatctcggcagacaagatcgccaagtgcagatccgtggataccgagttgaactgggcgacatcgaaagcctactgaaacagcatccggatattgctgatgcgtgggtcgatgtgcgacgcaatgcggcggcaacgccgctactagtggccttctattgcagcgtcaacggcgttgcgctggatgctcagcaattgcgcgtatggctcagcctgcggcttccgttgcatatgctgccgttgctctacgtgccgctgagcgccatgccgctaggtgtaaacggcaaaatcgatccccagtgcctgccgctggtcgatctgcggcagttggaagggccgggcgagtatgtccctccggcaaccgaactggaacagcgtctggcggagatctggcagcagttgcttggcctggagcgtgtcggcaccaccacaaatttcttcgatctcggtggacactcacttctgctagtacagatgcagcaatacatcgggcagcagtgcggtcagcacgtggcgttggttgacctgctgcgcttcactaccatcaaacgcttggcggaatttctgctggccccggacgcagcgcaagggaccacaggagatcagacacaactgcgtgcggcgaagcagcgtttggcctttggtcacacgcgctgggcagccacaaccgacagtcatcactga

SEQ ID NO: 302 - DNA, 3033 bp, synthetic construct
  misc_feature 1..3033: note = Synthetic: clbI
  source 1..3033: mol_type = other DNA
atggcagagaatgattttggtatagctatcattgggatggcggggcgtttccctcaagccgatacggtacaggcgttttgggaaaatcttctggcaagccgagagtgcatttccttttacagcgatgaggaactgctggcgatgggaatcagtcctgaatttgttcagcacccagattacgtcaaggccaaaggggaggtagctgacatcgataaatttgatgctgctttcttcggtatcgcaccccgagaggcggagctgatggatcctcagcaccgtgtcctgttggaaaccgcgtgggcagccttcgaagatgcgggctatgtggctgccgattatccgggggatgtgggtatttttgctggcaaaagcatggattcctatttgatgctgaacttgatgccgcactttaaacgcgttttctcttctggcagtttgcaggcggccattggtaacgacaaagacagtatcactactaccatcgcctatcacctcaacctgcgcggcccggcgatcacggtgcagacttcctcatcgacatccttggtggcggtgtgcgtggcatgccaaagtctgttgacttggcagtgtgacatggcgattgctggcggagtaacgctggggccaccagcaaaaaccggctacttgtcgcaggaaggtgggatcaccgccgccgatggccactgtcgggcgttttccgataacagcagcggatttgtgcccggcaccggcgcggggctggtggtgttaaagcgtgttgatgaagcgctgcgtgatggcgataacatctatgcggtcatcaaaggatttgccgtcaacaacgacggttcggagaagatcagttataccgcgccgagcgtggatgcccaagcgcgcgccattgctcaggcacagcggttggcaggtttgacaccgcaggatatcacttatgtggaagcccacggtaccggtacgcgtttgggcgatccggttgagttttctgcgctaagccaagcctttgcgggcgcgtcacaaaagcagtactgtgcgctggggtcggtaaaaaccaatatcggccatttggatacggcagcgggcgtcgcagggttgataaaaaccgcgctggcagtgcagcagggaataattcccgcaacattgcactttgaacggcccaatgcacagatcgatctgaccaatagtccgttttacatcaacactacctgtcaaccgtggcaaccggaaagcggcatccgtcgcgcgggcgtcacctcgcttggcatgggcggcaccaatgcccatgttgtgctggaacaagcgccagccgttgacttgcaagcgcgagcgcctgtacctgcctacagtattttgccgttttctgccaagaccgacagtgccttatcctcggggctggcgcgttttgccgattttctgcaacatgaatcgctgccggataggcgggatctggcgtggacactctcacagggacgtaaagccttcgcacatcgcgccgcgctcgtaaccagagatctacatgctgccgggacgctgttgcagcaggccgcgacagcgccgtttgctcgtggtgtagcgcagacacagctcgggctggggctgttgttttccgggcagggtagccaatatcagcgcatgggccatcagctctatcaggtttggccggcctacgccgatgccttcgaccgttgcgcgacgttactcgagcgtgaataccaactggatatccgccatgagttgttcagagcagaagtctcgttagcccaaggcgaacgccttgcgcaaacctgcctcacacaaccgctgctgttcagcgtcgagtatgccttagcccaattgtggctcagttggggaatcacgccaacggtaatgattggtcattcgctgggcgaatgggtggcagcgacgttggcaggcgtgttctctttagaggatgcgttgcgcttggtggcgcgtcgagcagagctgatgcatcaggcaccaagcggtgccatgttgatggtggcgctgcccgaagcacagattcgcgcactgattaccgccccgctggcgattgccgctgttaacgcccctgactattcggttatcgccgggcctacgtcggagatcctcgccgtcagccagcgtctgacggagcagaacatcatcaacaaacgattgcatacctctcatgccttccactctagcatgatgcaagatgcggcgcaggcgctacgtcaggcatttgagaatgtacgactgaacccgccgacgctcaccatcatctctaccgtaaccggcgcgcacgtcagtgccgacactctcaccacaccggactactggattgaacagatgctgatgcctgtgcagttctccgctgcactgcaagaggcgcaagctacatttgatgtcgattttctggaaatcgggccaggtgccaccctgacccagctgaccaacggtcatgcattaggtgatcgtctcgcgttcagctcgctgccagcaggtgcccgcagcagcgatgagcacaaacatatcttagataccgtggcagcgctttgggtgcgagggcataacatcgatttgtctgcgtttgcaggtgagcaaccgcgccgtgtctctctaccgacctacgcctttgacaaaatccgttactgggtcgacagtccagaagaacagaggagcgccgtaacgccggtggcggacgcgggaagtgtcatccctagcgaaccgtcggtgcgccgtcagccgcgtccggcgttttcggtgccttacgcggctccagagagcaaaacgcagcgcggcttagtggcgatttgtgaagcattgctgggaattgacggcttgggcattgacgacaacttcttcgaagctggcggacattcattgatgctgggcatgttgctggcgcaggtacaggaacggttcgccgtcactctttcctttttcgacgtgatggaggatgccagcgtgcgcgcgttagcgcagttggtcgagcaggagcagcaggatgatggagggtccgcacttgccgtgctagttaacgacatgattaatgagtga

SEQ ID NO: 303 - DNA, 6501 bp, synthetic construct
  misc_feature 1..6501: note = Synthetic: clbJ
  source 1..6501: mol_type = other DNA
atgacgatacatcatgccgcattggcgcgaatgttaccggcggaaaaaaaggaaaagctattacgacagttggcgcaatcaggcgtgtcgcccagccgcattcccatcatcaaagcggatccggcgcaggcaatcccgctgtcgtttaatcaggagcggttgtggtttttgcaaaaatacgacagtaccgccactaactacaacctttatgtggtgtatcgtttgcatggcgtggtggatatgccgatgctcacggaggcattgcgccatgtgcaagcacgccacgccatcttgcgtacccgtatcatagtacgtaacgatcggccttgccaggtgatagatgatgcgtcttcgttagttctcgatacggtcacgctggcagcgcaggccccaacttcagcgctagatgcggtaattcagcaggtgataaacacccggttcgatctggcacgcggtcctctgtggggggtaactcagattattcaacccgatcaaggttgtcatttggtgttttgcgcgcaccatatcatcatcgacggtatctcgttacggctattgttcgatgagttacagcagcagtatgctcgattgcatgctggtaatgaaacgtcgctgcctccgccgccgctgcaatatgccgattatgccttctggcagcgcgaatggttccaggataccttgctggcgaacgaacttgcctactggcgtgcgcgtttgcaagacgcgcctctgctgtcgacatttccatcgctgcacccgcgcccggcacaaccctctacacatggttcccggttcagcatcaccctggatgaaacattaagcctcgcgttgaaacacgtagcccgcacgcaggaaacaacgccgttcgtgctgatgctgactgccttccaactggtgctgatgcgttacgctcagcaacagcgattggtgataggcatgccagtatcggggcgcattcgcccggaattgcagagtagcattggttattatgccagcactgcggttatctacaccgattttaatggggttgaggtagggcgcgaggcgctccagcgtgtgaaggccagcgtgaaagagacgcaggggcgccagcaactgccgtttgaaaacttggtgaatatgctggatctccctcgcagcctttcccattcaccgttgttccagatcctctatatctaccataaccacgtgaccccacgcgcttttaccttggctggtgcgtattgggagcaggtgacgtatcacaatcagaccgtcaaatacgacatgacagtcgaggtgttccaaaacgacgccacgttcgacgtctcctttgagtacgatttggggttatacgatgctgatgtggtgaagcagattgccgaggcgctgcgtcagcactgcttatcgttgacatcatcactggagaccccgataggggcgatccctctgcatgcaccggagaccgcaacgccgcggcgtgatccgctcaacgccactaacgtcccgtggctcgggccgcaggatgtgctgcgtatcattgaacagcgctgtgtgcagcacccaaagcaactggcaatacagcagcatgacggcacactgacctacgctgagctctgggcgcgtgtgcagttcatcgcgatgcgttttcgagcgcatggcatacaaccgggcgatcgtattggcgtgctgttgccacgtcacagggatgtgattgcaaccatgttggcgacgtggtttgtgggggcgtgctacgtgccgttcgatattcatcagcctgccgcgcgtttgcaacgccttatgcaacgcgcgcgtttggtctgtctggtggtccgtcagccgggagagtggggtgaaattgtacaactgtcgttgccggaattgatgcaggacatgtcgaatgcgatccggtattctacaccttgcgcgctgttgccggatatgcaggcctacctgttgtttacctccggcagtaccggtgaacctaaaggcgtgtgcgttgttcaccgcgggttgctgaacttgttgttggatatgcagcgtacctttgcggttggctcgcaagaccggctgctctcggtgacgacgccaacatttgatatctcattcttggagtatctgttgccgctgatctccggtgcgagtctctatctgacagaggcggaacgcgccgcagacagcttccgtatgattccgctgattgccgactatcgaccaacgctgatgcaggcgacgccctcgttctggcacgggctgttgatggcgggttggcgtggcgacccggaactatgtgtgctggcaggcggtgaagcgctgccaacgaaagtggcggaagaactgttgcgctgttgtggttcattgtggaacctctacggtcccaccgaaacaaccatttggtcgctgaaatcgcagataacccaagcggaaaacatcaccctcggcgctcccattgccaatacccgtatatacattctggacaatgagggccatccagtgccgcaaggcgttgacggcgagctttacatcgccggggatggtgtggcgcaagggtatgatgggcagcctgagttgaacgcacagttcttcttgtcagaaccaggggtccccggtggcaggatgttccgcactggcgatctggtcaggagtgatgcgcaggggcagttgttttttgttgggcgcaaagatagccagatcaaactgcgcggttatcgtattgagttaggcgaaattgaacggacgttggcacggcatccgcatgtggacgccgcggtggtggcctgtattgaaagagcaccgttacacaaagcactggcagcattcatcatcaccagtgagcctccctcgctattcgagcaactgaaaaacgagcttcggcagcaactgccagactatatggtgccaacgctgtggcagagggtggccgacttcccgaacactgacaacggtaaaatcgatcgtaagcggttagcggaaaatttcgttgccgatagctcccttgtgtcgccgcagacgcaagcgctgagcgacacggaacagatgttgctggcgctgtggatgcgctatttgccgataaaaaacgtcgatcctgagtgcgatttctttcgcttggggggacattcgctgttggcggtaacgttggtagcagagatcaaccgcacttttcactgcgccttgaccctgaaggacattttccactactccacactacgggcactgagtgcgcgtattgcacagcaatctatcacggacgctgccgcgtctcaggatgactgggtgatagtgcacgaccctgagcatcgtcatcaaccgttcccattgacggatgttcagcgcgcctactggcttggacgacagacgggtgctacctcgatcgcgacccacatctaccatgaatttgacgtagaacactttaatgttacgcgttttacccatgcggtgaatgcgctgatcgctcgccatgaaatgctacgtgcgcgggtactccccgacggtactcagcagattctggcgcaagtgccggcgtatcagttagagcagcgcgatctgagtgctttgtcccctaacgcacgaaacgatgccttgatggcgatccgcgatcggctgtcgcatcatgtgcatcccgcagatcgttggccgctgtttgatttcagttattcggcttgcacggcgcaacatggccgcttgcatttcagtctcgatctgctgattgccgatgctctgagtatgcgcacgctacagcaggagttgatgatgctgtaccgtgagccccatgtgtcactgccgttgctaccgttctcttttcgtgactacgtgcaggcgctgttggtagagcaggcgagtgaagcctatgcacgcgatcaggcctattggcaacgggcgctgccgcagctgtatggcccaccaacgctgcccgtacagggcgatttggcgcaactgtctgcgatcaggttcgtacgtcgccgtcatcggctgtcagcccacaactggggagtgctgagcgcgctggcccaacgcacacgtatcaccaagacggcattgttgttgacagtctttagccaagtgctggcacgttggagccttagcccgacgtttacgctcaatctgacgttgttcaaccgcccgcagggttaccccaacgcagaggcagttattggtgattttaccgctgtcagcttgctgaatgtttgttacgacagccagcactcttatgcccacaacgctcagcgtattcaggtgcaactgtgggaagatctcgaacatcgtcgtttcagtgggatccgcgccagcgaggcgctgatccatagcggtcgtttccatgcgccgatgccggtggtattcactagtatgttggatatcgacggggagacgactgcgcaagaccctcgggacacaacccgttttactctgtgtccggacgccaatattacccaaacaccgcaggtgtggctcgatcaccaggtgatcgagttggctggggagttgcatttcaactgggacgcggtcgagcaactgtttgataccacgctgctggatcagatgtttggtgcttattgtcatgcgctgcaggcgctggttgccatgccgcaaagttggtggggggtaaatagttctctggcgctgcccaccgttagtgcaccggtcacgcaggctcctgcacctacggccttgttgcaccatggattactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacgctggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaagtcaaaagccaccttgaacagctcgacagcgtagggagtgccgccgtggtgtgccaccagggacagctgtatgcctttatcactgccgcagaaaacctgcatcctgacgatactgacgccctgttagcgcgtgttcgtgctcagttagccgtgcagttgccgtattacctgctgccccaacatttctttcttctcaaggtgctgccgatgacaggcaacggcaaaattgatcaggcggcaatggttcaagaggttatccaacgtatgtcgcaatctacatcacagaagtcgagggccctcgcgcacgcctcgccctatgaacagcaggtggccgctctctggtgcgaggtactacaacgagaacagatcggactgaatgacaacttttttgaagcggggggcggctcaatccagatcgtgctgttgcatcgccgtattgaggagatttttaaggttacggtacctattgctgagctatttcgcttaaccaccgtgaaaagaattgccggttatttgcaggctatgcaggacaatgcacgggcggtgaatcaaacacagcagcgtgatgcttcccgatcccgcgcccagcaacgacttgtacgtcgtcaccagcgtcagcgttaa

SEQ ID NO: 304 - DNA, 6464 bp, synthetic construct
  misc_feature 1..6464: note = Synthetic: clbK
  source 1..6464: mol_type = other DNA
atgacttacagtgaaagcgatattgccattgttggcatgaactgccggtaccccggcgtgcacagcgttgcggcattcgaaacggtgcttcgcaccggatgcaacattttggaccccaaagtgaccccgagcaacggccacaatcacattacgctgaacaacgtctatgagcacatggcggagttcgatgccaacttttttggctacagccgtgcagaagcagaaatcatggatccacagcagcgcgtatttctgacctgtgcttgggagatgtttgaacaaagcggttacaacccgaagcagcacgatgcgcgcgtcggcctatatgcgggcgtgagtaccagcttctatttgcttactcacctgatgaacaacccagacaagcttgcacaactcggtggtctccaaatcatggtcggcaatgacaaagatcatttgacgtcccaactggcttaccgcctgaatattaccggcccttgcgtgacggttcaggccagttgtgccacctcgctggtggcagtgcatctggcctgcgaagggctgctgagcggtcagtgcgatatggcgctagcgggtggcgtcacgtttcgcatggaggagcagcgcagctatgaatcgcacggtgatgggctgcaagcagaggatggcctgattcatacttttgatgcgcaggcgagcggtacggtctacagcagtggtttgggcatggtgctgctcaaacgggctacagacgcgcaggtgcaaggggataacatccttgccgtcatcaagggaagcgccatcaacaatgatggcggtgcgcgcagtggttataccgtgccgggtgttgatggtcaggaagctgtgatgattgaggcgcatagcttggcggaggtgacgccgcagcaaattcagtatctagaactgcatggcagcggcacgccgttgggtgacgctatcgaattcgcggccatcaaacgtgtgtttgggacgccagcgccgaatgcgacaccgtggcgtctgggggctgtgaaacctaacgtgggtcacgtagaaatggcatctggcatcaccagtctcataaaaacggtgctgagtcttactaaccgggtgttttaccctacgctgaattttcaacgggctaatccgcaactggggttggaggacagcccgtttgaagtggtgtcccgtttaacgccttggccggagggcaccacgccacgcaccgctggtgtgagtgcgttcggattgggcggcaccaacgcacatctggtggtacaagcgccgttatcgacgccgcaggcgagggcgcagcagatggggccttgtgtcgttgtactctcggcaaaaaaccacaatgccctggaacagatgcaaaacgccctgctggcgaaactggccgcacatccagagatacgcttacaggacgtggcttacaccctgcgtcacgggcgtttctccgccccggtacgtaagtgtgtcatcgcagagaattgcacccagctagcccgacaactccgcgacgcaccgatggtggaggcaaccacgggttgcacgatctactggagattggggcaccgtttcgttgttgcactcgagacgcttagtgactggctggcgtgctctgaggtgttgtcacaggcggtgggacaactgctcgagcattttccgctcgaaccagcctgtctgcaagatctctcccctgcgcaacggacgtttatcagccagtatgcgctgatcgcgttgattgacgaacgggaaacgttgaatgtggtgctatgcggcgacggtgatggtggctatgctgccgccgtactacgaggagactgcacactagagcaagcatggcacaggttgaatgcgggccaaccgtttgatgatgtgccgacgaatcccctcttgcagcccgatgtctgctctttgatgctggacgacgcggcgagtgatgccaatcgcaccgcgctagaggcgttagggcagttatggttggctggcgtcagcctagattggcgctgggtggatgcggcggagcgtatgttgggcagtcaacgcatcgccctgccggggaccgtttttacaccgcagcgctattgggtcgaggccgtacggcctgccacgttctcccacgaatcgtcgaataatctcttatcccgcgcaacaaaatccgatattattgcagtggtgacggagatatgggaacgcacgcttggtgtcagcattgatgatcaccacgccagcttcttcgaactgggtgggcattcgctgttagcgtcaaccattttatatgatattcagcaacgttacggcatcacctgtacgctaagcgcattctttgccgatcccaccatcgagggattaagttgctatctgctcgaacaaggcggcagtgagacagcggtttcagcattgcctgatacggtctttgcccctgaccagcagcacctaccttttccgctgactgacgtgcaacaggcctattgggttgggcggcgaaaatccttgggattgggcaatatttccacccatatctatgtggaatatgaactacaggggctagatgaaacagcgttcaaccgcgcgctcaatgcggtgattgcgcggcacagcatgttgcgtgcaattgtcaacgatgacggtatgcagcagatattgccaaacgtgccggaataccacgttgccttctataccacccagtgcgaggatgcgtttcaacagcgttgccgtgagctgcgtgacaccctctcacatcagatgattgattgtagtcgctggcctctgtttcagatggaggtcgtggttgatccgcagcaaaaagcccggctacatgtatctatcgacctgctgatagccgatgcatggagtctggagttgttcattcgggaactagcttatcactaccgccatccgcaggcggcattgcctacattgacgtacagtttccgcgactatgtgctgaccttaaaatcctacgagaaaacgccgcagtttgaacgggcacgtgactactggcgcgcccgcatcgaaactctgccgccggggccacgattgccgctgcgtaccgaccccacgaagctggaaaaccccacattcgtgcgccgtagctattgtctatctcgtgctatctggcagcgcttgaaaacgcaggcgggtcagatgagtatcacgcctactacactgctgctcacggggtttgctcaggtgttggcgcgctttagttcctcgccgcactttagccttaacctgacgctgtttaatcgtctgccgttgcacgcagatatcaatcacttaattggcgattttactgcactgacgttgctggagattgatatgtcgcagggagaaaccttgcaggcgcgggcaaacgtgatccactctcaactgtggcgcgatttggataaccgcttgtttggcggtatccaggtctctcgactgctggtacaaactcaccgcgatccggcgaaatcggtgattcctatcgtgttcaccagcttgctcaatcaatatgaggcgagctgggaaacggatgatacgctgttcaaccaaccgcaggatgatctgtacagtatttcacagacaccgcaggtgtggctcgatcatcaggtaatggagcgtaacggggaattgcactttaattgggacgtggtggaacaattgtttgaaccggcgctaatggatcagatgttccagtgttattgccaactcctgcacgccctagcccagcgaccacagctctggcatgagactcaagatgtgctggcgctgcccaccgttagtgcaccggtcacgcaggctcctgcacctacggccttgttgcaccatgggttactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacactggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaaatcgagcgctgcattgcgcgacatcccgatgtggagcagtcagtggtggtggcagtgggtaattctcaacatcgtcggctggtcgcttttgccaaactgcacgatcgccaccaggcgcaggcattgcaagctaaggaagcggaggcggcggcactggcgcagggtattattgtgaatccggcacagcgtctagcgttcaaactcaaggagccacatattcgcgcgctggatggtctgggcattgcactgacggcaccggcggatagcacacgttacatcaaacgccgcagctatcgtcatttcagcgcgcaaaaaaccacgctggcacagttggggcaattgctgtcgggcttggggcagatgcgtctacccggtctaccttttgccaagtatgcctatgcgtccgccggggggctatacccggtgcaaacctacgtgtacctgcatccagacaagatcgaagagggagtatccggtatttactacttcgacccgcgacagagctgtcttatgccggtagcaccagaagtcgagctgaacagtggttttcatgccggacctaatcagtctattgccgatcgggcggcattcacgctgtttatggtggctgatatggcggtgatctcgccattctatgggcaagaggcagcttggcacttctcggtgatggaagcaggtactctctgccatttactggaagaagatgcgccgcgctacggattggggctgtgccaacttgggatggcagacttttccgctgtggcatcgcattttcaattgtcgccacatcatcgctatgtccattgcaccgtggggggcgcgatagggcaagaggcggcaagtgctgcagcattgctgcgcgatttctccacctatgagaaaccgaaggaaaccgctgcgccgctggacatgcagagctacaaagatgccatgctgcgcggcctgcgtcagcaactgcctgactatatggtgccgagtgatctgatgttagcgaccgattttccgttaaccgctaacggcaagctagatcggcaaaaattacagctgcagggcgaacaaattgcccaccagcgtgacggcgtgggtccaatccaggtggacagtgcgttacaacagcggctggtggcgctctggcaggaggtactcggcgtgagccacgtgtcggccgaagacgatttcttctcgctggggggcagttctatagaattggtgcgtattcagcaggcacttgaggcgattatcgggcaggagattcccattgtcgatctgttccgtctgccaaccatcgcggatgtggcgcgttaccttgatgagcaactgcacaatctacccgccgcccacgatatcgtattagcgcaggctgaggtttcgcaggtcagcgctgcgcgcgagaatctggcgttgcggcgtaaacgtgcccaacagggagaaaagggcgatgagtg

SEQ ID NO: 305 - DNA, 1464 bp, synthetic construct
  misc_feature 1..1464: note = Synthetic: clbL
  source 1..1464: mol_type = other DNA
atgagtgagcagagctatcgtagcgcagggacactgttggcacagttggcgtccggagaaaccacctcagtggcgttggtgaatcactatttttcacgtatggcgcagtttaacaagccgttgaatgcggtggtgcagcagcactatgcgttggcgttggaggccgccgcgcgcgccgatcgtgagcgactggaggggcgcgcaaggggcgtgttgcacggattaccttgtactgttaaggagtcgtttgacgtgcaagggtggttaaccacgtccggtgcccattatctgaaagataaccgggcgacgcaggatgctccttccatcgcacgcttacgcgctgccggggcgatcctgatgggaaaaaccaatgtgccgatgatgactgccgattggcaaacctataacgatctgtatggcaccacccataacctgtgggataggcaacgttcacccggcggatcgtccggaggagcggcggtggcggtggcggcggatttcacccccgtggaatttggcagcgatttgtttggctcactgcgcattcccgcacactacacaggtgtctatgcccatcgttgtagcttggggttgatgtcggtcagaggacatgtgcccggtggtggtccgcaagcgactgatgagccggatctctcgacggcgggccccatggcgcgcagcgcggcggatctgcgtctgatgatgcgcgcattgagcacattctgggtggaaccgccgcgtattcccgattttagccgttatcaggccaaagcaaactaccgcgtgtgcacgtggtttagtgcgccccaccatgagatagatcaacagatcgcacagcgttttcaatcgtttatcgacaagttacgcgcacagcctggcgtggaggttgatgatgctatgcccgccgatatcgatcccgacgcactgtttgatatagcagtcaaactcagtggtcggctggtaagtacggcgctcaatggacgccagcgtcttacagccgggctggctgcgctgggcttcaggcttgtgggcaagctggcggatgtgcctgaggggataacgagctattaccagggcatgctgaaagacagtggcgaacagcggaatacagacaaactccgccacgaatacagccgggtgatagaaacccttttcgcgcgttatgacgtgttgttgacacctgtcagcccggtgctggcgtttgcgcacatgcagcagccggtgcgtaagcgcaagcttatcgtcaatggcgaaccgcaagattacaacgaacatttgttctggaacatgttagcaacggtttttggtttgcctgccaccgtttacccattggcgaaaacgatggatgagcttccgtgcggcatacagatcatttccgggcattttcacgatgatgtgacgattaactttgctgagttctgcgaaagcatcagtggcggattcacggtaccggaagggtactag

SEQ ID NO: 306 - DNA, 1440 bp, synthetic construct
  misc_feature 1..1440: note = Synthetic: clbM
  source 1..1440: mol_type = other DNA
atgacggtgaataagcttgagccagacaacggcacgccgaacgatgagctgttcacggtggcgggtatgtttgacggctcgctgtataaattactcctgcgcatggcgttgccgatgtttgtcggtatgttaacgcaggtgacctatgccatcgccgatattttttggctgagccacattgatgtcaccaacagcggcatcattgccggtgtggggctggttttccccgttggaatggggttatttgccatcgccaacggcattcaaatcggtatgggatctttgctgtcccgcgccatcggcatgcagcggttggatcgtgcacagcggattttgtcggtcggtatcatcatcgcgctattctttgcgatcgtgatcaccgtacttggctatgtttatgcccagccgctattgcgctcactgggggccacgaagagcatcatcggctacgcaacggagttctattattattcgctgctgaccgtgttcagcatcatgttgattggcgtcatgatggggctgtttcaaggcgcgggtaagatcatggtcatcatgaaggcctcattgttgggggcgttggtaaatattatgctcgatccaatcatgatctttgtattcgattttggtgtgaaaggggtggcattggcgtcgtttctggcgcaactgagtatggtcgcctatttcatctacacattgatgggattacatatcgggttgagtatacgcatcgcgttacgcccattctcctggaaaatctaccgagagtttctctccgtcggcatggcgcagatgctgatgcaactgatcattgcggtcggcatagtgatttataatttttttatcgtcaggctggacgtaaacgcgatggcagcgtttacgctcactggtcgcatcgactatttcatcatcacgccgatgctggctatcgcgaccgcgttgctgactgtggtggggcaaaactggggacacggaaatgtcacccgcacgctgaatgcctattgggccgccgtcgcgctggccttctctattgtgctggtgttagccgttatgcatattgtactggcaccctggatgtatcccctgtttactcgtgttgttgccgtgagtgactatgcggtactgcaaacccgtatcatggcactggcactaccctttgtcgctatcagcctgttagcaagtgaatattatcaggccattggtaagccttggtattcggtgttgttgacgctgatgcgtcacgtgtttatatctgtccctgtcgtatacctgctggcgattgtgttagagatgcgtattaccggcgtctattttggggcgatgagcggcacttttgtggctgcgttgttggcgtggcgtctgctgcgtctttcacctcgtctgctgcgatggaatcaggaggcggtgcgctcccaacacttggatatggaggtggcaccatga

SEQ ID NO: 307 - DNA, 4368 bp, synthetic construct
  misc_feature 1..4368: note = Synthetic: clbN
  source 1..4368: mol_type = other DNA
atgatgtcgggcaatccgttgtcgtggccacaggaacagtgccacatcattgatcaactgtatccttacagtgccgtaaacatcattggtggtgtggtcaccattgagggaattgtcgatctgccccgactacacgcggccatccagtccgccattcggcaatttgatgcgttgcgtatgtggtttgtgatgggagaggagagtgaagtggtcagccaagtgcagccttatcactggcgcgatatccgccatttgactttttccccagattatgacaaggagaacctacgcccagcggcgatcgaaacatttgttgatgagtggttccggcagccctttaccctgctggcgcacgatttgttcgagtttgtcacctttacgtgcggtgaacaatacagcggttatttatttaaagcccatcacggcattgctgatggttggtcaatggcgttgttgtcaaaccatgtcaagcgagcatacgaacagcaggacgtaccagatgatgcgtcaccagcctatagcgcatttctggcgcagcagcaatcttatcaggcgtcaacgcgctttgccgttgatcgtggctggtggcgtgactatatcgacgaataccgcgactgctttccagacagttcccccatcgttactactgagggcatcagttgttccacttggcttgagccagccatgattaaccgtctctacaggttgtgtaatcgttatggctgtacgcttaatacgctgtttattgcactcttcgctctctatcgcgccagagtatggggggaggaaaaaggcgtaataggcgtgccgttagccaatcggcatactcgcgaggcgcggcgttgctttggcatgtttactaatcaattgccgctggcctatcgactggtgcgcacggaacgattctgtgaacgggtggcctttttccagcgcgaactgaaacgcggttttaaacacagcaaatatcccattaccttattcaaccaggatttggcggaacaggggggagggaagttgcgggcgttcgattattgcgttaattattataactttacctatgaacgccacattgcaggtgccgcgcaacgcgtggagagttactacagcggcgagcagtcctataagctacagattgttttgcaaactgttaataatcataaagaaagccttaggttgagtctggaggcgctgcgtagcgctttcactccgcaccaactgacagcgatgaaaaatgggctgttggatttagtcactgcattggatcggcaacctgacgctcgtctaggcgatctcgaagtctatcctgcgccgcacgtcgcattggcatgtggctcactcaaaccctcatttacttcccgatttgcagcgcaagttgtggagcatggtgatcgcacagcactaattgataatgaacagtctttgacttatcggcagttggatgacgctgttgagcgggttgcgcgttatctgcgccaacaggggatcggacgcggtcaagtggttgggattatcgctgagcatagcgctcaaacggtgatggttatttatggtattttgcgctgcggcgcggcatttctgccgctaaatcccgcgttgccaacgacccgcctttacgccatgtgtcgtaaagcgcaggtagcgcatatcctttacgatccggcgatgcatgaattgacgcaggcgctggcgtttcccgcgtccagcttgctccaggccttagccacttcggcgcttgctagagaaccttggccagctattgaaccgcaggatctggcatatgtgctgtttacctccggatcgaccggcgagcctaagggcgtgcaagtctctcatggcaatctggctaactatctccatttcgccgccgaacgttactttacagcgcaggaccgggctgcgctctactcttcgctgtccttcgatttgaccatcactacgctgttcgcccccttatgtgtgggcgctagcatcagcgtttgtcggcacgccgagagcgaaacattgctgcgcatggccgtggttgatcagcccaacacagtgatcaaactgacgcctgcacacctgcggttgttgtgtgcggctggcataagcagtgagcagattcgtaccttggtggttggcggggaggatttcaagcgggatctggcgcgtaaggcggctgctctctttcctcaggccgtgatctataacgaatatgggccgacagaggccaccgttggttgcatgatctatcgctacaccgggcaagaaacgctgccttcattgcccatcggtatggcgatcgacggttgccaggtagcaatttgctccccttggggctgtccggtaccggaaggcgaaactggtgaattggtgatttatggcgcgtcagtgacgcagggctatatcgatgcgccgcaacaaacggccgccgcctaccttaaagataccaatggggtgatgattggctatcgcagcggtgatattggctacgccatcgctcctaatacgctggtttatcagggacgtaaagacgatcaggtcaaaattaatggctatcgtattgagctgtgcgaaatcgaacaagcgttgttgagcgcacctcaggtagaaagtgcggcggtggcggtgattgatgatgtgcaggggcagcacagcgggttgctagcctgcgtgacaccgtcatctgttgatgtagctaccgtaatgcaacatctgcgtcagcaattacccacctacatgcaacccaagcagtgctgtgctatcgcccaactgccgctatcgcacaatggcaaggtggacgtgcgtcagatggtcgcaacggtccgaaacaccgcgcctgcatcgggtagcgagcggctgggagatgcggcgataaggcattcagttcgggtatgtgtggaaggggcgctggagcaaactgagtttgatgataacgaaaatctttatgtcttggggttggactccatcaagagtatccagattgcagcgcagctcaggcaccacggctggacgatgtcagcggtgcaggtgatggagtgtggaacagtgaacgccatctgtgagtttctcgctagtcatacaacggtgtcacagctcgcgcagtatgctcataacacccgtatcgatctacccgcattgcgttggtttacgcagttggcgttgccagtgcccaatgtttataaccatgtcattgtgctgaaagtgttgccgggctgcccgctcgagcagttacacaacaggctgcatacattaatccagcagcagccagctttgcacagtgcgctggacgctgaggggcggttgctagtatgcgaccctaatgtgtgttatcccaacgaggtgctcacggaatattcgacggcgcagtggacgttggcagaggtgatcgctcagtgcaatagcatgcttgacgtgacaaatggtcgagtctttacggcagctctgttacacgccccgcagcctgcgtctagcacgctggtactctgtgcccatcacttgtgcgtcgatatgcactcttggtatttaatcctcagcacgctggacgccgtcagcactgtcaatggcaccagcaacagcggactgcatcgctggaatgactatctggcgagcaaaacggtagattccgccacacacgaaagctggcgcacagtatgtcaaacgctaccgctgcatttcccacccgtgtcattgccagatgactcattacctcgtacgcgcgcttggcgtgaagactttcgccatccttgcgtcaggaggttgtttgaatccagtggtaataccgcgtacagcgccgaaacctatgtgctaacggcattggcgctggtgctgcgttactacagcgaagagccttggtgtcgcatcgagatggaaggcatgggacgcggctgctggcccgatgagcctgacgttgcggatacggtcgggtggtttactctattttatccttgggcgatccctctgcatggcgatatggcgacactgttatctgcgatcgcttccgatttggcaaaacgcacgcatggtgggggagattacggtctgctgcaaatgcggcatgcaccggaagactctcttgcgcaagggatccgaatgaattacatcggtgtgcaggcgcaaccttcactccgctattttcatattgatcactttaatagtgatatctataccgcgcctgaaaacgcgctcggctgcgtgctggagtttaatattgcgcgctcggcagctgatggtctctccttccactgccgtttcgatcccacgcgcattgcgctgaatgatgtgcaactcctgctggcgcgctacaagaacagcctcactgatctggatgcctggctgtgccaacacagcgccacgctgacgggcgcgccgactttgtggacactataa

SEQ ID NO: 308 - DNA, 2460 bp, synthetic construct
  misc_feature 1..2460: note = Synthetic: clbO
  source 1..2460: mol_type = other DNA
atggcaaaggatgattttacctgtggctcactggatattgccattattggcatgagcgggcgtttttccggtgcggaatcggtgcccgagtggtgggataagttgctggcgggtgaggagttcactcaaccgacatgcacagaagacgataatgggaacccttggattaggctgcgcaatataatcaccgctccctatgacttcgatgctgcatttttcaatattccgcctggcgaagccctgttgatggatccgcaacaacgaatatttcttgaatgctgctacaacgctcttgagcatgcgggctatattcccacgcaacttaagcgagtcggcgtctatggggcgacctacgccaataactattttatcgatcgcgtctatccatacctgaagatgagcggcgatcaccattatcttcaggcgcagattggtaacgaaaaggattacttgtgtgctcaggttgcctacaagttggggtttaccggacccgctgtgagtgtacagaccgcctgttccagttcgttggtggcagcatatttagcatgcgaggggttattgacttttcaagccgatgttgcgttggcaggtggggtcaccttgggttttttacaggcgcacggctacagtccgcaaggtgataagctggtatcgcaagacggacactgcgccccgtttagcgctgaggcgaccggcacggtatacagcagcggtgcgggagttgtggtgctaaaacgtcttgaggatgcactacgcgaccaagacagagtgtatgcggtgattaaaggaggtgcggtaaataacgacggcgggcgtcggctggggtttgtcgcccccagcgtagaaggacaggtagaagcgatcaatacggcgcttgccgccgcagaagtcgtaccgactgatatcgcactgatcgaaacgcacggtaccggtaccccgttgggtgatgagattgagcttgaagctctgcaccgggttttcgcaccggcatgtgcgccgcacagcattcaattgggtgcggtaaaagccaacttgggtcacctgggagtcgcttccggtatcgtcagcttaatgaaaaccgcactgacgctatatactggtttagttcccccgcagatcaacctagtaaataaacataaaaaactgttgcagccagcctcgccattttatctcagtgatgtagtgacttctgtcccacaaacgaaaaggatccatgccaccgttagttcgtttgggctgggagggaccaatgctcatttggttctgcaaaactggtgcgagacgcctgcgcaagcggtgcaagaaaatgagcgccgcctgtttttcttcagcgccaaaacgccattagcccttagacaacagttggatgctcattatcacgctctggcgacctatgcggaagcagataaagatcgtatcgcctatactctcgcgcaacgccgggcacatttcccgtatcgctgtgcgctggcggcagacagtgtggtagcgttgcgtgcaagtttggcgaaactgcgggatgctgacatgtcttttacccctatcaatatggaaactaccttggtgttcctatatccggaccgggacgataagctggagagtgccctgacgcatttgctggcttgtcaacctaacttgcgccagcggcaccagcgcctgtctcaggatgttgcacaaatctgcgaaccagccgattggacgccagcattgcgtcagtttatccagcaggtgagtcttagtgaatggctgatcgaacaaagtatctcgcctgtgcagcacatcggttacctgacgggggctgcagcagcgcagtatgttgcgcgcatcatctcactggaaaacgctgttcagcaggtgattgtggctgaaacgacaccggagcagacgctggccggcaacagtgaactgagcgaaatattggctaatctggcggtgacggagggaacgctgatgctggaaatcggtcgcgcggggacgttttccatattgtatcaccagcatgcgcagtgggttgggcaaacagtattttcccccatgctgaatacggatacgcctgaggacatcctgccgctgttgggcactctttggcaacgaggagtgactatttgtttaccggaaatgccagccgtgcagactataggcttacccggttattcgtttgatcgcgtccggtatgaaattcagtccagtgatgcgagagagaacgctatgttaccagtgagttatttgtcggtcagcgactttgtggagaaaacctggcgttcattgttatgcatcgatcattatgacgaacacgcggttattttcgaatacggagcgacctcgatgcacgttatctctttcgtcgacagctgtaaccacatttataaaatcggactgactgccgccgatatttatgccagacccgctatccgtgagcacagtgaatttatctccgaatgtgttgatggtattttatga

SEQ ID NO: 309 - DNA, 1515 bp, synthetic construct
  misc_feature 1..1515: note = Synthetic: clbP
  source 1..1515: mol_type = other DNA
atgacaataatggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgagtaa

SEQ ID NO: 310 - DNA, 723 bp, synthetic construct
  misc_feature 1..723: note = Synthetic: clbQ
  source 1..723: mol_type = other DNA
atgagtaatatcagtttgtattgtttgccatattcaggtggttctgccgccatgtattataaatggcgtagcgtgctgtcggacaatattactttgcggcctttagaacctgcggggaggggaactagaatacgccagccgctgtgtcttacgatggtggatgccgtcgctgacctttatcaacaatttgtgaaacactacacaggtggagactacgccatttttgggcatagtctcggagggatcatggccttcgaactggtgcattatattctcgatcatggacatgacatgccatgcgcgctgtttttttccggctgtcgcccacccgatcgggcctctcatgaagtaatactgcataccttgcccgatcaggcgtttatggaagagatcgtcaagctgggcggaactccggttgatgtctttcgtaataaagagttaatgacaattttcacccccatcattaaaaacgattatcggctctatgagcagtatgtatttcaggccaaggcgcgcacattaacctgtccgatcgtgctatttcatggcgatgctgacaatctggtaatgcaggatgaattacttgcatgggaaaaattcaccacacgaaagacgcggactattatatttcctgctgccgatcatttttttgtcgataagcattttgaacaggtggtaggttatgtgaaccagacgattgaatcactcgaaatagtagggtag

SEQ ID NO: 311 - DNA, 213 bp, synthetic construct
  misc_feature 1..213: note = Synthetic: clbR
  source 1..213: mol_type = other DNA
atggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaa

SEQ ID NO: 312 - DNA, 513 bp, synthetic construct
  misc_feature 1..513: note = Synthetic: clbS
  source 1..513: mol_type = other DNA
atggctgttccatcatcaaaagaagagttaattaaagctattaatagtaatttttctttattaaataagaagctagaatctattacgccccaactcgcctttgaacctctattggaagggcacgcgaaggggactacgattagcgtagcgaatctggtttcctatctgattggctggggagagctggtgttacactggcatgaccaagaggcaaaaggaaaaactattatttttcctgaggaaggatttaaatggaatgaattggggcgtttagcacagaaattctaccgtgactatgaggatattacagagtacgaagttttattggcacggttaaaggaaaataagcagcaactcgtggctttgattgaacgattcagtaacgacgagctttacggtaaaccttggtataataaatggacccgaggtcgtatgattcaatttaataccgcctcgccttataaaaatgcttcggggaggttaaataaactgcagaaatgtcttgcagaatag

SEQ ID NO: 313 - protein, 244 aa, synthetic construct
  REGION 1..244: note = Synthetic: clbA
MRIDILIGHTSFFHQTSRDNFLHYLNEEEIKRYDQFHFVSDKELYILSRILLKTALKRYQPDVSLQSWQFSTCKYGKPFIVFPQLAKKIFFNLSHTIDTVAVAISSHCELGVDIEQIRDLDNSYLNISQHFFTPQEATNIVSLPRYEGQLLFWKMWTLKEAYIKYRGKGLSLGLDCIEFHLTNKKLTSKYRGSPVYFSQWKICNSFLALASPLITPKITIELFPMQSQLYHHDYQLIHSSNGQN

SEQ ID NO: 314 - protein, 3206 aa, synthetic construct
  REGION 1..3206: note = Synthetic: clbB
MDNTSGDFPCNKMDTRKQLPLTPSQQGFLFHSLKDKKRSNYHEHFTCIFSQHVDSAHFKWALETLFRKHECFRTDYNWEIDERPCQVVKTDVLPDIYVLDCEQEEIRFLLANDDIIIPVPQDDGIDAIIPQLLQADLKYPFSLKTIPVRAYLIQSTKESAFILSYHHIVMDGWSLSLFIKQLLQLYGAAVVSGVRDDSAIIPSSLKPLVDTLSARRHTFQHDYWAAYLREGTPTCIVPLSQYHTDTEAENNSYVNQTNHVEINLSPDVCQKIQTLCSDYRITPAVIFYVAWGILLQRWCYADDVLFGATISGRNIPIDGIEETLGLFINTLPLRLRDDGATLLQHLQRMHQTLIAHYSNEHDALASIQRLVHKEGHAGDLFNTLVVLENYPVDMTLLSCASPVAIRHLSVHEQTHYPLTLTITQQKGFRFSIAYALNYLTNNMAQALLMHLSYLLEQLVDNPQRPIAALVNLSPCQQAQVLQPYLERMACRDWDSQSNVIEQFHQVAATSPAQVAVVDELCALTYSELAAQAEQLAAYLVQQGVMVGDTVGIISERRVNTVVAIIAIMLIGAAYVPISPDYPVGRMQEIIDDSGLALLLVHGKPLDALNVAQSDLCAFPVAPSVVFPVITPDSRAYVIYSSGSTGKPKGIAVAHRGLLRLIQGDSPLKVESGETTLLTCPFEFDVSVFEMWSTLLNHGKLVLLSKQALLDINHIRRTIADEQVARAWFTSSLFNSYVAEGADFFGMLQHITVGGEAVSAWHVNDVMQKYPHLVVTNGYGPTENTIFTTAYRFNGLQPARVPIGYAVPGTSLYITDLHGHLLPIGATGELVAGGVGVAIGYQNNPALSATVFVPDPFIPGGMMYKTGDYARLLDDGCVDCFGRKDGQIKINGQRIETGEIEQRLLECSGIIEAVVVPYRVRETLHIAAVVCVNDSYDEVEVRGQLADRLPPFAIPESLVVVTEIAKSHSGKADLAQLRYLLPATQCNAVSTTISEVHSDMEHALHAIWQRVLDRQDIDSNASFFALGGTSLDTIRVKGDIKRQLGLEIDITDLFKYPTLTALAHFLDTAVSPEDAIPTRAVVYSDMPVAIVGMAGRFPGAANIAALWTLVVGGESGLTLFSDEELRAHGVTPDTLKQANYIKTKGIVDDHEWFDADFFGYTPNEAECMDPQIRLLHQCCWQTLEHAGCDPATFTGAIGIYAGLLTSPHWLNAVMQDTTDSTALYKASILNIHSVTALIAHALNLTGPAVTLDTACSTSAVAIHQACIALRNRDCDAALAGGVSIEMPAYRGYEYHEGMINARDGVCRPFDSQASGTVTGDGLGMLLLKRLDDALADRDCIYGVIKGSAVNNDGNNKIGYTAPSVIGQSTVIRTSLRRAGFDSDSIGLVEAHGTGTVLGDPIELRALNEVFGPTPVPFCVVSALKSNIGHLNSAAGVAGVIKTTLALHHQVLPPTAHFRQLNPAIDLSRSALYVNQQVQPWPSTRPRRALVSSFGIGGTNASIALEAHQHEDDPSATGVRDSYLLLFSAKTPAALELRVASTLEYVKHGVGVRLPDVAYTLQTGRTAFDHRRAYLVSRGSKIDLSCATILQAEIFNGQRTTAEICFMFPGQGSQYHGMASALYAHQPMFRQHMDRCFAAFQRYSTVDLKALLFDDEDTRDIDQTQFTQPALFCVEYSLARTLIDLGITPDSMIGHSLGEYVAACIAGVFTLEDALHVIEARGRLMQSMRPGSMMAVYLSREQLTPWLAAERGIELAANNSAHFCVVAGEQAAISRLSTRLVEGGIQHRRLKTSHAFHSAMMTPMLHDFAQLLGQIPMHAPHKRFISNVSGTWITEEQATSPDYWVQQVRNAVLFSEGAAQLLVQPTLFIECGPGNTLSTFIQGHNQYSDQPTLLTLRKANAAIDDEHMLHRTLAALWVRGENIDWRRFNQTALGKHIPLPDYPFEQTYYYRYGAALSGYRQYPNPLRRPQDEWLQRVLWRMHDTSLREAFYAPGELIIIISADGDKLQQTLMSSGVDSITMPLPISSEDDVWDNDRILTHFHDICALLAHKTYRQLHCLYAPGAEAGSSLTQSLSGLYRVARWCMHSTTPLASLTVLTHGAFRVQEEDNPEPTLAALSGAVNVFAQELHPTEVRLIDIDAQSSDENLNLLTQRLAPKQETVMALRQGMLYLRRFIPTRLLAHLPPQTGCIPGNVLWIIGGEKGIGRMIGEALAQREGVRVVLSSRTGYHHEAVQQDALDVIHCDVTQAEAVRACLATLLERYGRLDGVIFAADATTTLTLHQLSESALRDTLTVKERGTANVLHALAQRNLLDERLLLLFCNSLAAVNAEIGQTGYATASAYLDALAQQLRTRYKVNALSIGLDALREQGMLLDAINGSEYDVLRGLRPLMTGTLLQAYKQQGADTSYYARLSPESDWLLDEHRISGIATLPGTGYLALAYEALRHYFVQDQICIDELVFLAPLTVMDNCSVDVFVDISPNGQGVSVEVKSMTERFSGTLTTHARGRATRLMVDDNVVCDLTGLMREMHTITPPTKELSSTHFHYGPRWHSVQQLYGNTAQTQVFATLALPTVAANDTIALHPALLDIASSVVEQLPGFHTDSVPFLYQDLRLYRPLPNTLHVALTVNRHDEEGDSYAFTLYDMAGEMVARCAAMVKRKVQLHIQDVDDDTRLRVPSADNYQLRLAAEGEGAGKLALCPTPRLALGDSQVEIEVLATGLNFKDVLFTTGLLRQQPGEAPLQLGLECAGRITRVGKNVTEFAPGEDVMAVLNGGFVQYARVESDCVVRKPAHCRIEQAAALPIAYLTAYYALVVRANLQPGERVLIHSAAGGVGLAALHIAKRCGAQIFATAGSEQKRDYLLSLGVHAVADSHDEQFAATLLTASDGQGMDVILNSLTGRLLDASLALLAPLGRFLELGSKDIVEDKALPMRFFAQGGTFIPINFHAAHGAFSRYLQQIVAWIDDNTLPLLPCKSVPLPEVARAFATLTTPQHIGKVVVTHRTAAGMDRLNAMIAERRLGGYALSMSNAEVMRQLWPILNTRSPWAQLLLSPRAIDRLARGNRVDRGVPSAANDTITQQTVKKRPRPEIGVPYSPATREVERVLCQILEEYLGLDRVGIDDNYAELGATSLDMVQLSGQMARHYPQVSVVSLYNHATVRQLATFCQPPEGESNAPSPQPAVQTNTRANQIAKRALQIAKNTARSHTSLH

SEQ ID NO: 315 - protein, 866 aa, synthetic construct
  REGION 1..866: note = Synthetic: clbC
MEYASEMNGMEIAIIGMAVRFPQSRTLHEFWHNIVQGKECVTFFSEEELLAEGVEQSTLDNPAYVRAKPYIEGICDFDAAFFGYSHKEAQTLDPKSRVLHEVAYHALEDAGYAQRTSDLITGVFVGASEDVDWLRRSLSQIGGDALNRFESGIYGHKDLLAHLIAYSLNLNGPVYSLYTSCSTSLSATHIACRSLLFGECDLALAGGITIDLPQKSGYFCQQGMIHSTDGHCRPFDSQASGTLFGDGAGVVVLRRLEDALAAGDRIYAVIRGSAVNNDGKQKIGFVAPGHEGQKAVICAACHLAEVSPESIGYVETHGTGTRIGDPIEFAALTEAFDTSHRQYCALGAVKANIGHTHAAAGVAGLIKTALVLHHRTIPPLANYQMPNSKLDLAHSPFYIPIQPQEWPASRMPPRAGVSSFGIGGTNVHMILEGLNPAVRDDHDQVRAPVFIPLSAPSFEQLDELTQQLTPLLATLDASTLAYTQQVARPVFDCRRVIQVENDGTQAMLASLDNLMPDAPWGLHCPDLRTTNDCTYAQWLAHSAHYQREATALTALLDGMNIPPAYCHAETWAAQANSSLLIRGCQTIAALKTWMNLLPTLTLLSGAGTGLLPAAAASGMIATQDVLHLLWEMEQKALHLWLPERHEPIPGYVLAWQGNPITDAQRNDRGFWSEALLADTRELGEGVHSINWVRLPPEIREDVDVLRYVAQLWCAGINVDWAVWYGTPLPQRGSASAYPFAHNHYPLPGRVMGSVETQPEAGPETHHPYQARPVLSVPFVAAHSRGMQYITGLMELLLEISPVGVDDDFFELGGHSLLVTQLTSRLERDFNVHIDLLTLMENPNPRNIYAHIAAQLGGEDNLEIACQ

SEQ ID NO: 316 - protein, 289 aa, synthetic construct
  REGION 1..289: note = Synthetic: clbD
MMNVAVIGAGVMGTGVAHNMAQYGISTNVVDISQSQLDKCRQMIEANLRLYNFHPQHKKKTHSTAEIMENIRFTTELDDIVECDLVIENITEDIEKKNALYTRMNTICGASTVFGVNTSAISITALSKLMRHPENVVGVHFMNPVPLMHTVELIRGVHTAERTLNIFHHLFAQLNKTGIVVNDSPGFVTNRAMMIFVNEAIFMVQEQIARAEDIDTLFKTCFGHKMGPLQTADLIGLDTILQSLQVLYESFNDDKYRPSFLLKKMVDAGYLGVKSGQGFYRYQQTYAEQ

SEQ ID NO: 317 - protein, 82 aa, synthetic construct
  REGION 1..82: note = Synthetic: clbE
MKKQDMKAAIREFLSRSLRGHTLNDDDDIFSLGLVHSLFTVQIILFIEKNFQVELEVSELKTEQIATVNKIVELIQRQTGLE

SEQ ID NO: 318 - protein, 376 aa, synthetic construct
  REGION 1..376: note = Synthetic: clbF
MCTENYELAQQEAVLFAKQHLALAAQNIERQQFIVPDIISCVAQAGYLGASIPQKYGGRGYDSYQLCALHEVMAGVHGSLENLITVTGMVSTLLQRVGSAAQKAHYLPKLATGELIGAIALTEPNIGSDLVNVETELQQDGDGWRLNGKKKWITLGQIADFFIVLIHCGNQLATVLIDRNTDGFTITPLNDMLGLRGNMLAELHFNDCRLKEDALLGPLTPGVPLAVNFALNEGRFTTACGSLGLCRAAVDVAARYIRQRKQFKRRLFSHGIVQHLFATMLTQTRSAQLMCFSAAEYRETLHPAMINQILMAKYVASKAAVDVAGKAVQLLGANGCHADYAVERYYRDAKIMEIIEGTSQIHEIQIAMNYMMGSEA

SEQ ID NO: 319 - protein, 422 aa, synthetic construct
  REGION 1..422: note = Synthetic: clbG
MTKDVALMFPGSGSQYVGMARWLYERYPQVRTLFDEASQITERDMAALCLSGTLVQLAEPTAMALAIYTTSVAHFVAWQQFLAQTGVHVNLRYMLGHSLGEYAALTCSGALSFSQALALVAMRSRLASEIAREMDASTTIIKQGNQALVAAACEVAERETRQQVGIACFNSPQQFMLSGQNSAIIAAEQYLLDHDRQVEVVPLIGGVPYHSPLLKPCGQQLRKALDRCEWRRPCCPVISNVNAQPYPDTVTPVQWLEQQLSQPVQWQRSLTYLTGHLSPIAIEIGPQSVLKNLLLENRYPAPVYAFDNRHDRAQLALVLGDNMAVKTDPEAVRRQRITLLTNALTATRHHRAADVAASAQLKELLSRFFERIQQIEQRGTSSEEDIAFLHELLEQGFQLKGSSQAEIDACHARLASDNGGQA

SEQ ID NO: 320 - protein, 2310 aa, synthetic construct
  REGION 1..2310: note = Synthetic: clbH
MEQQGIMRQLPTDDQTIVDYLYRIAGEYGEKAAVLMGDAALSYHDLNARSNQLAHYLRGLGIGEDRVVAIRLPRGMAMLIAIFAIVKAGGAYLPLAYNAPRSRIENILSNSGAVCLIGTDDGDRWPIPRVEIDSAAVSAMPTTDLRYRPHARQLAYIIYTSGSTGVPKGVATEHAALLNRIVWMQNAYPISSQDVLFQKTVYTFDVSVWEMFWWAMYGASVVLLPSGLESDPRTLARLIQRHRVSVVHFVPSMLNLFVEYLEMKQDPRLTASLRLVFSSGEKLTVHSVARFYQSVAQGDLINLYGPTEAAIDVSHHRCLRGYDYDDIPIGQAIDGCRLYVLDDHGNPVADGEEGELYLAGIGLARGYLNNVALTDRCFTIHPTLRHLGKPERLYKTGDLVWRDGESQQIHYIGRNDFQIKIRGLRVELGEIEAHAMRFPGVQQAVVVADQDDPDNQLIYAFVVSSVPLNLAALMDALSKNLPAYMLPNRLLAMSELPLSDNGKCCRKTLLDLARAYSASRVDLRETPAVRYLPLSSAQSSMWFMQQLAPHTALYNNPTALLLEGELDRTRMDGAIRQLMSRHTLLRAMAETHNGQPVLAVPQCVSSQALLTIVPLPSVSDDNALQAMINQRAAHPMPLTSGTPLCRFELLTLDDDRSVLLIHLHHIISDGWSKGVLLRELQAAYNGESLTPEPLLEYADYMEYQEEWRQSDAYQDAMRYWQNTLAGTLPILDIPTDQPRQKVARYQGAFVAFALSANTCERVLAAARAQRVSLYNYLLTAFVLLLHRNARQQEYIVGMPIAARLTKEQEHMIAPLVNVLPLRLPLDEAASFSELVQTIRGILFAAFRHQRLEFTDIVRAVNVDRSAGTPCALLPDMQAYLLFTSGSTGEPKGVCVVHRGLLNLLLDMQRTFAVGSQDRLLSVTTPTFDISFLEYLLPLISGASLYLTEAERAADSFRMIPLIADYRPTLMQATPSFWHGLLMAGWRGDPELCVLAGGEALPTKVAEELLRCCGSLWNLYGPTETTIWSLKSQITQAENITLGAPIANTRIYILDNEGHPVPQGVDGELYIAGDGVAQGYDGQPELNAQFFLSEPGVPGGRMFRTGDLVRSDAQGQLFFVGRKDSQIKLRGYRIELGEIERTLARHPHVDAAVVACIERAPLHKALAAFIITSEPPSLFEQLKNELRQQLPDYMVPTLWQRVADFPNTDNGKIDRKRLAENFVADSSLVSPQTQALSDTEQMLLALWMRYLPIKNVDPECDFFRLGGHSLLAVTLVAEINRTFHCALTLKDIFHYSTLRALSARIAQQSITDAAASQDDWVIVHDPEHRHQPFPLTDVQRAYWLGRQTGATSIATHIYHEFDVEHFNVTRFTHAVNALIARHEMLRARVLPDGTQQILAQVPAYQLEQRDLSALSPNARNDALMAIRDRLSHHVHPADRWPLFDFSYSACTAQHGRLHFSLDLLIADALSMRTLQQELMMLYREPHVSLPLLPFSFRDYVQALLVEQASEAYARDQAYWQRALPQLYGPPTLPVQGDLAQLSAIRFVRRRHRLSAHNWGVLSALAQRTRITKTALLLTVFHFPIYQCMFQLDNMPLASPTLNGVNVTPLLLDTSASQVDISLSMQHIDGRITGTFEYDAGLYSADRIQHLVAQWRELLDEASSQPTQLVRDLIRFTPREHAWLARHNATEVALPPVDNLLALVLPHCQQRPTQVALRHADDAMTYGELQQATMQMCTWLRAQGVKRGESVALQLPFCFELIIAQLAILSLGASYVPLDGNAPAARNALILAQATPCMLLVAQPLESPHGLTIPWVLVPDWRSLLTEIPNLPVSVAPDALDCDAVVIFTSGTTGQPKGVRLSQRNLVNLTASFISSYQVTHQDVLLPITSVASASFVGEVLPLLAAGGTLVLAQKAQSLDSDALIALLASQRVTILSTTPSLSASLSVLAQSMGSLRLFLCGGEALEYEQIAPLLPHMAVVNGYGLTESGICSTYFPVAKRREQETGALPIGRPIQNTQAYVVDAYNRLVPPGACGELCFSGLGISPGYLDARQDPERFVELPEYPGVRVLKTGDRARWATDGMLFYLGRQDRQVQIRGYRVELGDIESLLKQHPDIADAWVDVRRNAAATPLLVAFYCSVNGVALDAQQLRVWLSLRLPLHMLPLLYVPLSAMPLGVNGKIDPQCLPLVDLRQLEGPGEYVPPATELEQRLAEIWQQLLGLERVGTTTNFFDLGGHSLLLVQMQQYIGQQCGQHVALVDLLRFTTIKRLAEFLLAPDAAQGTTGDQTQLRAAKQRLAFGHTRWAATTDSHH

SEQ ID NO: 321 - protein, 1010 aa, synthetic construct
  REGION 1..1010: note = Synthetic: clbI
MAENDFGIAIIGMAGRFPQADTVQAFWENLLASRECISFYSDEELLAMGISPEFVQHPDYVKAKGEVADIDKFDAAFFGIAPREAELMDPQHRVLLETAWAAFEDAGYVAADYPGDVGIFAGKSMDSYLMLNLMPHFKRVFSSGSLQAAIGNDKDSITTTIAYHLNLRGPAITVQTSSSTSLVAVCVACQSLLTWQCDMAIAGGVTLGPPAKTGYLSQEGGITAADGHCRAFSDNSSGFVPGTGAGLVVLKRVDEALRDGDNIYAVIKGFAVNNDGSEKISYTAPSVDAQARAIAQAQRLAGLTPQDITYVEAHGTGTRLGDPVEFSALSQAFAGASQKQYCALGSVKTNIGHLDTAAGVAGLIKTALAVQQGIIPATLHFERPNAQIDLTNSPFYINTTCQPWQPESGIRRAGVTSLGMGGTNAHVVLEQAPAVDLQARAPVPAYSILPFSAKTDSALSSGLARFADFLQHESLPDRRDLAWTLSQGRKAFAHRAALVTRDLHAAGTLLQQAATAPFARGVAQTQLGLGLLFSGQGSQYQRMGHQLYQVWPAYADAFDRCATLLEREYQLDIRHELFRAEVSLAQGERLAQTCLTQPLLFSVEYALAQLWLSWGITPTVMIGHSLGEWVAATLAGVFSLEDALRLVARRAELMHQAPSGAMLMVALPEAQIRALITAPLAIAAVNAPDYSVIAGPTSEILAVSQRLTEQNIINKRLHTSHAFHSSMMQDAAQALRQAFENVRLNPPTLTIISTVTGAHVSADTLTTPDYWIEQMLMPVQFSAALQEAQATFDVDFLEIGPGATLTQLTNGHALGDRLAFSSLPAGARSSDEHKHILDTVAALWVRGHNIDLSAFAGEQPRRVSLPTYAFDKIRYWVDSPEEQRSAVTPVADAGSVIPSEPSVRRQPRPAFSVPYAAPESKTQRGLVAICEALLGIDGLGIDDNFFEAGGHSLMLGMLLAQVQERFAVTLSFFDVMEDASVRALAQLVEQEQQDDGGSALAVLVNDMINE

SEQ ID NO: 322 - protein, 1454 aa, synthetic construct
  REGION 1..1454: note = Synthetic: clbJ
MTIHHAALARMLPAEKKEKLLRQLAQSGVSPSRIPIIKADPAQAIPLSFNQERLWFLQKYDSTATNYNLYVVYRLHGVVDMPMLTEALRHVQARHAILRTRIIVRNDRPCQVIDDASSLVLDTVTLAAQAPTSALDAVIQQVINTRFDLARGPLWGVTQIIQPDQGCHLVFCAHHIIIDGISLRLLFDELQQQYARLHAGNETSLPPPPLQYADYAFWQREWFQDTLLANELAYWRARLQDAPLLSTFPSLHPRPAQPSTHGSRFSITLDETLSLALKHVARTQETTPFVLMLTAFQLVLMRYAQQQRLVIGMPVSGRIRPELQSSIGYYASTAVIYTDFNGVEVGREALQRVKASVKETQGRQQLPFENLVNMLDLPRSLSHSPLFQILYIYHNHVTPRAFTLAGAYWEQVTYHNQTVKYDMTVEVFQNDATFDVSFEYDLGLYDADVVKQIAEALRQHCLSLTSSLETPIGAIPLHAPETATPRRDPLNATNVPWLGPQDVLRIIEQRCVQHPKQLAIQQHDGTLTYAELWARVQFIAMRFRAHGIQPGDRIGVLLPRHRDVIATMLATWFVGACYVPFDIHQPAARLQRLMQRARLVCLVVRQPGEWGEIVQLSLPELMQDMSNAIRYSSQVLARWSLSPTFTLNLTLFNRPQGYPNAEAVIGDFTAVSLLNVCYDSQHSYAHNAQRIQVQLWEDLEHRRFSGIRASEALIHSGRFHAPMPVVFTSMLDIDGETTAQDPRDTTRFTLCPDANITQTPQVWLDHQVIELAGELHFNWDAVEQLFDTTLLDQMFGAYCHALQALVAMPQSWWGVNSSLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEVKSHLEQLDSVGSAAVVCHQGQLYAFITAAENLHPDDTDALLARVRAQLAVQLPYYLLPQHFFLLKVLPMTGNGKIDQAAMVQEVIQRMSQSTSQKSRALAHASPYEQQVAALWCEVLQREQIGLNDNFFEAGGGSIQIVLLHRRIEEIFKVTVPIAELFRLTTVKRIAGYLQAMQDNARAVNQTQQRDASRSRAQQRLVRRHQRQR

SEQ ID NO: 323 - protein, 2154 aa, synthetic construct
  REGION 1..2154: note = Synthetic: clbK
MTYSESDIAIVGMNCRYPGVHSVAAFETVLRTGCNILDPKVTPSNGHNHITLNNVYEHMAEFDANFFGYSRAEAEIMDPQQRVFLTCAWEMFEQSGYNPKQHDARVGLYAGVSTSFYLLTHLMNNPDKLAQLGGLQIMVGNDKDHLTSQLAYRLNITGPCVTVQASCATSLVAVHLACEGLLSGQCDMALAGGVTFRMEEQRSYESHGDGLQAEDGLIHTFDAQASGTVYSSGLGMVLLKRATDAQVQGDNILAVIKGSAINNDGGARSGYTVPGVDGQEAVMIEAHSLAEVTPQQIQYLELHGSGTPLGDAIEFAAIKRVFGTPAPNATPWRLGAVKPNVGHVEMASGITSLIKTVLSLTNRVFYPTLNFQRANPQLGLEDSPFEVVSRLTPWPEGTTPRTAGVSAFGLGGTNAHLVVQAPLSTPQARAQQMGPCVVVLSAKNHNALEQMQNALLAKLAAHPEIRLQDVAYTLRHGRFSAPVRKCVIAENCTQLARQLRDAPMVEATTGCTIYWRLGHRFVVALETLSDWLACSEVLSQAVGQLLEHFPLEPACLQDLSPAQRTFISQYALIALIDERETLNVVLCGDGDGGYAAAVLRGDCTLEQAWHRLNAGQPFDDVPTNPLLQPDVCSLMLDDAASDANRTALEALGQLWLAGVSLDWRWVDAAERMLGSQRIALPGTVFTPQRYWVEAVRPATFSHESSNNLLSRATKSDIIAVVTEIWERTLGVSIDDHHASFFELGGHSLLASTILYDIQQRYGITCTLSAFFADPTIEGLSCYLLEQGGSETAVSALPDTVFAPDQQHLPFPLTDVQQAYWVGRRKSLGLGNISTHIYVEYELQGLDETAFNRALNAVIARHSMLRAIVNDDGMQQILPNVPEYHVAFYTTQCEDAFQQRCRELRDTLSHQMIDCSRWPLFQMEVVVDPQQKARLHVSIDLLIADAWSLELFIRELAYHYRHPQAALPTLTYSFRDYVLTLKSYEKTPQFERARDYWRARIETLPPGPRLPLRTDPTKLENPTFVRRSYCLSRAIWQRLKTQAGQMSITPTTLLLTGFAQVLARFSSSPHFSLNLTLFNRLPLHADINHLIGDFTALTLLEIDMSQGETLQARANVIHSQLWRDLDNRLFGGIQVSRLLVQTHRDPAKSVIPIVFTSLLNQYEASWETDDTLFNQPQDDLYSISQTPQVWLDHQVMERNGELHFNWDVVEQLFEPALMDQMFQCYCQLLHALAQRPQLWHETQDVLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEIERCIARHPDVEQSVVVAVGNSQHRRLVAFAKLHDRHQAQALQAKEAEAAALAQGIIVNPAQRLAFKLKEPHIRALDGLGIALTAPADSTRYIKRRSYRHFSAQKTTLAQLGQLLSGLGQMRLPGLPFAKYAYASAGGLYPVQTYVYLHPDKIEEGVSGIYYFDPRQSCLMPVAPEVELNSGFHAGPNQSIADRAAFTLFMVADMAVISPFYGQEAAWHFSVMEAGTLCHLLEEDAPRYGLGLCQLGMADFSAVASHFQLSPHHRYVHCTVGGAIGQEAASAAALLRDFSTYEKPKETAAPLDMQSYKDAMLRGLRQQLPDYMVPSDLMLATDFPLTANGKLDRQKLQLQGEQIAHQRDGVGPIQVDSALQQRLVALWQEVLGVSHVSAEDDFFSLGGSSIELVRIQQALEAIIGQEIPIVDLFRLPTIADVARYLDEQLHNLPAAHDIVLAQAEVSQVSAARENLALRRKRAQQGEKGDE

SEQ ID NO: 324 - protein, 487 aa, synthetic construct
  REGION 1..487: note = Synthetic: clbL
MSEQSYRSAGTLLAQLASGETTSVALVNHYFSRMAQFNKPLNAVVQQHYALALEAAARADRERLEGRARGVLHGLPCTVKESFDVQGWLTTSGAHYLKDNRATQDAPSIARLRAAGAILMGKTNVPMMTADWQTYNDLYGTTHNLWDRQRSPGGSSGGAAVAVAADFTPVEFGSDLFGSLRIPAHYTGVYAHRCSLGLMSVRGHVPGGGPQATDEPDLSTAGPMARSAADLRLMMRALSTFWVEPPRIPDFSRYQAKANYRVCTWFSAPHHEIDQQIAQRFQSFIDKLRAQPGVEVDDAMPADIDPDALFDIAVKLSGRLVSTALNGRQRLTAGLAALGFRLVGKLADVPEGITSYYQGMLKDSGEQRNTDKLRHEYSRVIETLFARYDVLLTPVSPVLAFAHMQQPVRKRKLIVNGEPQDYNEHLFWNMLATVFGLPATVYPLAKTMDELPCGIQIISGHFHDDVTINFAEFCESISGGFTVPEGY

SEQ ID NO: 325 - protein, 479 aa, synthetic construct
  REGION 1..479: note = Synthetic: clbM
MTVNKLEPDNGTPNDELFTVAGMFDGSLYKLLLRMALPMFVGMLTQVTYAIADIFWLSHIDVTNSGIIAGVGLVFPVGMGLFAIANGIQIGMGSLLSRAIGMQRLDRAQRILSVGIIIALFFAIVITVLGYVYAQPLLRSLGATKSIIGYATEFYYYSLLTVFSIMLIGVMMGLFQGAGKIMVIMKASLLGALVNIMLDPIMIFVFDFGVKGVALASFLAQLSMVAYFIYTLMGLHIGLSIRIALRPFSWKIYREFLSVGMAQMLMQLIIAVGIVIYNFFIVRLDVNAMAAFTLTGRIDYFIITPMLAIATALLTVVGQNWGHGNVTRTLNAYWAAVALAFSIVLVLAVMHIVLAPWMYPLFTRVVAVSDYAVLQTRIMALALPFVAISLLASEYYQAIGKPWYSVLLTLMRHVFISVPVVYLLAIVLEMRITGVYFGAMSGTFVAALLAWRLLRLSPRLLRWNQEAVRSQHLDMEVAP

SEQ ID NO: 326 - protein, 1455 aa, synthetic construct
  REGION 1..1455: note = Synthetic: clbN
MMSGNPLSWPQEQCHIIDQLYPYSAVNIIGGVVTIEGIVDLPRLHAAIQSAIRQFDALRMWFVMGEESEVVSQVQPYHWRDIRHLTFSPDYDKENLRPAAIETFVDEWFRQPFTLLAHDLFEFVTFTCGEQYSGYLFKAHHGIADGWSMALLSNHVKRAYEQQDVPDDASPAYSAFLAQQQSYQASTRFAVDRGWWRDYIDEYRDCFPDSSPIVTTEGISCSTWLEPAMINRLYRLCNRYGCTLNTLFIALFALYRARVWGEEKGVIGVPLANRHTREARRCFGMFTNQLPLAYRLVRTERFCERVAFFQRELKRGFKHSKYPITLFNQDLAEQGGGKLRAFDYCVNYYNFTYERHIAGAAQRVESYYSGEQSYKLQIVLQTVNNHKESLRLSLEALRSAFTPHQLTAMKNGLLDLVTALDRQPDARLGDLEVYPAPHVALACGSLKPSFTSRFAAQVVEHGDRTALIDNEQSLTYRQLDDAVERVARYLRQQGIGRGQVVGIIAEHSAQTVMVIYGILRCGAAFLPLNPALPTTRLYAMCRKAQVAHILYDPAMHELTQALAFPASSLLQALATSALAREPWPAIEPQDLAYVLFTSGSTGEPKGVQVSHGNLANYLHFAAERYFTAQDRAALYSSLSFDLTITTLFAPLCVGASISVCRHAESETLLRMAVVDQPNTVIKLTPAHLRLLCAAGISSEQIRTLVVGGEDFKRDLARKAAALFPQAVIYNEYGPTEATVGCMIYRYTGQETLPSLPIGMAIDGCQVAICSPWGCPVPEGETGELVIYGASVTQGYIDAPQQTAAAYLKDTNGVMIGYRSGDIGYAIAPNTLVYQGRKDDQVKINGYRIELCEIEQALLSAPQVESAAVAVIDDVQGQHSGLLACVTPSSVDVATVMQHLRQQLPTYMQPKQCCAIAQLPLSHNGKVDVRQMVATVRNTAPASGSERLGDAAIRHSVRVCVEGALEQTEFDDNENLYVLGLDSIKSIQIAAQLRHHGWTMSAVQVMECGTVNAICEFLASHTTVSQLAQYAHNTRIDLPALRWFTQLALPVPNVYNHVIVLKVLPGCPLEQLHNRLHTLIQQQPALHSALDAEGRLLVCDPNVCYPNEVLTEYSTAQWTLAEVIAQCNSMLDVTNGRVFTAALLHAPQPASSTLVLCAHHLCVDMHSWYLILSTLDAVSTVNGTSNSGLHRWNDYLASKTVDSATHESWRTVCQTLPLHFPPVSLPDDSLPRTRAWREDFRHPCVRRLFESSGNTAYSAETYVLTALALVLRYYSEEPWCRIEMEGMGRGCWPDEPDVADTVGWFTLFYPWAIPLHGDMATLLSAIASDLAKRTHGGGDYGLLQMRHAPEDSLAQGIRMNYIGVQAQPSLRYFHIDHFNSDIYTAPENALGCVLEFNIARSAADGLSFHCRFDPTRIALNDVQLLLARYKNSLTDLDAWLCQHSATLTGAPTLWTL

SEQ ID NO: 327 - protein, 819 aa, synthetic construct
  REGION 1..819: note = Synthetic: clbO
MAKDDFTCGSLDIAIIGMSGRFSGAESVPEWWDKLLAGEEFTQPTCTEDDNGNPWIRLRNIITAPYDFDAAFFNIPPGEALLMDPQQRIFLECCYNALEHAGYIPTQLKRVGVYGATYANNYFIDRVYPYLKMSGDHHYLQAQIGNEKDYLCAQVAYKLGFTGPAVSVQTACSSSLVAAYLACEGLLTFQADVALAGGVTLGFLQAHGYSPQGDKLVSQDGHCAPFSAEATGTVYSSGAGVVVLKRLEDALRDQDRVYAVIKGGAVNNDGGRRLGFVAPSVEGQVEAINTALAAAEVVPTDIALIETHGTGTPLGDEIELEALHRVFAPACAPHSIQLGAVKANLGHLGVASGIVSLMKTALTLYTGLVPPQINLVNKHKKLLQPASPFYLSDVVTSVPQTKRIHATVSSFGLGGTNAHLVLQNWCETPAQAVQENERRLFFFSAKTPLALRQQLDAHYHALATYAEADKDRIAYTLAQRRAHFPYRCALAADSVVALRASLAKLRDADMSFTPINMETTLVFLYPDRDDKLESALTHLLACQPNLRQRHQRLSQDVAQICEPADWTPALRQFIQQVSLSEWLIEQSISPVQHIGYLTGAAAAQYVARIISLENAVQQVIVAETTPEQTLAGNSELSEILANLAVTEGTLMLEIGRAGTFSILYHQHAQWVGQTVFSPMLNTDTPEDILPLLGTLWQRGVTICLPEMPAVQTIGLPGYSFDRVRYEIQSSDARENAMLPVSYLSVSDFVEKTWRSLLCIDHYDEHAVIFEYGATSMHVISFVDSCNHIYKIGLTAADIYARPAIREHSEFISECVDGIL

SEQ ID NO: 328 - protein, 504 aa, synthetic construct
  REGION 1..504: note = Synthetic: clbP
MTIMEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 329 - protein, 240 aa, synthetic construct
  REGION 1..240: note = Synthetic: clbQ
MSNISLYCLPYSGGSAAMYYKWRSVLSDNITLRPLEPAGRGTRIRQPLCLTMVDAVADLYQQFVKHYTGGDYAIFGHSLGGIMAFELVHYILDHGHDMPCALFFSGCRPPDRASHEVILHTLPDQAFMEEIVKLGGTPVDVFRNKELMTIFTPIIKNDYRLYEQYVFQAKARTLTCPIVLFHGDADNLVMQDELLAWEKFTTRKTRTIIFPAADHFFVDKHFEQVVGYVNQTIESLEIVG

SEQ ID NO: 330 - protein, 70 aa, synthetic construct
  REGION 1..70: note = Synthetic: clbR
MDKFKEKNPLSLRERQVLRMLAQGDEYSQISHNLNISINTVKFHVKNIKHKIQARNTNHAIHIANRNEII

SEQ ID NO: 331 - protein, 170 aa, synthetic construct
  REGION 1..170: note = Synthetic: clbS
MAVPSSKEELIKAINSNFSLLNKKLESITPQLAFEPLLEGHAKGTTISVANLVSYLIGWGELVLHWHDQEAKGKTIIFPEEGFKWNELGRLAQKFYRDYEDITEYEVLLARLKENKQQLVALIERFSNDELYGKPWYNKWTRGRMIQFNTASPYKNASGRLNKLQKCLAE

SEQ ID NO: 332 - DNA, 17 bp, synthetic construct
  misc_feature 1..17: note = Synthetic: Operator 1
  source 1..17: mol_type = other DNA
taacaccgtgcgtgttg

SEQ ID NO: 333 - DNA, 17 bp, synthetic construct
  misc_feature 1..17: note = Synthetic: Operator 2
  source 1..17: mol_type = other DNA
tacctctggcggtgata

SEQ ID NO: 334 - DNA, 169 bp, synthetic construct
  misc_feature 1..169: note = Synthetic: FNR responsive promoter
  source 1..169: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgc

SEQ ID NO: 335-499 - skipped (165 entries)

SEQ ID NO: 500 - protein, 490 aa, synthetic construct
  REGION 1..490: note = Synthetic: Tryptophan Decarboxylase (EC 4.1.1.28) Chain A, Ruminococcus gnavus Tryptophan
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 501 - DNA, 18 bp, synthetic construct
  misc_feature 1..18: note = Synthetic: Synthetic consensus sequence
  source 1..18: mol_type = other DNA
ttgttgayryrtcaacwa

SEQ ID NO: 502 - DNA, 15 bp, synthetic construct
  misc_feature 1..15: note = Synthetic: Synthetic consensus sequence
  source 1..15: mol_type = other DNA
ttataatnattataa

SEQ ID NO: 503 - skipped

SEQ ID NO: 504 - DNA, 1251 bp, synthetic construct
  misc_feature 1..1251: note = Synthetic: Pseudomonas fluorescens kynureninase, codon optimized for expression in E. coli
  source 1..1251: mol_type = other DNA
atgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 505 - DNA, 1354 bp, synthetic construct
  misc_feature 1..1354: note = Synthetic: Pseudomonas fluorescens kynureninase, codon optimized for expression in E. coli driven by a Tet inducible promoter
  source 1..1354: mol_type = other DNA
taattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 506 - DNA, 1985 bp, synthetic construct
  misc_feature 1..1985: note = Synthetic: Pseudomonas fluorescens kynureninase, codon optimized for expression in E. coli driven by a Tet inducible promoter with RBS and tetR in reverse orientation
  source 1..1985: mol_type = other DNA
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaaggag

SEQ ID NO: 507 - DNA, 1398 bp, synthetic construct
  misc_feature 1..1398: note = Synthetic: Human kynureninase codon optimized for expression in E. coli
  source 1..1398: mol_type = other DNA
atggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 508 - DNA, 1501 bp, synthetic construct
  misc_feature 1..1501: note = Synthetic: Human kynureninase, codon optimized for expression in E. coli driven by a Tet inducible promoter
  source 1..1501: mol_type = other DNA
taattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 509 - DNA, 2127 bp, synthetic construct
  misc_feature 1..2127: note = Synthetic: Human kynureninase codon optimized for expression in E. coli driven by a Tet inducible promoter with RBS and tetR in reverse orientation
  source 1..2127: mol_type = other DNA
taagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 510-511 - skipped (2 entries)

SEQ ID NO: 512 - DNA, 94 bp, synthetic construct
  misc_feature 1..94: note = Synthetic: SYN23119 promoter with RBS
  source 1..94: mol_type = other DNA
ggaaaatttttttaaaaaaaaaacttgacagctagctcagtccttggtataatgctagcacgaagtgaattatataaaagtgggaggtgcccga

SEQ ID NO: 513 - DNA, 951 bp, Escherichia coli
  misc_feature 1..951: note = DNA Escherichia coli clbA
  source 1..951: mol_type = genomic DNA
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagatttttttacctttccatactatagatacagtagccgtgctattagttctcactgcgagcttggtgtcgatattgaacaaatagagatttagacaactctatctgaatatcagtcagcattttttactccacaggaagctactacatagtttcacttcctcgttatgaaggtcaattactttttggaaatgtggacgctcaaagagcttacatcaatatcgaggtaaggcctatctttaggactggattgtattgaatttcatttaacaaataaaaactaactcaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 514 - DNA, 1225 bp, synthetic construct
  misc_feature 1..1225: note = Synthetic: Pseudomonas fluorescens kynureninase, codon optimized for expression in E. coli, driven by the SYN23119
  source 1..1225: mol_type = other DNA
ggaaaatttttttaaaaaaaaaacttgacagctagctcagtccttggtataatgctagcacgaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggt

SEQ ID NO: 515 - DNA, 1347 bp, Escherichia coli
  misc_feature 1..1347: note = Pseudomonas fluorescens kynureninase driven by Lpp promoter from E. coli
  source 1..1347: mol_type = genomic DNA
ataagtgccttcccatcaaaaaaatattctcaacataaaaaactttgtgtaatacttgtaacgctagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 516 - DNA, 2372 bp, Clostridium sporogenes
  misc_feature 1..2372: note = fbrAroG-Tdc (tdc from Clostridium sporogenes)
  source 1..2372: mol_type = genomic DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgaaattttggcgcaagtatacgcaacaggagatggatgagaaaatcacagaatcgcttgagaagacattaaattacgataacacgaaaaccatcggcatcccaggtactaagctggatgatactgtattttatgacgatcactccttcgttaagcactctccctatttacgtacgttcatccaaaaccctaatcacattggttgtcacacgtacgataaagcagacatcttgtttggcggcacgtttgacatcgaacgcgaactgattcagcttttggccatcgatgtcttaaacggaaatgatgaggaattcgatggatatgtgacacaggggggaaccgaggcgaatattcaggcaatgtgggtttatcgtaactatttcaaaaaagaacgtaaagcaaaacatgaggaaatcgcaatcatcacgagcgcggatacccattacagtgcatataaggggagcgacttgctgaacattgatattatcaaggtcccagtagacttctattcgcgtaagatccaggagaacacgttagactcgattgtcaaggaggcgaaggaaattggaaagaagtacttcattgtcatctcaaacatgggtacgactatgtttggcagtgtagacgaccctgatctttatgctaacatttttgataagtataacttagaatacaaaatccacgtcgatggagcttttgggggtttcatttatcctatcgataataaggagtgcaaaacagatttctcgaacaagaacgtctcatccatcacgcttgacggtcacaaaatgcttcaagccccctatgggactggtatcttcgtgtcacgtaagaacttgatccataacaccctgacaaaggaagcaacgtatattgaaaacctggacgttaccctgagtgggtcccgctccggatccaacgccgttgcgatctggatggttttagcctcttatggcccctacgggtggatggagaagattaacaagttgcgcaatcgcactaagtggctttgcaagcagcttaacgacatgcgcatcaaatactataaggaggatagcatgaatatcgtcacgattgaagagcaatacgtaaataaagagattgcagagaaatacttccttgtgcctgaagtacacaatcctaccaacaattggtacaagattgtagtcatggaacatgttgaacttgacatcttgaactcccttgtttatgatttacgtaaattcaacaaggagcacctgaaggcaatgtga

SEQ ID NO: 517 - DNA, 1254 bp, Clostridium sporogenes
  misc_feature 1..1254: note = Tdc (tdc from Clostridium sporogenes)
  source 1..1254: mol_type = genomic DNA
atgaaattttggcgcaagtatacgcaacaggagatggatgagaaaatcacagaatcgcttgagaagacattaaattacgataacacgaaaaccatcggcatcccaggtactaagctggatgatactgtattttatgacgatcactccttcgttaagcactctccctatttacgtacgttcatccaaaaccctaatcacattggttgtcacacgtacgataaagcagacatcttgtttggcggcacgtttgacatcgaacgcgaactgattcagcttttggccatcgatgtcttaaacggaaatgatgaggaattcgatggatatgtgacacaggggggaaccgaggcgaatattcaggcaatgtgggtttatcgtaactatttcaaaaaagaacgtaaagcaaaacatgaggaaatcgcaatcatcacgagcgcggatacccattacagtgcatataaggggagcgacttgctgaacattgatattatcaaggtcccagtagacttctattcgcgtaagatccaggagaacacgttagactcgattgtcaaggaggcgaaggaaattggaaagaagtacttcattgtcatctcaaacatgggtacgactatgtttggcagtgtagacgaccctgatctttatgctaacatttttgataagtataacttagaatacaaaatccacgtcgatggagcttttgggggtttcatttatcctatcgataataaggagtgcaaaacagatttctcgaacaagaacgtctcatccatcacgcttgacggtcacaaaatgcttcaagccccctatgggactggtatcttcgtgtcacgtaagaacttgatccataacaccctgacaaaggaagcaacgtatattgaaaacctggacgttaccctgagtgggtcccgctccggatccaacgccgttgcgatctggatggttttagcctcttatggcccctacgggtggatggagaagattaacaagttgcgcaatcgcactaagtggctttgcaagcagcttaacgacatgcgcatcaaatactataaggaggatagcatgaatatcgtcacgattgaagagcaatacgtaaataaagagattgcagagaaatacttccttgtgcctgaagtacacaatcctaccaacaattggtacaagattgtagtcatggaacatgttgaacttgacatcttgaactcccttgtttatgatttacgtaaattcaacaaggagcacctgaaggcaatgtga

SEQ ID NO: 518 - protein, 417 aa, Clostridium sporogenes
  REGION 1..417: note = misc_feature - TDC: Tryptophan decarboxylase from Clostridium sporogenes
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 519 - DNA, 60 bp, synthetic construct
  misc_feature 1..60: note = Synthetic: SR36 sequence
  source 1..60: mol_type = other DNA
tagaactgatgcaaaaagtgctcgacgaaggcacacagatgtgtaggctggagctgcttc

SEQ ID NO: 520 - DNA, 60 bp, synthetic construct
  misc_feature 1..60: note = Synthetic: SR38 sequence
  source 1..60: mol_type = other DNA
gtttcgtaattagatagccaccggcgctttaatgcccggacatatgaatatcctccttag

SEQ ID NO: 521 - DNA, 52 bp, synthetic construct
  misc_feature 1..52: note = Synthetic: SR33 sequence
  source 1..52: mol_type = other DNA
caacacgtttcctgaggaaccatgaaacagtatttagaactgatgcaaaaag

SEQ ID NO: 522 - DNA, 46 bp, synthetic construct
  misc_feature 1..46: note = Synthetic: SR34 sequence
  source 1..46: mol_type = other DNA
cgcacactggcgtcggctctggcaggatgtttcgtaattagatagc

SEQ ID NO: 523 - DNA, 1096 bp, synthetic construct
  misc_feature 1..1096: note = Synthetic: fbrAroG sequence
  source 1..1096: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaa

SEQ ID NO: 524 - DNA, 1053 bp, synthetic construct
  misc_feature 1..1053: note = Synthetic: fbrAroG sequence
  source 1..1053: mol_type = other DNA
atgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaa

SEQ ID NO: 525 - DNA, 2351 bp, synthetic construct
  misc_feature 1..2351: note = Synthetic: fbr-AroG-serA sequence
  source 1..2351: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaatcaggttgatctggatgcggcggcaaagcgcgggatcccggtatttaacgcaccgttctcaaatacgcgctctgttgcggagctggtgattggcgaactgctgctgctattgcgcggcgtgccagaagccaatgctaaagcgcatcgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaacaaactgccgctgggcaacgccactcaggtacagcatctttctgacctgctgaatatgagcgatgtggtgagtctgcatgtaccagagaatccgtccaccaaaaatatgatgggcgcgaaagagatttcgctaatgaagcccggctcgctgctgattaatgcttcgcgcggtactgtggtggatattccagcgctgtgtgacgcgctggcgagcaaacatctggcgggggcggcaatcgacgtattcccgacggaaccggcgaccaatagcgatccatttacctctccgctgtgtgaattcgacaatgtccttctgacgccacacattggcggttcgactcaggaagcgcaggagaatatcggcttggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctcaacaaaatttttgccgagcagggcgtcaacatcgccgcgcaatatctacaaacttccgcccagatgggttatgtagttattgatattgaagccgacgaagacgttgccgaaaaagcgctgcaggcaatgaaagctattccgggtaccattcgcgcccgtctgctgtactaa

SEQ ID NO: 526 - DNA, 1233 bp, synthetic construct
  misc_feature 1..1233: note = Synthetic: SerA sequence
  source 1..1233: mol_type = other DNA
atggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaatcaggttgatctggatgcggcggcaaagcgcgggatcccggtatttaacgcaccgttctcaaatacgcgctctgttgcggagctggtgattggcgaactgctgctgctattgcgcggcgtgccagaagccaatgctaaagcgcatcgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaacaaactgccgctgggcaacgccactcaggtacagcatctttctgacctgctgaatatgagcgatgtggtgagtctgcatgtaccagagaatccgtccaccaaaaatatgatgggcgcgaaagagatttcgctaatgaagcccggctcgctgctgattaatgcttcgcgcggtactgtggtggatattccagcgctgtgtgacgcgctggcgagcaaacatctggcgggggcggcaatcgacgtattcccgacggaaccggcgaccaatagcgatccatttacctctccgctgtgtgaattcgacaatgtccttctgacgccacacattggcggttcgactcaggaagcgcaggagaatatcggcttggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctcaacaaaatttttgccgagcagggcgtcaacatcgccgcgcaatatctacaaacttccgcccagatgggttatgtagttattgatattgaagccgacgaagacgttgccgaaaaagcgctgcaggcaatgaaagctattccgggtaccattcgcgcccgtctgctgtactaa

SEQ ID NO: 527 - DNA, 2621 bp, Catharanthus roseus
  misc_feature 1..2621: note = fbrAroG-Tdc (tdc from C. roseus)
  source 1..2621: mol_type = genomic DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgggttctattgactcgacgaatgtggccatgtctaattctcctgttggcgagtttaagccccttgaagcagaagagttccgtaaacaggcacaccgcatggtggattttattgcggattattacaagaacgtagaaacatacccggtcctttccgaggttgaacccggctatctgcgcaaacgtattcccgaaaccgcaccatacctgccggagccacttgatgatattatgaaggatattcaaaaggacattatccccggaatgacgaactggatgtccccgaacttttacgccttcttcccggccacagttagctcagcagctttcttgggggaaatgctttcaacggcccttaacagcgtaggatttacctgggtcagttccccggcagcgactgaattagagatgatcgttatggattggcttgcgcaaattttgaaacttccaaaaagctttatgttctccggaaccgggggtggtgtcatccaaaacactacgtcagagtcgatcttgtgcactattatcgcggcccgtgaacgcgccttggaaaaattgggccctgattcaattggtaagcttgtctgctatgggtccgatcaaacgcacacaatgtttccgaaaacctgtaagttagcaggaatttatccgaataatatccgccttatccctaccacggtagaaaccgactttggcatctcaccgcaggtacttcgcaagatggtcgaagacgacgtcgctgcggggtacgttcccttatttttgtgtgccaccttgggaacgacatcaactacggcaacagatcctgtagattcgctgtccgaaatcgcaaacgagtttggtatctggattcatgtcgacgccgcatatgctggatcggcttgcatctgcccagaatttcgtcactaccttgatggcatcgaacgtgtggattccttatcgctgtctccccacaaatggcttttagcatatctggattgcacgtgcttgtgggtaaaacaacctcacctgctgcttcgcgctttaacgactaatcccgaatacttgaagaataaacagagtgatttagataaggtcgtggattttaagaactggcagatcgcaacaggacgtaagttccgctctttaaaactttggttaattctgcgttcctacggggtagttaacctgcaaagtcatatccgtagtgatgtagcgatggggaagatgtttgaggaatgggtccgttccgatagccgctttgaaatcgtcgtgccacgtaatttttcgcttgtatgctttcgcttgaaaccggatgtatctagtttacatgtcgaggaggtcaacaagaagttgttggatatgcttaactccaccggtcgcgtatatatgacgcatacaattgttggcggaatctatatgttacgtttggctgtaggtagcagcttgacagaggaacatcacgtgcgccgcgtttgggacttgatccagaagcttacggacgacctgcttaaagaggcgtga

SEQ ID NO: 528 - DNA, 1503 bp, Catharanthus roseus
  misc_feature 1..1503: note = Tdc (tdc from C. roseus)
  source 1..1503: mol_type = genomic DNA
atgggttctattgactcgacgaatgtggccatgtctaattctcctgttggcgagtttaagccccttgaagcagaagagttccgtaaacaggcacaccgcatggtggattttattgcggattattacaagaacgtagaaacatacccggtcctttccgaggttgaacccggctatctgcgcaaacgtattcccgaaaccgcaccatacctgccggagccacttgatgatattatgaaggatattcaaaaggacattatccccggaatgacgaactggatgtccccgaacttttacgccttcttcccggccacagttagctcagcagctttcttgggggaaatgctttcaacggcccttaacagcgtaggatttacctgggtcagttccccggcagcgactgaattagagatgatcgttatggattggcttgcgcaaattttgaaacttccaaaaagctttatgttctccggaaccgggggtggtgtcatccaaaacactacgtcagagtcgatcttgtgcactattatcgcggcccgtgaacgcgccttggaaaaattgggccctgattcaattggtaagcttgtctgctatgggtccgatcaaacgcacacaatgtttccgaaaacctgtaagttagcaggaatttatccgaataatatccgccttatccctaccacggtagaaaccgactttggcatctcaccgcaggtacttcgcaagatggtcgaagacgacgtcgctgcggggtacgttcccttatttttgtgtgccaccttgggaacgacatcaactacggcaacagatcctgtagattcgctgtccgaaatcgcaaacgagtttggtatctggattcatgtcgacgccgcatatgctggatcggcttgcatctgcccagaatttcgtcactaccttgatggcatcgaacgtgtggattccttatcgctgtctccccacaaatggcttttagcatatctggattgcacgtgcttgtgggtaaaacaacctcacctgctgcttcgcgctttaacgactaatcccgaatacttgaagaataaacagagtgatttagataaggtcgtggattttaagaactggcagatcgcaacaggacgtaagttccgctctttaaaactttggttaattctgcgttcctacggggtagttaacctgcaaagtcatatccgtagtgatgtagcgatggggaagatgtttgaggaatgggtccgttccgatagccgctttgaaatcgtcgtgccacgtaatttttcgcttgtatgctttcgcttgaaaccggatgtatctagtttacatgtcgaggaggtcaacaagaagttgttggatatgcttaactccaccggtcgcgtatatatgacgcatacaattgttggcggaatctatatgttacgtttggctgtaggtagcagcttgacagaggaacatcacgtgcgccgcgtttgggacttgatccagaagcttacggacgacctgcttaaagaggcgtga

SEQ ID NO: 529 - DNA, 5377 bp, synthetic construct
  misc_feature 1..5377: note = Synthetic: fbrAroG-trpDH-ipdC-iad1 sequence
  source 1..5377: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatgcgtacaccctactgtgtcgccgattatcttttagatcgtctgacggactgcggggccgatcacctgtttggcgtaccgggcgattacaacttgcagtttctggaccacgtcattgactcaccagatatctgctgggtagggtgtgcgaacgagcttaacgcgagctacgctgctgacggatatgcgcgttgtaaaggctttgctgcacttcttactaccttcggggtcggtgagttatcggcgatgaacggtatcgcaggctcgtacgctgagcacgtcccggtattacacattgtgggagctccgggtaccgcagctcaacagcgcggagaactgttacaccacacgctgggcgacggagaattccgccacttttaccatatgtccgagccaattactgtagcccaggctgtacttacagagcaaaatgcctgttacgagatcgaccgtgttttgaccacgatgcttcgcgagcgccgtcccgggtatttgatgctgccagccgatgttgccaaaaaagctgcgacgcccccagtgaatgccctgacgcataaacaagctcatgccgattccgcctgtttaaaggcttttcgcgatgcagctgaaaataaattagccatgtcgaaacgcaccgccttgttggcggactttctggtcctgcgccatggccttaaacacgcccttcagaaatgggtcaaagaagtcccgatggcccacgctacgatgcttatgggtaaggggatttttgatgaacgtcaagcgggattttatggaacttattccggttcggcgagtacgggggcggtaaaggaagcgattgagggagccgacacagttctttgcgtggggacacgtttcaccgatacactgaccgctggattcacacaccaacttactccggcacaaacgattgaggtgcaaccccatgcggctcgcgtgggggatgtatggtttacgggcattccaatgaatcaagccattgagactcttgtcgagctgtgcaaacagcacgtccacgcaggactgatgagttcgagctctggggcgattccttttccacaaccagatggtagtttaactcaagaaaacttctggcgcacattgcaaacctttatccgcccaggtgatatcatcttagcagaccagggtacttcagcctttggagcaattgacctgcgcttaccagcagacgtgaactttattgtgcagccgctgtgggggtctattggttatactttagctgcggccttcggagcgcagacagcgtgtccaaaccgtcgtgtgatcgtattgacaggagatggagcagcgcagttgaccattcaggagttaggctcgatgttacgcgataagcagcaccccattatcctggtcctgaacaatgaggggtatacagttgaacgcgccattcatggtgcggaacaacgctacaatgacatcgctttatggaattggacgcacatcccccaagccttatcgttagatccccaatcggaatgttggcgtgtgtctgaagcagagcaactggctgatgttctggaaaaagttgctcatcatgaacgcctgtcgttgatcgaggtaatgttgcccaaggccgatatccctccgttactgggagccttgaccaaggctttagaagcctgcaacaacgcttaaaggttaagaaggagatatacatatgcccaccttgaacttggacttacccaacggtattaagagcacgattcaggcagaccttttcatcaataataagtttgtgccggcgcttgatgggaaaacgttcgcaactattaatccgtctacggggaaagagatcggacaggtggcagaggcttcggcgaaggatgtggatcttgcagttaaggccgcgcgtgaggcgtttgaaactacttggggggaaaacacgccaggtgatgctcgtggccgtttactgattaagcttgctgagttggtggaagcgaatattgatgagttagcggcaattgaatcactggacaatgggaaagcgttctctattgctaagtcattcgacgtagctgctgtggccgcaaacttacgttactacggcggttgggctgataaaaaccacggtaaagtcatggaggtagacacaaagcgcctgaactatacccgccacgagccgatcggggtttgcggacaaatcattccgtggaatttcccgcttttgatgtttgcatggaagctgggtcccgctttagccacagggaacacaattgtgttaaagactgccgagcagactcccttaagtgctatcaagatgtgtgaattaatcgtagaagccggctttccgcccggagtagttaatgtgatctcgggattcggaccggtggcgggggccgcgatctcgcaacacatggacatcgataagattgcctttacaggatcgacattggttggccgcaacattatgaaggcagctgcgtcgactaacttaaaaaaggttacacttgagttaggaggaaaatccccgaatatcattttcaaagatgccgaccttgaccaagctgttcgctggagcgccttcggtatcatgtttaaccacggacaatgctgctgcgctggatcgcgcgtatatgtggaagaatccatctatgacgccttcatggaaaaaatgactgcgcattgtaaggcgcttcaagttggagatcctttcagcgcgaacaccttccaaggaccacaagtctcgcagttacaatacgaccgtatcatggaatacatcgaatcagggaaaaaagatgcaaatcttgctttaggcggcgttcgcaaagggaatgaggggtatttcattgagccaactatttttacagacgtgccgcacgacgcgaagattgccaaagaggagatcttcggtccagtggttgttgtgtcgaaatttaaggacgaaaaagatctgatccgtatcgcaaatgattctatttatggtttagctgcggcagtcttttcccgcgacatcagccgcgcgatcgagacagcacacaaactgaaagcaggcacggtctgggtcaactgctataatcagcttattccgcaggtgccattcggagggtataaggcttccggtatcggccgtgagttgggggaatatgccttgtctaattacacaaatatcaaggccgtccacgttaacctttctcaaccggcgcccatttga

SEQ ID NO: 530 - DNA, 1080 bp, synthetic construct
  misc_feature 1..1080: note = Synthetic: trpDH sequence
  source 1..1080: mol_type = other DNA
taagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtga

SEQ ID NO: 531 - DNA, 1674 bp, synthetic construct
  misc_feature 1..1674: note = Synthetic: ipdC sequence
  source 1..1674: mol_type = other DNA
gaaggagatatacatatgcgtacaccctactgtgtcgccgattatcttttagatcgtctgacggactgcggggccgatcacctgtttggcgtaccgggcgattacaacttgcagtttctggaccacgtcattgactcaccagatatctgctgggtagggtgtgcgaacgagcttaacgcgagctacgctgctgacggatatgcgcgttgtaaaggctttgctgcacttcttactaccttcggggtcggtgagttatcggcgatgaacggtatcgcaggctcgtacgctgagcacgtcccggtattacacattgtgggagctccgggtaccgcagctcaacagcgcggagaactgttacaccacacgctgggcgacggagaattccgccacttttaccatatgtccgagccaattactgtagcccaggctgtacttacagagcaaaatgcctgttacgagatcgaccgtgttttgaccacgatgcttcgcgagcgccgtcccgggtatttgatgctgccagccgatgttgccaaaaaagctgcgacgcccccagtgaatgccctgacgcataaacaagctcatgccgattccgcctgtttaaaggcttttcgcgatgcagctgaaaataaattagccatgtcgaaacgcaccgccttgttggcggactttctggtcctgcgccatggccttaaacacgcccttcagaaatgggtcaaagaagtcccgatggcccacgctacgatgcttatgggtaaggggatttttgatgaacgtcaagcgggattttatggaacttattccggttcggcgagtacgggggcggtaaaggaagcgattgagggagccgacacagttctttgcgtggggacacgtttcaccgatacactgaccgctggattcacacaccaacttactccggcacaaacgattgaggtgcaaccccatgcggctcgcgtgggggatgtatggtttacgggcattccaatgaatcaagccattgagactcttgtcgagctgtgcaaacagcacgtccacgcaggactgatgagttcgagctctggggcgattccttttccacaaccagatggtagtttaactcaagaaaacttctggcgcacattgcaaacctttatccgcccaggtgatatcatcttagcagaccagggtacttcagcctttggagcaattgacctgcgcttaccagcagacgtgaactttattgtgcagccgctgtgggggtctattggttatactttagctgcggccttcggagcgcagacagcgtgtccaaaccgtcgtgtgatcgtattgacaggagatggagcagcgcagttgaccattcaggagttaggctcgatgttacgcgataagcagcaccccattatcctggtcctgaacaatgaggggtatacagttgaacgcgccattcatggtgcggaacaacgctacaatgacatcgctttatggaattggacgcacatcccccaagccttatcgttagatccccaatcggaatgttggcgtgtgtctgaagcagagcaactggctgatgttctggaaaaagttgctcatcatgaacgcctgtcgttgatcgaggtaatgttgcccaaggccgatatccctccgttactgggagccttgaccaaggctttagaagcctgcaacaacgcttaa

SEQ ID NO: 532 - DNA, 1509 bp, synthetic construct
  misc_feature 1..1509: note = Synthetic: Iad1 sequence
  source 1..1509: mol_type = other DNA
gaaggagatatacatatgcccaccttgaacttggacttacccaacggtattaagagcacgattcaggcagaccttttcatcaataataagtttgtgccggcgcttgatgggaaaacgttcgcaactattaatccgtctacggggaaagagatcggacaggtggcagaggcttcggcgaaggatgtggatcttgcagttaaggccgcgcgtgaggcgtttgaaactacttggggggaaaacacgccaggtgatgctcgtggccgtttactgattaagcttgctgagttggtggaagcgaatattgatgagttagcggcaattgaatcactggacaatgggaaagcgttctctattgctaagtcattcgacgtagctgctgtggccgcaaacttacgttactacggcggttgggctgataaaaaccacggtaaagtcatggaggtagacacaaagcgcctgaactatacccgccacgagccgatcggggtttgcggacaaatcattccgtggaatttcccgcttttgatgtttgcatggaagctgggtcccgctttagccacagggaacacaattgtgttaaagactgccgagcagactcccttaagtgctatcaagatgtgtgaattaatcgtagaagccggctttccgcccggagtagttaatgtgatctcgggattcggaccggtggcgggggccgcgatctcgcaacacatggacatcgataagattgcctttacaggatcgacattggttggccgcaacattatgaaggcagctgcgtcgactaacttaaaaaaggttacacttgagttaggaggaaaatccccgaatatcattttcaaagatgccgaccttgaccaagctgttcgctggagcgccttcggtatcatgtttaaccacggacaatgctgctgcgctggatcgcgcgtatatgtggaagaatccatctatgacgccttcatggaaaaaatgactgcgcattgtaaggcgcttcaagttggagatcctttcagcgcgaacaccttccaaggaccacaagtctcgcagttacaatacgaccgtatcatggaatacatcgaatcagggaaaaaagatgcaaatcttgctttaggcggcgttcgcaaagggaatgaggggtatttcattgagccaactatttttacagacgtgccgcacgacgcgaagattgccaaagaggagatcttcggtccagtggttgttgtgtcgaaatttaaggacgaaaaagatctgatccgtatcgcaaatgattctatttatggtttagctgcggcagtcttttcccgcgacatcagccgcgcgatcgagacagcacacaaactgaaagcaggcacggtctgggtcaactgctataatcagcttattccgcaggtgccattcggagggtataaggcttccggtatcggccgtgagttgggggaatatgccttgtctaattacacaaatatcaaggccgtccacgttaacctttctcaaccggcgcccatttga

SEQ ID NO: 533 - DNA, 6573 bp, synthetic construct
  misc_feature 1..6573: note = Synthetic: TrpEDCBA sequence
  source 1..6573: mol_type = other DNA
ctctagaaataattttgtttaactttaagaaggagatatacatatgcaaacacaaaaaccgactctcgaactgctaacctgcgaaggcgcttatcgcgacaacccgactgcgctttttcaccagttgtgtggggatcgtccggcaacgctgctgctggaatccgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttctaatggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagtta

SEQ ID NO: 534 - DNA, 1562 bp, synthetic construct
  misc_feature 1..1562: note = Synthetic: trpE sequence
  source 1..1562: mol_type = other DNA
atgcaaacacaaaaaccgactctcgaactgctaacctgcgaaggcgcttatcgcgacaacccgactgcgctttttcaccagttgtgtggggatcgtccggcaacgctgctgctggaatccgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttcta

SEQ ID NO: 535 - DNA, 1596 bp, synthetic construct
  misc_feature 1..1596: note = Synthetic: trpD sequence
  source 1..1596: mol_type = other DNA
atggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaa

SEQ ID NO: 536 - DNA, 355 bp, synthetic construct
  misc_feature 1..355: note = Synthetic: katG sequence
  source 1..355: mol_type = other DNA
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 537 - DNA, 228 bp, synthetic construct
  misc_feature 1..228: note = Synthetic: dps sequence
  source 1..228: mol_type = other DNA
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 538 - DNA, 334 bp, synthetic construct
  misc_feature 1..334: note = Synthetic: ahpC sequence
  source 1..334: mol_type = other DNA
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 539 - DNA, 134 bp, synthetic construct
  misc_feature 1..134: note = Synthetic: oxyS sequence
  source 1..134: mol_type = other DNA
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 540 - DNA, 36 bp, synthetic construct
  misc_feature 1..36: note = Synthetic: SR43 sequence
  source 1..36: mol_type = other DNA
atatcgtcgcagcccacagcaacacgtttcctgagg

SEQ ID NO: 541 - DNA, 47 bp, synthetic construct
  misc_feature 1..47: note = Synthetic: SR44 sequence
  source 1..47: mol_type = other DNA
aagaatttaacggagggcaaaaaaaaccgacgcacactggcgtcggc

SEQ ID NO: 542 - DNA, 1406 bp, synthetic construct
  misc_feature 1..1406: note = Synthetic: trpC sequence
  source 1..1406: mol_type = other DNA
atgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaaagaatttaacggagggcaaaaaaaaccgacgcacactggcgtcggc

SEQ ID NO: 543 - DNA, 1193 bp, synthetic construct
  misc_feature 1..1193: note = Synthetic: trpB sequence
  source 1..1193: mol_type = other DNA
atgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctg

SEQ ID NO: 544 - DNA, 807 bp, synthetic construct
  misc_feature 1..807: note = Synthetic: trpA sequence
  source 1..807: mol_type = other DNA
atggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 545 - DNA, 6574 bp, synthetic construct
  misc_feature 1..6574: note = Synthetic: fbrS40FTrpE-DCBA sequence
  source 1..6574: mol_type = other DNA
ctctagaaataattttgtttaactttaagaaggagatatacatatgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttctaatggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 546 - DNA, 1562 bp, synthetic construct
  misc_feature 1..1562: note = Synthetic: fbrTrpE sequence
  source 1..1562: mol_type = other DNA
atgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttcta

SEQ ID NO: 547 - DNA, 7615 bp, synthetic construct
  misc_feature 1..7615: note = Synthetic: fbrAroG-trpDH-fldABCDacuIfldH sequence
  source 1..7615: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatgaaggttaagaaggagatatacatatgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtgagctttaagaaggagatatacatatgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttgacgcttaagaaggagatatacatatgcgtgctgtcttaatcgagaagtcagatgacacccagagtgtttcagttacggagttggctgaagaccaattacccgaaggtgacgtccttgtggatgtcgcgtacagcacattgaattacaaggatgctcttgcgattactggaaaagcacccgttgtacgccgttttcctatggtccccggaattgactttactgggactgtcgcacagagttcccatgctgatttcaagccaggcgaccgcgtaattctgaacggatggggagttggtgagaaacactggggcggtcttgcagaacgcgcacgcgtacgtggggactggcttgtcccgttgccagcccccttagacttgcgccaggctgcaatgattggcactgcggggtacacagctatgctgtgcgtgcttgcccttgagcgccatggagtcgtacctgggaacggcgagattgtcgtctcaggcgcagcaggaggggtaggttctgtagcaaccacactgttagcagccaaaggctacgaagtggccgccgtgaccgggcgcgcaagcgaggccgaatatttacgcggattaggcgccgcgtcggtcattgatcgcaatgaattaacggggaaggtgcgtccattagggcaggaacgctgggcaggaggaatcgatgtagcaggatcaaccgtacttgctaatatgttgagcatgatgaaataccgtggcgtggtggcggcctgtggcctggcggctggaatggacttgcccgcgtctgtcgccccttttattctgcgtggtatgactttggcaggggtagattcagtcatgtgccccaaaactgatcgtctggctgcttgggcacgcctggcatccgacctggaccctgcaaagctggaagagatgacaactgaattaccgttctctgaggtgattgaaacggctccgaagttcttggatggaacagtgcgtgggcgtattgtcattccggtaacaccttgatacttaagaaggagatatacatatgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagatt

SEQ ID NO: 548 - DNA, 1239 bp, synthetic construct
  misc_feature 1..1239: note = Synthetic: fldA sequence
  source 1..1239: mol_type = other DNA
atggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatga

SEQ ID NO: 549 - DNA, 1224 bp, synthetic construct
  misc_feature 1..1224: note = Synthetic: fldB sequence
  source 1..1224: mol_type = other DNA
atgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtga

SEQ ID NO: 550 - DNA, 1124 bp, synthetic construct
  misc_feature 1..1124: note = Synthetic: fldC sequence
  source 1..1124: mol_type = other DNA
atgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttg

SEQ ID NO: 551 - DNA, 981 bp, synthetic construct
  misc_feature 1..981: note = Synthetic: Acul sequence
  source 1..981: mol_type = other DNA
atgcgtgctgtcttaatcgagaagtcagatgacacccagagtgtttcagttacggagttggctgaagaccaattacccgaaggtgacgtccttgtggatgtcgcgtacagcacattgaattacaaggatgctcttgcgattactggaaaagcacccgttgtacgccgttttcctatggtccccggaattgactttactgggactgtcgcacagagttcccatgctgatttcaagccaggcgaccgcgtaattctgaacggatggggagttggtgagaaacactggggcggtcttgcagaacgcgcacgcgtacgtggggactggcttgtcccgttgccagcccccttagacttgcgccaggctgcaatgattggcactgcggggtacacagctatgctgtgcgtgcttgcccttgagcgccatggagtcgtacctgggaacggcgagattgtcgtctcaggcgcagcaggaggggtaggttctgtagcaaccacactgttagcagccaaaggctacgaagtggccgccgtgaccgggcgcgcaagcgaggccgaatatttacgcggattaggcgccgcgtcggtcattgatcgcaatgaattaacggggaaggtgcgtccattagggcaggaacgctgggcaggaggaatcgatgtagcaggatcaaccgtacttgctaatatgttgagcatgatgaaataccgtggcgtggtggcggcctgtggcctggcggctggaatggacttgcccgcgtctgtcgccccttttattctgcgtggtatgactttggcaggggtagattcagtcatgtgccccaaaactgatcgtctggctgcttgggcacgcctggcatccgacctggaccctgcaaagctggaagagatgacaactgaattaccgttctctgaggtgattgaaacggctccgaagttcttggatggaacagtgcgtgggcgtattgtcattccggtaacaccttga

SEQ ID NO: 552 - DNA, 996 bp, synthetic construct
  misc_feature 1..996: note = Synthetic: fldH1 sequence
  source 1..996: mol_type = other DNA
atgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagattgcgggcgcggctctggatagctatgagtatgagcaaggtgtctttcacaacaataagatgaatgaaattatgcaggatgataccttggaacgtctgaaatcttttcccaacgtcgtgatcacgccgcatttgggtttttatactgatgaggcggtttccaatatggtagagatcacactgatgaaccttcaggaattcgagttgaaaggaacctgtaagaaccagcgtgtttgtaaatga

SEQ ID NO: 553 - DNA, 8008 bp, synthetic construct
  misc_feature 1..8008: note = Synthetic: fbrAroG-TrpDH-fldABCDH sequence
  source 1..8008: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatgaaggttaagaaggagatatacatatgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtgagctttaagaaggagatatacatatgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttgacgcttaagaaggagatatacatatgttctttacggagcaacacgaacttattcgcaaactggcgcgtgactttgccgaacaggaaatcgagcctatcgcagacgaagtagataaaaccgcagagttcccaaaagaaatcgtgaagaagatggctcaaaatggatttttcggcattaaaatgcctaaagaatacggaggggcgggtgcggataaccgcgcttatgtcactattatggaggaaatttcacgtgcttccggggtagcgggtatctacctgagctcgccgaacagtttgttaggaactcccttcttattggtcggaaccgatgagcaaaaagaaaagtaccttaagcctatgatccgcggcgagaagactctggcgttcgccctgacagagcctggtgctggctctgatgcgggtgcgttggctactactgcccgtgaagagggcgactattatatcttaaatggccgcaagacgtttattacaggggctcctattagcgacaatattattgtgttcgcaaaaaccgatatgagcaaagggaccaaaggtatcaccactttcattgtggactcaaagcaggaaggggtaagttttggtaagccagaggacaaaatgggaatgattggttgtccgacaagcgacatcatcttggaaaacgttaaagttcataagtccgacatcttgggagaagtcaataaggggtttattaccgcgatgaaaacactttccgttggtcgtatcggagtggcgtcacaggcgcttggaattgcacaggccgccgtagatgaggcggtaaagtacgccaagcaacgtaaacaattcaatcgcccaatcgcgaaatttcaggccattcaatttaaacttgccaatatggagactaaattaaatgccgctaaacttcttgtttataacgcagcgtacaaaatggattgtggagaaaaagccgacaaggaagcctctatggctaaatactttgctgctgaatcagcgatccaaatcgttaacgacgcgctgcaaatccatggcgggtatggctatatcaaagactacaagattgaacgtttgtaccgcgatgtgcgtgtgatcgctatttatgagggcacttccgaggtccaacagatggttatcgcgtccaatctgctgaagtaatacttaagaaggagatatacatatgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagattgcgggcgcggctctggatagctatgagtatgagcaaggtgtctttcacaacaataagatgaatgaaattatgcaggatgataccttggaacgtctgaaatcttttcccaacgtcgtgatcacgccgcatttgggtttttatactgatgaggcggtttccaatatggtagagatcacactgatgaaccttcaggaattcgagttgaaaggaacctgtaagaaccagcgtgtttgtaaatga

SEQ ID NO: 554 - DNA, 1134 bp, synthetic construct
  misc_feature 1..1134: note = Synthetic: FldD
  source 1..1134: mol_type = other DNA
atgttctttacggagcaacacgaacttattcgcaaactggcgcgtgactttgccgaacaggaaatcgagcctatcgcagacgaagtagataaaaccgcagagttcccaaaagaaatcgtgaagaagatggctcaaaatggatttttcggcattaaaatgcctaaagaatacggaggggcgggtgcggataaccgcgcttatgtcactattatggaggaaatttcacgtgcttccggggtagcgggtatctacctgagctcgccgaacagtttgttaggaactcccttcttattggtcggaaccgatgagcaaaaagaaaagtaccttaagcctatgatccgcggcgagaagactctggcgttcgccctgacagagcctggtgctggctctgatgcgggtgcgttggctactactgcccgtgaagagggcgactattatatcttaaatggccgcaagacgtttattacaggggctcctattagcgacaatattattgtgttcgcaaaaaccgatatgagcaaagggaccaaaggtatcaccactttcattgtggactcaaagcaggaaggggtaagttttggtaagccagaggacaaaatgggaatgattggttgtccgacaagcgacatcatcttggaaaacgttaaagttcataagtccgacatcttgggagaagtcaataaggggtttattaccgcgatgaaaacactttccgttggtcgtatcggagtggcgtcacaggcgcttggaattgcacaggccgccgtagatgaggcggtaaagtacgccaagcaacgtaaacaattcaatcgcccaatcgcgaaatttcaggccattcaatttaaacttgccaatatggagactaaattaaatgccgctaaacttcttgtttataacgcagcgtacaaaatggattgtggagaaaaagccgacaaggaagcctctatggctaaatactttgctgctgaatcagcgatccaaatcgttaacgacgcgctgcaaatccatggcgggtatggctatatcaaagactacaagattgaacgtttgtaccgcgatgtgcgtgtgatcgctatttatgagggcacttccgaggtccaacagatggttatcgcgtccaatctgctgaagtaa

SEQ ID NO: 555 - DNA, 1470 bp, synthetic construct
  misc_feature 1..1470: note = Synthetic: Tryptophan Decarboxylase (EC 4.1.1.28) Chain A, Ruminococcus gnavus Tryptophan Decarboxylase Rumgna_01526 (alpha-fmt); codon optimized for the expression in E. coli
  source 1..1470: mol_type = other DNA
atgagtcaagtgattaagaagaaacgtaacacctttatgatcggaacggagtacattcttaacagtacacaattggaggaagcgattaaatcattcgtacatgatttctgcgcagagaagcatgagatccatgatcaacctgtggtagtagaagctaaagaacatcaggaggacaaaatcaaacaaatcaaaatcccggaaaagggacgtcctgtaaatgaagtcgtttctgagatgatgaatgaagtgtatcgctaccgcggagacgccaaccatcctcgctttttttcttttgtgcccggacctgcaagcagtgtgtcgtggttgggggatattatgacgtccgcctacaatattcatgctggaggctcaaagctggcaccgatggttaactgcattgagcaggaagttctgaagtggttagcaaagcaagtggggttcacagaaaatccaggtggcgtatttgtgtcgggcggttcaatggcgaatattacggcacttactgcggctcgtgacaataaactgaccgacattaaccttcatttgggaactgcttatattagtgaccagactcatagttcagttgcgaaaggattacgcattattggaatcactgacagtcgcatccgtcgcattcccactaactcccacttccagatggataccaccaagctggaggaagccatcgagaccgacaagaagtctggctacattccgttcgtcgttatcggaacagcaggtaccaccaacactggttcgattgaccccctgacagaaatctctgcgttatgtaagaagcatgacatgtggtttcatatcgacggagcgtatggagctagtgttctgctgtcacctaagtacaagagccttcttaccggaaccggcttggctgacagtatttcgtgggatgctcataaatggttgttccaaacgtacggctgtgcaatggtacttgtcaaagatatccgtaatttattccactcttttcatgtgaatcccgagtatcttaaggatctggaaaacgacatcgataacgttaatacatgggacatcggcatggagctgacgcgccctgcacgcggtcttaaattgtggcttactttacaggtccttggatctgacttgattgggagtgccattgaacacggtttccagctggcagtttgggctgaggaagcattgaatccaaagaaagactgggagatcgtttctccagctcagatggctatgattaatttccgttatgcccctaaggatttaaccaaagaggaacaggatattctgaatgaaaagatctcccaccgcattttagagagcggatacgctgcaattttcactactgtattaaacggcaagaccgttttacgcatctgtgcaattcacccggaggcaactcaagaggatatgcaacacacaatcgacttattagaccaatacggtcgtgaaatctataccgagatgaagaaagcg

SEQ ID NO: 556 - protein, 444 aa, synthetic construct
  REGION 1..444: note = Synthetic: Trp aminotransferase (EC 2.6.1.27); tryptophan aminotransferase[Cryptococcus deuterogattii R265]
MTATTISIETVPQAPAAGTKTNGTSGKYNPRTYLSDRAKVTEIDGSDAGRPNPDTFPFNSITLNLKPPLGLPESSNNMPVSITIEDPDLATALQYAPSAGIPKLREWLADLQAHVHERPRGDYAISVGSGSQDLMFKGFQAVLNPGDPVLLETPMYSGVLPALRILKADYAEVDVDDQGLSAKNLEKVLSEWPADKKRPRVLYTSPIGSNPSGCSASKERKLEVLKVCKKYDVLIFEDDPYYYLAQELIPSYFALEKQVYPEGGHVVRFDSFSKLLSAGMRLGFATGPKEILHAIDVSTAGANLHTSAVSQGVALRLMQYWGIEGFLAHGRAVAKLYTERRAQFEATAHKYLDGLATWVSPVAGMFLWIDLRPAGIEDSYELIRHEALAKGVLGVPGMAFYPTGRKSSHVRVSFSIVDLEDESDLGFQRLAEAIKDKRKALGLA

SEQ ID NO: 557 - DNA, 1332 bp, synthetic construct
  misc_feature 1..1332: note = Synthetic: Trp aminotransferase (EC 2.6.1.27); tryptophan aminotransferase [Cryptococcus deuterogattii R265], codon optimized for expression in E. coli
  source 1..1332: mol_type = other DNA
atgacggcaactacaatttctattgagaccgtacctcaggccccggcggcggggaccaaaactaatgggacttcaggaaaatacaacccccgcacttacctgtccgaccgcgccaaagtcactgagattgatggatctgacgccggtcgccccaatcccgatactttcccatttaactcgattaccttaaatttgaaaccacctttaggcttgcccgagagttcaaataacatgccggtctctatcacgattgaagaccccgatttagcgacggccttacaatatgcacctagcgccggtattcctaagctgcgcgaatggctggctgacttacaagctcacgttcatgagcgcccccgtggcgattatgccatctcggtcgggtcggggtcacaggatttgatgtttaagggcttccaagctgtcttgaatccaggtgatccagtccttctggaaaccccaatgtattcaggtgttctgccagcgctgcgcattctgaaggcggattatgcagaagttgatgtagacgaccaggggttatctgctaaaaaccttgaaaaagttttatcagagtggcccgcagataagaagcgtcctcgtgtcctgtatacgtcgccaatcggctccaatccttccggatgttcagcatccaaggaacgcaagttagaggtactgaaagtctgtaagaagtacgatgtgctgatcttcgaagacgatccgtattattaccttgctcaagagcttattccatcctattttgcgttggaaaaacaagtttatccggagggtgggcacgttgtacgctttgactcatttagtaaattgctttctgctgggatgcgcttgggatttgctacagggccgaaggaaattcttcatgcgattgacgtcagtacagcaggcgcaaatttacatacttcagcggtctctcaaggtgtcgctcttcgcctgatgcagtattgggggatcgagggattccttgcacatggccgcgcggtggccaaactttacacggagcgccgcgctcagttcgaggcaaccgcacataagtacctggacgggctggccacttgggtatctcccgtagcgggaatgtttttatggatcgatcttcgtccagcaggaatcgaagattcttacgaattaattcgccatgaagcattagccaaaggcgttttaggcgttccagggatggcgttttatccgacaggccgtaagtcttcccatgttcgtgtcagtttcagtatcgtcgacctggaagacgaatctgaccttggttttcaacgcctggctgaagctattaaggataaacgcaaggctttagggctggct

SEQ ID NO: 558 - protein, 500 aa, Catharanthus roseus
  REGION 1..500: note = misc_feature - TDC: Tryptophan decarboxylase from Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 559 - protein, 757 aa, Escherichia coli
  REGION 1..757: note = misc_feature - TYNA: Monoamine oxidase from E. coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 560 - protein, 1368 aa, Arabidopsis thaliana
  REGION 1..1368: note = misc_feature - AAO1: Indole-3-acetaldehyde oxidase from Arabidopsis thaliana
MGEKAIDEDKVEAMKSSKTSLVFAINGQRFELELSSIDPSTTLVDFLRNKTPFKSVKLGCGEGGCGACVVLLSKYDPLLEKVDEFTISSCLTLLCSIDGCSITTSDGLGNSRVGFHAVHERIAGFHATQCGFCTPGMSVSMFSALLNADKSHPPPRSGFSNLTAVEAEKAVSGNLCRCTGYRPLVDACKSFAADVDIEDLGFNAFCKKGENRDEVLRRLPCYDHTSSHVCTFPEFLKKEIKNDMSLHSRKYRWSSPVSVSELQGLLEVENGLSVKLVAGNTSTGYYKEEKERKYERFIDIRKIPEFTMVRSDEKGVELGACVTISKAIEVLREEKNVSVLAKIATHMEKIANRFVRNTGTIGGNIMMAQRKQFPSDLATILVAAQATVKIMTSSSSQEQFTLEEFLQQPPLDAKSLLLSLEIPSWHSAKKNGSSEDSILLFETYRAAPRPLGNALAFLNAAFSAEVTEALDGIVVNDCQLVFGAYGTKHAHRAKKVEEFLTGKVISDEVLMEAISLLKDEIVPDKGTSNPGYRSSLAVTFLFEFFGSLTKKNAKTTNGWLNGGCKEIGFDQNVESLKPEAMLSSAQQIVENQEHSPVGKGITKAGACLQASGEAVYVDDIPAPENCLYGAFIYSTMPLARIKGIRFKQNRVPEGVLGIITYKDIPKGGQNIGTNGFFTSDLLFAEEVTHCAGQIIAFLVADSQKHADIAANLVVIDYDTKDLKPPILSLEEAVENFSLFEVPPPLRGYPVGDITKGMDEAEHKILGSKISFGSQYFFYMETQTALAVPDEDNCMVVYSSTQTPEFVHQTIAGCLGVPENNVRVITRRVGGGFGGKAVKSMPVAAACALAASKMQRPVRTYVNRKTDMITTGGRHPMKVTYSVGFKSNGKITALDVEVLLDAGLTEDISPLMPKGIQGALMKYDWGALSFNVKVCKTNTVSRTALRAPGDVQGSYIGEAIIEKVASYLSVDVDEIRKVNLHTYESLRLFHSAKAGEFSEYTLPLLWDRIDEFSGFNKRRKVVEEFNASNKWRKRGISRVPAVYAVNMRSTPGRVSVLGDGSIVVEVQGIEIGQGLWTKVKQMAAYSLGLIQCGTTSDELLKKIRVIQSDTLSMVQGSMTAGSTTSEASSEAVRICCDGLVERLLPVKTALVEQTGGPVTWDSLISQAYQQSINMSVSSKYMPDSTGEYLNYGIAASEVEVNVLTGETTILRTDIIYDCGKSLNPAVDLGQIEGAFVQGLGFFMLEEFLMNSDGLVVTDSTWTYKIPTVDTIPRQFNVEILNSGQHKNRVLSSKASGEPPLLLAASVHCAVRAAVKEARKQILSWNSNKQGTDMYFELPVPATMPIVKEFCGLDVVEKYLEWKIQQRKNV

SEQ ID NO: 561 - protein, 513 aa, Saccharomyces cerevisiae
  REGION 1..513: note = misc_feature - ARO9: L-tryptophan aminotransferase from S. cerevisae
MTAGSAPPVDYTSLKKNFQPFLSRRVENRSLKSFWDASDISDDVIELAGGMPNERFFPIESMDLKISKVPFNDNPKWHNSFTTAHLDLGSPSELPIARSFQYAETKGLPPLLHFVKDFVSRINRPAFSDETESNWDVILSGGSNDSMFKVFETICDESTTVMIEEFTFTPAMSNVEATGAKVIPIKMNLTFDRESQGIDVEYLTQLLDNWSTGPYKDLNKPRVLYTIATGQNPTGMSVPQWKREKIYQLAQRHDFLIVEDDPYGYLYFPSYNPQEPLENPYHSSDLTTERYLNDFLMKSFLTLDTDARVIRLETFSKIFAPGLRLSFIVANKFLLQKILDLADITTRAPSGTSQAIVYSTIKAMAESNLSSSLSMKEAMFEGWIRWIMQIASKYNHRKNLTLKALYETESYQAGQFTVMEPSAGMFIIIKINWGNFDRPDDLPQQMDILDKFLLKNGVKVVLGYKMAVCPNYSKQNSDFLRLTIAYARDDDQLIEASKRIGSGIKEFFDNYKS

SEQ ID NO: 562 - protein, 396 aa, Escherichia coli
  REGION 1..396: note = misc_feature - aspC: aspartate aminotransferase from E. coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 563 - protein, 391 aa, Arabidopsis thaliana
  REGION 1..391: note = misc_feature - TAA1: L-tryptophan-pyruvate aminotransferase from Arabidopsis thaliana
MVKLENSRKPEKISNKNIPMSDFVVNLDHGDPTAYEEYWRKMGDRCTVTIRGCDLMSYFSDMTNLCWFLEPELEDAIKDLHGVVGNAATEDRYIVVGTGSTQLCQAAVHALSSLARSQPVSVVAAAPFYSTYVEETTYVRSGMYKWEGDAWGFDKKGPYIELVTSPNNPDGTIRETVVNRPDDDEAKVIHDFAYYWPHYTPITRRQDHDIMLFTFSKITGHAGSRIGWALVKDKEVAKKMVEYIIVNSIGVSKESQVRTAKILNVLKETCKSESESENFFKYGREMMKNRWEKLREVVKESDAFTLPKYPEAFCNYFGKSLESYPAFAWLGTKEETDLVSELRRHKVMSRAGERCGSDKKHVRVSMLSREDVFNVFLERLANMKLIKSIDL

SEQ ID NO: 564 - protein, 504 aa, Streptomyces sp.
  REGION 1..504: note = misc_feature - STAO: L-tryptophan oxidase from streptomyces sp. TP-A0274
MTAPLQDSDGPDDAIGGPKQVTVIGAGIAGLVTAYELERLGHHVQIIEGSDDIGGRIHTHRFSGAGGPGPFAEMGAMRIPAGHRLTMHYIAELGLQNQVREFRTLFSDDAAYLPSSAGYLRVREAHDTLVDEFATGLPSAHYRQDTLLFGAWLDASIRAIAPRQFYDGLHNDIGVELLNLVDDIDLTPYRCGTARNRIDLHALFADHPRVRASCPPRLERFLDDVLDETSSSIVRLKDGMDELPRRLASRIRGKISLGQEVTGIDVHDDTVTLTVRQGLRTVTRTCDYVVCTIPFTVLRTLRLTGFDQDKLDIVHETKYWPATKIAFHCREPFWEKDGISGGASFTGGHVRQTYYPPAEGDPALGAVLLASYTIGPDAEALARMDEAERDALVAKELSVMHPELRRPGMVLAVAGRDWGARRWSRGAATVRWGQEAALREAERRECARPQKGLFFAGEHCSSKPAWIEGAIESAIDAAHEIEWYEPRASRVFAASRLSRSDRSA

SEQ ID NO: 565 - protein, 552 aa, Enterobacter cloacae
  REGION 1..552: note = misc_feature - ipdC: Indole-3-pyruvate decarboxylase from Enterobacter cloacae
MRTPYCVADYLLDRLTDCGADHLFGVPGDYNLQFLDHVIDSPDICWVGCANELNASYAADGYARCKGFAALLTTFGVGELSAMNGIAGSYAEHVPVLHIVGAPGTAAQQRGELLHHTLGDGEFRHFYHMSEPITVAQAVLTEQNACYEIDRVLTTMLRERRPGYLMLPADVAKKAATPPVNALTHKQAHADSACLKAFRDAAENKLAMSKRTALLADFLVLRHGLKHALQKWVKEVPMAHATMLMGKGIFDERQAGFYGTYSGSASTGAVKEAIEGADTVLCVGTRFTDTLTAGFTHQLTPAQTIEVQPHAARVGDVWFTGIPMNQAIETLVELCKQHVHAGLMSSSSGAIPFPQPDGSLTQENFWRTLQTFIRPGDIILADQGTSAFGAIDLRLPADVNFIVQPLWGSIGYTLAAAFGAQTACPNRRVIVLTGDGAAQLTIQELGSMLRDKQHPIILVLNNEGYTVERAIHGAEQRYNDIALWNWTHIPQALSLDPQSECWRVSEAEQLADVLEKVAHHERLSLIEVMLPKADIPPLLGALTKALEACNNA

SEQ ID NO: 566 - protein, 497 aa, Ustilago maydis
  REGION 1..497: note = misc_feature - IAD1: Indole-3-acetaldehyde dehydrogenase from Ustilago maydis
MPTLNLDLPNGIKSTIQADLFINNKFVPALDGKTFATINPSTGKEIGQVAEASAKDVDLAVKAAREAFETTWGENTPGDARGRLLIKLAELVEANIDELAAIESLDNGKAFSIAKSFDVAAVAANLRYYGGWADKNHGKVMEVDTKRLNYTRHEPIGVCGQIIPWNFPLLMFAWKLGPALATGNTIVLKTAEQTPLSAIKMCELIVEAGFPPGVVNVISGFGPVAGAAISQHMDIDKIAFTGSTLVGRNIMKAAASTNLKKVTLELGGKSPNIIFKDADLDQAVRWSAFGIMFNHGQCCCAGSRVYVEESIYDAFMEKMTAHCKALQVGDPFSANTFQGPQVSQLQYDRIMEYIESGKKDANLALGGVRKGNEGYFIEPTIFTDVPHDAKIAKEEIFGPVVVVSKFKDEKDLIRIANDSIYGLAAAVFSRDISRAIETAHKLKAGTVWVNCYNQLIPQVPFGGYKASGIGRELGEYALSNYTNIKAVHVNLSQPAPI

SEQ ID NO: 567 - protein, 415 aa, Arabidopsis thaliana
  REGION 1..415: note = misc_feature - YUC2: indole-3-pyruvate monoxygenase from Arabidopsis thaliana
MEFVTETLGKRIHDPYVEETRCLMIPGPIIVGSGPSGLATAACLKSRDIPSLILERSTCIASLWQHKTYDRLRLHLPKDFCELPLMPFPSSYPTYPTKQQFVQYLESYAEHFDLKPVFNQTVEEAKFDRRCGLWRVRTTGGKKDETMEYVSRWLVVATGENAEEVMPEIDGIPDFGGPILHTSSYKSGEIFSEKKILVVGCGNSGMEVCLDLCNFNALPSLVVRDSVHVLPQEMLGISTFGISTSLLKWFPVHVVDRFLLRMSRLVLGDTDRLGLVRPKLGPLERKIKCGKTPVLDVGTLAKIRSGHIKVYPELKRVMHYSAEFVDGRVDNFDAIILATGYKSNVPMWLKGVNMFSEKDGFPHKPFPNGWKGESGLYAVGFTKLGLLGAAIDAKKIAEDIEVQRHFLPLARPQHC

SEQ ID NO: 568 - protein, 557 aa, Pseudomonas savastanoi
  REGION 1..557: note = misc_feature - IaaM: Tryptophan 2-monooxygenase from Pseudomonas savastanoi
MYDHFNSPSIDILYDYGPFLKKCEMTGGIGSYSAGTPTPRVAIVGAGISGLVAATELLRAGVKDVVLYESRDRIGGRVWSQVFDQTRPRYIAEMGAMRFPPSATGLFHYLKKFGISTSTTFPDPGVVDTELHYRGKRYHWPAGKKPPELFRRVYEGWQSLLSEGYLLEGGSLVAPLDITAMLKSGRLEEAAIAWQGWLNVFRDCSFYNAIVCIFTGRHPPGGDRWARPEDFELFGSLGIGSGGFLPVFQAGFTEILRMVINGYQSDQRLIPDGISSLAARLADQSFDGKALRDRVCFSRVGRISREAEKIIIQTEAGEQRVFDRVIVTSSNRAMQMIHCLTDSESFLSRDVARAVRETHLTGSSKLFILTRTKFWIKNKLPTTIQSDGLVRGVYCLDYQPDEPEGHGVVLLSYTWEDDAQKMLAMPDKKTRCQVLVDDLAAIHPTFASYLLPVDGDYERYVLHHDWLTDPHSAGAFKLNYPGEDVYSQRLFFQPMTANSPNKDTGLYLAGCSCSFAGGWIEGAVQTALNSACAVLRSTGGQLSKGNPLDCINASYRY

SEQ ID NO: 569 - protein, 455 aa, Pseudomonas savastanoi
  REGION 1..455: note = misc_feature - iaaH: Indoleacetamide hydrolase from Pseudomonas savastanoi
MHEIITLESLCQALADGEIAAAELRERALDTEARLARLNCFIREGDAVSQFGEADHAMKGTPLWGMPVSFKDNICVRGLPLTAGTRGMSGFVSDQDAAIVSQLRALGAVVAGKNNMHELSFGVTSINPHWGTVGNPVAPGYCAGGSSGGSAAAVASGIVPLSVGTDTGGSIRIPAAFCGITGFRPTTGRWSTAGIIPVSHTKDCVGLLTRTAGDAGFLYGLLSGKQQSFPLSRTAPCRIGLPVSMWSDLDGEVERACVNALSLLRKTGFEFIEIDDADIVELNQTLTFTVPLYEFFADLAQSLLSLGWKHGIHHIFAQVDDANVKGIINHHLGEGAIKPAHYLSSLQNGELLKRKMDELFARHNIELLGYPTVPCRVPHLDHADRPEFFSQAIRNTDLASNAMLPSITIPVGPEGRLPVGLSFDALRGRDALLLSRVSAIEQVLGFVRKVLPHTT

SEQ ID NO: 570 - protein, 353 aa, unidentified
  REGION 1..353: note = misc_feature - TrpDH: Tryptophan dehydrogenase from Nostoc punctiforme NIES-2108
  source 1..353: note = Nostoc punctiforme
MLLFETVREMGHEQVLFCHSKNPEIKAIIAIHDTTLGPAMGATRILPYINEEAALKDALRLSRGMTYKAACANIPAGGGKAVIIANPENKTDDLLRAYGRFVDSLNGRFITGQDVNITPDDVRTISQETKYVVGVSEKSGGPAPITSLGVFLGIKAAVESRWQSKRLDGMKVAVQGLGNVGKNLCRHLHEHDVQLFVSDVDPIKAEEVKRLFGATVVEPTEIYSLDVDIFAPCALGGILNSHTIPFLQASIIAGAANNQLENEQLHSQMLAKKGILYSPDYVINAGGLINVYNEMIGYDEEKAFKQVHNIYDTLLAIFEIAKEQGVTTNDAARRLAEDRINNSKRSKSKAIAA

SEQ ID NO: 571 - protein, 541 aa, Arabidopsis thaliana
  REGION 1..541: note = misc_feature - CYP79B2: tryptophan N-monooxygenase from Arabidopsis thaliana
MNTFTSNSSDLTTTATETSSFSTLYLLSTLQAFVAITLVMLLKKLMTDPNKKKPYLPPGPTGWPIIGMIPTMLKSRPVFRWLHSIMKQLNTEIACVKLGNTHVITVTCPKIAREILKQQDALFASRPLTYAQKILSNGYKTCVITPFGDQFKKMRKVVMTELVCPARHRWLHQKRSEENDHLTAWVYNMVKNSGSVDFRFMTRHYCGNAIKKLMFGTRTFSKNTAPDGGPTVEDVEHMEAMFEALGFTFAFCISDYLPMLTGLDLNGHEKIMRESSAIMDKYHDPIIDERIKMWREGKRTQIEDFLDIFISIKDEQGNPLLTADEIKPTIKELVMAAPDNPSNAVEWAMAEMVNKPEILRKAMEEIDRVVGKERLVQESDIPKLNYVKAILREAFRLHPVAAFNLPHVALSDTTVAGYHIPKGSQVLLSRYGLGRNPKVWADPLCFKPERHLNECSEVTLTENDLRFISFSTGKRGCAAPALGTALTTMMLARLLQGFTWKLPENETRVELMESSHDMFLAKPLVMVGDLRLPEHLYPTVK

SEQ ID NO: 572 - protein, 543 aa, Arabidopsis thaliana
  REGION 1..543: note = misc_feature - CYP79B3: tryptophan N-monooxygenase from Arabidopsis thaliana
MDTLASNSSDLTTKSSLGMSSFTNMYLLTTLQALAALCFLMILNKIKSSSRNKKLHPLPPGPTGFPIVGMIPAMLKNRPVFRWLHSLMKELNTEIACVRLGNTHVIPVTCPKIAREIFKQQDALFASRPLTYAQKILSNGYKTCVITPFGEQFKKMRKVIMTEIVCPARHRWLHDNRAEETDHLTAWLYNMVKNSEPVDLRFVTRHYCGNAIKRLMFGTRTFSEKTEADGGPTLEDIEHMDAMFEGLGFTFAFCISDYLPMLTGLDLNGHEKIMRESSAIMDKYHDPIIDERIKMWREGKRTQIEDFLDIFISIKDEAGQPLLTADEIKPTIKELVMAAPDNPSNAVEWAIAEMINKPEILHKAMEEIDRVVGKERFVQESDIPKLNYVKAIIREAFRLHPVAAFNLPHVALSDTTVAGYHIPKGSQVLLSRYGLGRNPKVWSDPLSFKPERHLNECSEVTLTENDLRFISFSTGKRGCAAPALGTAITTMMLARLLQGFKWKLAGSETRVELMESSHDMFLSKPLVLVGELRLSEDLYPMVK

SEQ ID NO: 573 - protein, 503 aa, Arabidopsis thaliana
  REGION 1..503: note = misc_feature - CYP71A13: indoleacetaldoxime dehydratase from Arabidopis thaliana
MSNIQEMEMILSISLCLTTLITLLLLRRFLKRTATKVNLPPSPWRLPVIGNLHQLSLHPHRSLRSLSLRYGPLMLLHFGRVPILVVSSGEAAQEVLKTHDHKFANRPRSKAVHGLMNGGRDVVFAPYGEYWRQMKSVCILNLLTNKMVESFEKVREDEVNAMIEKLEKASSSSSSENLSELFITLPSDVTSRVALGRKHSEDETARDLKKRVRQIMELLGEFPIGEYVPILAWIDGIRGFNNKIKEVSRGFSDLMDKVVQEHLEASNDKADFVDILLSIEKDKNSGFQVQRNDIKFMILDMFIGGTSTTSTLLEWTMTELIRSPKSMKKLQDEIRSTIRPHGSYIKEKEVENMKYLKAVIKEVLRLHPSLPMILPRLLSEDVKVKGYNIAAGTEVIINAWAIQRDTAIWGPDAEEFKPERHLDSGLDYHGKNLNYIPFGSGRRICPGINLALGLAEVTVANLVGRFDWRVEAGPNGDQPDLTEAIGIDVCRKFPLIAFPSSVV

SEQ ID NO: 574 - protein, 560 aa, Arabidopsis thaliana
  REGION 1..560: note = misc_feature - PEN2: myrosinase from Arabidopsis thaliana
MAHLQRTFPTEMSKGRASFPKGFLFGTASSSYQYEGAVNEGARGQSVWDHFSNRFPHRISDSSDGNVAVDFYHRYKEDIKRMKDINMDSFRLSIAWPRVLPYGKRDRGVSEEGIKFYNDVIDELLANEITPLVTIFHWDIPQDLEDEYGGFLSEQIIDDFRDYASLCFERFGDRVSLWCTMNEPWVYSVAGYDTGRKAPGRCSKYVNGASVAGMSGYEAYIVSHNMLLAHAEAVEVFRKCDHIKNGQIGIAHNPLWYEPYDPSDPDDVEGCNRAMDFMLGWHQHPTACGDYPETMKKSVGDRLPSFTPEQSKKLIGSCDYVGINYYSSLFVKSIKHVDPTQPTWRTDQGVDWMKTNIDGKQIAKQGGSEWSFTYPTGLRNILKYVKKTYGNPPILITENGYGEVAEQSQSLYMYNPSIDTERLEYIEGHIHAIHQAIHEDGVRVEGYYVWSLLDNFEWNSGYGVRYGLYYIDYKDGLRRYPKMSALWLKEFLRFDQEDDSSTSKKEEKKESYGKQLLHSVQDSQFVHSIKDSGALPAVLGSLFVVSATVGTSLFFKGANN

SEQ ID NO: 575 - protein, 346 aa, Arabidopsis thaliana
  REGION 1..346: note = misc_feature - Nit1: Nitrilase from Arabidopsis thaliana
MSSTKDMSTVQNATPFNGVAPSTTVRVTIVQSSTVYNDTPATIDKAEKYIVEAASKGAELVLFPEGFIGGYPRGFRFGLAVGVHNEEGRDEFRKYHASAIHVPGPEVARLADVARKNHVYLVMGAIEKEGYTLYCTVLFFSPQGQFLGKHRKLMPTSLERCIWGQGDGSTIPVYDTPIGKLGAAICWENRMPLYRTALYAKGIELYCAPTADGSKEWQSSMLHIAIEGGCFVLSACQFCQRKHFPDHPDYLFTDWYDDKEHDSIVSQGGSVIISPLGQVLAGPNFESEGLVTADIDLGDIARAKLYFDSVGHYSRPDVLHLTVNEHPRKSVTFVTKVEKAEDDSNK

SEQ ID NO: 576 - protein, 403 aa, Homo sapiens [sequence omitted]

SEQ ID NO: 577 - protein, 406 aa, Homo sapiens
  REGION 1..406: note = misc_feature - TDO2: tryptophan 2,3-dioxygenase from Homo sapiens
MSGCPFLGNNFGYTFKKLPVEGSEEDKSQTGVNRASKGGLIYGNYLHLEKVLNAQELQSETKGNKIHDEHLFIITHQAYELWFKQILWELDSVREIFQNGHVRDERNMLKVVSRMHRVSVILKLLVQQFSILETMTALDFNDFREYLSPASGFQSLQFRLLENKIGVLQNMRVPYNRRHYRDNFKGEENELLLKSEQEKTLLELVEAWLERTPGLEPHGFNFWGKLEKNITRGLEEEFIRIQAKEESEEKEEQVAEFQKQKEVLLSLFDEKRHEHLLSKGERRLSYRALQGALMIYFYREEPRFQVPFQLLTSLMDIDSLMTKWRYNHVCMVHRMLGSKAGTGGSSGYHYLRSTVSDRYKVFVDLFNLSTYLIPRHWIPKMNPTIHKFLYTAEYCDSSYFSSDESD

SEQ ID NO: 578 - protein, 453 aa, Saccharomyces cerevisiae
  REGION 1..453: note = misc_feature - BNA2: indoleamine 2,3-dioxygenase from S. cerevisiae
MNNTSITGPQVLHRTKMRPLPVLEKYCISPHHGFLDDRLPLTRLSSKKYMKWEEIVADLPSLLQEDNKVRSVIDGLDVLDLDETILGDVRELRRAYSILGFMAHAYIWASGTPRDVLPECIARPLLETAHILGVPPLATYSSLVLWNFKVTDECKKTETGCLDLENITTINTFTGTVDESWFYLVSVRFEKIGSACLNHGLQILRAIRSGDKGDANVIDGLEGLAATIERLSKALMEMELKCEPNVFYFKIRPFLAGWTNMSHMGLPQGVRYGAEGQYRIFSGGSNAQSSLIQTLDILLGVKHTANAAHSSQGDSKINYLDEMKKYMPREHREFLYHLESVCNIREYVSRNASNRALQEAYGRCISMLKIFRDNHIQIVTKYIILPSNSKQHGSNKPNVLSPIEPNTKASGCLGHKVASSKTIGTGGTRLMPFLKQCRDETVATADIKNEDKN

SEQ ID NO: 579 - protein, 305 aa, Mus musculus
  REGION 1..305: note = misc_feature - Afmid: Kynurenine formamidase from mouse
MAFPSLSAGQNPWRNLSSEELEKQYSPSRWVIHTKPEEVVGNFVQIGSQATQKARATRRNQLDVPYGDGEGEKLDIYFPDEDSKAFPLFLFLHGGYWQSGSKDDSAFMVNPLTAQGIVVVIVAYDIAPKGTLDQMVDQVTRSVVFLQRRYPSNEGIYLCGHSAGAHLAAMVLLARWTKHGVTPNLQGFLLVSGIYDLEPLIATSQNDPLRMTLEDAQRNSPQRHLDVVPAQPVAPACPVLVLVGQHDSPEFHRQSKEFYETLLRVGWKASFQQLRGVDHFDIIENLTREDDVLTQIILKTVFQKL

SEQ ID NO: 580 - protein, 444 aa, Saccharomyces cerevisiae
  REGION 1..444: note = misc_feature - BNA3: kynurenine--oxoglutarate transaminase from S. cerevisae
MKQRFIRQFTNLMSTSRPKVVANKYFTSNTAKDVWSLTNEAAAKAANNSKNQGRELINLGQGFFSYSPPQFAIKEAQKALDIPMVNQYSPTRGRPSLINSLIKLYSPIYNTELKAENVTVTTGANEGILSCLMGLLNAGDEVIVFEPFFDQYIPNIELCGGKVVYVPINPPKELDQRNTRGEEWTIDFEQFEKAITSKTKAVIINTPHNPIGKVFTREELTTLGNICVKHNVVIISDEVYEHLYFTDSFTRIATLSPEIGQLTLTVGSAGKSFAATGWRIGWVLSLNAELLSYAAKAHTRICFASPSPLQEACANSINDALKIGYFEKMRQEYINKFKIFTSIFDELGLPYTAPEGTYFVLVDFSKVKIPEDYPYPEEILNKGKDFRISHWLINELGVVAIPPTEFYIKEHEKAAENLLRFAVCKDDAYLENAVERLKLLKDYL

SEQ ID NO: 581 - protein, 430 aa, Homo sapiens
  REGION 1..430: note = misc_feature - GOT2: Aspartate aminotransferase, mitochondrial from Homo sapiens
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 582 - protein, 425 aa, synthetic construct
  REGION 1..425: note = Synthetic: Description of Unknown: AADAT: Kynurenine/alpha-aminoadipate aminotransferase, mitochondrial
MNYARFITAASAARNPSPIRTMTDILSRGPKSMISLAGGLPNPNMFPFKTAVITVENGKTIQFGEEMMKRALQYSPSAGIPELLSWLKQLQIKLHNPPTIHYPPSQGQMDLCVTSGSQQGLCKVFEMIINPGDNVLLDEPAYSGTLQSLHPLGCNIINVASDESGIVPDSLRDILSRWKPEDAKNPQKNTPKFLYTVPNGNNPTGNSLTSERKKEIYELARKYDFLIIEDDPYYFLQFNKFRVPTFLSMDVDGRVIRADSFSKIISSGLRIGFLTGPKPLIERVILHIQVSTLHPSTFNQLMISQLLHEWGEEGFMAHVDRVIDFYSNQKDAILAAADKWLTGLAEWHVPAAGMFLWIKVKGINDVKELIEEKAVKMGVLMLPGNAFYVDSSAPSPYLRASFSSASPEQMDVAFQVLAQLIKESL

SEQ ID NO: 583 - protein, 422 aa, Homo sapiens
  REGION 1..422: note = misc_feature - CCLB1: Kynurenine--oxoglutarate transaminase 1 from Homo sapiens
MAKQLQARRLDGIDYNPWVEFVKLASEHDVVNLGQGFPDFPPPDFAVEAFQHAVSGDFMLNQYTKTFGYPPLTKILASFFGELLGQEIDPLRNVLVTVGGYGALFTAFQALVDEGDEVIIIEPFFDCYEPMTMMAGGRPVFVSLKPGPIQNGELGSSSNWQLDPMELAGKFTSRTKALVLNTPNNPLGKVFSREELELVASLCQQHDVVCITDEVYQWMVYDGHQHISIASLPGMWERTLTIGSAGKTFSATGWKVGWVLGPDHIMKHLRTVHQNSVFHCPTQSQAAVAESFEREQLLFRQPSSYFVQFPQAMQRCRDHMIRSLQSVGLKPIIPQGSYFLITDISDFKRKMPDLPGAVDEPYDRRFVKWMIKNKGLVAIPVSIFYSVPHQKHFDHYIRFCFVKDEATLQAMDEKLRKWKVEL

SEQ ID NO: 584 - protein, 454 aa, Homo sapiens
  REGION 1..454: note = misc_feature - CCLB2: kynurenine--oxoglutarate transaminase 3 from Homo sapiens
MFLAQRSLCSLSGRAKFLKTISSSKILGFSTSAKMSLKFTNAKRIEGLDSNVWIEFTKLAADPSVVNLGQGFPDISPPTYVKEELSKIAAIDSLNQYTRGFGHPSLVKALSYLYEKLYQKQIDSNKEILVTVGAYGSLFNTIQALIDEGDEVILIVPFYDCYEPMVRMAGATPVFIPLRSKPVYGKRWSSSDWTLDPQELESKFNSKTKAIILNTPHNPLGKVYNREELQVIADLCIKYDTLCISDEVYEWLVYSGNKHLKIATFPGMWERTITIGSAGKTFSVTGWKLGWSIGPNHLIKHLQTVQQNTIYTCATPLQEALAQAFWIDIKRMDDPECYFNSLPKELEVKRDRMVRLLESVGLKPIVPDGGYFIIADVSLLDPDLSDMKNNEPYDYKFVKWMTKHKKLSAIPVSAFCNSETKSQFEKFVRFCFIKKDSTLDAAEEIIKAWSVQKS

SEQ ID NO: 585 - protein, 471 aa, Escherichia coli
  REGION 1..471: note = misc_feature - TnaA: tryptophanase from E. coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 586 - protein, 412 aa, Clostridium sporogenes
  REGION 1..412: note = misc_feature - FldA: indole-3-propionyl-CoA:indole-3-lactate CoA transferase from Clostridium sporogenes
MENNTNMFSGVKVIELANFIAAPAAGRFFADGGAEVIKIESPAGDPLRYTAPSEGRPLSQEENTTYDLENANKKAIVLNLKSEKGKKILHEMLAEADILLTNWRTKALVKQGLDYETLKEKYPKLVFAQITGYGEKGPDKDLPGFDYTAFFARGGVSGTLYEKGTVPPNVVPGLGDHQAGMFLAAGMAGALYKAKTTGQGDKVTVSLMHSAMYGLGIMIQAAQYKDHGLVYPINRNETPNPFIVSYKSKDDYFVQVCMPPYDVFYDRFMTALGREDLVGDERYNKIENLKDGRAKEVYSIIEQQMVTKTKDEWDKIFRDADIPFAIAQTWEDLLEDEQAWANDYLYKMKYPTGNERALVRLPVFFKEAGLPEYNQSPQIAENTVEVLKEMGYTEQEIEELEKDKDIMVRKEK

SEQ ID NO: 587 - protein, 407 aa, Clostridium sporogenes
  REGION 1..407: note = misc_feature - FldB: subunit of indole-3-lactate dehydratase from Clostridium sporogenes
MSDRNKEVKEKKAKHYLREITAKHYKEALEAKERGEKVGWCASNFPQEIATTLGVKVVYPENHAAAVAARGNGQNMCEHAEAMGFSNDVCGYARVNLAVMDIGHSEDQPIPMPDFVLCCNNICNQMIKWYEHIAKTLDIPMILIDIPYNTENTVSQDRIKYIRAQFDDAIKQLEEITGKKWDENKFEEVMKISQESAKQWLRAASYAKYKPSPFSGFDLFNHMAVAVCARGTQEAADAFKMLADEYEENVKTGKSTYRGEEKQRILFEGIACWPYLRHKLTKLSEYGMNVTATVYAEAFGVIYENMDELMAAYNKVPNSISFENALKMRLNAVTSTNTEGAVIHINRSCKLWSGFLYELARRLEKETGIPVVSFDGDQADPRNFSEAQYDTRIQGLNEVMVAKKEAE

SEQ ID NO: 588 - protein, 374 aa, Clostridium sporogenes
  REGION 1..374: note = misc_feature - FldC: subunit of indole-3-lactate dehydratase from Clostridium sporogenes
MSNSDKFFNDFKDIVENPKKYIMKHMEQTGQKAIGCMPLYTPEELVLAAGMFPVGVWGSNTELSKAKTYFPAFICSILQTTLENALNGEYDMLSGMMITNYCDSLKCMGQNFKLTVENIEFIPVTVPQNRKMEAGKEFLKSQYKMNIEQLEKISGNKITDESLEKAIEIYDEHRKVMNDFSMLASKYPGIITPTKRNYVMKSAYYMDKKEHTEKVRQLMDEIKAIEPKPFEGKRVITTGIIADSEDLLKILEENNIAIVGDDIAHESRQYRTLTPEANTPMDRLAEQFANRECSTLYDPEKKRGQYIVEMAKERKADGIIFFMTKFCDPEEYDYPQMKKDFEEAGIPHVLIETDMQMKNYEQARTAIQAFSETL

SEQ ID NO: 589 - protein, 377 aa, Clostridium sporogenes
  REGION 1..377: note = misc_feature - FldD: indole-3-acrylyl-CoA reductase from Clostridium sporogenes
MFFTEQHELIRKLARDFAEQEIEPIADEVDKTAEFPKEIVKKMAQNGFFGIKMPKEYGGAGADNRAYVTIMEEISRASGVAGIYLSSPNSLLGTPFLLVGTDEQKEKYLKPMIRGEKTLAFALTEPGAGSDAGALATTAREEGDYYILNGRKTFITGAPISDNIIVFAKTDMSKGTKGITTFIVDSKQEGVSFGKPEDKMGMIGCPTSDIILENVKVHKSDILGEVNKGFITAMKTLSVGRIGVASQALGIAQAAVDEAVKYAKQRKQFNRPIAKFQAIQFKLANMETKLNAAKLLVYNAAYKMDCGEKADKEASMAKYFAAESAIQIVNDALQIHGGYGYIKDYKIERLYRDVRVIAIYEGTSEVQQMVIASNLLK

SEQ ID NO: 590 - protein, 331 aa, Clostridium sporogenes
  REGION 1..331: note = misc_feature - FldH1: indole-3-lactate dehydrogenase from Clostridium sporogenes
MKILAYCVRPDEVDSFKKFSEKYGHTVDLIPDSFGPNVAHLAKGYDGISILGNDTCNREALEKIKDCGIKYLATRTAGVNNIDFDAAKEFGINVANVPAYSPNSVSEFTIGLALSLTRKIPFALKRVELNNFALGGLIGVELRNLTLGVIGTGRIGLKVIEGFSGFGMKKMIGYDIFENEEAKKYIEYKSLDEVFKEADIITLHAPLTDDNYHMIGKESIAKMKDGVFIINAARGALIDSEALIEGLKSGKIAGAALDSYEYEQGVFHNNKMNEIMQDDTLERLKSFPNVVITPHLGFYTDEAVSNMVEITLMNLQEFELKGTCKNQRVCK

SEQ ID NO: 591 - protein, 334 aa, Clostridium sporogenes
  REGION 1..334: note = misc_feature - FldH2: indole-3-lactate dehydrogenase from Clostridium sporogenes
MKILMYSVREHEKPAIKKWLEANPGVQIDLCNNALSEDTVCKAKEYDGIAIQQTNSIGGKAVYSTLKEYGIKQIASRTAGVDMIDLKMASDSNILVTNVPAYSPNAIAELAVTHTMNLLRNIKTLNKRIAYGDYRWSADLIAREVRSVTVGVVGTGKIGRTSAKLFKGLGANVIGYDAYPDKKLEENNLLTYKESLEDLLREADVVTLHTPLLESTKYMINKNNLKYMKPDAFIVNTGRGGIINTEDLIEALEQNKIAGAALDTFENEGLFLNKVVDPTKLPDSQLDKLLKMDQVLITHHVGFFTTTAVQNIVDTSLDSVVEVLKTNNSVNKVN

SEQ ID NO: 592 - protein, 326 aa, Rhodobacter sphaeroides
  REGION 1..326: note = misc_feature - AcuI: acrylyl-CoA reductase from Rhodobacter sphaeroides
MRAVLIEKSDDTQSVSVTELAEDQLPEGDVLVDVAYSTLNYKDALAITGKAPVVRRFPMVPGIDFTGTVAQSSHADFKPGDRVILNGWGVGEKHWGGLAERARVRGDWLVPLPAPLDLRQAAMIGTAGYTAMLCVLALERHGVVPGNGEIVVSGAAGGVGSVATTLLAAKGYEVAAVTGRASEAEYLRGLGAASVIDRNELTGKVRPLGQERWAGGIDVAGSTVLANMLSMMKYRGVVAACGLAAGMDLPASVAPFILRGMTLAGVDSVMCPKTDRLAAWARLASDLDPAKLEEMTTELPFSEVIETAPKFLDGTVRGRIVIPVTP

SEQ ID NO: 593 - DNA, 1182 bp, Brevibacterium aurantiacum
  misc_feature 1..1182: note = DNA Brevibacterium aurantiacum
  source 1..1182: mol_type = genomic DNA
atgacgagccttcaccccgaaactttaatggtgcacgggggtatgaagggattgacagaggcgggtgttcacgtcccggcgatcgacctgtcgacaacgaatcctgttaatgacgtagcgacagggggtgattcttacgaatggcttgcaacgggtcacacgttgaaggatggtgattctgctgtttaccagcgtttatggcagcccggtgtagctcgtttcgagacggctctggctgggctggagcacgcggaggaggctgtagctttcgccacgggtatggccgccatgaccgcggccttattggcagcagtatcagctggcacgcctcatatcgtcgcagtccgcccattatacggcggttcagaccatcttttagagactggtttactggggacaactgtcacgtgggcgaaagaagctgacatcgccagtgcaatccaagacgatacagggttagtgatcgtggagacgccagccaatcctagtcttgatctggtagacctggattctgtcgtgtcagcggcagggaacgtccctgtccttgtagataatacgttttgtactcctgtccttcagcaacctatctcgcacggcgccgcacttgtattacattcggcaacaaagtatttaggggggcatggggacgccatgggcgggatcatcgcgactaatgctgattgggctatgcgcctgcgtcaggtccgtgcgatcactggtgcattactgcatccgatgggcgcgtacttgttacaccgcggtttgcgtactctggctgttcgcatgcgtgccgctcagacaactgctggtgagcttgctgagcgcttagacgctcatcccgctatttcagttgtgcactacccaggattgaaggggcaagatcctcgtggattgttaggccgtcagatgagtggaggtggggcaatgattgctatggagctggcagggggattcgatgcggcacgctcctttgttgagcattgtaacttggtcgtgcatgccgtatcacttgggggagcagacacattaattcaacatccggcaagtttgacacaccgcccggtcgcagcaaccgccaagcctggtgatggtttgatccgcttgagcgtggggcttgagcacgttgacgacttggccgatgacttgatcgctgctttggacgcctctcgcgctgctgcatga

SEQ ID NO: 594 - DNA, 1197 bp, Citrobacter freundii
  misc_feature 1..1197: note = DNA Citrobacter freundii
  source 1..1197: mol_type = genomic DNA
atgtctgattgtcgtgcctacggctttaacacacagatcgtccacgccggacaacaaccggacccaagcactggtgcattgtcgacgccaatctttcagacttctactttcgttttcgattcggcagaacagggggccgcacgtttcgcgttggaggaaccgggatacatctatacgcgcttgggcaatccaactactgatgcgcttgaaaagaaacttgcagtgctggaacgtggggacgcggcgttagccacagcgagcggcatctccgctattaccacaagtcttcttactctttgccagcaaggggaccatatcgtatcagctagtgcaatctacggttgcactcacgcattcttaagtcattcgcttcctaaatttggcattaacgtttcattcgtcgacgcagcgaagcccgaagaaatccgcgccgccatgcgtccagagacaaaagtggtatatatcgagacgcccgcgaaccctactttatccttagtggatattgagactgttgctggaatcgcgcaccagcaaggagccttgttagtcgtggacaacacttttatgtctccgtattgtcaacaaccattactgctgggtgctgacatcgtggtccatagtgttaccaagtacattaacgggcatggggatgttattggcggtgtcatcgtaggtaaacaggagttcatcgaccaagcacgctttgtgggattaaaggacattacgggcggatgcatgagcccctttaatgcatggctgacacttcgcggggtaaagacgttagggatccgtatggagcgtcattgcgaaaacgcattgaaaatcgcgcgttttcttgagggccatcctgctattacacgtgtctattatcctggattgccgtcacacccccaatacgaacttgggaagcgccaaatgtcgcttcctgggggaatcatttcgtttgaaattgctggtggccttgaagcaggtcgccgtatgattaacagcgtggagttatgtttgctggcagttagcttaggtgatacggagacgttgattcagcatccggcgagcatgacacattcccctgttgctcccgaggagcgtctgaaggctggcatcactgatggactgatccgtttgtccgtcggccttgaagatccggaagacatcattaacgacctggagcacgccattcgcaaggcaacgttttaa

SEQ ID NO: 595 - DNA, 1200 bp, Porphyromonas gingivalis
  misc_feature 1..1200: note = DNA Porphyromonas gingivalis
  source 1..1200: mol_type = genomic DNA
atgaagaaagaggacttaatgcgttcggggttcgcaactcgtgctattcatggtggagccatcgaaaatgcttttgggtgcctggccacccccatctaccaaacttcaacgtttgttttcgatacggctgaacagggtggacgtcgctttgcgggtgaagaagatgggtacatttacactcgcttaggaaacccgaactgtacccaggttgaggagaagctggcgatgttagaaggaggggaagcagcggcctccgcaagtagtggtatcggagcgatctcttcagctatctgggtgtgcgtcaaggcgggagatcatattgtagcgggaaaaactttgtacggttgcacgtttgcgttcttgacccacggactttctcgctatggagttgaggtcaccttggtggacacgcgccatccagaagaggttgaggccgctattcgtccaaacactaagttggtctatttggaaactcccgcaaaccctaacatgtaccttactgatatcaaagctgtgtgtgatatcgcgcacaagcatgagggtgttcgcgtcatggtggataatacctactgtacaccttacatctgtcgcccactggagttaggtgctgatattgttgtccattccgccacaaaatatctgaacggacatggagatgttatcgctgggtttgtagtagggaaagaggattacatcaaggaggtcaaattggttggtgttaaggacttgacaggagccaatatgtcacccttcgacgcataccttattagtcgtggtatgaagacgttacaaatccgtatggaacaacattgccgtaacgcacaaacagttgctgagtttctggagaagcatcctgcggttgaggcggtgtacttcccgggcttgccttcattcccgcaatatgagttggccaagaagcagatggcacttccgggagcgatgattgcgtttgaagttaagggaggctgcgaagccggtaagaaacttatgaacaatcttcatctgtgttccttagccgtctccttaggcgataccgagactttaatccaacacccggcctcaatgacgcactcgccctatacgcccgaggaacgcgccgcaagcgacatctcggaggggcttgtacgcctttctgttggattggaaaatgttgaggatattatcgcggacctgaagcatggcctggatagtcttatctaa

SEQ ID NO: 596 - DNA, 1674 bp, Streptomyces sp.
  misc_feature 1..1674: note = metDC Streptomyces sp.
  source 1..1674: mol_type = genomic DNA
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 597 - DNA, 1032 bp, Escherichia coli
  misc_feature 1..1032: note = metN Escherichia coli
  source 1..1032: mol_type = genomic DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctga

SEQ ID NO: 598 - DNA, 654 bp, Escherichia coli
  misc_feature 1..654: note = metI Escherichia coli
  source 1..654: mol_type = genomic DNA
atgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaa

SEQ ID NO: 599 - DNA, 816 bp, Escherichia coli
  misc_feature 1..816: note = metQEscherichia coli
  source 1..816: mol_type = genomic DNA
atggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 600 - protein, 393 aa, Brevibacterium aurantiacum
  REGION 1..393: note = misc_feature - PRT Brevibacterium aurantiacum
MTSLHPETLMVHGGMKGLTEAGVHVPAIDLSTTNPVNDVATGGDSYEWLATGHTLKDGDSAVYQRLWQPGVARFETALAGLEHAEEAVAFATGMAAMTAALLAAVSAGTPHIVAVRPLYGGSDHLLETGLLGTTVTWAKEADIASAIQDDTGLVIVETPANPSLDLVDLDSVVSAAGNVPVLVDNTFCTPVLQQPISHGAALVLHSATKYLGGHGDAMGGIIATNADWAMRLRQVRAITGALLHPMGAYLLHRGLRTLAVRMRAAQTTAGELAERLDAHPAISVVHYPGLKGQDPRGLLGRQMSGGGAMIAMELAGGFDAARSFVEHCNLVVHAVSLGGADTLIQHPASLTHRPVAATAKPGDGLIRLSVGLEHVDDLADDLIAALDASRAAA

SEQ ID NO: 601 - protein, 398 aa, Citrobacter freundii
  REGION 1..398: note = misc_feature - PRT Citrobacter freundii
MSDCRAYGFNTQIVHAGQQPDPSTGALSTPIFQTSTFVFDSAEQGAARFALEEPGYIYTRLGNPTTDALEKKLAVLERGDAALATASGISAITTSLLTLCQQGDHIVSASAIYGCTHAFLSHSLPKFGINVSFVDAAKPEEIRAAMRPETKVVYIETPANPTLSLVDIETVAGIAHQQGALLVVDNTFMSPYCQQPLLLGADIVVHSVTKYINGHGDVIGGVIVGKQEFIDQARFVGLKDITGGCMSPFNAWLTLRGVKTLGIRMERHCENALKIARFLEGHPAITRVYYPGLPSHPQYELGKRQMSLPGGIISFEIAGGLEAGRRMINSVELCLLAVSLGDTETLIQHPASMTHSPVAPEERLKAGITDGLIRLSVGLEDPEDIINDLEHAIRKATF

SEQ ID NO: 602 - protein, 399 aa, Porphyromonas gingivalis
  REGION 1..399: note = misc_feature - PRT Porphyromonas gingivalis
MKKEDLMRSGFATRAIHGGAIENAFGCLATPIYQTSTFVFDTAEQGGRRFAGEEDGYIYTRLGNPNCTQVEEKLAMLEGGEAAASASSGIGAISSAIWVCVKAGDHIVAGKTLYGCTFAFLTHGLSRYGVEVTLVDTRHPEEVEAAIRPNTKLVYLETPANPNMYLTDIKAVCDIAHKHEGVRVMVDNTYCTPYICRPLELGADIVVHSATKYLNGHGDVIAGFVVGKEDYIKEVKLVGVKDLTGANMSPFDAYLISRGMKTLQIRMEQHCRNAQTVAEFLEKHPAVEAVYFPGLPSFPQYELAKKQMALPGAMIAFEVKGGCEAGKKLMNNLHLCSLAVSLGDTETLIQHPASMTHSPYTPEERAASDISEGLVRLSVGLENVEDIIADLKHGLDSLI

SEQ ID NO: 603 - protein, 557 aa, Streptomyces sp.
  REGION 1..557: note = misc_feature - PRT Streptomyces sp.
MSPTAFPAAETATAPATAVDPGPELDGGDFALPEGGLDDDRRLRALDAVDEYLTRKRKHLVGYQATQDMQGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 604 - protein, 343 aa, Escherichia coli
  REGION 1..343: note = misc_feature - PRT Escherichia coli
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAPLLSETARRFNVNNNIISAQMDYAGGVKFGIMLTEMHGTQQDTQAAIAWLQEHHVKVEVLGYV

SEQ ID NO: 605 - skipped

SEQ ID NO: 606 - protein, 271 aa, Escherichia coli
  REGION 1..271: note = misc_feature - PRT Escherichia coli
MAFKFKTFAAVGALIGSLALVGCGQDEKDPNHIKVGVIVGAEQQVAEVAQKVAKDKYGLDVELVTFNDYVLPNEALSKGDIDANAFQHKPYLDQQLKDRGYKLVAVGNTFVYPIAGYSKKIKSLDELQDGSQVAVPNDPTNLGRSLLLLQKVGLIKLKDGVGLLPTVLDVVENPKNLKIVELEAPQLPRSLDDAQIALAVINTTYASQIGLTPAKDGIFVEDKESPYVNLIVTREDNKDAENVKKFVQAYQSDEVYEAANKVFNGGAVKGW

SEQ ID NO: 607 - DNA, 1257 bp, Escherichia coli
  misc_feature 1..1257: note = DNA Escherichia coli
  source 1..1257: mol_type = genomic DNA
atgagtggactcaaacaagaactggggctggcccagggcatcggcctactatcgacgtcattattaggcactggcgtgtttgccgttcctgcgttagctgcgctagtagcaggcaataacagcctgtgggcgtggcccgttttgattatcttagtgttcccgattgcgattgtgtttgcgattctgggtcgccactatcccagcgcaggcggcgtcgcacacttcgtcggtatggcgtttggttcgcggcttgagcgagtcaccggctggttgtttttatcggtcattcccgtgggtttgcctgccgcgctacaaattgctgccggattcggccaggcaatgtttggctggcatagcgggcaactgttgttggcagaactcggtacgctggcgctggtgtggtatatcggtactcgaggtgccagttccagtgctaatctacaaacagttattgccgggcttatcgtcgcactgattgtcgctatctggtgggcgggcgatatcaaacctgcgaatatccccttccctgcgccaggaaatatcgaacttaccgggttattcgctgcgttatcagtgatgttctggtgttttgtcggtctggaagcatttgcccatcttgcctcggaatttaaaaatccagagcgtgattttcctcgtgctttgatgattggcctgctgctggcaggattagtctattggggctgtacggtagtcgtcttacacttcgacgcctatggtgaacaaatggcggcggcagcatcgcttcccaaaattgtagtgcagttattcggtgtaggagcgttatggattgcctgcgtaattggctatctggcctgctttgccagtctcaacatttatatacagagcttcgcccgcctggtctggtcgcaggcgcaacataatcctgaccattacctggcacgcctctcttctcgccatattccgaataatgccctcaatgcggtgctcggctgctgcgtggtgagcacgttggtgattcatgctttagagatcaatctggacgctcttattatttatgccaatggcatctttattatgatttatctgttatgcatgctggcaggctgtaaattattgcaaggacgttatcgactactggcagtggttggcgggctattatgcgttctgttactggcaatggtcggctggaaaagtctctacgcgctgatcatgctggcggggttatggctgtttctgccaaaacgaaaaacgccggaaaatggcataaccacataa

SEQ ID NO: 608 - DNA, 1197 bp, Citrobacter freundii
  misc_feature 1..1197: note = DNA Citrobacter freundii
  source 1..1197: mol_type = genomic DNA
atgtctgactgtcgtgcttacggattcaatacccagatcgttcatgcgggccaacaacccgacccttccactggcgcgctcagtacgcctattttccagacgtcaaccttcgtttttgacagtgccgaacagggcgccgcccgctttgcgcttgaagaacccggctacatctatacgcgccttggaaaccccaccaccgacgcgctggagaaaaagctggctgtgctggaaagaggcgatgccgcgctggcaactgcatccggtatttcagccatcaccacctcgttgctgaccctttgccagcagggcgaccatatcgtttccgccagtgccatttacggctgcactcacgcttttctgtcacacagcctgccgaagttcggcattaacgtcagcttcgtcgacgccgccaagccggaagaaatccgcgcggccatgcgcccggaaaccaaagtggtgtatatcgaaacaccagcaaacccaacgctctcgctggttgatattgaaacggtcgccgggatcgcccatcagcaaggcgcattgctggtcgtggataacacctttatgtcaccctactgccagcaacctctgctgttaggtgccgacatcgtggtgcacagcgtgaccaagtacatcaacggccacggggacgtgattggcggtgtgattgttggcaagcaggaatttatcgaccaggcacgattcgtcgggctgaaagatatcaccggcggctgcatgagtccgttcaacgcctggctgacgctgcgcggcgtgaaaacgctgggcatccgaatggagcgccattgtgaaaacgcgttaaaaattgcccgcttcctggaagggcatccggccattacccgcgtgtattaccctggcttgccttcacatccgcagtatgagctgggtaaacgtcagatgagcctgccgggaggaattatcagcttcgaaatcgccggcggcctcgaggctggcagacggatgatcaattctgtagaattgtgcctcctcgcggtcagtctcggtgacaccgaaaccctcattcagcacccagcgtctatgacacattcgcccgttgcgccagaggaacggcttaaagcaggtattaccgacggacttatccgtctttctgttggtcttgaagatccagaagatattattaacgaccttgaacacgccatcagaaaggcaacattctga

SEQ ID NO: 609 - DNA, 1200 bp, Porphyromonas gingivalis
  misc_feature 1..1200: note = DNA Porphyromonas gingivalis
  source 1..1200: mol_type = genomic DNA
atgaaaaaagaagaccttatgcgtagtggctttgccacacgtgccatccatggaggcgctatcgagaacgccttcggctgcttagccactcccatctaccaaacatcgactttcgtttttgacactgccgaacagggaggccgccgctttgccggagaggaagacggatacatctatacccgtctgggcaaccccaactgcacccaagtggaagagaaactggccatgctcgagggcggagaagccgccgcatcggcctcatccggtatcggagccatcagctctgccatctgggtatgcgtgaaggccggcgaccatatcgtagccggcaagacgctctacggctgcaccttcgccttcctcacccacggactgagccgctacggtgtggaagtcaccctcgtggatacccgccatccggaagaggtggaggctgccattcgcccgaatacgaagctcgtctatctggagactccggccaaccccaatatgtacctgaccgacatcaaggcagtctgcgacatcgctcataagcacgaaggcgtacgcgtcatggtggacaatacctactgcacgccctatatctgccgtccgctggagctgggtgccgacatcgtggtacacagcgcgaccaagtacctgaacggacatggcgacgtcatcgccggattcgtcgttggtaaagaggactacatcaaggaggtgaagctcgtcggcgtcaaggacctcacgggggccaatatgagtccgttcgatgcttatctgatcagccgcggcatgaagacgctgcagatacgtatggagcagcactgccgcaatgctcagaccgtagccgaattcctcgaaaagcatccggccgtagaagcagtttatttccccggacttccgagcttcccccaatacgaattggccaagaagcagatggcactgcccggagccatgatcgccttcgaagtgaagggcggttgcgaagccggtaagaagctgatgaacaacctgcacctctgctccctcgccgtgagcttgggcgatacggaaaccctcatccagcatccggccagcatgacccactcgccctacacacccgaagagcgtgctgccagcgacatatccgaaggactggtacgcctgtccgtgggtctggagaacgtggaggacatcatcgccgacctcaaacacggtctggacagcctgatctaa

SEQ ID NO: 610 - DNA, 1182 bp, Brevibacterium aurantiacum
  misc_feature 1..1182: note = DNA Brevibacterium aurantiacum
  source 1..1182: mol_type = genomic DNA
atgacctcactgcacccagaaacgctcatggtccacggcggaatgaaaggcctcaccgaggcaggagtccacgtaccggccatcgacctctcgaccaccaacccagtcaacgatgtcgccaccggcggtgactcctacgaatggctcgccaccggccatacgctcaaggacggcgactcggccgtctaccagcgcctctggcagcccggtgtcgcacgcttcgagaccgcgctggccgggctcgaacacgctgaggaagcagtcgccttcgccacgggcatggccgcaatgactgccgcacttctcgcggccgtcagcgcaggaacaccccacatcgtcgcagtgcgtcccctctatggcggaagcgaccacctcctcgaaaccggactgctggggacaacagtcacatgggcaaaggaagccgacatcgcctcggcgatccaagatgacaccggactcgtcattgtcgagaccccggcaaaccccagcctggaccttgttgatctcgacagtgtcgtctcagccgccggcaacgtgcctgtgctggtggacaacacattctgcacacctgttctccagcagcccatctcccacggagcggccctcgtactgcacagcgcgacaaaatacctcggcggtcatggcgatgccatgggcggcatcatcgccaccaacgccgactgggcgatgcgcctgcgacaggtccgagccatcacaggagccctgctccaccccatgggcgcgtatctccttcatcggggcttgcgcactctggccgtgcgcatgcgcgcggctcagaccaccgccggtgagctcgctgagcgcctggacgcgcaccctgccatctccgtcgtccactacccgggactgaaaggccaggacccacgcggactgctcggacgccaaatgtccggtggtggtgcgatgatcgcgatggagctcgccggtggattcgacgccgcccgcagcttcgtcgaacactgcaacctcgtcgtccacgcggtgtccctgggcggcgctgacactctcatccagcatccggcgtcactgactcacaggccagttgcggccacggcgaagcccggcgatggtctcatccgactctctgtgggactcgaacacgtcgatgacctggcagacgatctcatcgctgccctcgacgcgagtcgggccgctgcctga

SEQ ID NO: 611 - DNA, 1674 bp, Streptomyces sp.
  misc_feature 1..1674: note = metDC Streptomyces sp.
  source 1..1674: mol_type = genomic DNA
atgagcccgaccgccttccccgccgccgagaccgcgaccgcgcccgcgaccgccgtcgatcccggtccggagctggacggcggtgacttcgccctccccgagggcggcctggacgacgaccggcggctgcgcgcgctcgacgccgtggacgagtacctgacccgcaagcgcaagcacctggtcggctaccaggccacccaggacatgcagggcaccgcactggacctcgcccggttcatgccgaacaacatcaacaacctcggcgacccgttccagagcggcggatacaagcccaacaccaaggtcgtcgagcgggccgtgctcgactactacgcgaagctctggcacgccgagcgcccgcacgacccggccgacccggagtcgtactggggctacatgctgtccatgggctcgaccgagggcaacatgtacgccctctggaacgccagggactacctgagcggcaaggcgctgatccagccgccgaccgcccccttcgacgcggtgcgctacgtcaaggccgaccccgaccgacggaacccgaacgcccaccacccggtggccttctactccgaggacacccactactccttcgccaaggccgtggccgtcctcggcgtggagaccttccacgccgtcggcctggagaagtacgccgacgagtgcccgctggtcgacccggtgaccgggctgcgcacctggcccaccgaggtgccctcccgcccgggtccgtccggcctgtcctgggacggccccggcgagatagacgtcgacgccctcgccgtactcgtcgagttcttcgccgccaagggtcacccggtcttcgtcaacctcaacctcggcagcaccttcaagggcgcccacgacgacgtccgcgccgtctgcgagcgcttgctgccgatcttcgagcggcacgggctcgtccagcgcgaggtggtctacggcagctgcccgcagaccggccggccgctggtggacgtgcgccgcggcttctggatccacgtggacggcgcgctcggcgccggctacgcgccgttcctgcggctggccgccgaggacccggaagggtacggctggacgcccgaggcggagctgcccgagttcgacttcggcctgcggctgcccaccgccgggcacggcgaggtggacatggtctcctcgatcgcgatgagcggccacaagtgggccggcgcgccgtggccgtgcggcatctacatgaccaaggtgaagtaccagatctcgccgccgtcccagccggactacatcggcgccccggacaccaccttcgccggctcccgcaacggcttctccccgctgatcctctgggaccacctgtcccggtactcctaccgggaccaggtggagcggatccgcgaggcccaggagctggccgcctacctggagcggcggctgaccgccatggagcgcgaactcggcgtcgagctctggccggcccgtaccccgggcgccgtcaccgtacggttccgcaagccgagcgccgagctggtggccaagtggtcgctgtcctcccaggacgtgctgatggtcccgggcgacgagaccacccggcgcagctacgtgcacgtcttcgtgatgccctcggtcgaccgggccaagctggacgcgctgctcgccgaactcgccgaggacccggtgatcctgggcgcaccgtag

SEQ ID NO: 612 - DNA, 4141 bp, synthetic construct
  misc_feature 1..4141: note = Synthetic: Synthetic polynucleotide
  source 1..4141: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaatctacgactcttgacgacgaggaggaaataaatgacgagccttcaccccgaaactttaatggtgcacgggggtatgaagggattgacagaggcgggtgttcacgtcccggcgatcgacctgtcgacaacgaatcctgttaatgacgtagcgacagggggtgattcttacgaatggcttgcaacgggtcacacgttgaaggatggtgattctgctgtttaccagcgtttatggcagcccggtgtagctcgtttcgagacggctctggctgggctggagcacgcggaggaggctgtagctttcgccacgggtatggccgccatgaccgcggccttattggcagcagtatcagctggcacgcctcatatcgtcgcagtccgcccattatacggcggttcagaccatcttttagagactggtttactggggacaactgtcacgtgggcgaaagaagctgacatcgccagtgcaatccaagacgatacagggttagtgatcgtggagacgccagccaatcctagtcttgatctggtagacctggattctgtcgtgtcagcggcagggaacgtccctgtccttgtagataatacgttttgtactcctgtccttcagcaacctatctcgcacggcgccgcacttgtattacattcggcaacaaagtatttaggggggcatggggacgccatgggcgggatcatcgcgactaatgctgattgggctatgcgcctgcgtcaggtccgtgcgatcactggtgcattactgcatccgatgggcgcgtacttgttacaccgcggtttgcgtactctggctgttcgcatgcgtgccgctcagacaactgctggtgagcttgctgagcgcttagacgctcatcccgctatttcagttgtgcactacccaggattgaaggggcaagatcctcgtggattgttaggccgtcagatgagtggaggtggggcaatgattgctatggagctggcagggggattcgatgcggcacgctcctttgttgagcattgtaacttggtcgtgcatgccgtatcacttgggggagcagacacattaattcaacatccggcaagtttgacacaccgcccggtcgcagcaaccgccaagcctggtgatggtttgatccgcttgagcgtggggcttgagcacgttgacgacttggccgatgacttgatcgctgctttggacgcctctcgcgctgctgcatgaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 613 - DNA, 4156 bp, synthetic construct
  misc_feature 1..4156: note = Synthetic: Synthetic polynucleotide
  source 1..4156: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaacggtctttcaacaaccacaggaggtattaatatgtctgattgtcgtgcctacggctttaacacacagatcgtccacgccggacaacaaccggacccaagcactggtgcattgtcgacgccaatctttcagacttctactttcgttttcgattcggcagaacagggggccgcacgtttcgcgttggaggaaccgggatacatctatacgcgcttgggcaatccaactactgatgcgcttgaaaagaaacttgcagtgctggaacgtggggacgcggcgttagccacagcgagcggcatctccgctattaccacaagtcttcttactctttgccagcaaggggaccatatcgtatcagctagtgcaatctacggttgcactcacgcattcttaagtcattcgcttcctaaatttggcattaacgtttcattcgtcgacgcagcgaagcccgaagaaatccgcgccgccatgcgtccagagacaaaagtggtatatatcgagacgcccgcgaaccctactttatccttagtggatattgagactgttgctggaatcgcgcaccagcaaggagccttgttagtcgtggacaacacttttatgtctccgtattgtcaacaaccattactgctgggtgctgacatcgtggtccatagtgttaccaagtacattaacgggcatggggatgttattggcggtgtcatcgtaggtaaacaggagttcatcgaccaagcacgctttgtgggattaaaggacattacgggcggatgcatgagcccctttaatgcatggctgacacttcgcggggtaaagacgttagggatccgtatggagcgtcattgcgaaaacgcattgaaaatcgcgcgttttcttgagggccatcctgctattacacgtgtctattatcctggattgccgtcacacccccaatacgaacttgggaagcgccaaatgtcgcttcctgggggaatcatttcgtttgaaattgctggtggccttgaagcaggtcgccgtatgattaacagcgtggagttatgtttgctggcagttagcttaggtgatacggagacgttgattcagcatccggcgagcatgacacattcccctgttgctcccgaggagcgtctgaaggctggcatcactgatggactgatccgtttgtccgtcggccttgaagatccggaagacatcattaacgacctggagcacgccattcgcaaggcaacgttttaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 614 - DNA, 4156 bp, synthetic construct
  misc_feature 1..4156: note = Synthetic: Synthetic polynucleotide
  source 1..4156: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagggtattattgtcgtaaggaggaatctatgaagaaagaggacttaatgcgttcggggttcgcaactcgtgctattcatggtggagccatcgaaaatgcttttgggtgcctggccacccccatctaccaaacttcaacgtttgttttcgatacggctgaacagggtggacgtcgctttgcgggtgaagaagatgggtacatttacactcgcttaggaaacccgaactgtacccaggttgaggagaagctggcgatgttagaaggaggggaagcagcggcctccgcaagtagtggtatcggagcgatctcttcagctatctgggtgtgcgtcaaggcgggagatcatattgtagcgggaaaaactttgtacggttgcacgtttgcgttcttgacccacggactttctcgctatggagttgaggtcaccttggtggacacgcgccatccagaagaggttgaggccgctattcgtccaaacactaagttggtctatttggaaactcccgcaaaccctaacatgtaccttactgatatcaaagctgtgtgtgatatcgcgcacaagcatgagggtgttcgcgtcatggtggataatacctactgtacaccttacatctgtcgcccactggagttaggtgctgatattgttgtccattccgccacaaaatatctgaacggacatggagatgttatcgctgggtttgtagtagggaaagaggattacatcaaggaggtcaaattggttggtgttaaggacttgacaggagccaatatgtcacccttcgacgcataccttattagtcgtggtatgaagacgttacaaatccgtatggaacaacattgccgtaacgcacaaacagttgctgagtttctggagaagcatcctgcggttgaggcggtgtacttcccgggcttgccttcattcccgcaatatgagttggccaagaagcagatggcacttccgggagcgatgattgcgtttgaagttaagggaggctgcgaagccggtaagaaacttatgaacaatcttcatctgtgttccttagccgtctccttaggcgataccgagactttaatccaacacccggcctcaatgacgcactcgccctatacgcccgaggaacgcgccgcaagcgacatctcggaggggcttgtacgcctttctgttggattggaaaatgttgaggatattatcgcggacctgaagcatggcctggatagtcttatctaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 615 - DNA, 4630 bp, synthetic construct
  misc_feature 1..4630: note = Synthetic: Synthetic polynucleotide
  source 1..4630: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaacaccagtagttcaaaaggaggtagaacagatgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 616 - DNA, 7482 bp, synthetic construct
  misc_feature 1..7482: note = Synthetic: Synthetic polynucleotide
  source 1..7482: mol_type = other DNA
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctaggttccaggcattcgaaatgccacgactaacttaatgacgataataaataatcaatgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcg

SEQ ID NO: 617 - protein, 393 aa, synthetic construct; identical to SEQ ID NO: 600
  REGION 1..393: note = Synthetic: Synthetic polypeptide

SEQ ID NO: 618 - protein, 398 aa, synthetic construct; identical to SEQ ID NO: 601
  REGION 1..398: note = Synthetic: Synthetic polypeptide

SEQ ID NO: 619 - protein, 399 aa, synthetic construct; identical to SEQ ID NO: 602
  REGION 1..399: note = Synthetic: Synthetic polypeptide

SEQ ID NO: 620 - protein, 557 aa, synthetic construct; identical to SEQ ID NO: 603
  REGION 1..557: note = Synthetic: Synthetic polypeptide

SEQ ID NO: 621 - protein, 207 aa, synthetic construct
  REGION 1..207: note = Synthetic: Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 622 - protein, 343 aa, synthetic construct; identical to SEQ ID NO: 604
  REGION 1..343: note = Synthetic: Synthetic polypeptide

SEQ ID NO: 623 - skipped

SEQ ID NO: 624 - protein, 271 aa, synthetic construct; identical to SEQ ID NO: 606
  REGION 1..271: note = Synthetic: Synthetic polypeptide

SEQ ID NO: 625 - protein, 316 aa, synthetic construct
  REGION 1..316: note = Synthetic: Synthetic polypeptide
MSELVVFKANELAISRYDLTEHETKLILCCVALLNPTIENPTRKERTVSFTYNQYAQMMNISRENAYGVLAKATRELMTRTVEIRNPLVKGFEIFQWTNYAKFSSEKLELVFSEEILPYLFQLKKFIKYNLEHVKSFENKYSMRIYEWLLKELTQKKTHKANIEISLDEFKFMLMLENNYHEFKRLNQWVLKPISKDLNTYSNMKLVVDKRGRPTDTLIFQVELDRQMDLVTELENNQIKMNGDKIPTTITSDSYLRNGLRKTLHDALTAKIQLTSFEAKFLSDMQSKHDLNGSFSWLTQKQRTTLENILAKYGRI

SEQ ID NO: 626 - protein, 286 aa, synthetic construct
  REGION 1..286: note = Synthetic: Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 627 - DNA, 1674 bp, synthetic construct
  misc_feature 1..1674: note = Synthetic: Synthetic polynucleotide
  source 1..1674: mol_type = other DNA
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatggacggaacggccttggatttagcccgtttcatgccccacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 628 - DNA, 1674 bp, synthetic construct
  misc_feature 1..1674: note = Synthetic: Synthetic polynucleotide
  source 1..1674: mol_type = other DNA
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctctgaccgtacgttttcgcaaaccctctccggagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 629 - DNA, 1674 bp, synthetic construct
  misc_feature 1..1674: note = Synthetic: Synthetic polynucleotide
  source 1..1674: mol_type = other DNA
atgtccccgacggcgtttccagcggccgaaacagctactgcccctgcaactgccgtcgatcctgggccagaactggacggcggagatttcgcccttccagagggcgggctggatgacgatcagcgcttacgtgcattggacgcagttgacgagtatttgacccgcaagcgcaagcatttggttgggtaccaagctacccaggatatggacggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 630 - DNA, 1674 bp, unidentified
  misc_feature 1..1674: note = Stanieria sp. NIES-3757 sequence
  source 1..1674: mol_type = genomic DNA; note = Stanieria sp.
atggggttccagttactgtctaaacataagctgtcagccgaggatcaacagaaacttgaccgcttttatcgtgatattcagacagaagcagaacgattcctgggttacccatgtaacgaactctttgactactcccccttgttccggttcctgcaatatccgctgaataacgtcggcgacccgtacctgccgagtaactaccacctgaacacgcacaactttgagtgcgaagtactggaaatcttccgtaccctgaccgaggctactgaaggttcgacttggggctacgtgaccaacggcggtacggaaggtaatcattatggtcttttcctggcgagagagctgctgcctgaaggccttgtttactattctcaggatgcgcactactcgatcgataaaatcctgaggtgcctcaacctccgtagcataatgattcgcagccacgacgacggacgcatggacctggatgatctgcgtgaaactctgcgtatccatcgcgacttgccgccgatcgtttgcgctaccattggtactacaatgaagggcgctgtagatgacatcgcaggcattaaaaagatcttcaaagatctggcaatacaccgtcactatatccatgctgacgcggccctaggtggcatgattttaccgttcctggataactccccaccgtggaattttaaagctggaatcgactctatcgctatctccggtcacaaaatggtgggcagtcctatcccgtgtggggttgtcctggctaaaaagtcgaacgttgaacgtattgcacagagcgtggaatacattggtactctggataccaccctgtctggctcccgtaacgccttgactccgttatttctgtggtacgcgttccacaccgttggtatcgaaggtttcaaacgtatcatcccggcatgcttaaaaatggcggactatgccatcgctcagctgaacaaaattaaccgcaatgcgtggcgctacccttacagcaacacggtagtcttcgatcgcccaagccccgaagtgactcgttattggcagctggcttgtcagggcaacctgagccacctaatcaccatgccacacgttacatctactcaaattgatcatctggttgctgacatcatcgcttctgagccgataccgccgctgccgaccctgtcagttactccggcatgcgaactgctgacttctaccccggaccaggatattacgctgatcggcaccgctaatcataatctgctctccgaagtatctaccgccctggctgccgagggtctgtcaattgaaaacctggctgctgtggcggtagaaagcgaggacgttgaagttgtaaggctccgcgttaacaaccgtgagcgtgcactgcaaatcctgaaccagaacctggatatcggtcgttgctacggtcaggctcgaccctttggcaacgaagaagcgacgcaggtactgtcccagctggaatatcaaagcgtgggggaggatgcactactggtccagcttgacgattgccctggcagcctggcggagctgttgaaggattgccgcaacgaagcggtaaaaatccgtaatatccgactgctttggcgtgggcacggtaagggcgtcgtagcaattgctaccacttctccagatgcgctgaaaacgctgctgaaagaccgtattcttttgagctaa

SEQ ID NO: 631 - DNA, 1278 bp, Mus musculus
  misc_feature 1..1278: note = DNA Mus musculus
  source 1..1278: mol_type = genomic DNA
atgtccacacctagtgaagtaaagaaggatttgctgggtgcagcagggtcattatggccgtcggagcccattacgctgggtccaggtgaaagtgcttggcagctggtattgaagaagatccaagagttgagtgacagcggtcatcaagacccgttcatggttgcagaccttgatgtccttgtgtctcgtcatcagacgttctgtcaagcactgcctagagtacaacccttctatgcagtaaagtgcaatagtaacccatgggtgttacgggtgttggcagctcttggcacgggatttgattgtgcttctcagggagaattggagcaagttttgggcttgggtgtagcgccgtcacggataatcttcgcaaatccctgtaaagcagtcagccacattcagtttgcagctcggtgcggtgtgcaattgttgacattcgacagcgaagaggagttaatcaaggttgcgcagtaccatccaggcgcacggttggtgcttcggattcaaacccaggactcacaatcaacgttcccactttccaccaagttcggtgcttctttagaagcatgtggacaccttctgcaggttgccagagagctgggtcttgccgtggtaggtgctagctttcatgtaggaagcgactgccacacacctcagagttttcgtcaggccatcgcagattgtcatcgtgtgttcgagatgggccgtaaggcaggtcatgatatgtcgcttcttgatttgggtggagggttcccaggtgtggaaggttccgaggcgaagtttgaggagatggcaagagtaatcaatgccgctcttgctcagtactttccggaagagactggcatcgaggtgatcgcggaacctggtcgtttctacgctgggtcggtgtgcactgcagctgtgaacatcatcgcgaagaagtctgtcttggaaccaggtggtcatcgtaagcttatgtactaccttaatgaaggacattacggttctttcagattgttcttgcgtgatccagtgcctcgtattcccatcgtggtgaaagagttcccatccgaaccaccactgtttccttgcactttgtacggtcccacatgtgacgcctatgatcggttgttttccgaagaggtacaattgccagagctggatgttggagattggttgatcttcccagatatgggtgcctatacctcctcaatgtcctcgaccttcaacggatttccaccggccaccgtgtattgcgcaatgtcaccgcagttacgctccctgttggagactgtaccataa

SEQ ID NO: 632 - DNA, 1278 bp, Mus musculus
  misc_feature 1..1278: note = DNA Mus musculus
  source 1..1278: mol_type = genomic DNA
atgaacacacctagtgaagtaaagaaggatttgctgggtgttgcagaacatttacgtccgtcggagcccattacgctgggtccaggtgcgagtgcttggcagctggtattgaagaagatcaaggagttgagtattagcggtcgtcaagacgctttcatggttgcagaccttgatgtccttgtgtctcgtcatcggacgttcttacaagcactgcctagagtacaacccttctatgcagtaaagtgcaatagtaacccatgggtgttacttgtgttggcagctcttggcacgggatttgattgtgcttctcagggagaattggagcaagttttgggcttgggtgtagcgccgtcacggataatcttcgcaaatccctgtaaagcagtcagccacattcagtttgcagctcggtgcggtgtgcaattgttgacattcgacaatgaagaggagttaatcaagttagcgcgttaccatccacgtgcacggttggtgcttcggattcaaaccctggactcacaatcaacgttcccacttagcaccaagttcggtgctcacttagaagcatgtggacaccttctgcaggttgccagagagctgggtcttgccgtggtaggtgctagctttcatgtaggaagcgactgccacacacctgagagttaccgtcaggccatcgcagattgtcatcgtgtgttcgagatgggctgtaaggcaggtcatcacatgtcgcttcttgatttgggtggagggttcccaggtgtgaaaggttccgaggcgaagtttgaggaggttgcaagagtaatcaataccgctcttgctcagtactttccggaagagactggcatcgaggtgatcgcggaacctggtcgtttctacgctgggtcggtgtgcactgcagctgtgaacatcatcgccaagaagtctagtttggacccaggtggtcatcgtaagcttgcttactaccttaatgaaggacattacggtgtattcagattgttcttgcgtgatccagtgcctcgtattcccatcgtggtgaaagagttcccatccgaaccaccactgtttccttgcactttgtacggtcccacatgtgacgcctatgatcggttgttttccaccgaggtacaattgccagagctggatgttggagattggttgatcttcccagatatgggtgcctattcgtcctcaatgtcctcgaccttcaacggatttccaatagccaccgtgtatgatgcaatgtcaccgcagttacgctccctgttggagactgtaccataa

SEQ ID NO: 633 - DNA, 1242 bp, Entamoeba histolytica
  misc_feature 1..1242: note = DNA Entamoeba histolytica
  source 1..1242: mol_type = genomic DNA
atgaaacaaacgtcccttgaggtgaaggaatttgccttgaatctcatttctcagttcgaaccagaaaaccagcctctgggtttctggatattcgacaccgaaggcgttgagaaagcggtagaacgctggaaaaagaacatgccgactgtccgtccctgttttgcagttaaatgcaacccggagccgcacctggtgaaattactgggggaactgggttgcggcttcgattgcgctagcctgaacgaaatcaaagaggtactggacttgggttttaatccggaagatatcacttatagtcagaccttcaaaccgtacaaccagttaattgaagcttcgcatctgggcatcaaccacacgatcgttgattcaatcgacgaagttcaaaaaattgctaaatacgcgcctaagatgggtatcatgattcggatcatggaaaatgacacaagcgcaggccacgtctttggagagaaattcggtctgcatgatgatgaagttgagatcgtactgaaggaaattaaagacaaaggtctgaacctggacggcgttcatttccacgttggctctgattcccacaacagcgaagtgtttactaaggcactgaccaaagctcgtaacactgtaaccctggccgaacagttcggcatgaaaccgtacctgatcgacattggtggcgggttctctcaggttgcgccgttcgaagaatttgctgctaccatcgaaaaaactataaaggaactggaatttccagagcgaactcgtttcattgcagagccgggtcgctatatggcatcaaatgcctttcaccttgtctcttcgctgcatggtaaaagggtgcgcatccagaacggtaagaaacagatcgaatacaccagcggcgatgggctgcacggctccttcggctgttgcatctggttcgaaaaacagaagtcttgcgaatgtataacacaaaaagtaaacgagaacaccaaaatgtatgaaagcatcatctacggcccatcttgcaacggttcggacaaagtggccacgcaggagttgccggaaatggagccgggtaaagattggctgctgttccccaatatgggtgcttacactatttccatggcgaccaactttaacggcttcgaagaacgtaaccatgtaatctatacgttaccactcaaaagtactaaaataattcagatccctaaaagcattgaatgcaactccgttccgtctttaaacggaatcccacactacgcgtaa

SEQ ID NO: 634 - DNA, 1509 bp, Flavobacterium segetis
  misc_feature 1..1509: note = DNA Flavobacterium segetis
  source 1..1509: mol_type = genomic DNA
atggggaccattaacacgaagatctataaatacatgagcatctggaaaacaaaacctctgtccgtgctcttgtctgaagcaactgaggatgaaaaaggcctgaagcgcactctgtcggcccgttcacttgttgcgctgggtgtcggtgctattatcggcgctggtttattctctctgaccggcatagctgcggcagacaatgctggaccggcagtaaccctgagctttatcctggcctccgttggttgcgcgttcgctggcctgtgttacgcagaatttgcttctatgattccagttgcgggtagcgcctacacttatagttatgctaccatgggcgagttcgtggcgtggatcatcggttgggatctggtactcgaatacgcattgggcgcagctactgttgccgttagctggtcccagtacgtggacaaattcttgcaaaactacggcatccatattccgaactctatcctccacgggccgtgggataccacccccggtattatcaatttaccgtcgatatttatcatctgcctgctgagcgtgctgctgattcgtggtactaaagaatctgctctgatcaacaacattctggtaatcctgaaagtcacggttgtcatcgtgttcattggcctgggctgggggttcatgaactccgcaaaccacacgccctttatcccggttaacgaaggtgaggctctactgtcttctggtgaaatgagtttcctcaactttttcagcagtgactactttggacactacggatggtccggtattcttcgcggcgctggtgtagtattcttcgcatttatcggcttcgacgcggtgagcactgcggcacaggaggccaaggatccgcagaaaggcatgccaatcggtattctgggctcactgatcatttgcaccgttctgtacgtgcttttcgctttcgttctgaccggtctggaaaactatctaaacttcaaaggtgacgcttctcctgtcaccactgcatttgccaaaacaggctatactttcctgaatagcggtctgacgatcgctatcatagcgggctacacatccgttatgctggtaatgttgatgggtcagtcccgtgtcttttatagtatgtctgtggatggcctgcttccgaagtttttctcgaccctgcataccaaaaacaggactccgtacaaaactaatttgctgttcatggttttcgtaagcctgttcgctggctttgttccggtcagcgacctgggccatatggtatccatcggtaccctcttcgctttctgcctggtgtgtatcggcgttatcgttatgcgaaaaaccaacccagacgccgttcgcggttttcgtgttccttttgtaccggttttcccgattatcggtgtagttatttgtctggttctaatggcgggcctgccgattgaatcttgggaacgtctggcgatctggatgattctgggtgtcgtgatctacttcttctactctaaaaagaactctaaactgaataaccccgaataa

SEQ ID NO: 635 - DNA, 1481 bp, Flavobacterium frigoris
  misc_feature 1..1481: note = DNA Flavobacterium frigoris
  source 1..1481: mol_type = genomic DNA
atggggacgatcaatactaagaccaacaaatatatgagcatttggaaaaccaaaccgttgtctgtactgttaaacgaggcctcagaagatgaaaagggcctgaaaaggactctgtcctctcgttccctcgtggctctgggtgtcggtgcgatcattggcgcaggtctgtttagcctaacaggcatcgcagctgcggaacatgctggtccagcggttactctgagtttcatactggccgctgttggttgtgctttcgcaggcctgtgctacgcggagtttgcgtcgatgatccctgtggctgggtctgcttacacctatagctacgcaaccatgggcgaatttatggcgtggatcattggctgggaccttgtactggaatacgctctgggtgcagcgactgttggtgtatcctggtcccgttacttactggaattgctgaacaaatatggtgttcacctgaacccgaaattcatctgctctccgtgggagacacttaccctgggcgacggcactattatcgatggcgggtacatcaatctgccggcaattctgatcgtgagcgccctcagcttgctgctgattagaggtacccaggaatctgcttctattaacaacatcctggttgtgctgaaagtaatagtcgtgatcatgttcatcgttttaggatgggactatatcgatcccgcaaattactcaccttacatcccggaaaacaccggcgtaaagggccaattcggttggtcgggtatcgctgcgggtgctggtacggttttctttgccttcattggtttcgacgccgtttccactgcggctcaggaggctaaaaacccgcagaaaggcatgccaattggcatcctggggtctttggtaatttgtacgatcctgtacgtcctttttgcccacgttatgacgggcctggtgccgtattataagttcgctggagatgctaaacccgctgcgacagcattcgcagtcaccggttacagttttctgcaaactggactgattgttgcgatcctggctggctatactagcgttatgctggtcatgctgatggggcagagtcgtgttttctacaccatgagcaaagacggtctgctaccaccgctgttcggtcagatccattcgaaatttcgcactccgtacaagactaacctgttctttatggtattcgtttctttattcgcgggtttcgtgccggttagcgacctcggccacatggtcagcatcggtaccctcctggcgtttgttcttgtgtgcataggtgtgctggtgatgcgaaaaaagatgccagatgctccgcgttctttcaaaaccccgttcgttccgtatgtacccatcgcaggcgtcctggtgtgcacttacctgatgtactccctcccttacgaatcctggattcgcttagtgctttggatggctatcggcgtagccctgtacttcgtgtatggaaaaaagcactcaaaactgaacaatccggataa

SEQ ID NO: 636 - DNA, 2533 bp, Escherichia coli
  misc_feature 1..2533: note = DNA Escherichia coli
  source 1..2533: mol_type = genomic DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 637 - DNA, 666 bp, Bacillus subtilis
  misc_feature 1..666: note = DNA Bacillus subtilis
  source 1..666: mol_type = genomic DNA
atgtttgagaagtattttccaaatgttgacttgaccgagttatggaatgccacatatgaaactctgtatatgacattgatttccttactgtttgccttcgtaatcggcgtcatcctgggattgctgttattcttaacatctaaggggtctctttggcaaaataaagcagtaaattccgttatcgcagccgttgtcaacatctttcgttcaattcccttccttattttaatcatcctgcttcttggtttcactaaattcttagtgggaacaattttgggaccaaatgcggctcttcccgcgttagtcatcggtagtgctcccttttatgctcgtctggtcgaaatcgcacttcgtgaagtggacaaaggagtgattgaggcggcgaaatcgatgggggctaagacgagcactattatttttaaggttcttatccccgagtccatgcccgcgctgatttccggaattacagtgactgcgattgcattgatcgggtcaaccgccatcgcaggagctattggttctggtggattgggaaacttagcatacgttgaaggctatcaatcgaataatgcggatgtgaccttcgtggccacagttttcatcctgattattgttttcatcattcagatcattggtgaccttattaccaacatcatcgataaacgc

SEQ ID NO: 638 - DNA, 2533 bp, synthetic construct
  misc_feature 1..2533: note = Synthetic: Synthetic polynucleotide
  source 1..2533: mol_type = other DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccggcctgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaagaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaagatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 639 - DNA, 2533 bp, synthetic construct
  misc_feature 1..2533: note = Synthetic: Synthetic polynucleotide
  source 1..2533: mol_type = other DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaagaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaagatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 640 - DNA, 2533 bp, synthetic construct
  misc_feature 1..2533: note = Synthetic: Synthetic polynucleotide
  source 1..2533: mol_type = other DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgcctccctgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaagaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaagatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 641 - protein, 557 aa, synthetic construct
  REGION 1..557: note = Synthetic: Synthetic polypeptide
MSPTAFPAAETATAPATAVDPGPELDGGDFALPEGGLDDDRRLRALDAVDEYLTRKRKHLVGYQATQDMDGTALDLARFMPHNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 642 - protein, 557 aa, Streptomyces sp.; identical to SEQ ID NO: 603
  REGION 1..557: note = misc_feature - PRT Streptomyces sp.

SEQ ID NO: 643 - protein, 557 aa, synthetic construct
  REGION 1..557: note = Synthetic: Synthetic polypeptide
MSPTAFPAAETATAPATAVDPGPELDGGDFALPEGGLDDDRRLRALDAVDEYLTRKRKHLVGYQATQDMQGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGALTVRFRKPSPELVAKWSLSSQDVLMVPGDETTRRSYVHVFVMPSVDRAKLDALLAELAEDPVILGAP

SEQ ID NO: 644 - protein, 556 aa, synthetic construct
  REGION 1..556: note = Synthetic: Synthetic polypeptide
SPTAFPAAETATAPATAVDPGPELDGGDFALPEGGLDDDQRLRALDAVDEYLTRKRKHLVGYQATQDMDGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 645 - protein, 557 aa, unidentified
  REGION 1..557: note = misc_feature - PRT Stanieria sp. NIES-3757
  source 1..557: note = Stanieria sp.
MGFQLLSKHKLSAEDQQKLDRFYRDIQTEAERFLGYPCNELFDYSPLFRFLQYPLNNVGDPYLPSNYHLNTHNFECEVLEIFRTLTEATEGSTWGYVTNGGTEGNHYGLFLARELLPEGLVYYSQDAHYSIDKILRCLNLRSIMIRSHDDGRMDLDDLRETLRIHRDLPPIVCATIGTTMKGAVDDIAGIKKIFKDLAIHRHYIHADAALGGMILPFLDNSPPWNFKAGIDSIAISGHKMVGSPIPCGVVLAKKSNVERIAQSVEYIGTLDTTLSGSRNALTPLFLWYAFHTVGIEGFKRIIPACLKMADYAIAQLNKINRNAWRYPYSNTVVFDRPSPEVTRYWQLACQGNLSHLITMPHVTSTQIDHLVADIIASEPIPPLPTLSVTPACELLTSTPDQDITLIGTANHNLLSEVSTALAAEGLSIENLAAVAVESEDVEVVRLRVNNRERALQILNQNLDIGRCYGQARPFGNEEATQVLSQLEYQSVGEDALLVQLDDCPGSLAELLKDCRNEAVKIRNIRLLWRGHGKGVVAIATTSPDALKTLLKDRILLS

SEQ ID NO: 646 - protein, 425 aa, Mus musculus
  REGION 1..425: note = misc_feature - PRT Mus musculus
MSTPSEVKKDLLGAAGSLWPSEPITLGPGESAWQLVLKKIQELSDSGHQDPFMVADLDVLVSRHQTFCQALPRVQPFYAVKCNSNPWVLRVLAALGTGFDCASQGELEQVLGLGVAPSRIIFANPCKAVSHIQFAARCGVQLLTFDSEEELIKVAQYHPGARLVLRIQTQDSQSTFPLSTKFGASLEACGHLLQVARELGLAVVGASFHVGSDCHTPQSFRQAIADCHRVFEMGRKAGHDMSLLDLGGGFPGVEGSEAKFEEMARVINAALAQYFPEETGIEVIAEPGRFYAGSVCTAAVNIIAKKSVLEPGGHRKLMYYLNEGHYGSFRLFLRDPVPRIPIVVKEFPSEPPLFPCTLYGPTCDAYDRLFSEEVQLPELDVGDWLIFPDMGAYTSSMSSTFNGFPPATVYCAMSPQLRSLLETVP

SEQ ID NO: 647 - protein, 425 aa, Mus musculus
  REGION 1..425: note = misc_feature - PRT Mus musculus
MNTPSEVKKDLLGVAEHLRPSEPITLGPGASAWQLVLKKIKELSISGRQDAFMVADLDVLVSRHRTFLQALPRVQPFYAVKCNSNPWVLLVLAALGTGFDCASQGELEQVLGLGVAPSRIIFANPCKAVSHIQFAARCGVQLLTFDNEEELIKLARYHPRARLVLRIQTLDSQSTFPLSTKFGAHLEACGHLLQVARELGLAVVGASFHVGSDCHTPESYRQAIADCHRVFEMGCKAGHHMSLLDLGGGFPGVKGSEAKFEEVARVINTALAQYFPEETGIEVIAEPGRFYAGSVCTAAVNIIAKKSSLDPGGHRKLAYYLNEGHYGVFRLFLRDPVPRIPIVVKEFPSEPPLFPCTLYGPTCDAYDRLFSTEVQLPELDVGDWLIFPDMGAYSSSMSSTFNGFPIATVYDAMSPQLRSLLETVP

SEQ ID NO: 648 - protein, 413 aa, Entamoeba histolytica
  REGION 1..413: note = misc_feature - PRT Entamoeba histolytica
MKQTSLEVKEFALNLISQFEPENQPLGFWIFDTEGVEKAVERWKKNMPTVRPCFAVKCNPEPHLVKLLGELGCGFDCASLNEIKEVLDLGFNPEDITYSQTFKPYNQLIEASHLGINHTIVDSIDEVQKIAKYAPKMGIMIRIMENDTSAGHVFGEKFGLHDDEVEIVLKEIKDKGLNLDGVHFHVGSDSHNSEVFTKALTKARNTVTLAEQFGMKPYLIDIGGGFSQVAPFEEFAATIEKTIKELEFPERTRFIAEPGRYMASNAFHLVSSLHGKRVRIQNGKKQIEYTSGDGLHGSFGCCIWFEKQKSCECITQKVNENTKMYESIIYGPSCNGSDKVATQELPEMEPGKDWLLFPNMGAYTISMATNFNGFEERNHVIYTLPLKSTKIIQIPKSIECNSVPSLNGIPHYA

SEQ ID NO: 649 - protein, 502 aa, Flavobacterium segetis
  REGION 1..502: note = misc_feature - PRT Flavobacterium segetis
MGTINTKIYKYMSIWKTKPLSVLLSEATEDEKGLKRTLSARSLVALGVGAIIGAGLFSLTGIAAADNAGPAVTLSFILASVGCAFAGLCYAEFASMIPVAGSAYTYSYATMGEFVAWIIGWDLVLEYALGAATVAVSWSQYVDKFLQNYGIHIPNSILHGPWDTTPGIINLPSIFIICLLSVLLIRGTKESALINNILVILKVTVVIVFIGLGWGFMNSANHTPFIPVNEGEALLSSGEMSFLNFFSSDYFGHYGWSGILRGAGVVFFAFIGFDAVSTAAQEAKDPQKGMPIGILGSLIICTVLYVLFAFVLTGLENYLNFKGDASPVTTAFAKTGYTFLNSGLTIAIIAGYTSVMLVMLMGQSRVFYSMSVDGLLPKFFSTLHTKNRTPYKTNLLFMVFVSLFAGFVPVSDLGHMVSIGTLFAFCLVCIGVIVMRKTNPDAVRGFRVPFVPVFPIIGVVICLVLMAGLPIESWERLAIWMILGVVIYFFYSKKNSKLNNPE

SEQ ID NO: 650 - protein, 493 aa, Flavobacterium frigoris
  REGION 1..493: note = misc_feature - PRT Flavobacterium frigoris
MGTINTKTNKYMSIWKTKPLSVLLNEASEDEKGLKRTLSSRSLVALGVGAIIGAGLFSLTGIAAAEHAGPAVTLSFILAAVGCAFAGLCYAEFASMIPVAGSAYTYSYATMGEFMAWIIGWDLVLEYALGAATVGVSWSRYLLELLNKYGVHLNPKFICSPWETLTLGDGTIIDGGYINLPAILIVSALSLLLIRGTQESASINNILVVLKVIVVIMFIVLGWDYIDPANYSPYIPENTGVKGQFGWSGIAAGAGTVFFAFIGFDAVSTAAQEAKNPQKGMPIGILGSLVICTILYVLFAHVMTGLVPYYKFAGDAKPAATAFAVTGYSFLQTGLIVAILAGYTSVMLVMLMGQSRVFYTMSKDGLLPPLFGQIHSKFRTPYKTNLFFMVFVSLFAGFVPVSDLGHMVSIGTLLAFVLVCIGVLVMRKKMPDAPRSFKTPFVPYVPIAGVLVCTYLMYSLPYESWIRLVLWMAIGVALYFVYGKKHSKLNNPD

SEQ ID NO: 651 - protein, 343 aa, synthetic construct; identical to SEQ ID NO: 604
  REGION 1..343: note = Synthetic: Synthetic polypeptide

SEQ ID NO: 652 - protein, 217 aa, synthetic construct
  REGION 1..217: note = Synthetic: Synthetic polypeptide
MSEPMMWLLVRGVWETLAMTFVSGFFGFVIGLPVGVLLYVTRPGQIIANAKLYRTISAIVNIFRSIPFIILLVWMIPFTRVIVGTSIGLQAAIVPLTVGAAPFIARMVENALLEIPTGLIEASRAMGATPMQIVRKVLLPEALPGLVNAATITLITLVGYSAMGGAVGAGGLGQIGYQYGYIGYNATVMNTVLVLLVILVYLIQFAGDRIVRAVTRK

SEQ ID NO: 653 - protein, 271 aa, synthetic construct; identical to SEQ ID NO: 606
  REGION 1..271: note = Synthetic: Synthetic polypeptide

SEQ ID NO: 654 - protein, 222 aa, Bacillus subtilis
  REGION 1..222: note = misc_feature - PRT Bacillus subtilis
MFEKYFPNVDLTELWNATYETLYMTLISLLFAFVIGVILGLLLFLTSKGSLWQNKAVNSVIAAVVNIFRSIPFLILIILLLGFTKFLVGTILGPNAALPALVIGSAPFYARLVEIALREVDKGVIEAAKSMGAKTSTIIFKVLIPESMPALISGITVTAIALIGSTAIAGAIGSGGLGNLAYVEGYQSNNADVTFVATVFILIIVFIIQIIGDLITNIIDKR

SEQ ID NO: 655 - protein, 343 aa, synthetic construct
  REGION 1..343: note = Synthetic: Synthetic polypeptide
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAGLLSETARRFNVNNNIISAQMDYAGGVKFGIMLTEMHGTQQDTQAAIAWLQEHHVKVEVLGYV

SEQ ID NO: 656 - protein, 343 aa, synthetic construct
  REGION 1..343: note = Synthetic: Synthetic polypeptide
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDASLLSETARRFNVNNNIISAQMDYAGGVKFGIMLTEMHGTQQDTQAAIAWLQEHHVKVEVLGYV

SEQ ID NO: 657 - DNA, 1029 bp, Escherichia coli
  misc_feature 1..1029: note = DL-methionine transporter subunit MetN (Escherichia coli K-12])
  source 1..1029: mol_type = genomic DNA
atgattaaactgagcaacattactaaggtgttccaccaaggtacacgtacgatccaggctcttaataatgtgtcactgcacgttcctgctggtcagatttatggggttatcggtgccagtggggctgggaagagcactctgatccgctgcgtcaatctgttagagcgccctacagagggctcggtactggtggacggtcaagagttgactactctgtcggagtccgagttgacaaaagcacgccgccagattggcatgattttccaacatttcaatttgttatcgagccgtacagttttcgggaacgtggccttaccactggagttggacaatactcccaaagacgaagtcaaacgtcgtgtgaccgaattattgtccttggtgggtcttggtgacaaacacgacagttatcccagtaatttgagtggcgggcaaaaacagcgtgttgccatcgcacgcgcattagcttcgaatcccaaggtgctgttatgtgatgaagcgaccagcgcccttgacccagccacaactcgtagcatcctggagcttttgaaagatatcaatcgtcgcctgggtttgaccatcttattgattacgcacgagatggacgttgtaaagcgtatctgtgactgtgtagcggtgatctccaacggtgaattaatcgaacaggacaccgtatcggaggtcttctcacatcctaagacaccccttgcacaaaaattcatccaaagcacgctgcatttagatattcctgaagattatcaggaacgcctgcaggctgaaccgtttactgattgcgttccaatgcttcgcttagagttcacagggcaatcggttgacgctcccttattgagtgaaaccgcccgccgtttcaatgttaataacaacatcatttccgcgcaaatggactacgcggggggtgttaaatttggaatcatgttaaccgaaatgcacggcacacagcaggatacacaggcggcgatcgcatggctgcaggaacatcatgttaaagtagaagtccttgggtatgtg

SEQ ID NO: 658 - DNA, 654 bp, Escherichia coli
  misc_feature 1..654: note = metI (Escherichia coli)
  source 1..654: mol_type = genomic DNA
atgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagctgtatcgtaccgtttctgcgattgtgaacattttccgttccatcccgttcattatcttgcttgtatggatgattccgtttacccgcgttattgtcggtacatcgattggtttgcaggcagcgattgttccgttaaccgttggtgcagcaccgtttattgcccgtatggtcgagaacgctctgctggagatcccaaccgggttaattgaagcttcccgcgcaatgggtgccacgccgatgcagatcgtccgtaaggtgctgttaccggaagcgctgccgggtctggtgaatgcggcaactatcaccctgattaccctggtcggttattccgcgatgggtggtgcagtcggtgccggtggtttaggtcagattggctatcagtatggctacatcggctataacgcgacggtgatgaatacggtactggtattgctggtcattctggtttatttaattcagttcgcaggcgaccgcatcgtccgggctgtcactcgcaagtaa

SEQ ID NO: 659 - DNA, 816 bp, Escherichia coli
  misc_feature 1..816: note = metQ (Escherichia coli)
  source 1..816: mol_type = genomic DNA
atggcgttcaaattcaaaacctttgcggcagtgggagccctgatcggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacttttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaaccccaaaaatctgaaaattgttgaactggaagcaccgcaactgccgcgttctctggacgacgcgcaaatcgctctggcagttatcaataccacctatgccagccagattggcctgactccggcgaaagacggtatctttgttgaagataaagagtccccgtacgtaaacctgatcgtgacgcgtgaagataacaaagacgccgagaacgtgaagaaattcgtccaggcttatcagtctgacgaagtttacgaagcagcaaacaaagtgtttaacggcggagctgttaaaggctggtaa

SEQ ID NO: 660 - DNA, 1271 bp, synthetic construct
  misc_feature 1..1271: note = Synthetic: lacI-Ptac
  source 1..1271: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaatt

SEQ ID NO: 661 - DNA, 1641 bp, synthetic construct
  misc_feature 1..1641: note = Synthetic: Ptac-metP
  source 1..1641: mol_type = other DNA
ttgacaattaatcatcggctcgtataatgtgtggaattgtgagcgctcacaattagctgtgaccagaggtaaggaggtaacaaccatgcgagtgttgaagaaacatcttaatcatgctgcggagggtttctaatggggaccattaacacgaagatctataaatacatgagcatctggaaaacaaaacctctgtccgtgctcttgtctgaagcaactgaggatgaaaaaggcctgaagcgcactctgtcggcccgttcacttgttgcgctgggtgtcggtgctattatcggcgctggtttattctctctgaccggcatagctgcggcagacaatgctggaccggcagtaaccctgagctttatcctggcctccgttggttgcgcgttcgctggcctgtgttacgcagaatttgcttctatgattccagttgcgggtagcgcctacacttatagttatgctaccatgggcgagttcgtggcgtggatcatcggttgggatctggtactcgaatacgcattgggcgcagctactgttgccgttagctggtcccagtacgtggacaaattcttgcaaaactacggcatccatattccgaactctatcctccacgggccgtgggataccacccccggtattatcaatttaccgtcgatatttatcatctgcctgctgagcgtgctgctgattcgtggtactaaagaatctgctctgatcaacaacattctggtaatcctgaaagtcacggttgtcatcgtgttcattggcctgggctgggggttcatgaactccgcaaaccacacgccctttatcccggttaacgaaggtgaggctctactgtcttctggtgaaatgagtttcctcaactttttcagcagtgactactttggacactacggatggtccggtattcttcgcggcgctggtgtagtattcttcgcatttatcggcttcgacgcggtgagcactgcggcacaggaggccaaggatccgcagaaaggcatgccaatcggtattctgggctcactgatcatttgcaccgttctgtacgtgcttttcgctttcgttctgaccggtctggaaaactatctaaacttcaaaggtgacgcttctcctgtcaccactgcatttgccaaaacaggctatactttcctgaatagcggtctgacgatcgctatcatagcgggctacacatccgttatgctggtaatgttgatgggtcagtcccgtgtcttttatagtatgtctgtggatggcctgcttccgaagtttttctcgaccctgcataccaaaaacaggactccgtacaaaactaatttgctgttcatggttttcgtaagcctgttcgctggctttgttccggtcagcgacctgggccatatggtatccatcggtaccctcttcgctttctgcctggtgtgtatcggcgttatcgttatgcgaaaaaccaacccagacgccgttcgcggttttcgtgttccttttgtaccggttttcccgattatcggtgtagttatttgtctggttctaatggcgggcctgccgattgaatcttgggaacgtctggcgatctggatgattctgggtgtcgtgatctacttcttctactctaaaaagaactctaaactgaataaccccgaataa

SEQ ID NO: 662 - DNA, 1766 bp, synthetic construct
  misc_feature 1..1766: note = Synthetic: Ptac-metDC
  source 1..1766: mol_type = other DNA
ttgacaattaatcatcggctcgtataatgtgtggaattgtgagcgctcacaattaagtgaattgccaataacaattactaaggaggttttttatgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatggacggaacggccttggatttagcccgtttcatgccccacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 663 - DNA, 329 bp, synthetic construct
  misc_feature 1..329: note = Synthetic: yjeH K/O
  source 1..329: mol_type = other DNA
gtgaaaaccctgattcacctcacatttcatcgctatttcttcatcaccgtcataagcgaatctgattgtgctaccatcgaaaatctacgcagttgcccaactgacatgggaattagccatggtccatatgaatatcctccttagttcctattccgaagttcctattctctagaaagtataggaacttcgaagcagctccagcctacacaatcgctcaagacgtgtaattccggcgtttcgacattaatcctggcgatcgtctttatgatcaaggcggtcgcggtcatcatcctttcgctggtactcaccatcaaaagtattaccgccac

SEQ ID NO: 664 - protein, 418 aa, synthetic construct
  REGION 1..418: note = Synthetic: YjeH
MSGLKQELGLAQGIGLLSTSLLGTGVFAVPALAALVAGNNSLWAWPVLIILVFPIAIVFAILGRHYPSAGGVAHFVGMAFGSRLERVTGWLFLSVIPVGLPAALQIAAGFGQAMFGWHSGQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEQMAAAASLPKIVVQLFGVGALWIACVIGYLACFASLNIYIQSFARLVWSQAQHNPDHYLARLSSRHIPNNALNAVLGCCVVSTLVIHALEINLDALIIYANGIFIMIYLLCMLAGCKLLQGRYRLLAVVGGLLCVLLLAMVGWKSLYALIMLAGLWLFLPKRKTPENGITT

(19) United States
(12) Patent Application Publication
(10) Pub. No.: US 2026/0199405 A1
(43) Pub. Date: Jul. 16, 2026

(54) RECOMBINANT BACTERIA ENGINEERED TO TREAT DISEASES ASSOCIATED WITH METHIONINE METABOLISM AND METHODS OF USE THEREOF

(71) Applicant: Synlogic Operating Company, Inc., Cambridge, MA (US)

(72) Inventors: Aoife Brennan, Dover, MA (US); Caroline Kurtz, Sudbury, MA (US); Neal Sondheimer, Toronto (CA); Michael James, Melrose, MA (US); David Lubkowicz, Somerville, MA (US)

(21) Appl. No.: 18/876,907

(22) PCT Filed: Jun. 23, 2023
(86) PCT No.: PCT/US2023/068978

§ 371 Date: Dec. 19, 2024

Related U.S. Application Data
(60) Provisional application No. 63/354,889, filed on Jun. 23, 2022.
(60) Provisional application No. 63/358,617, filed on Jul. 6, 2022.
(60) Provisional application No. 63/428,609, filed on Nov. 29, 2022.
(60) Provisional application No. 63/452,721, filed on Mar. 17, 2023.

(51) Int. Cl. A61K 35/741; A61P 3/00; C07K 14/195; C12N 9/88; C12N 15/70

(52) U.S. Cl. CPC A61K 35/741; A61P 3/00; C07K 14/195; C12N 9/88; C12N 15/70; C12Y 401/01057

(57) ABSTRACT

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject. These recombinant bacterial cells are designed to drive therapeutic effects throughout the body of a host from a point of origin of the microbiome. Specifically, the present disclosure provides recombinant bacterial cells that comprise a methionine decarboxylase enzyme for the treatment of diseases and disorders associated with amino acid metabolism, including homocystinuria, in a subject. The disclosure further provides pharmaceutical compositions and methods of treating disorders associated with amino acid metabolism, such as homocystinuria

### RELATED APPLICATIONS

This application is a 35 U.S.C. § 371 national stage filing of International Application No. PCT/US2023/068978, filed Jun. 23, 2023, which claims priority to U.S. Provisional Application No. 63/354,889, filed on Jun. 23, 2022, U.S. Provisional Application No. 63/358,617, filed on Jul. 6, 2022, U.S. Provisional Application No. 63/428,609, filed on Nov. 29, 2022, and U.S. Provisional Application No. 63/452,721, filed on Mar. 17, 2023. The entire contents of each of the foregoing applications are expressly incorporated herein by reference in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 3, 2025, is named “126046-07205.xml” and is 952,225 bytes in size. The sequence listing contained in this .XML file is part of the specification and is hereby incorporated by reference herein in its entirety.

### BACKGROUND

In healthy individuals, acquired dietary methionine is catabolized via the trans-sulfuration pathway, where mammalian cells catabolize methionine into homocysteine via S-Adenosyl-methionine and S-Adenosyl-homocysteine. The cystathionine β-synthase (CBS) enzyme then catalyzes the conversion of homocysteine to cystathionine using vitamin B6 (pyridoxal 5′-phosphate, PLP) as a co-enzyme. Another PLP-dependent enzyme, cystathionine γ-lyase, converts cystathionine into cysteine. Genetic mutations in one or more of these genes can cause metabolic perturbation in the trans-sulfuration pathway that leads to homocystinuria, also known as cystathionine beta synthase deficiency (“CBS deficiency”) (Garland et al., J. Ped. Child Health, 4 (8): 557-562, 1999). In homocystinuria patients, CBS enzyme deficiency causes elevated levels of homocysteine and low levels of cystathionine in the serum, which leads to excretion of homocysteine into the urine. Inherited homocystinuria, a serious life-threatening disease, results in high levels of homocysteine in plasma, tissues and urine. Some of the characteristics of the most common form of homocystinuria are myopia (nearsightedness), lens dislocation, higher risk of thromboembolism, and skeletal abnormalities. Homocystinuria may also cause developmental delay/intellectual disability (Mudd et al., Am. J. Hum. Genet., 37:1-31,1985).

A subpopulation of patients with homocystinuria can be treated with vitamin B₆ to increase the residual activity of the CBS enzyme. The B₆ non-responsive patients have to drastically limit the intake of dietary methionine to lower the levels of serum homocysteine. Compliance with a life-long low protein diet combined with methionine-free formula, is often poor, especially among the adults (Gupta et al., J. Inherit Metab. Dis., 2016, 39 (1): 3946; Mudd et al., Am. J. Hum. Genet., 37:1-31, 1985; Orgeron et al., Prog. Mol. Biol. Transl. Sci., 2014, 121:351-376). Hence, other options for treating homocystinuria are needed.

### SUMMARY

The present disclosure provides novel therapeutic treatment methods using recombinant microorganisms, e.g., bacteria, that have been engineered with optimized genetic circuitry, which allows the recombinant microorganism to have improved methionine consumption and 3-MTP production. In particular, the recombinant microorganisms disclosed herein have been engineered to comprise gene sequences encoding one or more optimized methionine decarboxylase (MetDC) enzymes and/or one or more optimized methionine importer(s), e.g., MetP. These optimized recombinant microorganisms demonstrate surprising therapeutic efficacy for modulating and treating diseases associated with methionine metabolism, such as homocystinuria, cystinuria, primary and secondary hypermethioninemia.

In some embodiments, the disclosure provides a recombinant bacterial cell comprising a heterologous methionine decarboxylase (metDC) gene operably linked to a promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NOs: 627, 628, 629, 630, 631, or 633. In some embodiments, the disclosure provides a recombinant bacterial cell comprising a heterologous methionine decarboxylase (metDC) gene operably linked to a promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NO: 596 or SEQ ID NO: 611.

In one embodiment, the metDC gene comprises a sequence that has at least 90%, 91%, 92%, 93%, 94%, 95%, 96%, 97%, 98%, or 99% identity to, comprises, or consists of SEQ ID NO: 596. In one embodiment, the metDC gene comprises a sequence that has at least 90%, 91%, 92%, 93%, 94%, 95%, 96%, 97%, 98%, or 99% identity to, comprises, or consists of SEQ ID NO: 611. In one embodiment, the metDC gene comprises a sequence that has at least 90%, 91%, 92%, 93%, 94%, 95%, 96%, 97%, 98%, or 99% identity to, comprises, or consists of SEQ ID NO: 627. In one embodiment, the metDC gene comprises a sequence that has at least 90%, 91%, 92%, 93%, 94%, 95%, 96%, 97%, 98%, or 99% identity to, comprises, or consists of SEQ ID NO: 628. In one embodiment, the metDC gene comprises a sequence that has at least 90%, 91%, 92%, 93%, 94%, 95%, 96%, 97%, 98%, or 99% identity to, comprises, or consists of SEQ ID NO: 1036. In one embodiment, the metDC gene comprises a sequence that has at least 90%, 91%, 92%, 93%, 94%, 95%, 96%, 97%, 98%, or 99% identity to, comprises, or consists of SEQ ID NO: 630. In one embodiment, the metDC gene comprises a sequence that has at least 90%, 91%, 92%, 93%, 94%, 95%, 96%, 97%, 98%, or 99% identity to, comprises, or consists of SEQ ID NO: 631. In one embodiment, the metDC gene comprises a sequence that has at least 90%, 91%, 92%, 93%, 94%, 95%, 96%, 97%, 98%, or 99% identity to, comprises, or consists of SEQ ID NO: 633.

In some embodiments, the recombinant bacterial further comprises a heterologous gene encoding a methionine importer. In some embodiments, the heterologous gene encoding the methionine importer is a metP gene. In some embodiments, the metP gene comprises a sequence having at least 90%, 91%, 92%, 93%, 94%, 95%, 96%, 97%, 98%, or 99% identity to, comprises, or consists of SEQ ID NO: 634. In some embodiments, the metP gene comprises a sequence having at least 90%, 91%, 92%, 93%, 94%, 95%, 96%, 97%, 98%, or 99% identity to, comprises, or consists of SEQ ID NO: 635. In some embodiments, the recombinant bacterial further comprises a heterologous gene encoding a methionine importer. In some embodiments, the heterologous gene encoding the methionine importer is a metP gene. In some embodiments, the metP gene comprises a sequence having at least 90%, 91%, 92%, 93%, 94%, 95%, 96%, 97%, 98%, or 99% identity to, comprises, or consists of SEQ ID NO: 637.

In some embodiments, the heterologous gene encoding the methionine importer is an metNIQ gene. In some embodiments, the metNIQ gene comprises a sequence having at least 90%, 91%, 92%, 93%, 94%, 95%, 96%, 97%, 98%, or 99% identity to, comprises, or consists of SEQ ID NO: 636. In some embodiments, the heterologous gene encoding the methionine importer is an metNIQ gene. In some embodiments, the metNIQ gene comprises a sequence having at least 90%, 91%, 92%, 93%, 94%, 95%, 96%, 97%, 98%, or 99% identity to, comprises, or consists of SEQ ID NO: 638. In some embodiments, the heterologous gene encoding the methionine importer is an metNIQ gene. In some embodiments, the metNIQ gene comprises a sequence having at least 90%, 91%, 92%, 93%, 94%, 95%, 96%, 97%, 98%, or 99% identity to, comprises, or consists of SEQ ID NO: 639. In some embodiments, the heterologous gene encoding the methionine importer is an metNIQ gene. In some embodiments, the metNIQ gene comprises a sequence having at least 90%, 91%, 92%, 93%, 94%, 95%, 96%, 97%, 98%, or 99% identity to, comprises, or consists of SEQ ID NO: 640.

In another aspect, the disclosure provides a recombinant bacterial cell comprising a heterologous metP gene operably linked to a promoter, wherein the metP gene comprises a sequence that has at least 90%, 91%, 92%, 93%, 94%, 95%, 96%, 97%, 98%, 99% identity to, comprises, or consists of SEQ ID NO: 634. In another aspect, the disclosure provides a recombinant bacterial cell comprising a heterologous metP gene operably linked to a promoter, wherein the metP gene comprises a sequence that has at least 90%, 91%, 92%, 93%, 94%, 95%, 96%, 97%, 98%, 99% identity to, comprises, or consists of SEQ ID NO: 635. In another aspect, the disclosure provides a recombinant bacterial cell comprising a heterologous metP gene operably linked to a promoter, wherein the metP gene comprises a sequence that has at least 90%, 91%, 92%, 93%, 94%, 95%, 96%, 97%, 98%, 99% identity to, comprises, or consists of SEQ ID NO: 637.

In some embodiments, the recombinant bacterial further comprises a heterologous methionine decarboxylase (metDC) gene. In some embodiments, the metDC gene comprises a sequence having at least 90%, 91%, 92%, 93%, 94%, 95%, 96%, 97%, 98%, or 99% identity to, comprises, or consists of any one of SEQ ID NOs: 627, 628, 629, 630, 631, or 633. In some embodiments, the recombinant bacterial further comprises a heterologous methionine decarboxylase (metDC) gene. In some embodiments, the metDC gene comprises a sequence having at least 90%, 91%, 92%, 93%, 94%, 95%, 96%, 97%, 98%, or 99% identity to, comprises, or consists of any one of SEQ ID NOs: 596 or 611.

In another aspect, the disclosure provides a recombinant bacterial cell comprising a heterologous metNIQ gene operably linked to a promoter, wherein the metNIQ gene comprises a sequence that has at least 90%, 91%, 92%, 93%, 94%, 95%, 96%, 97%, 98%, 99% identity to, comprises, or consists of SEQ ID NO: 636. In another aspect, the disclosure provides a recombinant bacterial cell comprising a heterologous metNIQ gene operably linked to a promoter, wherein the metNIQ gene comprises a sequence that has at least 90%, 91%, 92%, 93%, 94%, 95%, 96%, 97%, 98%, 99% identity to, comprises, or consists of SEQ ID NO: 638. In another aspect, the disclosure provides a recombinant bacterial cell comprising a heterologous metNIQ gene operably linked to a promoter, wherein the metNIQ gene comprises a sequence that has at least 90%, 91%, 92%, 93%, 94%, 95%, 96%, 97%, 98%, 99% identity to, comprises, or consists of SEQ ID NO: 639. In another aspect, the disclosure provides a recombinant bacterial cell comprising a heterologous metNIQ gene operably linked to a promoter, wherein the metNIQ gene comprises a sequence that has at least 90%, 91%, 92%, 93%, 94%, 95%, 96%, 97%, 98%, 99% identity to, comprises, or consists of SEQ ID NO: 640.

In some embodiments, the recombinant bacterial cell further comprises a heterologous methionine decarboxylase (metDC) gene. In some embodiments, the metDC gene comprises a sequence having at least 90%, 91%, 92%, 93%, 94%, 95%, 96%, 97%, 98%, or 99% identity to, comprises, or consists of any one of SEQ ID NOs: 627, 628, 629, 630, 631, or 633. In some embodiments, the recombinant bacterial cell further comprises a heterologous methionine decarboxylase (metDC) gene. In some embodiments, the metDC gene comprises a sequence having at least 90%, 91%, 92%, 93%, 94%, 95%, 96%, 97%, 98%, or 99% identity to, comprises, or consists of any one of SEQ ID NOs: 596 or 611.

In some embodiments, the recombinant bacterial cell further comprises a genetic modification that reduces export of methionine from the bacterial cell. In some embodiments, the genetic modification is a knock-out of an endogenous methionine efflux pump. In some embodiments, the endogenous methionine efflux pump is encoded by a yjeH gene. In some embodiments, the yjeH gene comprises a sequence having at least 90%, 91%, 92%, 93%, 94%, 95%, 96%, 97%, 98%, or 99% identity to, comprises, or consists of SEQ ID NO: 607.

In some embodiments, the recombinant bacterial cell further comprises an insertion, deletion or mutation of an endogenous phage gene. In some embodiments, the insertion, deletion or mutation is a deletion of the endogenous phage gene having the sequence of SEQ ID NO: 292.

In some embodiments, the recombinant bacterial cell further comprises a modified endogenous colibactin island. In some embodiments, the modified endogenous colibactin island comprises one or more modified clb sequences selected from clb sequences selected from clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304), clbL (SEQ ID NO: 305), clbM (SEQ ID NO: 306), clbN (SEQ ID NO: 307), clbO (SEQ ID NO: 308), clbP (SEQ ID NO: 309), clbQ (SEQ ID NO: 310), clbR (SEQ ID NO: 311), or clbS (SEQ ID NO: 312) gene. In some embodiments, the modified endogenous colibactin island comprises a deletion of clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304), clbL (SEQ ID NO: 305), clbM (SEQ ID NO: 306), clbN (SEQ ID NO: 307), clbO (SEQ ID NO: 30), clbP (SEQ ID NO: 309), clbQ (SEQ ID NO: 310), and clbR (SEQ ID NO: 311).

In some embodiments, the recombinant bacterial cell further comprises an auxotrophy. In some embodiments, the auxotrophy is a ΔdapA auxotrophy.

In some embodiments, the recombinant bacterial cell as disclosure herein comprises two or three copies of the metDC gene.

In some embodiments, the first promoter is an inducible promoter. In some embodiments, the inducible promoter is directly or indirectly induced by environmental conditions specific to the gut of a mammal, e.g., a human. In some embodiments, the inducible promoter is an IPTG-inducible promoter. In one embodiment, the IPTG-inducible promoter is a Ptac promoter. In one embodiment, the promoter is a lacI promoter.

In some embodiments, the heterologous gene is located on a plasmid or a chromosome in the bacterial cell.

In another aspect, the disclosure provides a recombinant bacterial cell comprising: a heterologous methionine decarboxylase (metDC) gene operably linked to a first promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NOs: 596, 611, 627, 628, 629, 630, 631 or 633, a heterologous gene encoding a methionine importer, wherein the heterologous gene encoding the methionine importer is a metP gene, and wherein the metP gene comprises a sequence having at least 90%, 91%, 92%, 93%, 94%, 95%, 96%, 97%, 98%, or 99% identity to, comprises, or consists of SEQ ID NO: 634, 635, or 637, a knock-out of an endogenous methionine efflux pump, wherein the endogenous methionine efflux pump is encoded by a yjeH gene, and wherein the yjeH gene comprises a sequence having at least 90%, 91%, 92%, 93%, 94%, 95%, 96%, 97%, 98%, or 99% identity to, comprises, or consists of SEQ ID NO: 607, a phage deletion, wherein the phage deletion is encoded by a gene sequence having at least 90%, 91%, 92%, 93%, 94%, 95%, 96%, 97%, 98%, or 99% identity to, comprises, or consists of SEQ ID NO: 292 and a ΔdapA auxotrophy.

In another aspect, the disclosure provides a recombinant bacterial cell comprising: a heterologous methionine decarboxylase (metDC) gene operably linked to a first promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NOs: 627, 628, 629, 630, 631, or 633, a heterologous gene encoding a methionine importer, wherein the heterologous gene encoding the methionine importer is a metP gene, and wherein the metP gene comprises a sequence having at least 90%, 91%, 92%, 93%, 94%, 95%, 96%, 97%, 98%, or 99% identity to, comprises, or consists of SEQ ID NO: 634 or SEQ ID NO: 635, a knock-out of an endogenous methionine efflux pump, wherein the endogenous methionine efflux pump is encoded by a yjeH gene, and wherein the yjeH gene comprises a sequence having at least 90%, 91%, 92%, 93%, 94%, 95%, 96%, 97%, 98%, or 99% identity to, comprises, or consists of SEQ ID NO: 607, a phage deletion, wherein the phage deletion is encoded by a gene sequence having at least 90%, 91%, 92%, 93%, 94%, 95%, 96%, 97%, 98%, or 99% identity to, comprises, or consists of SEQ ID NO: 292 and a ΔdapA auxotrophy.

In some embodiments, the recombinant bacterial cell comprises two or three copies of the metDC gene. In some embodiments, the recombinant bacterial cell further comprises a deletion of an endogenous colibactin island.

In another aspect, the disclosure provides a recombinant bacterial cell comprising: a heterologous methionine decarboxylase (metDC) gene operably linked to a promoter, wherein the heterologous metDC gene comprises a sequence that has at least 90%, 91%, 92%, 93%, 94%, 95%, 96%, 97%, 98%, or 99% identity to, comprises, or consists of SEQ ID NO: 627, wherein the recombinant bacterial cell comprises two or three copies of the metDC gene, a heterologous gene encoding a methionine importer, wherein the heterologous gene encoding the methionine importer is a metP gene, and wherein the metP gene comprises a sequence having at least 90%, 91%, 92%, 93%, 94%, 95%, 96%, 97%, 98%, or 99% identity to, comprises, or consists of SEQ ID NO: 634, a knock-out of an endogenous methionine efflux pump, wherein the endogenous methionine efflux pump is encoded by a yjeH gene, a phage deletion, a ΔdapA auxotrophy, and a deletion of an endogenous colibactin island.

In another aspect, the disclosure provides a recombinant bacterial cell comprising: a heterologous methionine decarboxylase (metDC) gene operably linked to a promoter, wherein the heterologous metDC gene comprises a sequence that has at least 90%, 91%, 92%, 93%, 94%, 95%, 96%, 97%, 98%, or 99% identity to, comprises, or consists of SEQ ID NO: 627, wherein the recombinant bacterial cell comprises three copies of the metDC gene, a heterologous gene encoding a methionine importer, wherein the heterologous gene encoding the methionine importer is a metP gene, and wherein the metP gene comprises a sequence having at least 90%, 91%, 92%, 93%, 94%, 95%, 96%, 97%, 98%, or 99% identity to, comprises, or consists of SEQ ID NO: 634, a knock-out of an endogenous methionine efflux pump, wherein the endogenous methionine efflux pump is encoded by a yjeH gene, a phage deletion, a ΔdapA auxotrophy, and a deletion of an endogenous colibactin island.

In some embodiments, the recombinant bacterial cell is a recombinant probiotic bacterial cell. In some embodiments, the recombinant bacterial cell is of the species Escherichia coli strain Nissle. In some embodiments, the recombinant bacterial cell is SYNB1353.

In another aspect, the disclosure provides a pharmaceutical composition comprising the recombinant bacterial cell of any one of the previous claims and a pharmaceutically acceptable carrier.

In another aspect, the disclosure provides a method for treating a disease associated with methionine metabolism in a subject, the method comprising administering the pharmaceutical composition disclosed herein to the subject.

In another aspect, the disclosure provides a method for reducing the levels of methionine in a subject, the method comprising administering to the subject the pharmaceutical composition disclosure herein, thereby reducing the levels of methionine in the subject. In another aspect, the disclosure provides a method for reducing the levels of cysteine in a subject, the method comprising administering to the subject the pharmaceutical composition disclosure herein, thereby reducing the levels of cysteine in the subject. In another aspect, the disclosure provides a method for reducing the levels of homocysteine in a subject, the method comprising administering to the subject the pharmaceutical composition disclosure herein, thereby reducing the levels of methionine in the subject.

In some embodiments, the subject has homocystinuria, cancer, or a metabolic disease.

In some embodiments, the pharmaceutical composition comprises about 3×10¹¹, about 5×10¹¹, about 6×10¹¹, about 1×10¹², about 2×10¹², about 3×10¹⁰ or about 2.8×10¹⁰ live recombinant bacterial cells/mL.

In some embodiments, about 0.1 g to about 1.5 g of methionine are degraded per day. In some embodiments, about 0.1 g to about 1.5 g of methionine are degraded when administered to the subject three times per day.

In some embodiments, methionine is metabolized at a rate of about 1.5 μmol/hr/1e9 cells. In some embodiments, methionine is metabolized at a rate of about 1.7 μmol/hr/1e9 cells. In some embodiments, methionine is metabolized at a rate of about 1.0 μmol/hr/1e9 cells, about 1.1 μmol/hr/1e9 cells, about 1.2 μmol/hr/1e9 cells, about 1.3 μmol/hr/1e9 cells, about 1.4 μmol/hr/1e9 cells, about 1.5 μmol/hr/1e9 cells, about 1.6 μmol/hr/1e9 cells, about 1.7 μmol/hr/1e9 cells, 1.8 μmol/hr/1e9 cells, about 1.9 μmol/hr/1e9 cells, or about 2.0 μmol/hr/1e9 cells. In some embodiments, methionine is metabolized at a rate of about 1.3 μmol/hr/1e9 cells. In some embodiments, methionine is metabolized at a rate of about 1.0 μmol/hr/1e9 cells to about 2.0 μmol/hr/1e9 cells. In some embodiments, methionine is metabolized at a rate of about 1.3 μmol/hr/1e9 cells to about 1.8 μmol/hr/1e9 cells. In some embodiments, methionine is metabolized at a rate of about 1.5 μmol/hr/1e9 cells to about 1.75 μmol/hr/1e9 cells.

In some embodiments, 3-MTP is produced at a rate of about 1.3 μmol/hr/1e9 cells. In some embodiments, 3-MTP is produced at a rate of about 1.0 μmol/hr/1e9 cells, about 1.1 μmol/hr/1e9 cells, about 1.2 μmol/hr/1e9 cells, about 1.3 μmol/hr/1e9 cells, about 1.4 μmol/hr/1e9 cells, about 1.5 μmol/hr/1e9 cells, about 1.6 μmol/hr/1e9 cells, about 1.7 μmol/hr/1e9 cells, 1.8 μmol/hr/1e9 cells, about 1.9 μmol/hr/1e9 cells, or about 2.0 μmol/hr/1e9 cells. In some embodiments, 3-MTP is produced at a rate of about 1.0 μmol/hr/1e9 cells to about 2.0 μmol/hr/1e9 cells. In some embodiments, 3-MTP is produced at a rate of about 1.2 μmol/hr/1e9 cells to about 1.8 μmol/hr/1e9 cells. In some embodiments, 3-MTP is produced at a rate of about 1.3 μmol/hr/1e9 cells to about 1.75 μmol/hr/1e9 cells. In some embodiments, 3-MTP is produced at a rate of about 1.3 μmol/hr/1e9 cells to about 1.5 μmol/hr/1e9 cells.

In some embodiments, the subject is fed a meal within one hour of administering the pharmaceutical composition. In some embodiments, the subject is fed a meal concurrently with administering the pharmaceutical composition. In some embodiments, the pharmaceutical composition is administered orally. In some embodiments, the subject is a human subject. In some embodiments, consumption of methionine is increased in the subject.

In another aspect, the disclosure provides a method for monitoring the effectiveness of a treatment of a subject or a method of measuring in vivo activity of the recombinant bacterium. The method comprises administering to the subject the recombinant bacterium disclosed herein or the pharmaceutical composition disclosed herein, and measuring a level of 3-MTP in urine or 3-MTP glycine in plasma or urine of the subject. In some embodiments, an increase in the level of 3-MTP in the urine of the subject after administration as compared to a level of 3-MTP in the urine of a control subject is an indication that the treatment is effective. In some embodiments, an increase in the level of 3-MTP glycine in plasma or urine of the subject after administration as compared to a level of 3-MTP glycine in plasma or urine of a control subject is an indication that the treatment is effective. In some embodiments, an increase of 3-MTP in urine or 3-MTP glycine in plasma or urine of a subject provides an indicator for strain activity in vivo. In some embodiments, the increase of 3-MTP or 3-MTP glycine is at least 1.2-fold, 1.5-fold, 2-fold, 2.5-fold, 3-fold, 3.5-fold, 4-fold, 4.5-fold or 5-fold.

In another aspect, disclosed herein is a method of treating a disease associated with methionine metabolism in a human subject, the method comprising orally administering to the subject a pharmaceutical composition comprising a pharmaceutically acceptable carrier and a recombinant bacterium, wherein the recombinant bacterium is an E. coli Nissle bacterium comprising: two copies of a metDC gene, wherein each copy of the metDC gene is operably linked to an IPTG inducible promoter; a metP-metDC gene cassette comprising one copy of a metP gene and a third copy of the metDC gene, wherein the metP-metDC gene cassette is operably linked to an IPTG inducible promoter; a deletion in yjeH gene; a deletion of the dapA gene; a deletion in the pks island; and an endogenous E. coli Nissle prophage gene deletion; and wherein the recombinant bacterium is administered to the subject at a dose of about 3×10¹¹, about 6×10¹¹, about 1×10¹², or about 2×10¹² live cells; thereby treating the disease associated with methionine metabolism in the subject.

In another aspect, the present disclosure provides for a method for reducing a level of methionine, cysteine and/or homocysteine in a human subject, the method comprising orally administering to the subject a pharmaceutical composition comprising a pharmaceutically acceptable carrier and a recombinant bacterium, wherein the recombinant bacterium is an E. coli Nissle bacterium comprising: two copies of a metDC gene, and wherein each copy of the metDC gene is operably linked to an IPTG inducible promoter; a metP-metDC gene cassette comprising one copy of a metP gene and a third copy of the metDC gene, wherein the metP-metDC gene cassette is operably linked to an IPTG inducible promoter; a deletion in yjeH gene; a deletion of the dapA gene; a deletion in the pks island; and an endogenous E. coli Nissle prophage gene deletion; and wherein the recombinant bacterium is administered to the subject at a dose of about 3×10¹¹, about 6×10¹¹, about 1×10¹², or about 2×10¹² live cells; thereby reducing the level of methionine, cysteine and/or homocysteine in the subject.

In another aspect, disclosed herein is a pharmaceutical composition comprising the recombinant bacterium disclosed herein and a pharmaceutically acceptable carrier. In one embodiment, disclosed herein is a pharmaceutical composition comprising a pharmaceutically acceptable carrier, 25 mM to 100 mM Tris, 5%-15% trehalose, pH 6.0-8.0, and a recombinant bacterium, wherein the recombinant bacterium is an E. coli Nissle bacterium comprising: two copies of a metDC gene, wherein each copy of the metDC gene is operably linked to an IPTG inducible promoter; a metP-metDC gene cassette comprising one copy of a metP gene and a third copy of the metDC gene, wherein the metP-metDC gene cassette is operably linked to an IPTG inducible promoter; a deletion in yjeH gene; a deletion of the dapA gene; a deletion in the pks island; and an endogenous E. coli Nissle prophage gene deletion.

In one embodiment, the metDC gene is from Streptomyces sp. 590. In one embodiment, the metDC gene encodes a MetDC polypeptide. In one embodiment, the MetDC polypeptide comprises two modifications (Q70D and N82H). In one embodiment, the metP gene encodes a MetP polypeptide. In one embodiment, the metP gene is from Flavobacterium segetis.

In some embodiments, each copy of the metDC gene comprises a sequence that has at least 90%, 91%, 92%, 93%, 94%, 95%, 96%, 97%, 98%, or 99% identity to, comprises, or consists of SEQ ID NO: 627. In some embodiments, the metP gene comprises a sequence having at least 90%, 91%, 92%, 93%, 94%, 95%, 96%, 97%, 98%, or 99% identity to, comprises, or consists of SEQ ID NO: 634.

In another aspect, the present disclosure provides for a method of treating a disease associated with methionine metabolism in a human subject, the method comprising orally administering to the subject a pharmaceutical composition comprising a pharmaceutically acceptable carrier and a recombinant bacterium, wherein the recombinant bacterium is an E. coli Nissle bacterium comprising: two copies of a metDC gene, wherein each copy of the metDC gene is operably linked to an IPTG inducible promoter; a metP-metDC gene cassette comprising one copy of a metP gene encoding a MetP polypeptide and a third copy of the metDC gene encoding a MetDC polypeptide, wherein the metP-metDC gene cassette is operably linked to an IPTG inducible promoter; a deletion in yjeH gene; a deletion of the dapA gene; a deletion in the pks island; and an endogenous E. coli Nissle prophage gene deletion; and wherein the recombinant bacterium is administered to the subject at a dose of about 3×10¹¹, about 6×10¹¹, about 1×10¹², or about 2×10¹² live cells; thereby treating the disease associated with methionine metabolism in the subject. In one embodiment, the metDC gene is from Streptomyces sp. 590. In one embodiment, the metDC gene encodes a MetDC polypeptide. In one embodiment, the MetDC polypeptide comprises two modifications (Q70D and N82H). In one embodiment, the metP gene encodes a MetP polypeptide. In one embodiment, the metP gene is from Flavobacterium segetis.

In another aspect, the present disclosure provides for a method for reducing a level of methionine, cysteine and/or homocysteine in a human subject, the method comprising orally administering to the subject a pharmaceutical composition comprising a pharmaceutically acceptable carrier and a recombinant bacterium, wherein the recombinant bacterium is an E. coli Nissle bacterium comprising: two copies of a metDC gene, wherein each copy of the metDC gene is operably linked to an IPTG inducible promoter; a metP-metDC gene cassette comprising one copy of a metP gene encoding a MetP polypeptide and a third copy of the metDC gene encoding a MetDC polypeptide, wherein the metP-metDC gene cassette is operably linked to an IPTG inducible promoter; a deletion in yjeH gene; a deletion of the dapA gene; a deletion in the pks island; and an endogenous E. coli Nissle prophage gene deletion; and wherein the recombinant bacterium is administered to the subject at a dose of about 3×10¹¹, about 6×10¹¹, about 1×10¹², or about 2×10¹² live cells; thereby reducing the level of methionine, cysteine and/or homocysteine in the subject. In one embodiment, the metDC gene is from Streptomyces sp. 590. In one embodiment, the metDC gene encodes a MetDC polypeptide. In one embodiment, the MetDC polypeptide comprises two modifications (Q70D and N82H). In one embodiment, the metP gene encodes a MetP polypeptide. In one embodiment, the metP gene is from Flavobacterium segetis.

In another aspect, disclosed herein is a pharmaceutical composition comprising the recombinant bacterium disclosed herein and a pharmaceutically acceptable carrier. In one embodiment, disclosed herein is a pharmaceutical composition comprising a pharmaceutically acceptable carrier, 25 mM to 100 mM Tris, 5%-15% trehalose, pH 6.0-8.0, and a recombinant bacterium, wherein the recombinant bacterium is an E. coli Nissle bacterium comprising: two copies of a metDC gene, wherein each copy of the metDC gene is operably linked to an IPTG inducible promoter; a metP-metDC gene cassette comprising one copy of a metP gene and a third copy of the metDC gene, wherein the metP-metDC gene cassette is operably linked to an IPTG inducible promoter; a deletion in yjeH gene; a deletion of the dapA gene; a deletion in the pks island; and an endogenous E. coli Nissle prophage gene deletion. In one embodiment, the metDC gene is from Streptomyces sp. 590. In one embodiment, the metDC gene encodes a MetDC polypeptide. In one embodiment, the MetDC polypeptide comprises two modifications (Q70D and N82H). In one embodiment, the metP gene encodes a MetP polypeptide. In one embodiment, the metP gene is from Flavobacterium segetis.

In some embodiments, each copy of the metDC gene encodes the MetDC polypeptide comprising a sequence that has at least 90%, 91%, 92%, 93%, 94%, 95%, 96%, 97%, 98%, or 99% identity to, comprises, or consists of SEQ ID NO: 641. In some embodiments, the metP gene encodes the MetP polypeptide comprising a sequence having at least 90%, 91%, 92%, 93%, 94%, 95%, 96%, 97%, 98%, or 99% identity to, comprises, or consists of SEQ ID NO: 649.

In some embodiments, the yjeH gene comprises a sequence having at least 90%, 91%, 92%, 93%, 94%, 95%, 96%, 97%, 98%, or 99% identity to, comprises, or consists of SEQ ID NO: 607.

In some embodiments, the endogenous E. coli Nissle prophage gene deletion is a deletion of the endogenous phage gene comprising a sequence of SEQ ID NO: 292.

In some embodiments, the deletion in the pks island comprises a deletion of clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304), clbL (SEQ ID NO: 305), clbM (SEQ ID NO: 306), clbN (SEQ ID NO: 307), clbO (SEQ ID NO: 308), clbP (SEQ ID NO: 309), clbQ (SEQ ID NO: 310), and/or clbR (SEQ ID NO: 311.

In some embodiments, the two copies of the metDC gene and the metP-metDC gene cassette are located on a plasmid or a chromosome in the engineered microbial cell.

In some embodiments, the recombinant bacterium is SYNB1353.

In some embodiments, the pharmaceutical composition is formulated for oral administration.

In some embodiments, the pharmaceutical composition is a lyophilized formulation, a reconstituted lyophilized formulation, a solid formulation, or a solid oral formulation.

In some embodiments, the pharmaceutical composition further comprises 25 mM to 100 mM Tris. In some embodiments, the pharmaceutical composition comprises 50 mM Tris. In some embodiments, the pH is between 6.0-8.0. In some embodiments, the pH is 7.5.

In some embodiments, the pharmaceutical composition further comprises 1%-20% w/v trehalose. In some embodiments, the pharmaceutical composition comprises 10% w/v trehalose.

In some embodiments, the pharmaceutical composition is resuspended in bicarbonate.

In some embodiments, the pharmaceutical composition further comprises flavoring. In some embodiments, the flavoring is selected from the group consisting of strawberry, vanilla, lemon, grape, bubble gum, and cherry.

In some embodiments, the pharmaceutical composition further comprises vitamin B₆, pyridoxal 5 phosphate (PLP), and/or pyridoxine (PN). In some embodiments, the composition comprises vitamin B₆ and/or PLP and/or PN at an amount of at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 35 mg, at least about 36 mg, at least about 37 mg, at least about 38 mg, at least about 39 mg, or at least about 40 mg.

In some embodiments, the pharmaceutical composition comprises vitamin B₆ and/or PLP and/or PN at an amount of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 250 mg, or at least about 300 mg.

In some embodiments, the pharmaceutical composition comprises vitamin B₆ and/or PLP and/or PN at an amount of about 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg.

In some embodiments, the pharmaceutical composition comprises vitamin B₆ and/or PLP and/or PN at an amount of about 50 mg to about 75 mg, about 75 mg to about 100 mg, about 100 mg to about 125 mg, about 125 mg to about 150 mg, about 150 mg to about 175 mg, or about 175 mg to about 200 mg.

In some embodiments, the pharmaceutical composition comprises vitamin B₆ and/or PLP and/or PN at an amount of 1 mg to about 50 mg, about 50 mg to about 100 mg, about 100 mg to about 150 mg, about 150 mg to about 200 mg, about 200 mg to about 300 mg, about 300 mg to about 400 mg, or about 400 mg to about 500 mg.

In some specific embodiments, the genetically engineered bacteria may be formulated into a pharmaceutical composition comprising vitamin B₆, pyridoxal 5 phosphate (PLP), and/or pyridoxine (PN) at less than 100 mg. In some specific embodiments, the genetically engineered bacteria may be formulated into a pharmaceutical composition comprising vitamin B₆, pyridoxal 5 phosphate (PLP), and/or pyridoxine (PN) at 25 mg.

In any of these embodiments, the pharmaceutical compositions into which the engineered bacteria are formulated and which comprise vitamin B₆, PLP and/or PN, may further comprise sodium bicarbonate and a flavoring agent.

Alternatively, in some embodiments, the novel therapeutic treatment methods described herein further comprise the administration of vitamin B₆, pyridoxal 5 phosphate, and/or pyridoxine prior to, concurrently or directly after administration of the bacteria. For example, in one aspect a method for treating a disease associated with methionine metabolism in a subject, comprising administering orally a pharmaceutical composition disclosed herein comprising a recombinant bacterium described herein to the subject may further comprise the administration of vitamin B₆, pyridoxal 5 phosphate, and/or pyridoxine prior to, concurrently or directly after administration of the pharmaceutical composition. In another aspect, the present disclosure provides for a method for reducing a level of methionine, cysteine and/or homocysteine in a human subject, the method comprising orally administering to the subject a pharmaceutical composition comprising a recombinant bacterium described herein, further comprises the administration of vitamin B₆, pyridoxal 5 phosphate, and/or pyridoxine prior to, concurrently or directly after administration of the bacteria. For example, an amount of at least about 1 mg, at least about 2 mg, at least about 3, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 35 mg, at least about 36 mg, at least about 37 mg, at least about 38 mg, at least about 39 mg, or at least about 40 mg vitamin B₆, pyridoxal 5 phosphate (PLP), and/or pyridoxine may be administered, e.g., per day or at least once daily prior to, concurrently with, or after each bacterial dose.

In some embodiments, an amount of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 250 mg, or at least about 300 mg vitamin B₆, pyridoxal 5 phosphate (PLP), and/or pyridoxine are administered, e.g., per day at least once daily or prior to, concurrently with, or after each bacterial dose.

In some embodiments an amount of at about 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg vitamin B₆, pyridoxal 5 phosphate (PLP), and/or pyridoxine are administered, e.g., per day or at least once daily prior to, concurrently with, or after each bacterial dose.

In some embodiments, an amount of about 50 mg to about 75 mg, about 75 mg to about 100 mg, about 100 mg to about 125 mg, about 125 mg to about 150 mg, about 150 mg to about 175 mg, or about 175 mg to about 200 mg vitamin B₆, pyridoxal 5 phosphate (PLP), and/or pyridoxine are administered, e.g., per day or at least once daily prior to, concurrently with, or after each bacterial dose.

In some embodiments, an amount of about 1 mg to about 50 mg, about 50 mg to about 100 mg, about 100 mg to about 150 mg, about 150 mg to about 200 mg, about 200 mg to about 300 mg, about 300 mg to about 400 mg, or about 400 mg to about 500 mg vitamin B₆, pyridoxal 5 phosphate (PLP), and/or pyridoxine are administered, e.g., per day at least once daily or prior to, concurrently with, or after each bacterial dose.

In some specific embodiments, an amount of about 100 mg or less than about 100 mg vitamin B₆, pyridoxal 5 phosphate (PLP), and/or pyridoxine is administered, e.g., per day or at least once daily or prior to, concurrently with, or after each bacterial dose. In some specific embodiments, an amount of about 25 mg vitamin B₆, pyridoxal 5 phosphate (PLP) or pyridoxine may be administered, e.g., per day or at least once daily or prior to, concurrently with, or after each dose.

In some embodiments, the subject has homocystinuria, cystinuria, or a metabolic disease.

In some embodiments, the pharmaceutical composition is administered to the subject once per day, twice per day, or three times per day.

In some embodiments, the subject is fed a meal within one hour of administering the pharmaceutical composition.

In some embodiments, the subject is fed a meal concurrently with administering the pharmaceutical composition.

In some embodiments, the pharmaceutical composition further comprises administering a proton pump inhibitor (PPI) to the subject. In some embodiments, the PPI is esomeprazole. In some embodiments, the administering of the PPI is once a day.

In some embodiments, consumption of methionine is increased in the subject.

In some embodiments, about 0.1 g to about 1.5 g of methionine are degraded per day.

In some embodiments, methionine is metabolized at a rate of about 1.5 μmol/hr/1e9 cells. In some embodiments, methionine is metabolized at a rate of about 1.7 μmol/hr/1e9 cells. In some embodiments, methionine is metabolized at a rate of about 1.3 μmol/hr/1e9 cells. In some embodiments, methionine is metabolized at a rate of about 1.0 μmol/hr/1e9 cells, about 1.1 μmol/hr/1e9 cells, about 1.2 μmol/hr/1e9 cells, about 1.3 μmol/hr/1e9 cells, about 1.4 μmol/hr/1e9 cells, about 1.5 μmol/hr/1e9 cells, about 1.6 μmol/hr/1e9 cells, about 1.7 μmol/hr/1e9 cells, 1.8 μmol/hr/1e9 cells, about 1.9 μmol/hr/1e9 cells, or about 2.0 μmol/hr/1e9 cells. In some embodiments, methionine is metabolized at a rate of about 1.3 μmol/hr/1e9 cells. In some embodiments, methionine is metabolized at a rate of about 1.0 μmol/hr/1e9 cells to about 2.0 μmol/hr/1e9 cells. In some embodiments, methionine is metabolized at a rate of about 1.3 μmol/hr/1e9 cells to about 1.8 μmol/hr/1e9 cells. In some embodiments, methionine is metabolized at a rate of about 1.5 μmol/hr/1e9 cells to about 1.75 μmol/hr/1e9 cells.

In some embodiments, 3-MTP is produced at a rate of about 1.3 μmol/hr/1e9 cells. In some embodiments, 3-MTP is produced at a rate of about 1.0 μmol/hr/1e9 cells, about 1.1 μmol/hr/1e9 cells, about 1.2 μmol/hr/1e9 cells, about 1.3 μmol/hr/1e9 cells, about 1.4 μmol/hr/1e9 cells, about 1.5 μmol/hr/1e9 cells, about 1.6 μmol/hr/1e9 cells, about 1.7 μmol/hr/1e9 cells, 1.8 μmol/hr/1e9 cells, about 1.9 μmol/hr/1e9 cells, or about 2.0 μmol/hr/1e9 cells. In some embodiments, 3-MTP is produced at a rate of about 1.3 μmol/hr/1e9 cells to about 1.75 μmol/hr/1e9 cells. In some embodiments, 3-MTP is produced at a rate of about 1.3 μmol/hr/1e9 cells to about 1.5 μmol/hr/1e9 cells.

In some embodiments, the method further comprises selecting a subject who would benefit from reduced methionine levels.

### BRIEF DESCRIPTION OF THE DRAWINGS

FIG. 1 provides an overview for homocystinuria, a disorder of methionine metabolism caused by a defect in cystathionine beta-synthase (CBS), which leads to the accumulation of homocysteine in blood and urine.

FIG. 2 is a schematic of an exemplary engineered E. coli Nissle capable of consuming methionine. Optimal metP and metDC were identified metagenomic, codebase and protein engineering libraries. 3-MTP=3-methylthiolpropylamine; MetDC=methionine decarboxylase; MetP=methionine importer; Ptac=IPTG-inducible promoter; YjeH=methionine/branched chain amino acid exporter.

FIG. 3 depicts a schematic of the overall design of the clinical study.

FIG. 4A and FIG. 4B depict graphs showing rate of SYNB 1353 Met consumption (whole cell, FIG. 4A) and 2.5e9 lysed cells (lysate FIG. 4B) in the presence of various concentrations of pyridoxal 5′ phosphate (PLP), the active form of vitamin B₆.

FIG. 5 depicts a graph showing the rate of SYNB 1353 Met consumption (live whole cells) in the presence of various concentrations of pyridoxine, the precursor form of vitamin B₆ (Pyridoxine is converted to PLP in the cell via PdxK and PdxH).

FIG. 6A depicts graphs showing rate of Met consumption by lyophilized SYNB1353, comparing activity in the presence (+VitB₆) or absence of 0.1 mM PLP (−VitB₆).

FIG. 6B depicts graphs showing Met concentration over time in assay medium comprising lyophilized SYNB1353, in the presence (SYNB1353+VitB₆) or absence of 0.1 mM PLP SYNB1353).

FIG. 7A depicts a structural formula of 3-MTP glycine.

FIG. 7B Depict a chromatogram showing a sample derived from 3-MTP spike NHPs (upper panel), in which 3-MTP glycine was validated against a synthetic standard (lower panel).

FIG. 8A and FIG. 8B depict graphs showing the concentration of 3-MTP glycine in plasma (FIG. 8A) and urine (FIG. 8B) upon administration of 10, 30, or 100 mg/kg 3-MTP.

FIG. 9A and FIG. 9B depict graphs showing the concentration of 3-MTP glycine in plasma (FIG. 9A) and urine (FIG. 9B) upon administration of a bolus of 100 mg/kg Met and SYNB1353 (1×10¹² cells).

FIG. 9C and FIG. 9D depict graphs showing the concentration of methionine in plasma (FIG. 9C) and plasma homocysteine (FIG. 9D), in which the 100 mg/kg values correspond to the 3-MTP and 3-MTP glycine data shown in FIG. 9A and FIG. 9B.

FIG. 10A and FIG. 10B depict schematics showing a study design for SYNB 1353 Phase 1 in healthy volunteers, in which a methionine meal challenge used to simulate severely elevated methionine, homocysteine in HCU. FIG. 10A depicts the overall Phase 1 MAD design in healthy volunteers and FIG. 10B depicts the dosing schedule for each week within the MAD design.

FIG. 11A and FIG. 11B depict graphs showing % change in methionine (FIG. 11A) and homocysteine (FIG. 11B) from baseline, measured following a methionine meal challenge as Area Under the Curve (AUC) over 24 hours in healthy volunteers administered a bolus of 30 mg/kg methionine and 1×10¹² formulated with (Formulation 2, Form 2) or without vitamin B₆ (Formulation 1, Form. 1). LS mean change, 95% CI; * p<0.05; Form.=formulation; placebo n=8, 1×10¹² form. 1 n=6, 1×10¹² form. 2 n=5.

FIG. 12A and FIG. 12B depict plasma methionine (FIG. 12A) and total homocysteine levels (FIG. 12B) of all subjects (n=30) from all cohorts over 24 h after receiving a 30 mg/kg methionine load challenge. Data represents the mean±SD.

FIG. 13A shows urinary 3MTP-glycine levels in SYNB 1353 dosed subjects. Data represents the mean and SD of all positive samples collected (n=4/30 pre-dose, n=4/8 placebo and n=10/22 SYNB 1353). Ae: Amount excreted in urine.

FIG. 13B shows urinary 3MTP-glycine levels in SYNB1353 dosed subjects from the same study shown in FIG. 13A, separating out Formulation 1 (without B6) and Formulation 2 (with B6). 3-MTP-glycine recovery in subjects with positive samples (n=4/30 predose, n=4/8 placebo and n=10/22 SYNB1353) is shown. Total amount of excreted (Ae) 3-MTP-glycine in 24 hours urine was determined for cohorts receiving 1×10¹² live cell of SYNB1353. Form=formulation. LS mean change, 95% CI *p<0.05.

FIG. 14A is a graph showing in vitro methionine consumption (solid line) and 3-MTP production (dotted line) by EcN (unengineered bacteria) or SYNB1353 ((lacI-Ptac, IPTG) 3× metDC (Q70D N82H; SEQ ID NO: 641; engineered library); 1× metP (metagenomics library; F. segetis; SEQ ID NO: 649); Δφ; Δdap; ΔyjeH; Δpks). Cells were incubated for the indicated time in M9 medium with 0.5% glucose and 10 mM methionine at 37° C., supernatant was collected for methionine (HPLC) and 3-MTP (LC-MS/MS) measurements. * p<0.05 versus EcN. Met: methionine, metP: methionine importer, metDC: methionine decarboxylase, YjeH: methionine exporter.

FIG. 14B depicts Met consumption by E. coli strains. SYNB1353 (metDC and metP intergrated into bacterial chromosome) is compared to the prototype strain (metDC and metP plasmid-based). * p<0.05 versus prototype.

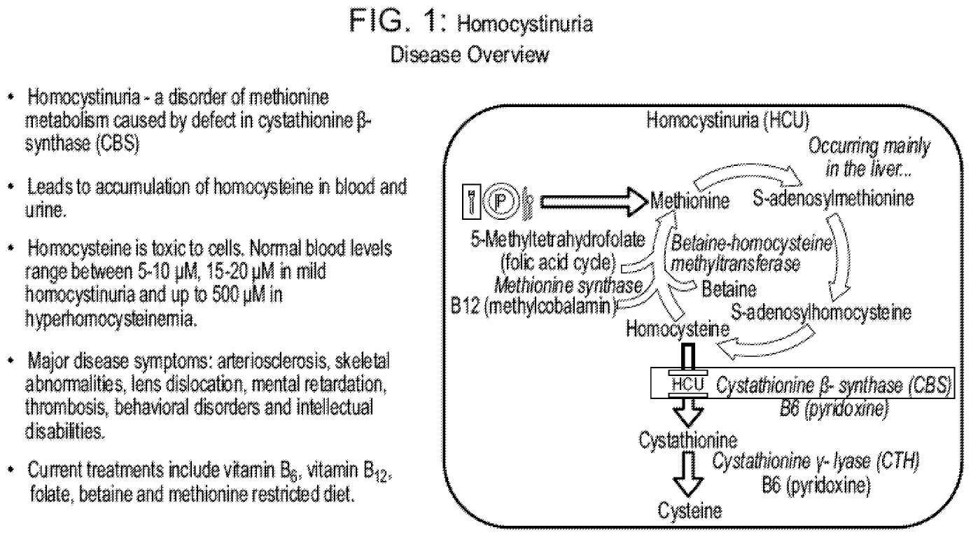

### DETAILED DESCRIPTION

Methionine is an essential amino acid and, as such, must be acquired through diet. It is a sulfur-containing proteinogenic amino acid and precursor to several molecules, including the amino acids cysteine and taurine, the antioxidant glutathione and the methyl group donor SAM. Despite its biological indispensability, the negative effects of excessive dietary or endogenous methionine are well characterized. Methionine is a precursor to the amino acid homocysteine which accumulates in CBS deficiency (or classic HCU) and causes dysregulation of multiple critical physiological systems. Dietary restriction of methionine increases lifespan, improves metabolic health and is highly effective at lowering plasma homocysteine and preventing the complications associated with CBS deficiency, but proper adherence is extremely poor. Thus, the identification of a safe, easy to use, orally available approach capable of detoxifying methionine and decreasing the burden associated with dietary methionine restriction is needed.

The present disclosure provides recombinant bacterial cells that have been engineered with optimized genetic circuitry which allow the recombinant bacterial cells to turn on and off an engineered metabolic pathway by sensing a patient's internal environment or by chemical induction during, for example, manufacturing. When turned on, the recombinant bacterial cells complete all of the steps in a metabolic pathway to achieve a therapeutic effect in a host subject and are designed to drive therapeutic effects throughout the body of a host from a point of origin of the microbiome.

Specifically, the present disclosure provides recombinant bacterial cells, pharmaceutical compositions thereof, and methods of modulating and treating diseases associated with amino acid metabolism, such as homocystinuria. Specifically, the recombinant bacteria disclosed herein have been constructed to comprise genetic circuits composed of, for example, a methionine decarboxylase to treat disease, as well as other circuitry in order to guarantee the safety and non-colonization of the subject that is administered the recombinant bacteria, such as auxotrophies, etc. These recombinant bacteria are safe and well tolerated and augment the innate activities of the subject's microbiome to achieve a therapeutic effect.

In some embodiments, a bacterial cell disclosed herein has been genetically engineered to comprise a heterologous gene sequence encoding one or more methionine decarboxylases and is capable of processing (e.g., metabolizing) and reducing levels of methionine. In some embodiments, a bacterial cell disclosed herein has been genetically engineered to comprise a heterologous gene sequence encoding one or more methionine decarboxylases and is capable of processing and reducing levels of methionine in low-oxygen environments, e.g., the gut. Thus, the genetically engineered bacterial cells and pharmaceutical compositions comprising the bacterial cells disclosed herein may be used to convert excess methionine into non-toxic molecules in order to treat and/or prevent diseases associated with amino acid metabolism, such as homocystinuria, cystinuria, primary and secondary hypermethioninemia, cancer, and metabolic syndromes/diseases.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant bacterial cell” or “recombinant bacteria” (also referred to herein as a “genetically engineered bacterial cell”) refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells of the disclosure may comprise exogenous or heterologous nucleotide sequences on plasmids. Alternatively, recombinant bacterial cells may comprise exogenous or heterologous nucleotide sequences stably incorporated into their chromosome.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature. As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous gene” or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “bacteriostatic” or “cytostatic” refers to a molecule or protein which is capable of arresting, retarding, or inhibiting the growth, division, multiplication or replication of a recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding at least one methionine decarboxylase, that is joined to a regulatory sequence in a manner which allows expression of the nucleic acid sequence, e.g., the gene(s) encoding the methionine decarboxylase. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Examples of inducible promoters include, but are not limited to, an FNR promoter, a P_{araC} promoter, a P_{araBAD} promoter, a propionate promoter, and a P_{TetR} promoter, each of which are described in more detail herein. Examples of other inducible promoters are provided herein below.

As used herein, “stably maintained” or “stable” bacterium is used to refer to a bacterial host cell carrying non-native genetic material, e.g., a methionine decarboxylase, that is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising an amino acid catabolism gene, in which the plasmid or chromosome carrying the amino acid catabolism gene is stably maintained in the bacterium, such that the methionine decarboxylase can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding at least one methionine decarboxylase.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one methionine decarboxylase operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of an asparagine. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254 (2): 157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

“Exogenous environmental condition(s)” or “environmental conditions” refer to settings or circumstances under which the promoter described herein is directly or indirectly induced. The phrase is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions refer to the presence of molecules or metabolites that are specific to the mammalian gut in a healthy or disease-state, e.g., propionate. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

As used herein, “exogenous environmental conditions” or “environmental conditions” also refers to settings or circumstances or environmental conditions external to the engineered microorganism, which relate to in vitro culture conditions of the microorganism. “Exogenous environmental conditions” may also refer to the conditions during growth, production, and manufacture of the organism. Such conditions include aerobic culture conditions, anaerobic culture conditions, low oxygen culture conditions and other conditions under set oxygen concentrations. Such conditions also include the presence of a chemical and/or nutritional inducer, such as tetracycline, arabinose, IPTG, rhamnose, and the like in the culture medium. Such conditions also include the temperatures at which the microorganisms are grown prior to in vivo administration. For example, using certain promoter systems, certain temperatures are permissive to expression of a payload, while other temperatures are non-permissive. Oxygen levels, temperature and media composition influence such exogenous environmental conditions. Such conditions affect proliferation rate, rate of induction of the payload or gene of interest, e.g., amino acid catabolism gene, other regulators (e.g., FNRS24Y), and overall viability and metabolic activity of the strain during strain production.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut).

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003). Non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic and/or low oxygen conditions by the global transcriptional regulator FNR that is naturally found in Nissle. Under anaerobic and/or low oxygen conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and
responsive genes and regulatory regions

| Transcription | Examples of responsive genes, promoters, and/or |
|---|---|
| factor | regulatory regions: |
| FNR | nirB, ydfZ, pdhR, focA, ndH, hlyE, narK, narX, narG, |
| | yfiD, tdcD |
| DNR | norb, norC |

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions. In some embodiments, the genetically engineered bacteria of the invention comprise a gene encoding a methionine-metabolizing enzyme that is operably linked to a directly or indirectly inducible promoter that is not associated with said gene in nature, e.g., an FNR promoter operably linked to a gene encoding an amino acid metabolism gene.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

In some embodiments, the genetically engineered bacteria are active in the gut. In some embodiments, the genetically engineered bacteria are active in the large intestine. In some embodiments, the genetically engineered bacteria are active in the small intestine. In some embodiments, the genetically engineered bacteria are active in the small intestine and in the large intestine. In some embodiments, the genetically engineered bacteria transit through the small intestine. In some embodiments, the genetically engineered bacteria have increased residence time in the small intestine. In some embodiments, the genetically engineered bacteria colonize the small intestine. In some embodiments, the genetically engineered bacteria do not colonize the small intestine. In some embodiments, the genetically engineered bacteria have increased residence time in the gut. In some embodiments, the genetically engineered bacteria colonize the small intestine. In some embodiments, the genetically engineered bacteria do not colonize the gut.

As used herein, the term “low oxygen” is meant to refer to a level, amount, or concentration of oxygen (O₂) that is lower than the level, amount, or concentration of oxygen that is present in the atmosphere (e.g., <21% O_{2;} <160 torr O₂₎). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg 02, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂₎. The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147 (5): 1055-1063 (2014); Bergofsky et al., J Clin. Invest., 41 (11): 1971-1980 (1962); Crompton et al., J Exp. Biol., 43:473-478 (1965); He et al., PNAS (USA), 96:4586-4591 (1999); Mckeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (μmole) (1 μmole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%, 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%, 0.44%, 0.3%, 0.25%, 0.2%, 0.1%, 0.08%, 0.075%, 0.058%, 0.04%, 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂,etc.). The exemplary fractions and ranges listed here are for illustrative purposes and not meant to be limiting in any way.

TABLE 2

| Compartment | Oxygen Tension |
|---|---|
| stomach | ~60 torr (e.g., 58 +/− 15 torr) |
| duodenum and first | ~30 torr (e.g., 32 +/− 8 torr); ~20% |
| part of jejunum | oxygen in ambient air |
| Ileum (mid- small | ~10 torr; ~6% oxygen in ambient air (e.g., |
| intestine) | 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., amino acid metabolism gene, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in the gut. For example, the stable bacterium may be a genetically modified bacterium comprising an amino acid metabolism gene, in which the plasmid or chromosome carrying the amino acid metabolism gene is stably maintained in the host cell, such that amino acid metabolism gene can be expressed in the host cell, and the host cell is capable of survival and/or growth in vitro and/or in vivo. In some embodiments, copy number affects the stability of expression of the non-native genetic material, e.g., an amino acid metabolism gene. In some embodiments, copy number affects the level of expression of the non-native genetic material, e.g., heterologous gene.

As used herein, the term “auxotroph” or “auxotrophic” refers to an organism that requires a specific factor, e.g., an amino acid, a sugar, or other nutrient, to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders associated with or involved with amino acid metabolism, e.g., homocystinuria, may be caused by inborn genetic mutations for which there are no known cures. Diseases can also be secondary to other conditions, e.g., an intestinal disorder or a bacterial infection. Treating diseases associated with amino acid metabolism may encompass reducing normal levels of one or more amino acids, reducing excess levels of one or more amino acids, or eliminating one or more amino acids, and does not necessarily encompass the elimination of the underlying disease.

As used herein the terms “disease associated with amino acid metabolism” or a “disorder associated with amino acid metabolism” is a disease or disorder involving the abnormal, e.g., increased, levels of one or more amino acids, e.g., methionine, in a subject. In one embodiment, a disease or disorder associated with amino acid metabolism, e.g., methionine metabolism, is homocystinuria. In another embodiment, a disease or disorder associated with amino acid metabolism, e.g., methionine metabolism, is cancer. In another embodiment, a disease or disorder associated with amino acid metabolism, e.g., methionine metabolism, is a metabolic disease or a metabolic syndrome.

As used herein, the term “amino acid” refers to a class of organic compounds that contain at least one amino group and one carboxyl group. Amino acids include leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline.

As used herein, the term “amino acid catabolism” or “amino acid metabolism” refers to the processing, breakdown and/or degradation of an amino acid molecule (e.g., methionine, asparagine, lysine or arginine) into other compounds that are not associated with the disease associated with amino acid metabolism, such as homocystinuria, or other compounds which can be utilized by the bacterial cell.

In another embodiment, the term “methionine catabolism” refers to the processing, breakdown, and/or degradation of methionine into 3-methylthiopropylamine. In yet another embodiment, the term “methionine catabolism” refers to the processing, breakdown, and/or degradation of methionine to sulfate. In one embodiment, the term “methionine catabolism” refers to the processing, breakdown, and/or degradation of methionine into methanethiol and 2-aminobut-2-enoate. In another embodiment, the term “methionine catabolism” refers to the processing, breakdown, and/or degradation of methionine into 3-methylthio-2-oxobutyric acid.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu. For example, a methionine transporter such as MetP imports methionine into the microorganism.

As used herein, “payload” refers to one or more molecules of interest to be produced by a genetically engineered microorganism, such as bacteria or a virus. In some embodiments, the payload is a therapeutic payload, e.g., an amino acid catabolic enzyme or an amino acid transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In some embodiments, the payload is encoded by a gene or multiple genes or an operon. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disease or condition associated with excess amino acid levels. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US20140079701, the contents of which are herein incorporated by reference in its entirety.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. The term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. In some embodiments, the improvement of transcription and/or translation involves increasing the level of transcription and/or translation. In some embodiments, the improvement of transcription and/or translation involves decreasing the level of transcription and/or translation. In some embodiments, codon optimization is used to fine-tune the levels of expression from a construct of interest. Codon optimization includes, but is not limited to, processes including selecting codons for the coding sequence to suit the codon preference of the expression host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent, inter alia, on the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

The terms “phage” and “bacteriophage” are used interchangeably herein. Both terms refer to a virus that infects and replicates within a bacterium. As used herein “phage” or bacteriophage” collectively refers to prophage, lysogenic, dormant, temperate, intact, defective, cryptic, and satellite phage, phage tail bacteriocins, tailiocins, and gene transfer agents. As used therein the term “prophage” refers to the genomic material of a bacteriophage, which is integrated into a replicon of the host cell and replicates along with the host. The prophage may be able to produce phages if specifically activated. In some cases, the prophage is not able to produce phages or has never done so (i.e., defective or cryptic prophages). In some cases, prophage also refers to satellite phages. The terms “prophage” and “endogenous phage” are used interchangeably herein. “Endogenous phage” or “endogenous prophage” also refers to a phage that is present in the natural state of a bacterium (and its parental strain). As used herein the term “phage knockout” or “inactivated phage” refers to a phage which has been modified so that it can either no longer produce and/or package phage particles or it produces fewer phage particles than the wild type phage sequence. In some embodiments, the inactivated phage or phage knockout refers to the inactivation of a temperate phage in its lysogenic state, i.e., to a prophage. Such a modification refers to a mutation in the phage; such mutations include insertions, deletions (partial or complete deletion of phage genome), substitutions, inversions, at one or more positions within the phage genome, e.g., within one or more genes within the phage genome. As used herein the adjectives “phage-free”, “phage free” and “phageless” are used interchangeably to characterize a bacterium or strain which contains one or more prophages, one or more of which have been modified. The modification can result in a loss of the ability of the prophage to be induced or release phage particles. Alternatively, the modification can result in less efficient or less frequent induction or less efficient or less frequent phage release as compared to the isogenic strain without the modification. Ability to induce and release phage can be measured using a plaque assay as described herein. As used herein phage induction refers to the part of the life cycle of a lysogenic prophage, in which the lytic phage genes are activated, phage particles are produced and lysis occurs.

As used herein a “pharmaceutical composition” refers to a preparation of bacterial cells disclosed herein with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The articles “a” and “an,” as used herein, should be understood to mean “at least one,” unless clearly indicated to the contrary. For example, as used herein, “a heterologous gene encoding a methionine decarboxylase” should be understood to mean “at least one heterologous gene encoding at least one methionine decarboxylase.” Similarly, as used herein, “a heterologous gene encoding an amino acid transporter” should be understood to mean “at least one heterologous gene encoding at least one amino acid transporter.”

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Bacterial Strains

The disclosure provides a bacterial cell that comprises a heterologous gene encoding a methionine catabolism enzyme. In some embodiments, the bacterial cell is a non-pathogenic bacterial cell. In some embodiments, the bacterial cell is a commensal bacterial cell. In some embodiments, the bacterial cell is a probiotic bacterial cell.

In certain embodiments, the bacterial cell is selected from the group consisting of a Bacteroides fragilis, Bacteroides thetaiotaomicron, Bacteroides subtilis, Bifidobacterium animalis, Bifidobacterium bifidum, Bifidobacterium infantis, Bifidobacterium lactis, Clostridium butyricum, Clostridium scindens, Escherichia coli, Lactobacillus acidophilus, Lactobacillus plantarum, Lactobacillus reuteri, Lactococcus lactis, and Oxalobacter formigenes bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium animalis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is a Clostridium scindens bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In one embodiment, the bacterial cell is a Gram positive bacterial cell. In another embodiment, the bacterial cell is a Gram negative bacterial cell.

In some embodiments, the bacterial cell is Escherichia coli strain Nissle 1917 (E. coli Nissle), a Gram-positive bacterium of the Enterobacteriaceae family that “has evolved into one of the best characterized probiotics” (Ukena et al., 2007). The strain is characterized by its “complete harmlessness” (Schultz, 2008), and “has GRAS (generally recognized as safe) status” (Reister et al., 2014, emphasis added). Genomic sequencing confirmed that E. coli Nissle “lacks prominent virulence factors (e.g., E. coli a-hemolysin, P-fimbrial adhesins)” (Schultz, 2008), and E. coli Nissle “does not carry pathogenic adhesion factors and does not produce any enterotoxins or cytotoxins, it is not invasive, not uropathogenic” (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's “therapeutic efficacy and safety have convincingly been proven” (Ukena et al., 2007).

In one embodiment, the recombinant bacterial cell does not colonize the subject.

One of ordinary skill in the art would appreciate that the genetic modifications disclosed herein may be adapted for other species, strains, and subtypes of bacteria. Furthermore, genes from one or more different species can be introduced into one another, e.g., an amino acid catabolism gene from Klebsiella quasipneumoniae can be expressed in Escherichia coli.

In some embodiments, the bacterial cell is a genetically engineered bacterial cell. In another embodiment, the bacterial cell is a recombinant bacterial cell. In some embodiments, the disclosure comprises a colony of bacterial cells.

In another aspect, the disclosure provides a recombinant bacterial culture which comprises bacterial cells disclosed herein. In one aspect, the disclosure provides a recombinant bacterial culture which reduces levels of an amino acid, e.g., methionine, in the media of the culture. In one embodiment, the levels of an amino acid are reduced by about 50%, about 75%, or about 100% in the media of the cell culture. In another embodiment, the levels of an amino acid are reduced by about two-fold, three-fold, four-fold, five-fold, six-fold, seven-fold, eight-fold, nine-fold, or ten-fold in the media of the cell culture. In one embodiment, the levels of an amino acid, e.g., methionine, are reduced below the limit of detection in the media of the cell culture.

In some embodiments of the above described genetically engineered bacteria, the gene encoding a methionine decarboxylase is present on a plasmid in the bacterium and operatively linked on the plasmid to the promoter that is induced under low-oxygen or anaerobic conditions. In other embodiments, the gene encoding a methionine decarboxylase is present in the bacterial chromosome and is operatively linked in the chromosome to the promoter that is induced under low-oxygen or anaerobic conditions.

In some embodiments of the above described genetically engineered bacteria, the gene encoding a methionine decarboxylase is present on a plasmid in the bacterium and operatively linked on the plasmid to the promoter that is chemically induced. In other embodiments, the gene encoding a methionine decarboxylase is present in the bacterial chromosome and is operatively linked in the chromosome to the promoter that is chemically induced.

In some embodiments of the above described genetically engineered bacteria, the gene encoding a methionine importer is present on a plasmid in the bacterium and operatively linked on the plasmid to the promoter that is chemically induced. In other embodiments, the gene encoding a methionine importer is present in the bacterial chromosome and is operatively linked in the chromosome to the promoter that is chemically induced.

In some embodiments of the above described genetically engineered bacteria, the gene encoding a methionine decarboxylase is present on a plasmid in the bacterium and operatively linked on the plasmid to the promoter that is chemically induced and the gene encoding a methionine importer is present on a plasmid in the bacterium and operatively linked on the plasmid to the promoter that is chemically induced. In other embodiments, the gene encoding a methionine decarboxylase is present in the bacterial chromosome and is operatively linked in the chromosome to the promoter that is chemically induced and the gene encoding a methionine importer is present in the bacterial chromosome and is operatively linked in the chromosome to the promoter that is chemically induced.

In some embodiments of the above described genetically engineered bacteria, the gene encoding a methionine decarboxylase is present on a plasmid in the bacterium and operatively linked on the plasmid to the promoter that is chemically induced and the gene encoding a methionine importer is present in the bacterial chromosome and is operatively linked in the chromosome to the promoter that is chemically induced. In other embodiments, the gene encoding a methionine decarboxylase is present in the bacterial chromosome and is operatively linked in the chromosome to the promoter that is chemically induced and the gene encoding a methionine importer is present on a plasmid in the bacterium and operatively linked on the plasmid to the promoter that is chemically induced.

In some embodiments, the genetically engineered bacteria is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thiI auxotroph. In some embodiments, the engineered bacteria have more than one auxotrophy, for example, they may be a ΔthyA and ΔdapA auxotroph.

In some embodiments of the above described genetically engineered bacteria, the gene encoding a methionine decarboxylase is present on a plasmid in the bacterium and operatively linked on the plasmid to the promoter that is induced under low-oxygen or anaerobic conditions. In other embodiments, the gene encoding a methionine decarboxylase is present in the bacterial chromosome and is operatively linked in the chromosome to the promoter that is induced under low-oxygen or anaerobic conditions.

### A. Methionine Catabolism Enzymes

Methionine catabolism enzymes may be expressed or modified in the bacteria disclosed herein in order to enhance catabolism of methionine. For example, the genetically engineered bacteria comprising at least one heterologous gene encoding a methionine catabolism enzyme can catabolize methionine to treat a disease associated with methionine, including, but not limited to homocystinuria, cystinuria, primary and secondary hypermethioninemia, cystathionine β-synthase (CBS) deficiency, or cancer, e.g., lymphoblastic leukemia.

As used herein, the term “methionine catabolism enzyme” refers to an enzyme involved in the catabolism of methionine. Specifically, when a methionine catabolism enzyme is expressed in a recombinant bacterial cell, the bacterial cell hydrolyzes more methionine into 3-methylthiopropylamine (3-MTP) when the catabolism enzyme is expressed than unmodified bacteria of the same bacterial subtype under the same conditions. In some embodiments, methionine transporters may also be expressed or modified in the recombinant bacteria to enhance methionine import into the cell in order to increase the catabolism of methionine by the methionine catabolism enzyme. In other embodiments, methionine exporters may be knocked-out in the recombinant bacteria to decrease export of methionine and/or increase cytoplasmic concentration of methionine.

In one embodiment, the methionine catabolism enzyme increases the rate of methionine catabolism in the cell. In one embodiment, the methionine catabolism enzyme decreases the level of methionine in the cell. In another embodiment, the methionine catabolism enzyme increases the level of 3-methylthiopropylamine in the cell. In one embodiment, 3-methylthiopropylamine is not toxic to the cell.

Methionine catabolism enzymes are well known to those of skill in the art (see, e.g., Huang et al., Mar. Drugs, 13 (8): 5492-5507, 2015). For example, the adenosylmethionine synthase pathway has been identified in Anabaena cylindrica. In the adenosylmethionine synthase pathway, methionine is catabolized into S-adenosyl-L-homocysteine by an S-adenosylmethionine synthase enzyme, followed by conversion of the S-adenosyl-L-homocysteine into L-homocysteine by an adenosylhomocysteinase enzyme. As another example, two methionine aminotransferase enzymes (including Aro8 and Aro9), and one decarboxylase gene (Aro10) have been identified in Saccharomyces cerevisiae which catabolize methionine (Yin et al. (2015) FEMS Microbiol. Lett. 362 (5) pii: fnu043). Methionine aminotransferase enzymes catabolize methionine and 2-oxo carboxylate into 2-oxo-4-methylthiobutanoate and an L-amino acid.

In some embodiments, a methionine catabolism enzyme is encoded by a gene encoding a methionine catabolism enzyme derived from a bacterial species. In some embodiments, a methionine catabolism enzyme is encoded by a gene encoding a methionine catabolism enzyme derived from a non-bacterial species. In some embodiments, a methionine catabolism enzyme is encoded by a gene derived from a eukaryotic species, e.g., a yeast species or a plant species. In one embodiment, the gene encoding the methionine catabolism enzyme is derived from an organism of the genus or species that includes, but is not limited to, Klebsiella quasipneumoniae, Bacillus subtilis, Caenorhabditis elegans, Entamoeba histolytica, Bacillus halodurans, Methylobacterium aquaticum, Saccharomyces cerevisiae, Escherichia coli, and Anabaena cylindrica.

In one embodiment, the methionine catabolism enzyme is a methionine decarboxylase (MDC). In one embodiment, the methionine decarboxylase gene is a MDC gene from Streptomyces sp. 590. On example of such a MDC gene is described, for example, in Misono et al., Bull. Inst. Chem. Res., Kyoto Univ., 58 (3): 323-333, 1980. In one embodiment, the methionine decarboxylase gene is a metDC from Stanieria sp. NIES-3757. In one embodiment, the methionine decarboxylase gene is a metDC from Mus musculus. In one embodiment, the methionine decarboxylase gene is a metDC from Entamoeba histolytica.

In one embodiment, the methionine decarboxylase gene encodes a polypeptide with a Q70D mutation referenced by the polypeptide encoded by the gene sequence having the sequence of SEQ ID NO: 596. In one embodiment, the methionine decarboxylase gene encodes a polypeptide with a N82H mutation referenced by the polypeptide encoded by the gene sequence having the sequence of SEQ ID NO: 596. In one embodiment, the methionine decarboxylase gene encodes a polypeptide with a Q70D N82H mutations referenced by the polypeptide encoded by the gene sequence having the sequence of SEQ ID NO: 596.

In one embodiment, the methionine decarboxylase gene encodes a polypeptide with a V49I mutation referenced by the polypeptide encoded by the gene sequence having the sequence of SEQ ID NO: 596. In one embodiment, the methionine decarboxylase gene encodes a polypeptide with a A500P mutation referenced by the polypeptide encoded by the gene sequence having the sequence of SEQ ID NO: 596. In one embodiment, the methionine decarboxylase gene encodes a polypeptide with a V49I A500P mutations referenced by the polypeptide encoded by the gene sequence having the sequence of SEQ ID NO: 596.

In one embodiment, the methionine decarboxylase gene encodes a polypeptide with a R41Q mutation referenced by the polypeptide encoded by the gene sequence having the sequence of SEQ ID NO: 596. In one embodiment, the methionine decarboxylase gene encodes a polypeptide with a Q70D mutation referenced by the polypeptide encoded by the gene sequence having the sequence of SEQ ID NO: 596. In one embodiment, the methionine decarboxylase gene encodes a polypeptide with a R41Q Q70D mutations referenced by the polypeptide encoded by the gene sequence having the sequence of SEQ ID NO: 596.

In one embodiment, the methionine decarboxylase gene has at least about 80% identity with the sequence of SEQ ID NO: 596. Accordingly, in one embodiment, the methionine decarboxylase gene has at least about 90% identity with the sequence of SEQ ID NO: 596. Accordingly, in one embodiment, the methionine decarboxylase gene has at least about 95% identity with the sequence of SEQ ID NO: 596. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 596. In another embodiment, the methionine decarboxylase gene comprises the sequence of SEQ ID NO: 596. In yet another embodiment the methionine decarboxylase gene consists of the sequence of SEQ ID NO: 596.

In one embodiment, the methionine decarboxylase gene has at least about 80% identity with the sequence of SEQ ID NO: 611. Accordingly, in one embodiment, the methionine decarboxylase gene has at least about 90% identity with the sequence of SEQ ID NO: 611. Accordingly, in one embodiment, the methionine decarboxylase gene has at least about 95% identity with the sequence of SEQ ID NO: 611. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 611. In another embodiment, the methionine decarboxylase gene comprises the sequence of SEQ ID NO: 611. In yet another embodiment the methionine decarboxylase gene consists of the sequence of SEQ ID NO: 611.

In one embodiment, the methionine decarboxylase gene has at least about 80% identity with the sequence of SEQ ID NO: 627. Accordingly, in one embodiment, the methionine decarboxylase gene has at least about 90% identity with the sequence of SEQ ID NO: 627. Accordingly, in one embodiment, the methionine decarboxylase gene has at least about 95% identity with the sequence of SEQ ID NO: 627. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 627. In another embodiment, the methionine decarboxylase gene comprises the sequence of SEQ ID NO: 627. In yet another embodiment the methionine decarboxylase gene consists of the sequence of SEQ ID NO: 627.

In one embodiment, the methionine decarboxylase gene has at least about 80% identity with the sequence of SEQ ID NO: 628. Accordingly, in one embodiment, the methionine decarboxylase gene has at least about 90% identity with the sequence of SEQ ID NO: 628. Accordingly, in one embodiment, the methionine decarboxylase gene has at least about 95% identity with the sequence of SEQ ID NO: 628. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 628. In another embodiment, the methionine decarboxylase gene comprises the sequence of SEQ ID NO: 628. In yet another embodiment the methionine decarboxylase gene consists of the sequence of SEQ ID NO: 628.

In one embodiment, the methionine decarboxylase gene has at least about 80% identity with the sequence of SEQ ID NO: 629. Accordingly, in one embodiment, the methionine decarboxylase gene has at least about 90% identity with the sequence of SEQ ID NO: 629. Accordingly, in one embodiment, the methionine decarboxylase gene has at least about 95% identity with the sequence of SEQ ID NO: 629. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 629. In another embodiment, the methionine decarboxylase gene comprises the sequence of SEQ ID NO: 629. In yet another embodiment the methionine decarboxylase gene consists of the sequence of SEQ ID NO: 629.

In one embodiment, the methionine decarboxylase gene has at least about 80% identity with the sequence of SEQ ID NO: 630. Accordingly, in one embodiment, the methionine decarboxylase gene has at least about 90% identity with the sequence of SEQ ID NO: 630. Accordingly, in one embodiment, the methionine decarboxylase gene has at least about 95% identity with the sequence of SEQ ID NO: 630. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 630. In another embodiment, the methionine decarboxylase gene comprises the sequence of SEQ ID NO: 630. In yet another embodiment the methionine decarboxylase gene consists of the sequence of SEQ ID NO: 630.

In one embodiment, the methionine decarboxylase gene has at least about 80% identity with the sequence of SEQ ID NO: 631. Accordingly, in one embodiment, the methionine decarboxylase gene has at least about 90% identity with the sequence of SEQ ID NO: 631. Accordingly, in one embodiment, the methionine decarboxylase gene has at least about 95% identity with the sequence of SEQ ID NO: 631. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 631. In another embodiment, the methionine decarboxylase gene comprises the sequence of SEQ ID NO: 631. In yet another embodiment the methionine decarboxylase gene consists of the sequence of SEQ ID NO: 631.

In one embodiment, the methionine decarboxylase gene has at least about 80% identity with the sequence of SEQ ID NO: 633. Accordingly, in one embodiment, the methionine decarboxylase gene has at least about 90% identity with the sequence of SEQ ID NO: 633. Accordingly, in one embodiment, the methionine decarboxylase gene has at least about 95% identity with the sequence of SEQ ID NO: 633. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 633. In another embodiment, the methionine decarboxylase gene comprises the sequence of SEQ ID NO: 633. In yet another embodiment the methionine decarboxylase gene consists of the sequence of SEQ ID NO: 633.

In one embodiment, the recombinant bacteria comprise a leucine decarboxylase. As used herein “leucine decarboxylase” (referred to herein also as LDC or LeuDC) refers to any polypeptide having enzymatic activity that catalyzes the conversion of leucine to isopentylamine. The bacterial cells disclosed herein may comprise a heterologous gene encoding a leucine decarboxylase enzyme and are capable of converting leucine into isopentylamine.

In some embodiment, the leucine decarboxylase gene has at least about 80% with the sequence of SEQ ID NO: 632. Accordingly, in one embodiment, the leucine decarboxylase gene has at least about 90% identity with the sequence of SEQ ID NO: 632. Accordingly, in one embodiment, the c leucine decarboxylase gene has at least about 95% identity with the sequence of SEQ ID NO: 632. Accordingly, in one embodiment, the leucine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 632. In another embodiment, the leucine decarboxylase gene comprises the sequence of SEQ ID NO: 632. In yet another embodiment the leucine decarboxylase gene consists of the sequence of SEQ ID NO: 632.

The present disclosure further comprises genes encoding functional fragments of a methionine decarboxylase enzyme.

Assays for testing the activity of a methionine catabolism enzyme, a methionine catabolism enzyme functional variant, or a methionine catabolism enzyme functional fragment are well known to one of ordinary skill in the art. For example, methionine catabolism can be assessed by expressing the protein, functional variant, or fragment thereof, in a recombinant bacterial cell that lacks endogenous methionine catabolism enzyme activity. Other methods are also well known to one of ordinary skill in the art (see, e.g., Dolzan et al., FEBS Letters, 574:141-146, 2004, the entire contents of which are incorporated by reference).

In some embodiments, the genetically engineered bacteria comprise a stably maintained plasmid or chromosome carrying a gene for producing a methionine decarboxylase, such that the methionine decarboxylase can be expressed in the host cell, and the host cell is capable of survival and/or growth in vitro, e.g., in medium, and/or in vivo. In some embodiments, a bacterium may comprise multiple copies of the gene encoding the methionine decarboxylase. In some embodiments, the gene encoding the methionine decarboxylase is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the methionine decarboxylase is expressed on a high-copy plasmid. In some embodiments, the high-copy plasmid may be useful for increasing expression of the methionine decarboxylase. In some embodiments, the gene encoding the methionine decarboxylase is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular methionine decarboxylase inserted at four different insertion sites. Alternatively, the genetically engineered bacteria may include three copies of the gene encoding a particular methionine decarboxylase inserted at three different insertion sites and three copies of the gene encoding a different methionine decarboxylase inserted at three different insertion sites.

In some embodiments, under conditions where the methionine decarboxylase is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the methionine decarboxylase, and/or transcript of the gene(s) in the operon as compared to unmodified bacteria of the same subtype under the same conditions.

In some embodiments, quantitative PCR (qPCR) is used to amplify, detect, and/or quantify mRNA expression levels of the methionine decarboxylase gene(s). Primers specific for methionine decarboxylase the gene(s) may be designed and used to detect mRNA in a sample according to methods known in the art. In some embodiments, a fluorophore is added to a sample reaction mixture that may contain methionine decarboxylase mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the methionine decarboxylase gene(s).

In some embodiments, quantitative PCR (qPCR) is used to amplify, detect, and/or quantify mRNA expression levels of the methionine decarboxylase gene(s). Primers specific for methionine decarboxylase the gene(s) may be designed and used to detect mRNA in a sample according to methods known in the art. In some embodiments, a fluorophore is added to a sample reaction mixture that may contain methionine decarboxylase mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the methionine decarboxylase gene(s).

In one embodiment, the bacterial cell comprises a heterologous gene encoding a methionine catabolism enzyme. In one embodiment, the bacterial cell comprises a heterologous gene encoding a transporter of methionine and a heterologous gene encoding a methionine catabolism enzyme. In one embodiment, the bacterial cell comprises a heterologous gene encoding a methionine catabolism enzyme and a genetic modification that reduces export of methionine. In one embodiment, the bacterial cell comprises a heterologous gene encoding a transporter of methionine, a heterologous gene encoding a methionine catabolism enzyme, and a genetic modification that reduces export of methionine. Transporters and exporters are described in more detail in the subsections, below.

### B. Transporters of Methionine

Methionine transporters, or importers, may be expressed or modified in the recombinant bacteria described herein in order to enhance methionine transport into the cell. Specifically, when the transporter of methionine is expressed in the recombinant bacterial cells described herein, the bacterial cells import more methionine into the cell when the transporter is expressed than unmodified bacteria of the same bacterial subtype under the same conditions. Thus, the genetically engineered bacteria comprising a heterologous gene encoding transporter of methionine which may be used to import methionine into the bacteria so that any gene encoding a methionine catabolism enzyme expressed in the organism can catabolize the methionine to treat a disease associated with methionine, such as homocystinuria.

The uptake of methionine into bacterial cells is mediated by proteins well known to those of skill in the art. For example, a methionine transporter operon has been identified in Corynebacterium glutamicum (Trotschel et al., J. Bacteriology, 187 (11): 3786-3794, 2005). In addition, the high affinity MetD ABC transporter system has been characterized in Escherichia coli (Kadaba et al. (2008) Science 5886:250-253; Kadner and Watson (1974) J. Bacteriol. 119:401-9). The MetD transporter system is capable of mediating the translocation of several substrates across the bacterial membrane, including methionine. The MetD system of Escherichia coli consists of MetN (encoded by metN), which comprises the ATPase domain, MetI (encoded by metI), which comprises the transmembrane domain, and MetQ (encoded by metQ), the cognate binding protein which is located in the periplasm. Orthologues of the genes encoding the E. coli MetD transporter system have been identified in multiple organisms including, e.g., Yersinia pestis, Vibrio cholerae, Pasteurella multocida, Haemophilus influenza, Agrobacterium tumefaciens, Sinorhizobium meliloti, Brucella meliloti, and Mesorhizobium loti (Merlin et al. (2002) J. Bacteriol. 184:5513-7).

In one embodiment, the at least one gene encoding a transporter of methionine is a metN gene, a metI gene, and/or a metQ gene from Corynebacterium glutamicum, Escherichia coli, and Bacillus subtilis (Trotschel et al., J. Bacteriology, 187 (11): 3786-3794, 2005).

In one embodiment, the metN gene has at least about 80% identity with the sequence of SEQ ID NOs: 657 or 597. Accordingly, in one embodiment, the metN gene has at least about 90% identity with the sequence of SEQ ID NOs: 657 or 597. Accordingly, in one embodiment, the metN gene has at least about 95% identity with the sequence of SEQ ID NOs: 657 or 597. Accordingly, in one embodiment, the metN gene has at least about 85%, 86%, 87%, 88%, 89%, 90%, 91%, 92%, 93%, 94%, 95%, 96%, 97%, 98%, or 99% identity with the sequence of SEQ ID NOs: 657 or 597. In another embodiment, the metN gene comprises the sequence of SEQ ID NOs: 657 or 597. In yet another embodiment the metN gene consists of the sequence of SEQ ID NOs: 657 or 597.

In one embodiment, the metI gene has at least about 80% identity with the sequence of SEQ ID NOs: 658 or 598. Accordingly, in one embodiment, the metI gene has at least about 90% identity with the sequence of SEQ ID NOs: 658 or 598. Accordingly, in one embodiment, the metI gene has at least about 95% identity with the sequence of SEQ ID NOs: 658 or 598. Accordingly, in one embodiment, the metI gene has at least about 85%, 86%, 87%, 88%, 89%, 90%, 91%, 92%, 93%, 94%, 95%, 96%, 97%, 98%, or 99% identity with the sequence of SEQ ID NOs: 658 or 598. In another embodiment, the metI gene comprises the sequence of SEQ ID NOs: 658 or 598. In yet another embodiment the metI gene consists of the sequence of SEQ ID NOs: 658 or 598.

In one embodiment, the metQ gene has at least about 80% identity with the sequence of SEQ ID NOs: 659 or 599. Accordingly, in one embodiment, the metQ gene has at least about 90% identity with the sequence of SEQ ID NOs: 659 or 599. Accordingly, in one embodiment, the metQ gene has at least about 95% identity with the sequence of SEQ ID NOs: 659 or 599. Accordingly, in one embodiment, the metQ gene has at least about 85%, 86%, 87%, 88%, 89%, 90%, 91%, 92%, 93%, 94%, 95%, 96%, 97%, 98%, or 99% identity with the sequence of SEQ ID NOs: 659 or 599. In another embodiment, the metQ gene comprises the sequence of SEQ ID NOs: 659 or 599. In yet another embodiment the metQ gene consists of the sequence of SEQ ID NOs: 659 or 599.

In one embodiment, the metNIQ gene has at least about 80% identity with the sequence of SEQ ID NOs: 636, 638, 639, or 640. Accordingly, in one embodiment, the metNIQ gene has at least about 90% identity with the sequence of SEQ ID NOs: 636, 638, 639, or 640. Accordingly, in one embodiment, the metNIQ gene has at least about 95% identity with the sequence of SEQ ID NOs: 636, 638, 639, or 640. Accordingly, in one embodiment, the metNIQ gene has at least about 85%, 86%, 87%, 88%, 89%, 90%, 91%, 92%, 93%, 94%, 95%, 96%, 97%, 98%, or 99% identity with the sequence of SEQ ID NOs: 636, 638, 639, or 640. In another embodiment, the metNIQ gene comprises the sequence of SEQ ID NOs: 636, 638, 639, or 640. In yet another embodiment the metNIQ gene consists of the sequence of SEQ ID NOs: 636, 638, 639, or 640.

In one embodiment, the metNIQ gene encodes a polypeptide with a P281G mutation in the MetN polypeptide referenced by the MetN polypeptide encoded by the gene sequence having the sequence of SEQ ID NO: 597. In one embodiment, the metNIQ gene encodes a polypeptide with a P281S mutation in the MetN polypeptide referenced by the MetN polypeptide encoded by the gene sequence having the sequence of SEQ ID NO: 597.

In one embodiment, at least one gene encoding a transporter of methionine is a metP gene. In one embodiment, the metP gene is from Flavobacterium segetis. In one embodiment, the metP gene is from Flavobacterium frigoris.

In one embodiment, the metP gene has at least about 80% identity with the sequence of SEQ ID NO: 634. Accordingly, in one embodiment, the metP gene has at least about 90% identity with the sequence of SEQ ID NO: 634. Accordingly, in one embodiment, the metP gene has at least about 95% identity with the sequence of SEQ ID NO: 634. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 634. In another embodiment, the metP gene comprises the sequence of SEQ ID NO: 634. In yet another embodiment the metP gene consists of the sequence of SEQ ID NO: 634.

In one embodiment, the metP gene has at least about 80% identity with the sequence of SEQ ID NO: 635. Accordingly, in one embodiment, the metP gene has at least about 90% identity with the sequence of SEQ ID NO: 635. Accordingly, in one embodiment, the metP gene has at least about 95% identity with the sequence of SEQ ID NO: 635. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 635. In another embodiment, the metP gene comprises the sequence of SEQ ID NO: 635. In yet another embodiment the metP gene consists of the sequence of SEQ ID NO: 635.

In one embodiment, the metP gene has at least about 80% identity with the sequence of SEQ ID NO: 637. Accordingly, in one embodiment, the metP gene has at least about 90% identity with the sequence of SEQ ID NO: 637. Accordingly, in one embodiment, the metP gene has at least about 95% identity with the sequence of SEQ ID NO: 637. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 637. In another embodiment, the metP gene comprises the sequence of SEQ ID NO: 637. In yet another embodiment the metP gene consists of the sequence of SEQ ID NO: 637.

In some embodiments, the transporter of methionine is encoded by a transporter of methionine gene derived from a bacterial genus or species, including but not limited to, Corynebacterium glutamicum, Escherichia coli, and Bacillus subtilis. In some embodiments, the bacterial species is Escherichia coli strain Nissle.

Assays for testing the activity of a transporter of methionine, a functional variant of a transporter of methionine, or a functional fragment of transporter of methionine are well known to one of ordinary skill in the art. For example, import of methionine may be determined using the methods as described in Trotschel et al., J. Bacteriology, 187 (11): 3786-3794, 2005, the entire contents of which are expressly incorporated by reference herein.

In one embodiment, when the transporter of a methionine is expressed in the recombinant bacterial cells described herein, the bacterial cells import 10% more methionine into the bacterial cell when the transporter is expressed than unmodified bacteria of the same bacterial subtype under the same conditions. In another embodiment, when the transporter of methionine is expressed in the recombinant bacterial cells described herein, the bacterial cells import 20%, 30%, 40%, 50%, 60%, 70%, 80%, 90% or 100% more methionine into the bacterial cell when the transporter is expressed than unmodified bacteria of the same bacterial subtype under the same conditions. In yet another embodiment, when the transporter of methionine is expressed in the recombinant bacterial cells described herein, the bacterial cells import two-fold more methionine into the cell when the transporter is expressed than unmodified bacteria of the same bacterial subtype under the same conditions. In yet another embodiment, when the transporter of methionine is expressed in the recombinant bacterial cells described herein, the bacterial cells import three-fold, four-fold, five-fold, six-fold, seven-fold, eight-fold, nine-fold, or ten-fold more methionine into the cell when the transporter is expressed than unmodified bacteria of the same bacterial subtype under the same conditions.

### C. Exporters of Methionine

Methionine exporters may be modified in the recombinant bacteria described herein in order to reduce methionine export from the cell. Specifically, when the recombinant bacterial cells described herein comprise a genetic modification that reduces export of methionine, the bacterial cells retain more methionine in the bacterial cell than unmodified bacteria of the same bacterial subtype under the same conditions. Thus, the recombinant bacteria comprising a genetic modification that reduces export of methionine may be used to retain more methionine in the bacterial cell so that any methionine catabolism enzyme expressed in the organism, e.g., co-expressed methionine catabolism enzyme, can catabolize the methionine.

Exporters of methionine are well known to one of ordinary skill in the art. For example, the MetE methionine exporter from Bacillus atrophaeus, and the BrnFE methionine exporter from Corynebacterium glutamicum have been described (Trotschel et al., J. Bacteriology, 187 (11): 3786-3794, 2005). The YjeH methionine exporter from E. coli has also been described (Liu et al., 2015: Applied and Environmental Microbiology, 81 (22): 7753-7766).

In one embodiment, the methionine exporter is yjeH. In one embodiment, the yjeH gene has at least about 80% identity with the sequence of SEQ ID NO: 607. Accordingly, in one embodiment, the yjeH gene has at least about 90% identity with the sequence of SEQ ID NO: 607. Accordingly, in one embodiment, the yjeH gene has at least about 95% identity with the sequence of SEQ ID NO: 607. Accordingly, in one embodiment, the yjeH gene has at least about 85%, 86%, 87%, 88%, 89%, 90%, 91%, 92%, 93%, 94%, 95%, 96%, 97%, 98%, or 99% identity with the sequence of SEQ ID NO: 607. In another embodiment, the yjeH gene comprises the sequence of SEQ ID NO: 607. In yet another embodiment the yjeH gene consists of the sequence of SEQ ID NO: 607. In one embodiment, the yjeH gene is deleted. In another embodiment, a point mutation in the yjeH gene prevents export of methionine from the cell.

In one embodiment, the genetic modification is a mutation in an endogenous gene encoding an exporter of methionine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export methionine from the bacterial cell.

In another embodiment, the genetic modification is a mutation in a promoter of an endogenous gene encoding an exporter of methionine.

In yet another embodiment, the genetic modification is an overexpression of a repressor of an exporter of methionine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

### D. Inducible Promoters

In some embodiments, the bacterial cell comprises a stably maintained plasmid or chromosome carrying the gene(s) encoding the methionine decarboxylase(s), such that the methionine decarboxylase(s) can be expressed in the host cell, and the host cell is capable of survival and/or growth in vitro, e.g., in medium, and/or in vivo, e.g., in the gut. In some embodiments, bacterial cell comprises two or more distinct methionine decarboxylases or operons, e.g., two or more methionine decarboxylase genes. In some embodiments, bacterial cell comprises three or more distinct methionine decarboxylases or operons, e.g., three or more methionine decarboxylase genes. In some embodiments, bacterial cell comprises 4, 5, 6, 7, 8, 9, 10, or more distinct methionine decarboxylases or operons, e.g., 4, 5, 6, 7, 8, 9, 10, or more methionine decarboxylase genes.

In some embodiments, the genetically engineered bacteria comprise multiple copies of the same methionine decarboxylase gene(s). In some embodiments, the gene encoding the methionine decarboxylase is present on a plasmid and operably linked to a directly or indirectly inducible promoter. In some embodiments, the gene encoding the methionine decarboxylase is present on a plasmid and operably linked to a promoter that is induced under low-oxygen or anaerobic conditions. In some embodiments, the gene encoding the methionine decarboxylase is present on a chromosome and operably linked to a directly or indirectly inducible promoter. In some embodiments, the gene encoding the methionine decarboxylase is present in the chromosome and operably linked to a promoter that is induced under low-oxygen or anaerobic conditions. In some embodiments, the gene encoding the methionine decarboxylase is present on a plasmid and operably linked to a promoter that is induced by exposure to tetracycline, arabinose or Isopropyl β-D-1-thiogalactopyranoside (IPTG).

In some embodiments, the inducible promoter is a IPTG inducible promoter. In one embodiment, the IPTG inducible promoter comprises a sequence having at least 90%, 91%, 92%, 93%, 94%, 95%, 96%, 97%, 98%, or 99% identity to, comprises, or consists of SEQ ID NO: 17. In some embodiments, the recombinant bacterium further comprises a gene sequence encoding a repressor of the Lac promoter. In some embodiments, the gene sequence encoding a repressor comprises a sequence having at least 90%, 91%, 92%, 93%, 94%, 95%, 96%, 97%, 98%, or 99% identity to, comprises, or consists of SEQ ID NO: 15. In some embodiments, the repressor comprises a sequence having at least 90%, 91%, 92%, 93%, 94%, 95%, 96%, 97%, 98%, or 99% identity to, comprises, or consists of SEQ ID NO: 16.

TABLE 3
IPTG inducible promoter and LacI sequences

| Description | |
|---|---|
| SEQ ID NO | Sequences |
| LacI in reverse | TCACTGCCCGCTTTCCAGTCGGGAAACCTG |
| orientation | TCGTGCCAGCTGCATTAATGAATCGGCCAA |
| SEQ ID NO: 15 | CGCGCGGGGAGAGGCGGTTTGCGTATTGGG |
| | CGCCAGGGTGGTTTTTCTTTTCACCAGTGA |
| | GACTGGCAACAGCTGATTGCCCTTCACCGC |
| | CTGGCCCTGAGAGAGTTGCAGCAAGCGGTC |
| | CACGCTGGTTTGCCCCAGCAGGCGAAAATC |
| | CTGTTTGATGGTGGTTAACGGCGGGATATA |
| | ACATGAGCTATCTTCGGTATCGTCGTATCC |
| | CACTACCGAGATATCCGCACCAACGCGCAG |
| | CCCGGACTCGGTAATGGCGCGCATTGCGCC |
| | CAGCGCCATCTGATCGTTGGCAACCAGCAT |
| | CGCAGTGGGAACGATGCCCTCATTCAGCAT |
| | TTGCATGGTTTGTTGAAAACCGGACATGGC |
| | ACTCCAGTCGCCTTCCCGTTCCGCTATCGG |
| | CTGAATTTGATTGCGAGTGAGATATTTATG |
| | CCAGCCAGCCAGACGCAGACGCGCCGAGAC |
| | AGAACTTAATGGGCCCGCTAACAGCGCGAT |
| | TTGCTGGTGACCCAATGCGACCAGATGCTC |
| | CACGCCCAGTCGCGTACCGTCCTCATGGGA |
| | GAAAATAATACTGTTGATGGGTGTCTGGTC |
| | AGAGACATCAAGAAATAACGCCGGAACATT |
| | AGTGCAGGCAGCTTCCACAGCAATGGCATC |
| | CTGGTCATCCAGCGGATAGTTAATGATCAG |
| | CCCACTGACGCGTTGCGCGAGAAGATTGTG |
| | CACCGCCGCTTTACAGGCTTCGACGCCGCT |
| | TCGTTCTACCATCGACACCACCACGCTGGC |
| | ACCCAGTTGATCGGCGCGAGATTTAATCGC |
| | CGCGACAATTTGCGACGGCGCGTGCAGGGC |
| | CAGACTGGAGGTGGCAACGCCAATCAGCAA |
| | CGACTGTTTGCCCGCCAGTTGTTGTGCCAC |
| | GCGGTTGGGAATGTAATTCAGCTCCGCCAT |
| | CGCCGCTTCCACTTTTTCCCGCGTTTTCGC |
| | AGAAACGTGGCTGGCCTGGTTCACCACGCG |
| | GGAAACGGTCTGATAAGAGACACCGGCATA |
| | CTCTGCGACATCGTATAACGTTACTGGTTT |
| | CAT |
| LacI | MKPVTLYDVAEYAGVSYQTVSRVVNQASHV |
| SEQ ID NO: 16 | SAKTREKVEAAMAELNYIPNRVAQQLAGKQ |
| | SLLIGVATSSLALHAPSQIVAAIKSRADQL |
| | GASVVVSMVERSGVEACKAAVHNLLAQRVS |
| | GLIINYPLDDQDAIAVEAACTNVPALFLDV |
| | SDQTPINSIIFSHEDGTRLGVEHLVALGHQ |
| | QIALLAGPLSSVSARLRLAGWHKYLTRNQI |
| | QPIAEREGDWSAMSGFQQTMQMLNEGIVPT |
| | AMLVANDQMALGAMRAITESGLRVGADISV |
| | VGYDDTEDSSCYIPPLTTIKQDFRLLGQTS |
| | VDRLLQLSQGQAVKGNQLLPVSLVKRKTTL |
| | APNTQTASPRALADSLMQLARQVSRLESGQ |
| Lac operator | aattgtgagcgctcacaatt |
| SEQ ID NO: 17 | |
| Promoter | ATTCACCACCCTGAATTGACTCTCTTCCGG |
| comprising Lac | GCGCTATCATGCCATACCGCGAAAGGTTTT |
| operon | GCGCCATTCGATGGCGCGCCGCTTCGTCAG |
| SEQ ID NO: 18 | GCCACATAGCTTTCTTGTTCTGATCGGAAC |
| | GATCGTTGGCTGtgttgacaattaatcatc |
| | ggctcgtataatgtgtggaattgtgagcgc |
| | tcacaattagctgtcaccggatgtgctttc |
| | cggtctgatgagtccgtgaggacgaaacag |
| | cctctacaaataattttgtttaa |
| RBS | Gaccagaggtaaggaggtaacaaccatgcg |
| SEQ ID NO: | agtgttgaagaaacatcttaatcatgctgg |
| 291 | ggagggtttcta |
| Operator 1 | taacaccgtgcgtgttg |
| SEQ ID NO: | |
| 332 | |
| Operator 2 | tacctctggcggtgata |
| SEQ ID NO: | |
| 333 | |

In some embodiments, the promoter that is operably linked to the gene encoding the methionine decarboxylase is directly induced by exogenous environmental conditions. In some embodiments, the promoter that is operably linked to the gene encoding the methionine decarboxylase is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal. In some embodiments, the promoter is directly or indirectly induced by a molecule that is co-administered with the bacterial cell. In one embodiment, the inducible promoter is an anhydrotetracycline (ATC)-inducible promoter. In one embodiment, the inducible promoter is an IPTG promoter. In one embodiment, the IPTG promoter is Ptac.

In certain embodiments, the bacterial cell comprises a gene encoding a methionine decarboxylase expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters listed in the chart, below. Underlined sequences are predicted ribosome binding sites, and bolded sequences are restriction sites used for cloning.

TABLE 4

| FNR | |
|---|---|
| Responsive | |
| Promoter | Sequence |
| SEQ ID | GTCAGCATAACACCCTGACCTCTCATTAATTGTTCATGCC |
| NO: 1 | GGGCGGCACTATCGTCGTCCGGCCTTTTCCTCTCTTACTC |
| | TGCTACGTACATCTATTTCTATAAATCCGTTCAATTTGTC |
| | TGTTTTTTGCACAAACATGAAATATCAGACAATTCCGTGA |
| | CTTAAGAAAATTTATACAAATCAGCAATATACCCCTTAAG |
| | GAGTATATAAAGGTGAATTTGATTTACATCAATAAGCGGG |
| | GTTGCTGAATCGTTAAGGTAGGCGGTAATAGAAAAGAAAT |
| | CGAGGCAAAA |
| SEQ ID | ATTTCCTCTCATCCCATCCGGGGTGAGAGTCTTTTCCCCC |
| NO: 2 | GACTTATGGCTCATGCATGCATCAAAAAAGATGTGAGCTT |
| | GATCAAAAACAAAAAATATTTCACTCGACAGGAGTATTTA |
| | TATTGCGCCCGTTACGTGGGCTTCGACTGTAAATCAGAAA |
| | GGAGAAAACACCT |
| SEQ ID | GTCAGCATAACACCCTGACCTCTCATTAATTGTTCATGCC |
| NO: 3 | GGGCGGCACTATCGTCGTCCGGCCTTTTCCTCTCTTACTC |
| | TGCTACGTACATCTATTTCTATAAATCCGTTCAATTTGTC |
| | TGTTTTTTGCACAAACATGAAATATCAGACAATTCCGTGA |
| | CTTAAGAAAATTTATACAAATCAGCAATATACCCCTTAAG |
| | GAGTATATAAAGGTGAATTTGATTTACATCAATAAGCGGG |
| | GTTGCTGAATCGTTAAGGATCCCTCTAGAAATAATTTTGT |
| | TTAACTTTAAGAAGGAGATATACAT |
| SEQ ID | CATTTCCTCTCATCCCATCCGGGGTGAGAGTCTTTTCCCC |
| NO: 4 | CGACTTATGGCTCATGCATGCATCAAAAAAGATGTGAGCT |
| | TGATCAAAAACAAAAAATATTTCACTCGACAGGAGTATTT |
| | ATATTGCGCCCGGATCCCTCTAGAAATAATTTTGTTTAAC |
| | TTTAAGAAGGAGATATACAT |
| SEQ ID | AGTTGTTCTTATTGGTGGTGTTGCTTTATGGTTGCATCGT |
| NO: 5 | AGTAAATGGTTGTAACAAAAGCAATTTTTCCGGCTGTCTG |
| | TATACAAAAACGCCGTAAAGTTTGAGCGAAGTCAATAAAC |
| | TCTCTACCCATTCAGGGCAATATCTCTCTTGGATCCCTCT |
| | AGAAATAATTTTGTTTAACTTTAAGAAGGAGATATACAT |

In one embodiment, the FNR responsive promoter comprises SEQ ID NO: 1. In another embodiment, the FNR responsive promoter comprises SEQ ID NO: 2. In another embodiment, the FNR responsive promoter comprises SEQ ID NO: 3. In another embodiment, the FNR responsive promoter comprises SEQ ID NO: 4. In yet another embodiment, the FNR responsive promoter comprises SEQ ID NO: 5.

In some embodiments, multiple distinct FNR nucleic acid sequences are inserted in the genetically engineered bacteria. In alternate embodiments, the genetically engineered bacteria comprise a gene encoding a methionine decarboxylase expressed under the control of an alternate oxygen level-dependent promoter, e.g., DNR (Trunk et al., 2010) or ANR (Ray et al., 1997). In these embodiments, expression of the methionine decarboxylase gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the methionine decarboxylase, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the methionine decarboxylase, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the methionine decarboxylase, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006).

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the methionine decarboxylase are present on different plasmids. In some embodiments, the gene encoding the oxygen level-sensing transcriptional regulator and the gene encoding the methionine decarboxylase are present on the same plasmid. In some embodiments, the gene encoding the oxygen level-sensing transcriptional regulator is present on a chromosome. In some embodiments, the gene encoding the oxygen level-sensing transcriptional regulator and the gene encoding the methionine decarboxylase are present on different chromosomes. In some embodiments, the gene encoding the oxygen level-sensing transcriptional regulator and the gene encoding the methionine decarboxylase are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the methionine decarboxylase. In some embodiments, expression of the transcriptional regulator is controlled by the same promoter that controls expression of the methionine decarboxylase. In some embodiments, the transcriptional regulator and the methionine decarboxylase are divergently transcribed from a promoter region.

In some embodiments, any of the gene(s) of the present disclosure may be integrated into the bacterial chromosome at one or more integration sites. For example, one or more copies of one or more encoding a methionine decarboxylase gene(s) may be integrated into the bacterial chromosome. Having multiple copies of the gene or gene(s) integrated into the chromosome allows for greater production of the methionine decarboxylase(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

### E. Temperature Dependent Regulation

In some instances, thermoregulators may be advantageous because of strong transcriptional control without the use of external chemicals or specialized media. Thermoregulated protein expression using the mutant c1857 repressor and the pL and/or pR phage λ promoters have been used to engineer recombinant bacterial strains. For example, a gene of interest cloned downstream of the λ promoters can be efficiently regulated by the mutant thermolabile cI857 repressor of bacteriophage λ. At temperatures below 37° C., c1857 binds to the oL or oR regions of the pR promoter and inhibits transcription by RNA polymerase. At higher temperatures, the functional cI857 dimer is destabilized, binding to the oL or oR DNA sequences is abrogated, and mRNA transcription is initiated. In certain instances, it may be advantageous to reduce, diminish, or shut off production of one or more protein(s) of interest. This can be done in a thermoregulated system by growing a bacterial strain at temperatures at which the temperature regulated system is not optimally active. Temperature regulated expression can then be induced as desired by changing the temperature to a temperature where the system is more active or optimally active.

For example, a thermoregulated promoter may be induced in culture, e.g., grown in a flask, fermenter or other appropriate culture vessel, e.g., used during cell growth, cell expansion, fermentation, recovery, purification, formulation, and/or manufacture. Bacteria comprising gene sequences or gene cassettes either indirectly or directly operably linked to a temperature sensitive system or promoter may, for example, could be induced by temperatures between 37° C. and 42° C. In some instances, the cultures may be grown aerobically. Alternatively, the cultures are grown anaerobically.

In some embodiments, the bacteria described herein comprise one or more gene sequence(s) or gene cassette(s) which are directly or indirectly operably linked to a temperature regulated promoter. In some embodiments, the gene sequence(s) or gene cassette(s) are induced in vitro during growth, preparation, or manufacturing of the strain prior to in vivo administration. In some embodiments, the gene sequence(s) are induced upon or during in vivo administration. In some embodiments, the gene sequence(s) are induced during in vitro growth, preparation, or manufacturing of the strain prior to in vivo administration and upon or during in vivo administration. In some embodiments, the genetically engineered bacteria further comprise gene sequence(s) encoding a transcription factor which is capable of binding to the temperature sensitive promoter. In some embodiments, the transcription factor is a repressor of transcription.

In one embodiment, the thermoregulated promoter is operably linked to a construct having gene sequence(s) or gene cassette(s) encoding one or more protein(s) of interest jointly with a second promoter, e.g., a second constitutive or inducible promoter. In some embodiments, two promoters are positioned proximally to the construct and drive its expression, wherein the thermoregulated promoter is induced under a first set of exogenous conditions, and the second promoter is induced under a second set of exogenous conditions. In a non-limiting example, the first and second conditions may be two sequential culture conditions (i.e., during preparation of the culture in a flask, fermenter or other appropriate culture vessel, e.g., thermoregulation and arabinose or IPTG). In another non-limiting example, the first inducing conditions may be culture conditions, e.g., permissive temperature, and the second inducing conditions may be in vivo conditions. Such in vivo conditions include low-oxygen, microaerobic, or anaerobic conditions, presence of gut metabolites, and/or metabolites administered in combination with the bacterial strain. In some embodiments, one or more thermoregulated promoters drive expression of one or more protein(s) of interest in combination with an oxygen regulated promoter, e.g., FNR, driving the expression of the same gene sequence(s).

In some embodiments, the thermoregulated promoter drives the expression of one or more protein(s) of interest from a low-copy plasmid or a high copy plasmid or a biosafety system plasmid described herein. In some embodiments, the thermoregulated promoter drives the expression of one or more protein(s) of interest from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 19. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 22. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 25. In some embodiments, the thermoregulated construct further comprises a gene encoding mutant cI857 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 20. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 21. In some embodiments, the thermoregulated construct further comprises a gene encoding mutant cI38 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 23. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 24. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 25.

SEQ ID NOs: 19-25 are shown in Table 5.

TABLE 5
Inducible promoter construct sequences and related elements

| Description | Sequence |
|---|---|
| Region comprising | ACGTTAAATCTATCACCGCAAGGGATAAAT |
| Temperature sensitive | ATCTAACACCGTGCGTGTTGACTATTTTAC |
| promoter | CTCTGGCGGTGATAATGGTTGCATAGCTGT |
| SEQ ID NO: 19 | CACCGGATGTGCTTTCCGGTCTGATGAGTC |
| | CGTGAGGACGAAACAGCCTCTACAAATAAT |
| | TTTGTTTAAAACAACACCCACTAAGATAAC |
| | TCTAGAAATAATTTTGTTTAACTTTAAGAA |
| | GGAGATATACAT |
| mutant cI857 repressor | TCAGCCAAACGTCTCTTCAGGCCACTGACT |
| nucleotide sequence | AGCGATAACTTTCCCCACAACGGAACAACT |
| SEQ ID NO: 20 | CTCATTGCATGGGATCATTGGGTACTGTGG |
| | GTTTAGTGGTTGTAAAAACACCTGACCGCT |
| | ATCCCTGATCAGTTTCTTGAAGGTAAACTC |
| | ATCACCCCCAAGTCTGGCTATGCAGAAATC |
| | ACCTGGCTCAACAGCCTGCTCAGGGTCAAC |
| | GAGAATTAACATTCCGTCAGGAAAGCTTGG |
| | CTTGGAGCCTGTTGGTGCGGTCATGGAATT |
| | ACCTTCAACCTCAAGCCAGAATGCAGAATC |
| | ACTGGCTTTTTTGGTTGTGCTTACCCATCT |
| | CTCCGCATCACCTTTGGTAAAGGTTCTAAG |
| | CTTAGGTGAGAACATCCCTGCCTGAACATG |
| | AGAAAAAACAGGGTACTCATACTCACTTCT |
| | AAGTGACGGCTGCATACTAACCGCTTCATA |
| | CATCTCGTAGATTTCTCTGGCGATTGAAGG |
| | GCTAAATTCTTCAACGCTAACTTTGAGAAT |
| | TTTTGTAAGCAATGCGGCGTTATAAGCATT |
| | TAATGCATTGATGCCATTAAATAAAGCACC |
| | AACGCCTGACTGCCCCATCCCCATCTTGTC |
| | TGCGACAGATTCCTGGGATAAGCCAAGTTC |
| | ATTTTTCTTTTTTTCATAAATTGCTTTAAG |
| | GCGACGTGCGTCCTCAAGCTGCTCTTGTGT |
| | TAATGGTTTCTTTTTTGTGCTCAT |
| mutant cI857 repressor | MSTKKKPLTQEQLEDARRLKAIYEKKKNEL |
| polypeptide sequence | GLSQESVADKMGMGQSGVGALFNGINALNA |
| SEQ ID NO: 21 | YNAALLTKILKVSVEEFSPSIAREIYEMYE |
| | AVSMQPSLRSEYEYPVFSHVQAGMFSPKLR |
| | TFTKGDAERWVSTTKKASDSAFWLEVEGNS |
| | MTAPTGSKPSFPDGMLILVDPEQAVEPGDF |
| | CIARLGGDEFTFKKLIRDSGQVFLQPLNPQ |
| | YPMIPCNESCSVVGKVIASQWPEETFG |
| Pr/Pl promoter | ACGTTAAATCTATCACCGCAAGGGATAAAT |
| SEQ ID NO: 22 | ATCTAACACCGTGCGTGTTGACTATTTTAC |
| | CTCTGGCGGTGATAATGGTTGCAT |
| mutant cI38 repressor | Atgagcacaaaaaagaaaccattaacacaa |
| nucleotide sequence | gagcagcttgaggacgcacgtcgccttaaa |
| SEQ ID NO: 23 | gcaatttatgaaaaaaagaaaaatgaactt |
| | ggcttatcccaggaatctgtcgcagacaag |
| | atggggatggggcagtcaggcgttggtgcc |
| | ttatttaatggcatcaatgcattaaatgct |
| | tataacgccgcatcgcttacaagaattctc |
| | aaagttagcgttgaagaatttagcccttca |
| | atcgccagagaaatctacgagatgtatgaa |
| | gcggttagtatgcagccgtcacttagaagt |
| | gagtatgagtaccctgttttttctcatgtt |
| | caggcagggatgctctcacctgagcttaga |
| | acctttaccaaaggtggtgcggaaaggtgg |
| | gtaagcacaaccaaaaaagccagtgattct |
| | gcattctggcttgaggttgaaggtaattcc |
| | atgacagcaccaacaggctccaagccaagc |
| | tttcctgacggaatgttaattctcgttgac |
| | cctgagcaggctgttgagccaggtgatttc |
| | tgcatagccagactcgggggtggtgagttt |
| | accttcaagaaactgatcagggatagcggt |
| | caggtgtttttacaaccactaaacccacag |
| | tacccaatgatcccatgcaatgagagttgt |
| | tccgttgtggggaaagttatcgctagtcag |
| | tggcctgaagagacgtttggctga |
| mutant cI38 repressor | MSTKKKPLTQEQLEDARRLKAIYEKKKNEL |
| polypeptide sequence | GLSQESVADKMGMGQSGVGALFNGINALNA |
| SEQ ID NO: 24 | YNAASLTRILKVSVEEFSPSIAREIYEMYE |
| | AVSMQPSLRSEYEYPVFSHVQAGMLSPELR |
| | TFTKGGAERWVSTTKKASDSAFWLEVEGNS |
| | MTAPTGSKPSFPDGMLILVDPEQAVEPGDF |
| | CIARLGGGEFTFKKLIRDSGQVFLQPLNPQ |
| | YPMIPCNESCSVVGKVIASQWPEETFG |
| Temperature sensitive | aaatctatcaccgcaagggataaatatcta |
| promoter | acaccgtgcgtgttgactattttacctctg |
| SEQ ID NO: 25 | gcggtgataatggttgcATagctgtcaccg |
| | gatgtgctttccggtctgatgagtccgtga |
| | ggacgaaacagcctctacaaataattttgt |
| | ttaaAACAACACCCACTAAGATAAGGTAGA |
| | AAC |

### F. Phage Deletion

In some embodiments, the genetically engineered bacteria comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 1064 is deleted from the Phage 3 genome. In one embodiment, a sequence comprising SEQ ID NO: 1064 is deleted from the Phage 3 genome.

### G. Colibactin Island (Also Known as Pks Island)

In some embodiments, the engineered bacterium further comprises a modified pks island (colibactin island). Non-limiting examples are described in PCT/US2021/061579, the contents of which are herein incorporated by reference in their entirety. Colibactin is a cyclomodulin that is synthetized by enzymes encoded by the pks genomic island. See Fais 2018. The pks genomic island is “highly conserved” in Enterobacteriaceae. Id. In Escherichia coli, a 54-kilobase pks genomic island contains 19 genes, clbA to clbS, and encodes various enzymes that have been described as an “assembly line responsible for colibactin synthesis.” Id. The pks genomic island assembly line for colibactin synthesis includes three polyketide synthases (ClbC, ClbI, ClbO), three non-ribosomal peptide synthases (ClbH, ClbJ, ClbN), two hybrid non-ribosomal peptide/polyketide synthases (ClbB, ClbK), and nine accessory, tailoring, and editing proteins. The polyketide synthases, non-ribosomal peptide synthases, and hybrid enzymes “are usually organized in mega-complexes as an assembly line, in which the synthesized compound is transferred from one enzymatic module to the following one.” Id. Colibactin undergoes a prodrug activation mechanism that incorporates an N-terminal structural motif, which is removed during the final stage of biosynthesis.

In some embodiments, the engineered microorganism, e.g., engineered bacterium, comprises a modified pks island (colibactin island). In some embodiments, the engineered microorganism, e.g., engineered bacterium, comprises a modified clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences, as compared to a suitable control, e.g., the native pks island in an unmodified bacterium of the same strain and/or subtype. In some embodiments, the modified clb sequence is an insertion, a substitution, and/or a deletion as compared to the control. In some embodiments, the modified clb sequence is a deletion of the clb island, e.g., clbA, clbB, clbC, clbD, clbE, clbF, clbG, clbH, clbI, clbJ, clbK, clbL, clbM, clbN, clbO, clbP, clbQ, clbR, and clbS. In one embodiment, the colibactin deletion is the whole island except for the clbS gene, e.g., a deletion of clbA, clbB, clbC, clbD, clbE, clbF, clbG, clbH, clbI, clbJ, clbK, clbL, clbM, clbN, clbO, clbP, clbQ, and clbR.

In some embodiments, the modified endogenous colibactin island comprises one or more modified clb sequences selected from clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304), clbL (SEQ ID NO: 305), clbM (SEQ ID NO: 306), clbN (SEQ ID NO: 307), clbO (SEQ ID NO: 308), clbP (SEQ ID NO: 309), clbQ (SEQ ID NO: 310), clbR (SEQ ID NO: 311), or clbS (SEQ ID NO: 312) gene. In some embodiments, the modified endogenous colibactin island comprises a deletion of clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304, clbL (SEQ ID NO: 305), clbM (SEQ ID NO: 306), clbN (SEQ ID NO: 307), clbO (SEQ ID NO: 308), clbP (SEQ ID NO: 309), clbQ (SEQ ID NO: 310), and clbR (SEQ ID NO: 311).

### H. Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37: D455-D458 and Gerdes et al., Essential genes on metabolic maps, Curr. Opin. Biotechnol., 17 (5): 448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thiI, as long as the corresponding wild-type gene product is not produced in the bacteria. For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, IpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, ftsB, eno, pyrG, chpR, Igt, fbaA, pgk, yqgD, metK, yqgF, plsC, ygiT, pare, ribB, cca, ygjD, tdcF, yraL, yihA, ftsN, mur, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, IspA, ispH, dapB, folA, imp, yabQ, ftsL, ftsI, murE, murF, mraY, murD, ftsW, murG, murC, ftsQ, ftsA, ftsZ, lpxC, secM, secA, can, folk, hemL, yadR, dapD, map, rpsB, infB, nusA, ftsH, obgE, rpmA, rplU, ispB, murA, yrbB, yrbK, yhbN, rpsI, rpIM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, IpxD, fabZ, lpxA, IpxB, dnaE, accA, tilS, proS, yafF, tsf, pyrH, olA, rlpB, leuS, Int, glnS, fldA, cydA, infA, cydC, ftsK, lolA, serS, rpsA, msbA, IpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLIDE) bacterial cell. SLIDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference).

In some embodiments, the SLIDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, 1317S, F319V, L340T, V347I, and S345C. In some embodiments, the essential gene is dnaN comprising the mutations H191N, R240C, 1317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, 149G, and A51C. In some embodiments, the essential gene is metG comprising the mutations E45Q, N47R, 149G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, 149G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4:307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

### Isolated Plasmids

In other embodiments, the disclosure provides an isolated plasmid comprising a first nucleic acid encoding a methionine decarboxylase operably linked to a first inducible promoter. In another embodiment, the disclosure provides an isolated plasmid comprising a second nucleic acid encoding at least one additional methionine decarboxylase. In one embodiment, the first nucleic acid and the second nucleic acid are operably linked to the first promoter. In another embodiment, the second nucleic acid is operably linked to a second inducible promoter. In one embodiment, the first inducible promoter and the second inducible promoter are separate copies of the same inducible promoter. In another embodiment, the first inducible promoter and the second inducible promoter are different inducible promoters. In one embodiment, the first promoter, the second promoter, or the first promoter and the second promoter, are each directly or indirectly induced by low-oxygen or anaerobic conditions. In another embodiment, the first promoter, the second promoter, or the first promoter and the second promoter, are each a fumarate and nitrate reduction regulator (FNR) responsive promoter. In another embodiment, the first promoter, the second promoter, or the first promoter and second promoter are each a ROS-inducible regulatory region. In another embodiment, the first promoter, the second promoter, or the first promoter and second promoter are each a RNS-inducible regulatory region.

In one embodiment, the isolated plasmid comprises at least one heterologous gene encoding a methionine decarboxylase operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene encoding a methionine decarboxylase operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a P_{TetR} promoter.

In any of the above-described embodiments, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

In some embodiments, the recombinant bacterial cell comprises a gene encoding a L-amino acid deaminase (LAAD) polypeptide.

### Integration

In some embodiments, any of the gene(s) or gene cassette(s) of the present disclosure may be integrated into the bacterial chromosome at one or more integration sites. One or more copies of the gene (for example, an amino acid catabolism gene) or gene cassette (for example, a gene cassette comprising an amino acid catabolism gene and an amino acid transporter gene) may be integrated into the bacterial chromosome. Having multiple copies of the gene or gene cassette integrated into the chromosome allows for greater production of the methionine decarboxylase, and other enzymes of the gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

In one non-limiting example, the metP and metDC genes are integrated to facilitate Met import and metabolism. In one embodiment, metP is derived from Flavobacterium segetis and facilitates the uptake of Met into the cell. In one embodiment, MetDC is derived from Streptomyces sp. 590 and includes two modifications (Q70D and N82H). In one embodiment, both genes are operably linked to a chemically inducible promoter. In some embodiments the promoter is induced by the compound Isopropyl β-D-1-thiogalactopyranoside (IPTG) e.g., PTac promoter. In one embodiment, both genes are arranged in a cassette. In some embodiments, both gene s operably linked to the same promoter. In some embodiments, the metP gene and the metDC gene are each are operably linked to separate promoters, i.e., the cassette comprises two promoters, which can be the same or different. A non-limiting example of such a cassette is P_{tac}-metP-P_{tac}-metDC, e.g., wherein both metP and metDC are each operably linked to separate versions of the same inducible promoter, such as an IPTG inducible promoter, or a different inducible promoter described herein. In some embodiments, the genetically engineered bacterium comprises a single integrated copy of metDC. In some embodiments, the genetically engineered bacterium comprises multiple integrated copies of metDC. The multiple copies are present at the same integration site arranged in a cassette and linked to the same promoter. Alternatively, multiple copies of the metDC gene may be integrated at multiple different sites and each metDC gene may be linked to a separate instance of a promoter (which can be the same or different between the different copies), e.g., an inducible promoter such as an IPTG inducible promoter or a different promoter described herein. In one embodiment, genetically engineered bacterium comprises three integrated copies of the metCD gene, which are integrated at three separate integration sites. In some embodiments, these three separate copies of the MetDC gene may be operably linked to separate instances of the same promoter or the promoters may differ between for one or more of the metDC genes. In some embodiments, each of the three copies of the MetDC genes, which are integrated at three separate and distinct sites, are linked to different copies of the same promoter. In some embodiments, the promoter is an inducible promoter, such as an IPTG inducible promoter. In some embodiments, the promoter is a different promoter described herein. In some embodiments, one of the three metDC gene copies is present in a cassette further comprising metP. In one example of such a cassette, metP and metDC are each operably linked to a separate instance of the same promoter, e.g., an IPTG-inducible promoter. In one embodiment, the cassette is P_{tac}-metP-P_{tac}-metDC.

In some embodiments, the bacterium comprises one or more integrated copies of the metP gene, integrated at one or more integration sites. In some embodiments, the MetP is the only gene present at a particular integration site. In some embodiments, the integrated metP gene is part of a cassette further comprising a MetDC gene. In some embodiments, the MetP gene is operably linked to an inducible promoter, e.g., an IPTG inducible promoter.

In one embodiment, the bacterium comprises three integrated copies of the metDC gene, wherein each copy is integrated at a separate integration site. In one embodiments, two integrate copies of the metDC gene are the only gene present at the particular integration site and one integrated copy of the metDC gene present in a cassette, further comprising metP. In some embodiments, the cassette is a P_{tac}-metP-P_{tac}-metDC cassette. In one embodiment, each of three integrated copies of the metDC gene are operably linked to a different instance of the same inducible promoter, wherein the promoter is an IPTG inducible promoter. In one embodiment, the bacterium further comprises a metP gene, wherein the metP gene is operably linked to an inducible promoter, and wherein the inducible promoter is an IPTG inducible promoter. In one embodiment, the metP gene is present in a P_{tac}-metP-P_{tac}-metDC cassette.

In any of these embodiments, the genetically engineered bacterium may further comprise one or more of (1) a deletion in yjeH gene that encodes a Met/branched chain amino acid exporter (2) a deletion of the dapA gene that encodes for dihydrodipicolinate synthase (3) a deletion in the pks island which encodes colibactin and (4) an endogenous Nissle prophage gene deletion.

In one specific embodiment, the bacterium comprises three copies of MetDC gene derived from Streptomyces sp. 590 and comprising two modifications (Q70D and N82H) one copy of a metP gene derived from Flavobacterium segetis, wherein the three copies of the metDC gene and the metP gene are each operably linked to an IPTG inducible promoter, wherein the metP gene is present in a metP-metDC gene cassette, and wherein the bacterium further comprises a deletion in yjeH gene, a deletion of the dapA gene, a deletion in the pks island, and an endogenous Nissle prophage gene deletion.

### In Vivo Models

The recombinant bacteria may be evaluated in vivo, e.g., in an animal model. Any suitable animal model of a disease or condition associated with amino acid metabolism, such as homocystinuria, may be used.

### Pharmaceutical Compositions and Formulations

Pharmaceutical compositions comprising the genetically engineered bacteria described herein may be used to treat, manage, ameliorate, and/or prevent a disorder associated with amino acid catabolism e.g., homocystinuria. Pharmaceutical compositions comprising one or more genetically engineered bacteria, alone or in combination with prophylactic agents, therapeutic agents, and/or pharmaceutically acceptable carriers are provided.

Pharmaceutical compositions comprising the genetically engineered microorganisms of the invention may be used to treat, manage, ameliorate, and/or prevent a disorder associated with amino acid catabolism or symptom(s) associated with diseases or disorders associated with amino acid catabolism. Pharmaceutical compositions of the invention comprising one or more genetically engineered bacteria, and/or one or more genetically engineered virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to express a methionine decarboxylase. In alternate embodiments, the pharmaceutical composition comprises two or more species, strains, and/or subtypes of bacteria that are each engineered to comprise the genetic modifications described herein, e.g., to express a methionine decarboxylase.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In some embodiments, the genetically engineered bacteria are formulated into a pharmaceutical composition comprising vitamin B₆, pyridoxal 5 phosphate (PLP), and/or pyridoxine (PN). In some embodiments, vitamin B₆ and/or PLP and/or PN is added to the pharmaceutical composition at an amount of at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 35 mg, at least about 36 mg, at least about 37 mg, at least about 38 mg, at least about 39 mg, or at least about 40 mg.

In some embodiments, vitamin B₆ and/or PLP and/or PN is added to the pharmaceutical composition at an amount of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 250 mg, or at least about 300 mg.

In some embodiments, vitamin B₆ and/or PLP and/or PN is added to the pharmaceutical composition at an amount of at about 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg.

In some embodiments, vitamin B₆ and/or PLP and/or PN is added to the pharmaceutical composition at an amount of at about 50 mg to about 75 mg, about 75 mg to about 100 mg, about 100 mg to about 125 mg, about 125 mg to about 150 mg, about 150 mg to about 175 mg, or about 175 mg to about 200 mg.

In some embodiments, vitamin B₆ and/or PLP and/or PN is added to the pharmaceutical composition at an amount of at about 1 mg to about 50 mg, about 50 mg to about 100 mg, about 100 mg to about 150 mg, about 150 mg to about 200 mg, about 200 mg to about 300 mg, about 300 mg to about 400 mg, or about 400 mg to about 500 mg.

In some specific embodiments, the genetically engineered bacteria may be formulated into a pharmaceutical composition comprising vitamin B₆, pyridoxal 5 phosphate (PLP), and/or pyridoxine (PN) at an amount equal to or less than 100 mg. In some specific embodiments, the genetically engineered bacteria may be formulated into a pharmaceutical composition comprising vitamin B₆, pyridoxal 5 phosphate (PLP), and/or pyridoxine (PN) at about 25 mg.

In any of these embodiments, the pharmaceutical compositions into which the engineered bacteria are formulated, and which comprise vitamin B₆, PLP and/or PN, may further comprise sodium bicarbonate and a flavoring agent.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to adult subjects or pediatric subjects. As is well known in the art, children differ from adults in many aspects, including different rates of gastric emptying, pH, gastrointestinal permeability, etc. (Ivanovska et al., Pediatrics, 134 (2): 361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Methods of Treatment

Methionine is an essential amino acid that is primarily catabolized via the methionine cycle and the transsulfuration pathway and plays a central role in sulfur metabolism and redox regulation in cells. Methionine processing through these pathways results in the formation of critical intermediates like S-adenosylmethionine (SAM), homocysteine, and cysteine and involves the transfer of sulfur from homocysteine to cysteine via cystathionine. Like other amino acids, including phenylalanine and leucine, methionine is vital for normal growth and development, but its excess can lead to serious deleterious effects such as brain damage and death. In fact, methionine restriction has been shown to extend lifespan and metabolic health in rodents and nonhuman primates (NHP) and to inhibit cancer cell growth in vitro and in laboratory animals. Furthermore, inborn errors of transsulfuration resulting from severe loss of function in cystathionine β-synthase (CBS) cause classic homocystinuria (HCU), which must be managed by a methionine-restricted diet. A tightly controlled balance in the levels of methionine is required to maintain normal cellular functions and growth while avoiding the direct and indirect toxic effects associated with methionine metabolism.

Further disclosed herein are methods of treating diseases associated with methionine metabolism. In some embodiments, disclosed herein are methods for reducing, ameliorating, or eliminating one or more symptom(s) associated with these diseases or disorders.

As used herein the terms “disease associated with amino acid metabolism” or a “disorder associated with amino acid metabolism” is a disease or disorder involving the abnormal, e.g., increased, levels of one or more amino acids in a subject. In one embodiment, a disease or disorder associated with amino acid metabolism is homocystinuria, cancer, or a metabolic syndrome/disease. For example, for metabolic indications, a methionine-restricted diet has been shown to increase lifespan, reduce adiposity, decrease systemic inflammation, and improve insulin sensitivity in rodent and some large animal models (see, for example, Dong et al., E Clinical Medicine, 2019). For indications in immune-oncology and cancer, there is preclinical data supporting a link between tumoral methionine restriction and antitumor activity (see, for example, Gay et al., Cancer Medicine, 2017, 6 (6): 1437-1452).

In some embodiments, the disclosure provides methods for reducing, ameliorating, or eliminating one or more symptom(s) associated with these diseases.

The method may comprise preparing a pharmaceutical composition with at least one genetically engineered species, strain, or subtype of bacteria described herein, and administering the pharmaceutical composition to a subject in a therapeutically effective amount. In some embodiments, the genetically engineered bacteria disclosed herein are administered orally, e.g., in a liquid suspension. In some embodiments, the genetically engineered bacteria are lyophilized in a gel cap and administered orally. In some embodiments, the genetically engineered bacteria are administered via a feeding tube or gastric shunt. In some embodiments, the genetically engineered bacteria are administered rectally, e.g., by enema. In some embodiments, the genetically engineered bacteria are administered topically, intraintestinally, intrajejunally, intraduodenally, intraileally, and/or intracolically. In one embodiment, the genetically engineered bacteria are injected directly into a tumor.

In certain embodiments, administering the pharmaceutical composition to the subject reduces the level of an amino acid, e.g., methionine, in a subject. In some embodiments, the methods of the present disclosure may reduce the level of an amino acid, e.g., methionine in a subject by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing the amino acid concentration in a subject before and after administration of the pharmaceutical composition. In some embodiments, the method of treating or ameliorating a disease or disorder allows one or more symptoms of the condition or disorder to improve by at least about 10%, 20%, 30%, 40%, 50%, 60%, 70%, 80%, 90%, 95%, or more as compared to levels in an untreated or control subject, or as compared to levels in the subject prior to administration. Amino acid levels may be measured by methods known in the art (see methionine decarboxylase section, supra).

Before, during, and after the administration of the pharmaceutical composition, methionine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., methionine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

Before, during, and after the administration of the pharmaceutical composition, homocysteine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., homocysteine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

Before, during, and after the administration of the pharmaceutical composition, cysteine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., cysteine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

The methods disclosed herein may further comprise isolating a sample from the subject prior to administration of a composition and determining the level of the amino acid(s) in the sample. In some embodiments, the methods may further comprise isolating a sample from the subject after to administration of a composition and determining the level of amino acid(s) in the sample.

In certain embodiments, the genetically engineered bacteria comprising a methionine decarboxylase is E. coli Nissle. The genetically engineered bacteria may be destroyed, e.g., by defense factors in the gut or blood serum (Sonnenborn et al., 2009), or by activation of a kill switch, several hours or days after administration. Thus, the pharmaceutical composition comprising the methionine decarboxylase may be re-administered at a therapeutically effective dose and frequency. In alternate embodiments, the genetically engineered bacteria are not destroyed within hours or days after administration and may propagate and colonize the gut.

The methods disclosed herein may comprise administration of a composition alone or in combination with one or more additional therapies. The pharmaceutical composition may be administered alone or in combination with one or more additional therapeutic agents, including but not limited to, sodium phenylbutyrate, sodium benzoate, and glycerol phenylbutyrate. The methods may also comprise following an dietary restriction of methionine (supplemented with a methionine-free amino acid mixture), and/or administration of betaine, pyridoxine, folate, and vitamin B12, and/or other enzyme replacement-based therapies such as OT-58 or AGLE-177. OT-58 represents a therapeutic approach incorporating the use of a modified version of the native human CBS enzyme.

The goal of this treatment is to introduce the CBS enzyme into circulation, resulting in reduced Hcy levels, increased crystalthionine levels, and normalized cysteine levels. AGLE-177 is an engineered human enzyme designed to degrade both homocysteine and homocysteine (two homocysteine molecules bound together) to lower abnormally high levels of homocysteine in the blood. The methods may also include dietary management measures, including maintaining target homocysteine levels of <50 μM or <100 μM in pyridoxine-responsive or pyridoxine-unresponsive patients, respectively.

Alternatively, in some embodiments, the novel therapeutic treatment methods described herein further comprise the administration of vitamin B₆, pyridoxal 5 phosphate, and/or pyridoxine prior to, concurrently or directly after administration of the bacteria. For example, in one aspect a method for treating a disease associated with methionine metabolism in a subject, comprising administering orally a pharmaceutical composition disclosed herein comprising a recombinant bacterium described herein to the subject may further comprise the administration of vitamin B₆, pyridoxal 5 phosphate, and/or pyridoxine prior to, concurrently or directly after administration of the pharmaceutical composition. In another aspect, the present disclosure provides for a method for reducing a level of methionine, cysteine and/or homocysteine in a human subject, the method comprising orally administering to the subject a pharmaceutical composition comprising a recombinant bacterium described herein, further comprises the administration of vitamin B₆, pyridoxal 5 phosphate, and/or pyridoxine prior to, concurrently or directly after administration of the bacteria. For example, an amount of at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 35 mg, at least about 36 mg, at least about 37 mg, at least about 38 mg, at least about 39 mg, or at least about 40 mg vitamin B₆, pyridoxal 5 phosphate (PLP), and/or pyridoxine are administered, e.g., per day or at least once daily prior to, concurrently with, or after each bacterial dose.

In some embodiments, an amount of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 250 mg, or at least about 300 mg vitamin B₆, pyridoxal 5 phosphate (PLP), and/or pyridoxine are administered, e.g., per day or at least once daily prior to, concurrently with, or after each bacterial dose.

In some embodiments an amount of at about 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg vitamin B₆, pyridoxal 5 phosphate (PLP), and/or pyridoxine are administered, e.g., per day or at least once daily prior to, concurrently with, or after each bacterial dose.

In some embodiments, an amount of about 50 mg to about 75 mg, about 75 mg to about 100 mg, about 100 mg to about 125 mg, about 125 mg to about 150 mg, about 150 mg to about 175 mg, or about 175 mg to about 200 mg vitamin B₆, pyridoxal 5 phosphate (PLP), and/or pyridoxine are administered, e.g., per day or at least once daily prior to, concurrently with, or after each bacterial dose.

In some embodiments, an amount of about 1 mg to about 50 mg, about 50 mg to about 100 mg, about 100 mg to about 150 mg, about 150 mg to about 200 mg, about 200 mg to about 300 mg, about 300 mg to about 400 mg, or about 400 mg to about 500 mg vitamin B₆, pyridoxal 5 phosphate (PLP), and/or pyridoxine are administered, e.g., per day or at least once daily prior to, concurrently with, or after each bacterial dose.

In some specific embodiments, an amount of about 100 mg or less than 100 mg vitamin B₆, pyridoxal 5 phosphate (PLP), and/or pyridoxine is administered, e.g., per day or at least once daily or prior to, concurrently with, or after each bacterial dose. In some specific embodiments, about 25 mg vitamin B₆, pyridoxal 5 phosphate (PLP), and/or pyridoxine may be administered, e.g., per day or at least once daily prior to, concurrently with, or after each dose.

Methionine abundance in natural sources of protein ranges from 1-2% (or 1-2 g/100 g protein intake). Assuming the average human subject needs to degrade about 1.0 g methionine per day with meals, and assuming the recombinant bacteria provides 3 hours of activity per dose, that leaves 3× doses per day at 5×10¹¹ dose and 1.0 g methionine per day (0.33 g/dose). 0.33 g methionine/dose=2230 μmol methionine. 2230 μmol/3 hours/5×10¹¹ cells leads to 1.49 μmol/hr/1×10⁹ cells. The target dose is 5×10¹¹ live recombinant bacterial cells/mL.

For human subjects on a low protein diet eating 10 g protein/day, the subject needs to degrade about 0.1-1 g, e.g. 0.1 g, 0.2 g, 0.3 g, 0.4 g, 0.5 g, 0.6 g, 0.7 g, 0.8 g, 0.9 g or 1 g, methionine per day with meals. Assuming the recombinant bacteria provides 3 hours of activity per dose, that leaves 3× doses per day at 5×10¹¹ dose and 0.1 g per day (0.033 g/dose). 0.033 g methionine/dose=223 μmol methionine. 223 μmol/3 hours/5×10¹¹ cells leads to 0.15 μmol/hr/1×10⁹ cells. The target dose is 5×10¹¹ live recombinant bacterial cells/mL.

Accordingly, in one embodiment, the recombinant bacteria disclosed herein has a methionine degradation activity of about 0.1 μmol/hr/1×10⁹ cells. Accordingly, in one embodiment, the recombinant bacteria disclosed herein has a methionine degradation activity of about 0.15 μmol/hr/1×10⁹ cells. Accordingly, in one embodiment, the recombinant bacteria disclosed herein has a methionine degradation activity of about 0.2 μmol/hr/1×10⁹ cells. Accordingly, in one embodiment, the recombinant bacteria disclosed herein has a methionine degradation activity of about 0.25 μmol/hr/1×10⁹ cells. Accordingly, in one embodiment, the recombinant bacteria disclosed herein has a methionine degradation activity of about 0.3 μmol/hr/1×10⁹ cells. Accordingly, in one embodiment, the recombinant bacteria disclosed herein has a methionine degradation activity of about 0.4 μmol/hr/1×10⁹ cells. Accordingly, in one embodiment, the recombinant bacteria disclosed herein has a methionine degradation activity of about 0.5 μmol/hr/1×10⁹ cells. Accordingly, in one embodiment, the recombinant bacteria disclosed herein has a methionine degradation activity of about 0.6 μmol/hr/1×10⁹ cells. Accordingly, in one embodiment, the recombinant bacteria disclosed herein has a methionine degradation activity of about 0.7 μmol/hr/1×10⁹ cells. Accordingly, in one embodiment, the recombinant bacteria disclosed herein has a methionine degradation activity of about 0.8 μmol/hr/1×10⁹ cells. Accordingly, in one embodiment, the recombinant bacteria disclosed herein has a methionine degradation activity of about 0.9 μmol/hr/1×10⁹ cells. Accordingly, in one embodiment, the recombinant bacteria disclosed herein has a methionine degradation activity of about 1.0 μmol/hr/1×10⁹ cells. Accordingly, in one embodiment, the recombinant bacteria disclosed herein has a methionine degradation activity of about 1.10 μmol/hr/1×10⁹ cells. Accordingly, in one embodiment, the recombinant bacteria disclosed herein has a methionine degradation activity of about 1.30 μmol/hr/1×10⁹ cells. Accordingly, in one embodiment, the recombinant bacteria disclosed herein has a methionine degradation activity of about 1.30 μmol/hr/1×10⁹ cells. Accordingly, in one embodiment, the recombinant bacteria disclosed herein has a methionine degradation activity of about 1.40 μmol/hr/1×10⁹ cells. Accordingly, in one embodiment, the recombinant bacteria disclosed herein has a methionine degradation activity of about 1.45 μmol/hr/1×10⁹ cells. Accordingly, in one embodiment, the recombinant bacteria disclosed herein has a methionine degradation activity of about 1.50 μmol/hr/1×10⁹ cells. Accordingly, in one embodiment, the recombinant bacteria disclosed herein has a methionine degradation activity of about 1.55 μmol/hr/1×10⁹ cells. Accordingly, in one embodiment, the recombinant bacteria disclosed herein has a methionine degradation activity of about 1.60 μmol/hr/1×10⁹ cells. Accordingly, in one embodiment, the recombinant bacteria disclosed herein has a methionine degradation activity of about 1.65 μmol/hr/1×10⁹ cells. Accordingly, in one embodiment, the recombinant bacteria disclosed herein has a methionine degradation activity of about 1.70 μmol/hr/1×10⁹ cells. Accordingly, in one embodiment, the recombinant bacteria disclosed herein has a methionine degradation activity of about 1.75 μmol/hr/1×10⁹ cells. Accordingly, in one embodiment, the recombinant bacteria disclosed herein has a methionine degradation activity of about 1.80 μmol/hr/1×10⁹ cells. Accordingly, in one embodiment, the recombinant bacteria disclosed herein has a methionine degradation activity of about 1.85 μmol/hr/1×10⁹ cells. Accordingly, in one embodiment, the recombinant bacteria disclosed herein has a methionine degradation activity of about 1.90 μmol/hr/1×10⁹ cells. Accordingly, in one embodiment, the recombinant bacteria disclosed herein has a methionine degradation activity of about 1.95 μmol/hr/1×10⁹ cells. Accordingly, in one embodiment, the recombinant bacteria disclosed herein has a methionine degradation activity of about 2.0 μmol/hr/1×10⁹ cells.

Accordingly, in one embodiment, the recombinant bacteria disclosed herein has a methionine degradation activity of about 0.1 μmol/hr/1×10⁹ cells to about 2.0 μmol/hr/1×10⁹ cells. Accordingly, in one embodiment, the recombinant bacteria disclosed herein has a methionine degradation activity of about 0.2 μmol/hr/1×10° cells to about 2.0 μmol/hr/1×10⁹ cells. Accordingly, in one embodiment, the recombinant bacteria disclosed herein has a methionine degradation activity of about 0.3 μmol/hr/1×10⁹ cells to about 2.0 μmol/hr/1×10⁹ cells. Accordingly, in one embodiment, the recombinant bacteria disclosed herein has a methionine degradation activity of about 0.4 μmol/hr/1×10⁹ cells to about 2.0 μmol/hr/1×10⁹ cells. Accordingly, in one embodiment, the recombinant bacteria disclosed herein has a methionine degradation activity of about 0.5 μmol/hr/1×10⁹ cells to about 2.0 μmol/hr/1×10⁹ cells. Accordingly, in one embodiment, the recombinant bacteria disclosed herein has a methionine degradation activity of about 0.6 μmol/hr/1×10° cells to about 2.0 μmol/hr/1×10⁹ cells. Accordingly, in one embodiment, the recombinant bacteria disclosed herein has a methionine degradation activity of about 0.7 μmol/hr/1×10⁹ cells to about 2.0 μmol/hr/1×10⁹ cells. Accordingly, in one embodiment, the recombinant bacteria disclosed herein has a methionine degradation activity of about 0.8 μmol/hr/1×10⁹ cells to about 2.0 μmol/hr/1×10⁹ cells. Accordingly, in one embodiment, the recombinant bacteria disclosed herein has a methionine degradation activity of about 0.9 μmol/hr/1×10⁹ cells to about 2.0 μmol/hr/1×10⁹ cells. Accordingly, in one embodiment, the recombinant bacteria disclosed herein has a methionine degradation activity of about 1.0 μmol/hr/1×10⁹ cells to about 2.0 μmol/hr/1×10⁹ cells. Accordingly, in one embodiment, the recombinant bacteria disclosed herein has a methionine degradation activity of about 1.1 μmol/hr/1×10⁹ cells to about 2.0 μmol/hr/1×10⁹ cells. Accordingly, in one embodiment, the recombinant bacteria disclosed herein has a methionine degradation activity of about 1.2 μmol/hr/1×10⁹ cells to about 2.0 μmol/hr/1×10⁹ cells. Accordingly, in one embodiment, the recombinant bacteria disclosed herein has a methionine degradation activity of about 1.3 μmol/hr/1×10⁹ cells to about 2.0 μmol/hr/1×10⁹ cells. Accordingly, in one embodiment, the recombinant bacteria disclosed herein has a methionine degradation activity of about 1.4 μmol/hr/1×10⁹ cells to about 2.0 μmol/hr/1×10° cells. Accordingly, in one embodiment, the recombinant bacteria disclosed herein has a methionine degradation activity of about 1.5 μmol/hr/1×10⁹ cells to about 2.0 μmol/hr/1×10⁹ cells. Accordingly, in one embodiment, the recombinant bacteria disclosed herein has a methionine degradation activity of about 1.6 μmol/hr/1×10⁹ cells to about 2.0 μmol/hr/1×10⁹ cells. Accordingly, in one embodiment, the recombinant bacteria disclosed herein has a methionine degradation activity of about 1.7 μmol/hr/1×10⁹ cells to about 2.0 μmol/hr/1×10⁹ cells. Accordingly, in one embodiment, the recombinant bacteria disclosed herein has a methionine degradation activity of about 1.8 μmol/hr/1×10⁹ cells to about 2.0 μmol/hr/1×10⁹ cells. Accordingly, in one embodiment, the recombinant bacteria disclosed herein has a methionine degradation activity of about 1.9 μmol/hr/1×10⁹ cells to about 2.0 μmol/hr/1×10⁹ cells. Accordingly, in one embodiment, the recombinant bacteria disclosed herein has a methionine degradation activity of about 0.1 μmol/hr/1×10⁹ cells to about 1.9 μmol/hr/1×10⁹ cells. Accordingly, in one embodiment, the recombinant bacteria disclosed herein has a methionine degradation activity of about 0.2 μmol/hr/1×10⁹ cells to about 1.9 μmol/hr/1×10⁹ cells. Accordingly, in one embodiment, the recombinant bacteria disclosed herein has a methionine degradation activity of about 0.3 μmol/hr/1×10⁹ cells to about 1.9 μmol/hr/1×10⁹ cells. Accordingly, in one embodiment, the recombinant bacteria disclosed herein has a methionine degradation activity of about 0.4 μmol/hr/1×10⁹ cells to about 1.9 μmol/hr/1×10° cells. Accordingly, in one embodiment, the recombinant bacteria disclosed herein has a methionine degradation activity of about 0.5 μmol/hr/1×10⁹ cells to about 1.9 μmol/hr/1×10⁹ cells. Accordingly, in one embodiment, the recombinant bacteria disclosed herein has a methionine degradation activity of about 0.6 μmol/hr/1×10⁹ cells to about 1.9 μmol/hr/1×10⁹ cells. Accordingly, in one embodiment, the recombinant bacteria disclosed herein has a methionine degradation activity of about 0.7 μmol/hr/1×10⁹ cells to about 1.9 μmol/hr/1×10⁹ cells. Accordingly, in one embodiment, the recombinant bacteria disclosed herein has a methionine degradation activity of about 0.8 μmol/hr/1×10⁹ cells to about 1.9 μmol/hr/1×10⁹ cells. Accordingly, in one embodiment, the recombinant bacteria disclosed herein has a methionine degradation activity of about 0.9 μmol/hr/1×10⁹ cells to about 1.9 μmol/hr/1×10⁹ cells. Accordingly, in one embodiment, the recombinant bacteria disclosed herein has a methionine degradation activity of about 1.0 μmol/hr/1×10⁹ cells to about 1.9 μmol/hr/1×10⁹ cells. Accordingly, in one embodiment, the recombinant bacteria disclosed herein has a methionine degradation activity of about 1.1 μmol/hr/1×10⁹ cells to about 1.9 μmol/hr/1×10⁹ cells. Accordingly, in one embodiment, the recombinant bacteria disclosed herein has a methionine degradation activity of about 1.2 μmol/hr/1×10⁹ cells to about 1.9 μmol/hr/1×10⁹ cells. Accordingly, in one embodiment, the recombinant bacteria disclosed herein has a methionine degradation activity of about 1.3 μmol/hr/1×10⁹ cells to about 1.9 μmol/hr/1×10⁹ cells. Accordingly, in one embodiment, the recombinant bacteria disclosed herein has a methionine degradation activity of about 1.4 μmol/hr/1×10⁹ cells to about 1.9 μmol/hr/1×10⁹ cells. Accordingly, in one embodiment, the recombinant bacteria disclosed herein has a methionine degradation activity of about 1.5 μmol/hr/1×10⁹ cells to about 1.9 μmol/hr/1×10⁹ cells. Accordingly, in one embodiment, the recombinant bacteria disclosed herein has a methionine degradation activity of about 1.6 μmol/hr/1×10⁹ cells to about 1.9 μmol/hr/1×10⁹ cells. Accordingly, in one embodiment, the recombinant bacteria disclosed herein has a methionine degradation activity of about 1.7 μmol/hr/1×10⁹ cells to about 1.9 μmol/hr/1×10⁹ cells. Accordingly, in one embodiment, the recombinant bacteria disclosed herein has a methionine degradation activity of about 1.8 μmol/hr/1×10⁹ cells to about 1.9 μmol/hr/1×10⁹ cells. Accordingly, in one embodiment, the recombinant bacteria disclosed herein has a methionine degradation activity of about 0.1 μmol/hr/1×10⁹ cells to about 1.8 μmol/hr/1×10⁹ cells. Accordingly, in one embodiment, the recombinant bacteria disclosed herein has a methionine degradation activity of about 0.2 μmol/hr/1×10⁹ cells to about 1.8 μmol/hr/1×10⁹ cells. Accordingly, in one embodiment, the recombinant bacteria disclosed herein has a methionine degradation activity of about 0.3 μmol/hr/1×10⁹ cells to about 1.8 μmol/hr/1×10⁹ cells. Accordingly, in one embodiment, the recombinant bacteria disclosed herein has a methionine degradation activity of about 0.4 μmol/hr/1×10⁹ cells to about 1.8 μmol/hr/1×10⁹ cells. Accordingly, in one embodiment, the recombinant bacteria disclosed herein has a methionine degradation activity of about 0.5 μmol/hr/1×10⁹ cells to about 1.8 μmol/hr/1×10⁹ cells. Accordingly, in one embodiment, the recombinant bacteria disclosed herein has a methionine degradation activity of about 0.6 μmol/hr/1×10⁹ cells to about 1.8 μmol/hr/1×10⁹ cells. Accordingly, in one embodiment, the recombinant bacteria disclosed herein has a methionine degradation activity of about 0.7 μmol/hr/1×10⁹ cells to about 1.8 μmol/hr/1×10⁹ cells. Accordingly, in one embodiment, the recombinant bacteria disclosed herein has a methionine degradation activity of about 0.8 μmol/hr/1×10⁹ cells to about 1.8 μmol/hr/1×10⁹ cells. Accordingly, in one embodiment, the recombinant bacteria disclosed herein has a methionine degradation activity of about 0.9 μmol/hr/1×10⁹ cells to about 1.8 μmol/hr/1×10⁹ cells. Accordingly, in one embodiment, the recombinant bacteria disclosed herein has a methionine degradation activity of about 1.0 μmol/hr/1×10⁹ cells to about 1.8 μmol/hr/1×10⁹ cells. Accordingly, in one embodiment, the recombinant bacteria disclosed herein has a methionine degradation activity of about 1.1 μmol/hr/1×10⁹ cells to about 1.8 μmol/hr/1×10⁹ cells. Accordingly, in one embodiment, the recombinant bacteria disclosed herein has a methionine degradation activity of about 1.2 μmol/hr/1×10⁹ cells to about 1.8 μmol/hr/1×10⁹ cells. Accordingly, in one embodiment, the recombinant bacteria disclosed herein has a methionine degradation activity of about 1.3 μmol/hr/1×10⁹ cells to about 1.8 μmol/hr/1×10⁹ cells. Accordingly, in one embodiment, the recombinant bacteria disclosed herein has a methionine degradation activity of about 1.4 μmol/hr/1×10⁹ cells to about 1.8 μmol/hr/1×10⁹ cells. Accordingly, in one embodiment, the recombinant bacteria disclosed herein has a methionine degradation activity of about 1.5 μmol/hr/1×10⁹ cells to about 1.8 μmol/hr/1×10⁹ cells. Accordingly, in one embodiment, the recombinant bacteria disclosed herein has a methionine degradation activity of about 1.6 μmol/hr/1×10⁹ cells to about 1.8 μmol/hr/1×10⁹ cells. Accordingly, in one embodiment, the recombinant bacteria disclosed herein has a methionine degradation activity of about 1.7 μmol/hr/1×10⁹ cells to about 1.8 μmol/hr/1×10⁹ cells. Accordingly, in one embodiment, the recombinant bacteria disclosed herein has a methionine degradation activity of about 0.1 μmol/hr/1×10⁹ cells to about 1.7 μmol/hr/1×10⁹ cells. Accordingly, in one embodiment, the recombinant bacteria disclosed herein has a methionine degradation activity of about 0.2 μmol/hr/1×10⁹ cells to about 1.7 μmol/hr/1×10⁹ cells. Accordingly, in one embodiment, the recombinant bacteria disclosed herein has a methionine degradation activity of about 0.3 μmol/hr/1×10⁹ cells to about 1.7 μmol/hr/1×10⁹ cells. Accordingly, in one embodiment, the recombinant bacteria disclosed herein has a methionine degradation activity of about 0.4 μmol/hr/1×10⁹ cells to about 1.7 μmol/hr/1×10⁹ cells. Accordingly, in one embodiment, the recombinant bacteria disclosed herein has a methionine degradation activity of about 0.5 μmol/hr/1×10⁹ cells to about 1.7 μmol/hr/1×10⁹ cells. Accordingly, in one embodiment, the recombinant bacteria disclosed herein has a methionine degradation activity of about 0.6 μmol/hr/1×10⁹ cells to about 1.7 μmol/hr/1×10⁹ cells. Accordingly, in one embodiment, the recombinant bacteria disclosed herein has a methionine degradation activity of about 0.7 μmol/hr/1×10⁹ cells to about 1.7 μmol/hr/1×10⁹ cells. Accordingly, in one embodiment, the recombinant bacteria disclosed herein has a methionine degradation activity of about 0.8 μmol/hr/1×10⁹ cells to about 1.7 μmol/hr/1×10⁹ cells. Accordingly, in one embodiment, the recombinant bacteria disclosed herein has a methionine degradation activity of about 0.9 μmol/hr/1×10⁹ cells to about 1.7 μmol/hr/1×10⁹ cells. Accordingly, in one embodiment, the recombinant bacteria disclosed herein has a methionine degradation activity of about 1.0 μmol/hr/1×10⁹ cells to about 1.7 μmol/hr/1×10⁹ cells. Accordingly, in one embodiment, the recombinant bacteria disclosed herein has a methionine degradation activity of about 1.1 μmol/hr/1×10⁹ cells to about 1.7 μmol/hr/1×10⁹ cells. Accordingly, in one embodiment, the recombinant bacteria disclosed herein has a methionine degradation activity of about 1.2 μmol/hr/1×10⁹ cells to about 1.7 μmol/hr/1×10⁹ cells. Accordingly, in one embodiment, the recombinant bacteria disclosed herein has a methionine degradation activity of about 1.3 μmol/hr/1×10⁹ cells to about 1.7 μmol/hr/1×10⁹ cells. Accordingly, in one embodiment, the recombinant bacteria disclosed herein has a methionine degradation activity of about 1.4 μmol/hr/1×10⁹ cells to about 1.7 μmol/hr/1×10⁹ cells. Accordingly, in one embodiment, the recombinant bacteria disclosed herein has a methionine degradation activity of about 1.5 μmol/hr/1×10⁹ cells to about 1.7 μmol/hr/1×10⁹ cells. Accordingly, in one embodiment, the recombinant bacteria disclosed herein has a methionine degradation activity of about 1.6 μmol/hr/1×10⁹ cells to about 1.7 μmol/hr/1×10⁹ cells. Accordingly, in one embodiment, the recombinant bacteria disclosed herein has a methionine degradation activity of about 0.1 μmol/hr/1×10⁹ cells to about 1.6 μmol/hr/1×10⁹ cells. Accordingly, in one embodiment, the recombinant bacteria disclosed herein has a methionine degradation activity of about 0.2 μmol/hr/1×10⁹ cells to about 1.6 μmol/hr/1×10⁹ cells. Accordingly, in one embodiment, the recombinant bacteria disclosed herein has a methionine degradation activity of about 0.3 μmol/hr/1×10⁹ cells to about 1.6 μmol/hr/1×10⁹ cells. Accordingly, in one embodiment, the recombinant bacteria disclosed herein has a methionine degradation activity of about 0.4 μmol/hr/1×10⁹ cells to about 1.6 μmol/hr/1×10⁹ cells. Accordingly, in one embodiment, the recombinant bacteria disclosed herein has a methionine degradation activity of about 0.5 μmol/hr/1×10⁹ cells to about 1.6 μmol/hr/1×10⁹ cells. Accordingly, in one embodiment, the recombinant bacteria disclosed herein has a methionine degradation activity of about 0.6 μmol/hr/1×10⁹ cells to about 1.6 μmol/hr/1×10⁹ cells. Accordingly, in one embodiment, the recombinant bacteria disclosed herein has a methionine degradation activity of about 0.7 μmol/hr/1×10⁹ cells to about 1.6 μmol/hr/1×10⁹ cells. Accordingly, in one embodiment, the recombinant bacteria disclosed herein has a methionine degradation activity of about 0.8 μmol/hr/1×10⁹ cells to about 1.6 μmol/hr/1×10⁹ cells. Accordingly, in one embodiment, the recombinant bacteria disclosed herein has a methionine degradation activity of about 0.9 μmol/hr/1×10⁹ cells to about 1.6 μmol/hr/1×10⁹ cells. Accordingly, in one embodiment, the recombinant bacteria disclosed herein has a methionine degradation activity of about 1.0 μmol/hr/1×10⁹ cells to about 1.6 μmol/hr/1×10⁹ cells. Accordingly, in one embodiment, the recombinant bacteria disclosed herein has a methionine degradation activity of about 1.1 μmol/hr/1×10⁹ cells to about 1.6 μmol/hr/1×10⁹ cells. Accordingly, in one embodiment, the recombinant bacteria disclosed herein has a methionine degradation activity of about 1.2 μmol/hr/1×10⁹ cells to about 1.6 μmol/hr/1×10⁹ cells. Accordingly, in one embodiment, the recombinant bacteria disclosed herein has a methionine degradation activity of about 1.3 μmol/hr/1×10⁹ cells to about 1.6 μmol/hr/1×10⁹ cells. Accordingly, in one embodiment, the recombinant bacteria disclosed herein has a methionine degradation activity of about 1.4 μmol/hr/1×10⁹ cells to about 1.6 μmol/hr/1×10⁹ cells. Accordingly, in one embodiment, the recombinant bacteria disclosed herein has a methionine degradation activity of about 1.5 μmol/hr/1×10⁹ cells to about 1.6 μmol/hr/1×10⁹ cells. Accordingly, in one embodiment, the recombinant bacteria disclosed herein has a methionine degradation activity of about 0.1 μmol/hr/1×10⁹ cells to about 1.5 μmol/hr/1×10⁹ cells. Accordingly, in one embodiment, the recombinant bacteria disclosed herein has a methionine degradation activity of about 0.2 μmol/hr/1×10⁹ cells to about 1.5 μmol/hr/1×10⁹ cells. Accordingly, in one embodiment, the recombinant bacteria disclosed herein has a methionine degradation activity of about 0.3 μmol/hr/1×10⁹ cells to about 1.5 μmol/hr/1×10⁹ cells. Accordingly, in one embodiment, the recombinant bacteria disclosed herein has a methionine degradation activity of about 0.4 μmol/hr/1×10⁹ cells to about 1.5 μmol/hr/1×10⁹ cells. Accordingly, in one embodiment, the recombinant bacteria disclosed herein has a methionine degradation activity of about 0.5 μmol/hr/1×10⁹ cells to about 1.5 μmol/hr/1×10⁹ cells. Accordingly, in one embodiment, the recombinant bacteria disclosed herein has a methionine degradation activity of about 0.6 μmol/hr/1×10⁹ cells to about 1.5 μmol/hr/1×10⁹ cells. Accordingly, in one embodiment, the recombinant bacteria disclosed herein has a methionine degradation activity of about 0.7 μmol/hr/1×10⁹ cells to about 1.5 μmol/hr/1×10⁹ cells. Accordingly, in one embodiment, the recombinant bacteria disclosed herein has a methionine degradation activity of about 0.8 μmol/hr/1×10⁹ cells to about 1.5 μmol/hr/1×10⁹ cells. Accordingly, in one embodiment, the recombinant bacteria disclosed herein has a methionine degradation activity of about 0.9 μmol/hr/1×10⁹ cells to about 1.5 μmol/hr/1×10⁹ cells. Accordingly, in one embodiment, the recombinant bacteria disclosed herein has a methionine degradation activity of about 1.0 μmol/hr/1×10⁹ cells to about 1.5 μmol/hr/1×10⁹ cells. Accordingly, in one embodiment, the recombinant bacteria disclosed herein has a methionine degradation activity of about 1.1 μmol/hr/1×10⁹ cells to about 1.5 μmol/hr/1×10⁹ cells. Accordingly, in one embodiment, the recombinant bacteria disclosed herein has a methionine degradation activity of about 1.2 μmol/hr/1×10⁹ cells to about 1.5 μmol/hr/1×10⁹ cells. Accordingly, in one embodiment, the recombinant bacteria disclosed herein has a methionine degradation activity of about 1.3 μmol/hr/1×10⁹ cells to about 1.5 μmol/hr/1×10⁹ cells. Accordingly, in one embodiment, the recombinant bacteria disclosed herein has a methionine degradation activity of about 1.4 μmol/hr/1×10⁹ cells to about 1.5 μmol/hr/1×10⁹ cells. Accordingly, in one embodiment, the recombinant bacteria disclosed herein has a methionine degradation activity of about 0.1 μmol/hr/1×10⁹ cells to about 1.4 μmol/hr/1×10⁹ cells. Accordingly, in one embodiment, the recombinant bacteria disclosed herein has a methionine degradation activity of about 0.2 μmol/hr/1×10⁹ cells to about 1.4 μmol/hr/1×10⁹ cells. Accordingly, in one embodiment, the recombinant bacteria disclosed herein has a methionine degradation activity of about 0.3 μmol/hr/1×10⁹ cells to about 1.4 μmol/hr/1×10⁹ cells. Accordingly, in one embodiment, the recombinant bacteria disclosed herein has a methionine degradation activity of about 0.4 μmol/hr/1×10⁹ cells to about 1.4 μmol/hr/1×10⁹ cells. Accordingly, in one embodiment, the recombinant bacteria disclosed herein has a methionine degradation activity of about 0.5 μmol/hr/1×10⁹ cells to about 1.4 μmol/hr/1×10⁹ cells. Accordingly, in one embodiment, the recombinant bacteria disclosed herein has a methionine degradation activity of about 0.6 μmol/hr/1×10⁹ cells to about 1.4 μmol/hr/1×10⁹ cells. Accordingly, in one embodiment, the recombinant bacteria disclosed herein has a methionine degradation activity of about 0.7 μmol/hr/1×10⁹ cells to about 1.4 μmol/hr/1×10⁹ cells. Accordingly, in one embodiment, the recombinant bacteria disclosed herein has a methionine degradation activity of about 0.8 μmol/hr/1×10⁹ cells to about 1.4 μmol/hr/1×10⁹ cells. Accordingly, in one embodiment, the recombinant bacteria disclosed herein has a methionine degradation activity of about 0.9 μmol/hr/1×10⁹ cells to about 1.4 μmol/hr/1×10⁹ cells. Accordingly, in one embodiment, the recombinant bacteria disclosed herein has a methionine degradation activity of about 1.0 μmol/hr/1×10⁹ cells to about 1.4 μmol/hr/1×10⁹ cells. Accordingly, in one embodiment, the recombinant bacteria disclosed herein has a methionine degradation activity of about 1.1 μmol/hr/1×10⁹ cells to about 1.4 μmol/hr/1×10⁹ cells. Accordingly, in one embodiment, the recombinant bacteria disclosed herein has a methionine degradation activity of about 1.2 μmol/hr/1×10⁹ cells to about 1.4 μmol/hr/1×10⁹ cells. Accordingly, in one embodiment, the recombinant bacteria disclosed herein has a methionine degradation activity of about 1.3 μmol/hr/1×10⁹ cells to about 1.4 μmol/hr/1×10⁹ cells. Accordingly, in one embodiment, the recombinant bacteria disclosed herein has a methionine degradation activity of about 0.1 μmol/hr/1×10⁹ cells to about 1.3 μmol/hr/1×10⁹ cells. Accordingly, in one embodiment, the recombinant bacteria disclosed herein has a methionine degradation activity of about 0.2 μmol/hr/1×10⁹ cells to about 1.3 μmol/hr/1×10⁹ cells. Accordingly, in one embodiment, the recombinant bacteria disclosed herein has a methionine degradation activity of about 0.3 μmol/hr/1×10⁹ cells to about 1.3 μmol/hr/1×10⁹ cells. Accordingly, in one embodiment, the recombinant bacteria disclosed herein has a methionine degradation activity of about 0.4 μmol/hr/1×10⁹ cells to about 1.3 μmol/hr/1×10⁹ cells. Accordingly, in one embodiment, the recombinant bacteria disclosed herein has a methionine degradation activity of about 0.5 μmol/hr/1×10⁹ cells to about 1.3 μmol/hr/1×10⁹ cells. Accordingly, in one embodiment, the recombinant bacteria disclosed herein has a methionine degradation activity of about 0.6 μmol/hr/1×10⁹ cells to about 1.3 μmol/hr/1×10⁹ cells. Accordingly, in one embodiment, the recombinant bacteria disclosed herein has a methionine degradation activity of about 0.7 μmol/hr/1×10⁹ cells to about 1.3 μmol/hr/1×10⁹ cells. Accordingly, in one embodiment, the recombinant bacteria disclosed herein has a methionine degradation activity of about 0.8 μmol/hr/1×10⁹ cells to about 1.3 μmol/hr/1×10⁹ cells. Accordingly, in one embodiment, the recombinant bacteria disclosed herein has a methionine degradation activity of about 0.9 μmol/hr/1×10⁹ cells to about 1.3 μmol/hr/1×10⁹ cells. Accordingly, in one embodiment, the recombinant bacteria disclosed herein has a methionine degradation activity of about 1.0 μmol/hr/1×10⁹ cells to about 1.3 μmol/hr/1×10⁹ cells. Accordingly, in one embodiment, the recombinant bacteria disclosed herein has a methionine degradation activity of about 1.1 μmol/hr/1×10⁹ cells to about 1.3 μmol/hr/1×10⁹ cells. Accordingly, in one embodiment, the recombinant bacteria disclosed herein has a methionine degradation activity of about 1.2 μmol/hr/1×10⁹ cells to about 1.3 μmol/hr/1×10⁹ cells. Accordingly, in one embodiment, the recombinant bacteria disclosed herein has a methionine degradation activity of about 0.1 μmol/hr/1×10⁹ cells to about 1.2 μmol/hr/1×10° cells. Accordingly, in one embodiment, the recombinant bacteria disclosed herein has a methionine degradation activity of about 0.2 μmol/hr/1×10⁹ cells to about 1.2 μmol/hr/1×10⁹ cells. Accordingly, in one embodiment, the recombinant bacteria disclosed herein has a methionine degradation activity of about 0.3 μmol/hr/1×10⁹ cells to about 1.2 μmol/hr/1×10⁹ cells. Accordingly, in one embodiment, the recombinant bacteria disclosed herein has a methionine degradation activity of about 0.4 μmol/hr/1×10⁹ cells to about 1.2 μmol/hr/1×10⁹ cells. Accordingly, in one embodiment, the recombinant bacteria disclosed herein has a methionine degradation activity of about 0.5 μmol/hr/1×10⁹ cells to about 1.2 μmol/hr/1×10⁹ cells. Accordingly, in one embodiment, the recombinant bacteria disclosed herein has a methionine degradation activity of about 0.6 μmol/hr/1×10⁹ cells to about 1.2 μmol/hr/1×10⁹ cells. Accordingly, in one embodiment, the recombinant bacteria disclosed herein has a methionine degradation activity of about 0.7 μmol/hr/1×10⁹ cells to about 1.2 μmol/hr/1×10⁹ cells. Accordingly, in one embodiment, the recombinant bacteria disclosed herein has a methionine degradation activity of about 0.8 μmol/hr/1×10⁹ cells to about 1.2 μmol/hr/1×10⁹ cells. Accordingly, in one embodiment, the recombinant bacteria disclosed herein has a methionine degradation activity of about 0.9 μmol/hr/1×10⁹ cells to about 1.2 μmol/hr/1×10⁹ cells. Accordingly, in one embodiment, the recombinant bacteria disclosed herein has a methionine degradation activity of about 1.0 μmol/hr/1×10⁹ cells to about 1.2 μmol/hr/1×10⁹ cells. Accordingly, in one embodiment, the recombinant bacteria disclosed herein has a methionine degradation activity of about 1.1 μmol/hr/1×10⁹ cells to about 1.2 μmol/hr/1×10⁹ cells. Accordingly, in one embodiment, the recombinant bacteria disclosed herein has a methionine degradation activity of about 0.1 μmol/hr/1×10⁹ cells to about 1.1 μmol/hr/1×10⁹ cells. Accordingly, in one embodiment, the recombinant bacteria disclosed herein has a methionine degradation activity of about 0.2 μmol/hr/1×10⁹ cells to about 1.1 μmol/hr/1×10⁹ cells. Accordingly, in one embodiment, the recombinant bacteria disclosed herein has a methionine degradation activity of about 0.3 μmol/hr/1×10⁹ cells to about 1.1 μmol/hr/1×10⁹ cells. Accordingly, in one embodiment, the recombinant bacteria disclosed herein has a methionine degradation activity of about 0.4 μmol/hr/1×10⁹ cells to about 1.1 μmol/hr/1×10⁹ cells. Accordingly, in one embodiment, the recombinant bacteria disclosed herein has a methionine degradation activity of about 0.5 μmol/hr/1×10⁹ cells to about 1.1 μmol/hr/1×10⁹ cells. Accordingly, in one embodiment, the recombinant bacteria disclosed herein has a methionine degradation activity of about 0.6 μmol/hr/1×10⁹ cells to about 1.1 μmol/hr/1×10⁹ cells. Accordingly, in one embodiment, the recombinant bacteria disclosed herein has a methionine degradation activity of about 0.7 μmol/hr/1×10⁹ cells to about 1.1 μmol/hr/1×10⁹ cells. Accordingly, in one embodiment, the recombinant bacteria disclosed herein has a methionine degradation activity of about 0.8 μmol/hr/1×10⁹ cells to about 1.1 μmol/hr/1×10⁹ cells. Accordingly, in one embodiment, the recombinant bacteria disclosed herein has a methionine degradation activity of about 0.9 μmol/hr/1×10⁹ cells to about 1.1 μmol/hr/1×10⁹ cells. Accordingly, in one embodiment, the recombinant bacteria disclosed herein has a methionine degradation activity of about 1.0 μmol/hr/1×10⁹ cells to about 1.1 μmol/hr/1×10⁹ cells. Accordingly, in one embodiment, the recombinant bacteria disclosed herein has a methionine degradation activity of about 0.1 μmol/hr/1×10⁹ cells to about 1.0 μmol/hr/1×10⁹ cells. Accordingly, in one embodiment, the recombinant bacteria disclosed herein has a methionine degradation activity of about 0.2 μmol/hr/1×10⁹ cells to about 1.0 μmol/hr/1×10⁹ cells. Accordingly, in one embodiment, the recombinant bacteria disclosed herein has a methionine degradation activity of about 0.3 μmol/hr/1×10⁹ cells to about 1.0 μmol/hr/1×10⁹ cells. Accordingly, in one embodiment, the recombinant bacteria disclosed herein has a methionine degradation activity of about 0.4 μmol/hr/1×10⁹ cells to about 1.0 μmol/hr/1×10⁹ cells. Accordingly, in one embodiment, the recombinant bacteria disclosed herein has a methionine degradation activity of about 0.5 μmol/hr/1×10⁹ cells to about 1.0 μmol/hr/1×10⁹ cells. Accordingly, in one embodiment, the recombinant bacteria disclosed herein has a methionine degradation activity of about 0.6 μmol/hr/1×10⁹ cells to about 1.0 μmol/hr/1×10⁹ cells. Accordingly, in one embodiment, the recombinant bacteria disclosed herein has a methionine degradation activity of about 0.7 μmol/hr/1×10⁹ cells to about 1.0 μmol/hr/1×10⁹ cells. Accordingly, in one embodiment, the recombinant bacteria disclosed herein has a methionine degradation activity of about 0.8 μmol/hr/1×10⁹ cells to about 1.0 μmol/hr/1×10⁹ cells. Accordingly, in one embodiment, the recombinant bacteria disclosed herein has a methionine degradation activity of about 0.9 μmol/hr/1×10⁹ cells to about 1.0 μmol/hr/1×10⁹ cells. Accordingly, in one embodiment, the recombinant bacteria disclosed herein has a methionine degradation activity of about 0.1 μmol/hr/1×10⁹ cells to about 0.9 μmol/hr/1×10⁹ cells. Accordingly, in one embodiment, the recombinant bacteria disclosed herein has a methionine degradation activity of about 0.2 μmol/hr/1×10⁹ cells to about 0.9 μmol/hr/1×10⁹ cells. Accordingly, in one embodiment, the recombinant bacteria disclosed herein has a methionine degradation activity of about 0.3 μmol/hr/1×10⁹ cells to about 0.9 μmol/hr/1×10⁹ cells. Accordingly, in one embodiment, the recombinant bacteria disclosed herein has a methionine degradation activity of about 0.4 μmol/hr/1×10⁹ cells to about 0.9 μmol/hr/1×10⁹ cells. Accordingly, in one embodiment, the recombinant bacteria disclosed herein has a methionine degradation activity of about 0.5 μmol/hr/1×10⁹ cells to about 0.9 μmol/hr/1×10⁹ cells. Accordingly, in one embodiment, the recombinant bacteria disclosed herein has a methionine degradation activity of about 0.6 μmol/hr/1×10⁹ cells to about 0.9 μmol/hr/1×10⁹ cells. Accordingly, in one embodiment, the recombinant bacteria disclosed herein has a methionine degradation activity of about 0.7 μmol/hr/1×10⁹ cells to about 0.9 μmol/hr/1×10⁹ cells. Accordingly, in one embodiment, the recombinant bacteria disclosed herein has a methionine degradation activity of about 0.8 μmol/hr/1×10⁹ cells to about 0.9 μmol/hr/1×10⁹ cells. Accordingly, in one embodiment, the recombinant bacteria disclosed herein has a methionine degradation activity of about 0.1 μmol/hr/1×10⁹ cells to about 0.8 μmol/hr/1×10⁹ cells. Accordingly, in one embodiment, the recombinant bacteria disclosed herein has a methionine degradation activity of about 0.2 μmol/hr/1×10⁹ cells to about 0.8 μmol/hr/1×10⁹ cells. Accordingly, in one embodiment, the recombinant bacteria disclosed herein has a methionine degradation activity of about 0.3 μmol/hr/1×10⁹ cells to about 0.8 μmol/hr/1×10⁹ cells. Accordingly, in one embodiment, the recombinant bacteria disclosed herein has a methionine degradation activity of about 0.4 μmol/hr/1×10⁹ cells to about 0.8 μmol/hr/1×10⁹ cells. Accordingly, in one embodiment, the recombinant bacteria disclosed herein has a methionine degradation activity of about 0.5 μmol/hr/1×10⁹ cells to about 0.8 μmol/hr/1×10⁹ cells. Accordingly, in one embodiment, the recombinant bacteria disclosed herein has a methionine degradation activity of about 0.6 μmol/hr/1×10⁹ cells to about 0.8 μmol/hr/1×10⁹ cells. Accordingly, in one embodiment, the recombinant bacteria disclosed herein has a methionine degradation activity of about 0.7 μmol/hr/1×10⁹ cells to about 0.8 μmol/hr/1×10⁹ cells. Accordingly, in one embodiment, the recombinant bacteria disclosed herein has a methionine degradation activity of about 0.1 μmol/hr/1×10⁹ cells to about 0.7 μmol/hr/1×10⁹ cells. Accordingly, in one embodiment, the recombinant bacteria disclosed herein has a methionine degradation activity of about 0.2 μmol/hr/1×10⁹ cells to about 0.7 μmol/hr/1×10⁹ cells. Accordingly, in one embodiment, the recombinant bacteria disclosed herein has a methionine degradation activity of about 0.3 μmol/hr/1×10⁹ cells to about 0.7 μmol/hr/1×10⁹ cells. Accordingly, in one embodiment, the recombinant bacteria disclosed herein has a methionine degradation activity of about 0.4 μmol/hr/1×10⁹ cells to about 0.7 μmol/hr/1×10⁹ cells. Accordingly, in one embodiment, the recombinant bacteria disclosed herein has a methionine degradation activity of about 0.5 μmol/hr/1×10⁹ cells to about 0.7 μmol/hr/1×10⁹ cells. Accordingly, in one embodiment, the recombinant bacteria disclosed herein has a methionine degradation activity of about 0.6 μmol/hr/1×10⁹ cells to about 0.7 μmol/hr/1×10⁹ cells. Accordingly, in one embodiment, the recombinant bacteria disclosed herein has a methionine degradation activity of about 0.1 μmol/hr/1×10⁹ cells to about 0.6 μmol/hr/1×10⁹ cells. Accordingly, in one embodiment, the recombinant bacteria disclosed herein has a methionine degradation activity of about 0.2 μmol/hr/1×10⁹ cells to about 0.6 μmol/hr/1×10⁹ cells. Accordingly, in one embodiment, the recombinant bacteria disclosed herein has a methionine degradation activity of about 0.3 μmol/hr/1×10⁹ cells to about 0.6 μmol/hr/1×10⁹ cells. Accordingly, in one embodiment, the recombinant bacteria disclosed herein has a methionine degradation activity of about 0.4 μmol/hr/1×10⁹ cells to about 0.6 μmol/hr/1×10⁹ cells. Accordingly, in one embodiment, the recombinant bacteria disclosed herein has a methionine degradation activity of about 0.5 μmol/hr/1×10⁹ cells to about 0.6 μmol/hr/1×10⁹ cells. Accordingly, in one embodiment, the recombinant bacteria disclosed herein has a methionine degradation activity of about 0.1 μmol/hr/1×10⁹ cells to about 0.5 μmol/hr/1×10⁹ cells. Accordingly, in one embodiment, the recombinant bacteria disclosed herein has a methionine degradation activity of about 0.2 μmol/hr/1×10⁹ cells to about 0.5 μmol/hr/1×10⁹ cells. Accordingly, in one embodiment, the recombinant bacteria disclosed herein has a methionine degradation activity of about 0.3 μmol/hr/1×10⁹ cells to about 0.5 μmol/hr/1×10⁹ cells. Accordingly, in one embodiment, the recombinant bacteria disclosed herein has a methionine degradation activity of about 0.4 μmol/hr/1×10⁹ cells to about 0.5 μmol/hr/1×10⁹ cells. Accordingly, in one embodiment, the recombinant bacteria disclosed herein has a methionine degradation activity of about 0.1 μmol/hr/1×10⁹ cells to about 0.4 μmol/hr/1×10⁹ cells. Accordingly, in one embodiment, the recombinant bacteria disclosed herein has a methionine degradation activity of about 0.2 μmol/hr/1×10⁹ cells to about 0.4 μmol/hr/1×10⁹ cells. Accordingly, in one embodiment, the recombinant bacteria disclosed herein has a methionine degradation activity of about 0.3 μmol/hr/1×10⁹ cells to about 0.4 μmol/hr/1×10⁹ cells. Accordingly, in one embodiment, the recombinant bacteria disclosed herein has a methionine degradation activity of about 0.1 μmol/hr/1×10⁹ cells to about 0.3 μmol/hr/1×10⁹ cells. Accordingly, in one embodiment, the recombinant bacteria disclosed herein has a methionine degradation activity of about 0.2 μmol/hr/1×10⁹ cells to about 0.3 μmol/hr/1×10⁹ cells. Accordingly, in one embodiment, the recombinant bacteria disclosed herein has a methionine degradation activity of about 0.1 μmol/hr/1×10⁹ cells to about 0.2 μmol/hr/1×10⁹ cells. Accordingly, in one embodiment, the recombinant bacteria disclosed herein has a methionine degradation activity of about 0.1 μmol/hr/1×10⁹ cells to about 1.5 μmol/hr/1×10⁹ cells. Accordingly, in one embodiment, the recombinant bacteria disclosed herein has a methionine degradation activity of about 0.2 μmol/hr/1×10⁹ cells to about 1.5 μmol/hr/1×10⁹ cells. Accordingly, in one embodiment, the recombinant bacteria disclosed herein has a methionine degradation activity of about 0.1 μmol/hr/1×10⁹ cells to about 1.4 μmol/hr/1×10⁹ cells. Accordingly, in one embodiment, the recombinant bacteria disclosed herein has a methionine degradation activity of about 0.4 μmol/hr/1×10⁹ cells to about 1.1 μmol/hr/1×10⁹ cells. Accordingly, in one embodiment, the recombinant bacteria disclosed herein has a methionine degradation activity of about 0.1 μmol/hr/1×10⁹ cells to about 1.0 μmol/hr/1×10⁹ cells. Accordingly, in one embodiment, the recombinant bacteria disclosed herein has a methionine degradation activity of about 0.5 μmol/hr/1×10⁹ cells to about 1.5 μmol/hr/1×10⁹ cells. Accordingly, in one embodiment, the recombinant bacteria disclosed herein has a methionine degradation activity of about 0.75 μmol/hr/1×10⁹ cells to about 1.25 μmol/hr/1×10⁹ cells.

In one embodiment, about 0.1 g to about 1.0 g of methionine is degraded per day. In one embodiment, about 0.01 to about 1.5 g of methionine is degraded per day. In one embodiment, about 0.1 g of methionine is degraded per day. In one embodiment, about 0.2 g of methionine is degraded per day. In one embodiment, about 0.3 g of methionine is degraded per day. In one embodiment, about 0.4 g of methionine is degraded per day. In one embodiment, about 0.5 g of methionine is degraded per day. In one embodiment, about 0.6 g of methionine is degraded per day. In one embodiment, about 0.7 g of methionine is degraded per day. In one embodiment, about 0.8 g of methionine is degraded per day. In one embodiment, about 0.9 g of methionine is degraded per day. In one embodiment, about 1.0 g of methionine is degraded per day. In one embodiment, about 1.1 g of methionine is degraded per day. In one embodiment, about 1.2 g of methionine is degraded per day. In one embodiment, about 1.3 g of methionine is degraded per day. In one embodiment, about 1.4 g of methionine is degraded per day. In one embodiment, about 1.5 g of methionine is degraded per day.

An important consideration in the selection of the one or more additional therapeutic agents is that the agent(s) should be compatible with the genetically engineered bacteria disclosed herein, e.g., the agent(s) must not kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-protein diet or amino acid supplementation. The dosage of the pharmaceutical composition and the frequency of administration may be selected based on the severity of the symptoms and the progression of the disorder. The appropriate therapeutically effective dose and/or frequency of administration can be selected by a treating clinician.

Administering the bacteria to the subject may result in gastrointestinal-related adverse events, e.g., bloating, nausea, and/or change in bowel habits. In some embodiments, the adverse effects can be reduced by using a dose ramp. Thus, in some embodiments, the method of treatment, e.g., for HCU, and/or method of reducing levels of methionine, cysteine and/or homocysteine in a subject comprises administering to a subject escalating dose levels (dose ramp) of the bacteria described herein to obtain an individually titrated dose (iTD). In some embodiments, dose is escalated based on tolerability. In some embodiments, the dose ramp (e.g., a gradual and extended dose ramp) improves the dosing experience and helps more patients benefit from the treatment.

In some embodiments, a dose is considered intolerable is a subject experiences Grade 3 or more severe adverse events or Grade 2 gastrointestinal adverse events that do not improve with continued dosing at the same dose over time. In some embodiments, the method comprises administering to a subject a first dose of the bacteria for a first dosing interval (e.g., 2, 3, or 4 weeks), followed by a second dose of the bacteria that is higher than the first dose for a second dosing interval (e.g., 2, 3, or 4 weeks). In some embodiments, this is followed by a third dose of the bacteria that is higher than the second dose for a third dosing interval (e.g., 2, 3, or 4 weeks). In some embodiments, where the subject tolerates the first dose during the first dosing interval, e.g., after 2, 3, or 4 weeks according to the metrics described herein, the subject escalates to the second dose for a second dosing interval. In some embodiments, where the subject does not tolerate the second dose during the second dosing interval, e.g., after 2, 3, or 4 weeks according to the metrics described herein, the subject de-escalates to the first dose. In some embodiments, where the subject tolerates the second dose during the second dosing interval, e.g., after 2, 3, or 4 weeks according to the metrics described herein, the subject escalates to the third dose for the third dosing interval. In some embodiments, where the subject does not tolerate the third dose during the third dosing interval, e.g., after 2, 3, or 4 weeks according to the metrics described herein, the subject de-escalates to the second dose.

In some embodiments, where the subject tolerates the first dose during a first dosing interval of 3 weeks according to the metrics described herein, the subject escalates to the second dose for a second dosing interval of 3 weeks. In some embodiments, where the subject does not tolerate the second dose during the second dosing interval of 3 weeks according to the metrics described herein, the subject de-escalates to the first dose. In some embodiments, where the subject tolerates the second dose during the second dosing interval of 3 weeks according to the metrics described herein, the subject escalates to the third dose for a third dosing interval of 3 weeks. In some embodiments, where the subject does not tolerate the third dose during the third dosing interval of 3 weeks according to the metrics described herein, the subject de-escalates to the second dose.

In some embodiments, during each dosing interval (e.g., the first dosing interval, the second dosing interval, the third dosing interval), the bacteria are first administered once per day (QD) for a set amount of days, then twice per day (BID) for a set amount of days, and then three times per day (TID) for the remainder of the dosing interval. In some embodiments, a patient may initially take a half-dose, e.g., by mixing the entire contents of a sachet in which the bacteria are provided and then discarding half, e.g., for one or two days. In some embodiments, a patient may initially take a one third of a whole dose, e.g., by mixing the entire contents of a sachet in which the bacteria are provided and then discarding two thirds, e.g., for one or two days.

In specific embodiments, during each dosing interval (e.g., the first dosing interval, the second dosing interval, the third dosing interval), the bacteria are administered once per day (QD) for two days, twice per day (BID) for 2 days, and then three times per day (TID) for the remainder of the dosing interval.

In some embodiments, the first dose is about 1×10¹¹, about 2×10¹¹, about 3×10¹¹, about 4×10¹¹, or about 5×10¹¹ of the bacteria described herein (e.g., SYNB1353) as determined by live cell counting. In some embodiments, the first dose is about 3×10¹¹ of the bacteria described herein (e.g., SYNB1353) as determined by live cell counting. In some embodiments, the first dose is 3×10¹¹ of the bacteria described herein (e.g., SYNB1353) as determined by live cell counting. In some embodiments, the second dose is about 4×10¹¹, about 5×10¹¹, about 6×10¹¹, about 7×10¹¹, or about 8×10¹¹ of the bacteria described herein (e.g., SYNB1353) as determined by live cell counting. In some embodiments, the second dose is about 6×10¹¹ of the bacteria described herein (e.g., SYNB1353) as determined by live cell counting. In some embodiments, the second dose is 6×10¹¹ of the bacteria described herein (e.g., SYNB1353) as determined by live cell counting. In some embodiments, the third dose is about 8×10¹¹, about 9×10¹¹, about 1×10¹², about 1.1×10¹², or about 1.2×10¹² of the bacteria described herein (e.g., SYNB1353) as determined by live cell counting. In some embodiments, the third dose is about 1×10¹² of the bacteria described herein (e.g., SYNB1353) as determined by live cell counting. In some embodiments, the third dose is 1×10¹² of the bacteria described herein (e.g., SYNB1353) as determined by live cell counting. In some embodiments, a subject is administered a first dose of 3×10¹¹, a second dose of 6×10¹¹, and a third dose of 1×10¹² of the bacteria described herein (e.g., SYNB1353) as determined by live cell counting.

In certain embodiments, the method of treatment, e.g., for HCU, and/or method of reducing levels of methionine, cysteine and/or homocysteine in a subject may comprise genetically engineered bacteria that are capable of metabolizing methionine, cysteine and/or homocysteine in the diet or gut-resident free methionine, cysteine and/or homocysteine present in the small intestine. Studies have shown that pancreatic and other glandular secretions into the intestine contain high levels of proteins, enzymes, and polypeptides, and that the amino acids produced as a result of their catabolism are reabsorbed back into the blood in a process known as “enterorecirculation” (Chang, 2007; Sarkissian et al., 1999). Thus, high intestinal levels of methionine, cysteine and/or homocysteine may be partially independent of food intake and are available for breakdown by a methionine, cysteine and/or homocysteine metabolizing enzyme, e.g., MetDC, e.g., as expressed in a genetically engineered bacterium disclosed herein. In some embodiments, the genetically engineered bacteria and dietary protein are delivered after a period of fasting or methionine-restricted dieting. In some embodiments, the genetically engineered bacteria may be capable of metabolizing methionine, cysteine and/or homocysteine enterorecirculating from the blood. In these embodiments, the genetically engineered bacteria need not be delivered simultaneously with dietary protein. A methionine, cysteine and/or homocysteine gradient is generated, e.g., from blood to gut, where the genetically engineered bacteria metabolize methionine, cysteine and/or homocysteine. A patient suffering from homocystinuria (HCU) may be able to resume a substantially normal diet, or a diet that is less restrictive than the stringent low-methionine diet recommended for example to reach/maintain a target methionine, cysteine and/or homocysteine of <50 μM or <100 μM. In some embodiments, the genetically engineered bacteria are delivered simultaneously or right after dietary protein. In other embodiments, the genetically engineered bacteria are not delivered simultaneously with dietary protein.

In some embodiments, the method of treatment, e.g., for HCU, comprises measuring homocysteine baseline levels and/or dietary intake of homocysteine prior to administration of the genetically engineered bacteria. In some embodiments, the baseline measurement is made in a fasted state, e.g., prior to a meal, e.g., in a subject having HCU. In some embodiments, the baseline homocysteine dietary intake or baseline plasma levels is recorded for 10, 9, 8, 7, 6, 5, 4, 3, 2, or 1 days prior to administration of the genetically engineered bacteria, e.g., for 3 days. In some embodiments, the method of treatment comprises measuring methionine, cysteine and/or homocysteine at various time points while a subject is being treated with the genetically engineered bacteria. Dietary homocysteine or methionine intake during treatment is determined using the baseline measurement, e.g., dietary homocysteine or methionine intake may be within ±5%, ±10%, ±15%, or ±20% of the subject's baseline homocysteine or methionine intake. Baseline dietary deviations of homocysteine or methionine may be <10% during diet run-in or <25% during diet run-in. A subject may record a 3 day dietary intake regularly, i.e., prior and/or during the administration period. In some instances, dietary intake may be recorded daily during the administration period. In some embodiments, the period of time at which a measurement is taken is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of the genetically engineered bacterium.

### EXAMPLES

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1: Strain Development and Testing

Medium copy plasmids were used. These plasmids contain either Methionine gamma lyase (MGL) or Methionine decarboxylase (MDC) under the control of an anhydrotetracycline (ATC)-inducible promoter. Plasmids were constructed through TypeIIS cloning of synthesized gBlock fragments (IDT, Coralville, IA) containing these genes, followed by Sanger sequencing for sequence verification. Plasmids were used to transform E. coli Nissle (EcN). Genotypes are provided in Table 6 and Table 12, herein.

TABLE 6
E. coli Strains

| | | Antibiotic |
|---|---|---|
| Strain No. | Background/genotype | resistance |
| SYN7328 | SYN001 (WT EcN); Logic2375(pSC101; Ptet:metNIQ) | carbenicillin |
| | | (carb) |
| SYN7344 | SYN001; Logic2279(p15a; Ptet:metDC) | kanamycin |
| | | (kan) |
| SYN7345 | SYN001; ΔyjeH | chloramphenicol |
| | | (cam) |
| SYN7346 | SYN001; ΔyjeH; Logic2279(p15a; Ptet:metDC) | cam, kan |
| SYN7347 | SYN001; ΔyjeH; Logic2375(pSC101; Ptet:metNIQ) | cam, carb |
| SYN7348 | SYN001; | kan, carb |
| | Logic2279(p15a; Ptet:metDC); Logic2375(pSC101; Ptet:metNIQ) | |
| SYN7349 | SYN001; ΔyjeH; | cam, carb, kan |
| | Logic2279(p15a; Ptet:metDC); Logic2375(pSC101; Ptet:metNIQ) | |

Genes encoding metP and metDC were integrated to facilitate methionine import and metabolism. The importer metP is derived from Flavobacterium segetis and facilitates the uptake of methionine into the cell. metDC is derived from Streptomyces sp. 590 and includes two modifications (Q70D and N82H) to improve its activity at converting methionine to 3-MTP and CO₂. Both genes are under the regulatory control of a chemically inducible promoter (Ptac), which is induced by IPTG.

To prevent release of methionine from the bacteria in the GI tract once it enters the cell, the yjeH gene that encodes a methionine/branched chain amino acid exporter was deleted. To control growth in vivo and in the environment, strains were engineered to be an auxotrophic strain through deletion of the dapA gene that encodes for dihydrodipicolinate synthase, which is essential for the outer membrane. This deletion renders the bacteria unable to synthesize DAP, thereby preventing the proper formation of bacterial cell wall unless the strain is supplemented with DAP exogenously.

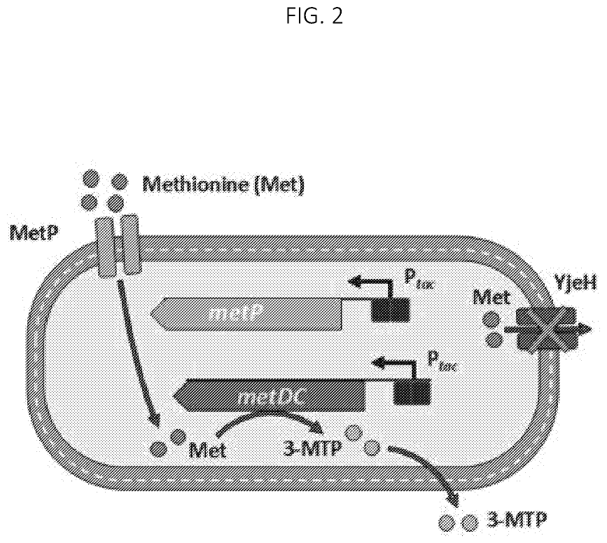

Other strains produced are listed as follows: SYN094 (control), SYN7642 (metDC (Q70D N82H) ΔyjeH), SYN7970 (2 copies metDC, metP, ΔyjeH, Δdap, Δφ), SYN8002 (1 copies metDC, metP, ΔyjeH, Δdap, Δφ), and SYN8003 (3 copies metDC, metP, ΔyjeH, Δdap, Δφ), and SYN8070 (aka SYNB1353; 3 copies metDC, metP, ΔyjeH, Δdap, Δclb). SYNB1353, subsequently used in clinical studies, comprises a metP gene, metDC gene, and deletion of the yjeH gene, as shown in FIG. 2.

To assess the in vitro activity of the final integrated clinical candidate strain, SYNB 1353 was incubated in minimal media with 10 mM methionine and supernatants were collected up to either 60 or 120 minutes. SYNB 1353 significantly degraded methionine and concomitantly produced 3-MTP under those conditions as compared to un-engineered EcN control. SYNB1353 degraded methionine at a rate of 1.72 μmol/h/1×10⁹ cells.

### Example 2. A Phase 1, Dose-Escalation, Randomized, Placebo-Controlled Study to Assess the Safety, Tolerability, and Pharmacodynamics of SYNB1353 in Healthy Volunteers

SYNB1353 is an orally administered, non-systemically absorbed live biotherapeutic engineered to consume Met in the GI tract for the treatment of HCU. SYNB 1353 will be administered orally immediately following meals according to the study schedule. Placebo will be matching in appearance and delivery method to SYNB1353.

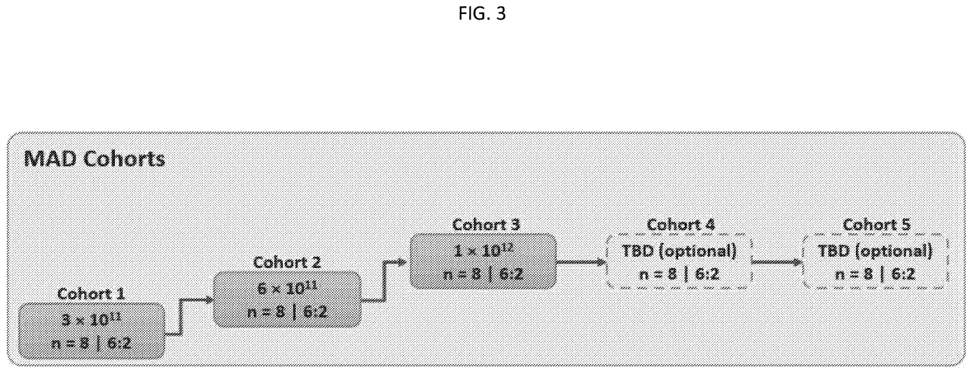

This phase 1, double-blind (Sponsor-open), placebo-controlled, randomized, dose-escalation, inpatient study will assess the safety, tolerability, and PD of SYNB1353 in healthy volunteers (HVs). The study will include up to 5 cohorts of HVs. In each cohort, HVs will be randomly assigned to IMP, according to a MAD design, to receive either SYNB1353 or placebo (6 active: 2 placebo per cohort) (see FIG. 3).

The Screening Period will occur up to 30 days before the first day of study drug dosing (Day 1). HVs will report to the CRU for admission on Day −1 (or on Day −2, if preferred by the CRU). On Day −1, baseline evaluations will be performed, and subjects will be started on a controlled diet (details to be provided in the study-specific Diet Manual). A PPI [esomeprazole 20 mg]) will be initiated once daily (QD), 60 to 90 minutes before breakfast, starting 2 days before the first dose of IMP (i.e., subjects who are not yet admitted to the CRU will be instructed to begin taking the PPI with breakfast on Day −2 at home). IMP refers to both SYNB1353 and placebo. A Methionine loading study will be performed on Day −1 as well as baseline safety laboratory evaluations. Subjects will be randomly assigned to IMP according to the MAD design on Day 1 of the Treatment Period and will be administered IMP during the Treatment Period immediately following meals. On Day 1 subjects will receive a single dose of IMP, on Days 2 and 3 subjects will receive up to 2 doses of IMP (BID), and on Days 4 to 7 subjects will receive IMP up to 3 times per day (TID). Fasting blood samples will be collected on the morning of Days 5 and 6. A second Methionine loading study will be performed on Day 7. Safety laboratory evaluations will be repeated on Day 4 and on Day 8. Subjects will be released from the CRU on the morning of Day 8 following completion of all Day 8 assessments.

### Methionine Loading Study

A Methionine loading study will be performed on Day-1 and Day 7 after an overnight fast (starting at approximately 10 PM the previous day). After baseline blood and spot urine samples have been collected, subjects will receive a standard breakfast meal replacement shake followed by an oral dose of Methionine at a dose of up to 100 mg/kg dissolved in diluent. The standard breakfast meal replacement shake, Methionine, and IMP (if applicable) are to be consumed over a 15-minute period. Blood and urine samples will be taken at intervals over the following 24 hours. Following the collection of the initial 4 hours of samples, subjects will resume a normal diet. At each IMP dose level, a dose of methionine of 30 mg/kg will be evaluated and only following demonstration of safety by the Safety Review Committee will the dose of methionine be increased to but not exceeding 100 mg/kg. Note: if following the first cohort at 30 mg/kg of methionine this dose is considered adequate to evaluate the PD of SYNB1353 in HVs, subsequent cohorts will be dosed at increasing doses of IMP but with up to 30 mg/kg of methionine.

### Dose, Cohorts, and Dose Escalation

The starting dose of SYNB1353 will be 3×10¹¹ live cells, based on clinical and nonclinical safety and tolerability of previously tested similar EcN-based products. Dose-escalation decisions will be made once at least 6 subjects in a cohort have received IMP and have had at least 24 hours of post-dose observation. Dose escalation will be up to approximately 3-fold per cohort. Decisions will be made based on a blinded review of tolerability, clinical observations, safety laboratory assessments, and, optionally, PD. During the assessment of dose escalation, the dosing schedule may be modified, not to exceed TID on days scheduled for administration of IMP. The interval between subsequent cohorts must be at least 72 hours.

The dose cohorts that were completed this study are listed in Table 9, and Tables 7 and 8 show alternative dosing cohorts.

TABLE 7

| | | SYNB1353 Dose |
|---|---|---|
| Cohort ^{a} | Number of Subjects | (# live cells) |
| 1 | 8 (6 active:2 placebo) | 3 × 10¹¹ |
| 2 | 8 (6 active:2 placebo) | 6 × 10¹¹ |
| 3 | 8 (6 active:2 placebo) | 1 × 10¹² |
| Optional 4 | 8 (6 active:2 placebo) | TBD ≤ 2 × 10¹² |
| Optional 5 | 8 (6 active:2 placebo) | TBD ≤ 2 × 10¹² |

BID = twice daily; IMP = investigational medicinal product; TBD = to be determined; TID = 3 times per day.
^{a} For each cohort, subjects will receive a single dose of IMP on the first day of dosing (Day 1), on Days 2 and 3 subjects will receive up to 2 doses of IMP (BID), and on Days 4 to 7 subjects will receive up to 3 doses of IMP (TID).

TABLE 8

| | | | Methionine dose for |
|---|---|---|---|
| | | SYNB1353 Dose | methionine loading |
| Cohort ^{a} | Number of Subjects | (# live cells) | study* |
| 1 | 8 (6 active:2 placebo) | 3 × 10¹¹ | 30 mg/kg |
| 2 | 8 (6 active:2 placebo) | 3 × 10¹¹ | up to 100 mg/kg |
| 3 | 8 (6 active:2 placebo) | 6 × 10¹¹ | 30 mg/kg |
| 4 | 8 (6 active:2 placebo) | 6 × 10¹¹ | up to 100 mg/kg |
| 5 | 8 (6 active:2 placebo) | 1 × 10¹² | 30 mg/kg |
| 6 | 8 (6 active:2 placebo) | 1 × 10¹² | up to 100 mg/kg |
| 7 | 8 (6 active:2 placebo) | TBD ≤ 2 × 10¹² | 30 mg/kg |
| 8 | 8 (6 active:2 placebo) | TBD ≤ 2 × 10¹² | up to 100 mg/kg |

BID = twice daily; IMP = investigational medicinal product; TBD = to be determined; TID = 3 times per day.
^{a} For each cohort, subjects will receive a single dose of IMP on the first day of dosing (Day 1), on Days 2 and 3 subjects will receive up to 2 doses of IMP (BID), and on Days 4 to 7 subjects will receive up to 3 doses of IMP (TID).
*A methionine loading study will be performed on Day −1 and Day 7 after an overnight fast.

TABLE 9

| | | | Methionine dose for |
|---|---|---|---|
| | | SYNB1353 Dose | methionine loading |
| Cohort ^{a} | Number of Subjects | (# live cells) | study* |
| 1 | 8 (6 active:2 placebo) | 3 × 10¹¹ | 30 mg/kg |
| 3 | 8 (6 active:2 placebo) | 6 × 10¹¹ | 30 mg/kg |
| 5 | 8 (6 active:2 placebo) | 1 × 10¹² | 30 mg/kg |
| 7 | 8 (6 active:2 placebo) | 1 × 10¹² | 30 mg/kg |
| | | formulated with | |
| | | vitamin B₆ | |

BID = twice daily; IMP = investigational medicinal product; TBD = to be determined; TID = 3 times per day.
^{a} For each cohort, subjects will receive a single dose of IMP on the first day of dosing (Day 1), on Days 2 and 3 subjects will receive up to 2 doses of IMP (BID), and on Days 4 to 7 subjects will receive up to 3 doses of IMP (TID).
*A methionine loading study will be performed on Day −1 and Day 7 after an overnight fast.

The maximum tolerated dose (MTD) is defined as the dose immediately preceding the dose level at which either of the following criteria are met: 1) ≥4 subjects experience an IMP-related Common Terminology Criteria for Adverse Events (CTCAE) Grade 2. For the purposes of this criterion nausea and vomiting requiring oral antiemetic will not be considered Grade 2 unless other Grade 2 criteria are met; and 2) ≥2 subjects experience a treatment-related Grade 3 or higher toxicity.

Up to two optional cohorts of 8 subjects each (6 active: 2 placebo) may be added at doses not exceeding 2×10¹² live cells.

Before proceeding to the next dose level, there must be agreement that the safety and tolerability data support dose escalation. The CRU Investigator, the clinical research organization's (CRO) pharmacovigilance physician, and the Sponsor's medical monitor may recommend dose level expansion at the current dose, escalation to the next higher dose, reduction to a lower dose, changes to the Dose-ramp design (including option to prolong the Treatment Period), or declaration that the MTD has been achieved.

### Stopping Rules

If any of the following events occur, enrollment will halt immediately, and subjects already participating in the study at the time of study stopping will discontinue IMP dosing but otherwise remain on the protocol schedule, unless the Sponsor, Investigator, and medical monitor advise otherwise.

Both dosing and enrollment in the study will pause pending safety review if any of the following criteria are met: 1) any SAE that is at least possibly related to the study product; 2) if 2 or more subjects experience an AE ≥Grade 3 (using the National Cancer Institute [NCI] CTCAE grading scales) that is related to the IMP as assessed by the Investigator. GI AEs ≥Grade 3 will not meet this criterion; or 3) the Investigator, the CRO's pharmacovigilance physician, and the Sponsor's medical monitor determine that an event or current data warrant further evaluation and/or stopping the study.

### Duration of Study Participation

The anticipated time of study participation for a participant is planned to be approximately 68 days: 1) Screening Period: up to 30 days; 2) Treatment Period: up to 7 days of planned IMP dosing; and 3) Safety Follow-up Period: at least 28 days (+3 days after last dose of IMP)

### Study Population

This study will enroll up to approximately 40 adult male and female HVs, regardless of race/ethnicity.

### Inclusion Criteria

1. Age ≥18 to ≤64 years.
2. Able and willing to voluntarily complete the informed consent process.
3. Available for and agree to all study procedures, including feces, urine, and blood collection and adherence to diet control, inpatient monitoring, follow-up visits, and compliance with all study procedures.
4. Male subjects who are sexually abstinent or surgically sterilized (vasectomy), or those who are sexually active with a female partner(s) and agree to use an acceptable method of contraception (such as a condom with spermicide) combined with an acceptable method of contraception for their non-pregnant female partner(s) (as defined in Inclusion Criterion #5) after informed consent, throughout the study, and for a minimum of 3 months after the last dose of IMP, and who do not intend to donate sperm in the period from Screening until 3 months following administration of the IMP.
5. Female subjects who meet 1 of the following:
a. Women of childbearing potential (WOCBP) must have a negative serum pregnancy test (human chorionic gonadotropin) at Screening and a negative urine pregnancy test at baseline prior to the start of IMP and must agree to use acceptable method(s) of contraception, combined with an acceptable method of contraception for their male partner(s) (as defined in Inclusion Criterion #4) after informed consent, throughout the study and for a minimum of 3 months after the last dose of IMP. Acceptable methods of contraception include hormonal contraception, hormonal or non-hormonal intrauterine device, bilateral tubal occlusion, complete abstinence, vasectomized partner with documented azoospermia 3 months after procedure, diaphragm with spermicide, cervical cap with spermicide, vaginal sponge with spermicide, or male or female condom with or without spermicide.
b. WOCBP must not be breastfeeding.
c. Premenopausal women with at least 1 of the following: i. Documented hysterectomy; ii. Documented bilateral salpingectomy; iii. Documented bilateral oophorectomy; iv. Documented tubal ligation/occlusion; and v. Sexual abstinence is preferred or usual lifestyle of the subject.
d. Postmenopausal women (12 months or more amenorrhea verified by follicle-stimulating hormone [FSH] assessment and over 45 years of age in the absence of other biological or physiological causes).

### Exclusion Criteria

1. Acute or chronic medical, surgical, psychiatric, or social condition or laboratory abnormality that may increase subject risk associated with study participation, compromise adherence to study procedures and requirements, or may confound interpretation of study safety or PD results and, in the judgment of the Investigator, would make the subject inappropriate for enrollment.
2. Body mass index <18.5 or ≥35 kg/m2.
3. History of or current immunodeficiency disorder including human immunodeficiency virus (HIV) antibody positivity.
4. Hepatitis B surface antigen positivity (subjects with hepatitis B surface antibody positivity and hepatitis B core antibody positivity are not excluded, provided that the hepatitis B surface antigen is negative).
5. Hepatitis C antibody positivity, unless a hepatitis C virus ribonucleic acid test is performed, and the result is negative.
6. History of febrile illness, confirmed bacteremia, or other active infection deemed clinically significant by the Investigator within 30 days prior to the anticipated first dose of IMP.
7. History of (within the past month) passage of 3 or more loose stools per day, where “loose stool” is defined as a Type 6 or Type 7 on the Bristol Stool Chart.
8. Inflammatory or irritable bowel disorder of any grade experienced within the previous 60 days.
9. Active or past history of GI bleeding within 60 days prior to the Screening Visit as confirmed by hospitalization-related event(s) or medical history of hematemesis or hematochezia.
10. Intolerance of or allergic reaction to EcN, esomeprazole and all other PPIs, or any of the ingredients in SYNB1353 or placebo formulations.
11. Allergy or intolerance to multiple antibiotics which would preclude use of antibiotics for eradication of SYNB1353 in case of colonization.
12.Underlying cardiovascular disease or uncontrolled gastroesophageal reflux disease
13. Currently taking or plans to take Methotrexate, Azuridine, Nitrous Oxide, Phenytoin, or Carbamazepine.
14. Currently taking or plans to take any type of systemic (e.g., oral or intravenous) antibiotic within 28 days prior to the first anticipated dose of IMP through final assessment, including planned surgery, hospitalizations, dental procedures, or interventional studies that are expected to require antibiotics. Exception: topical antibiotics are allowed.
15. Major surgery (an operation upon an organ within the cranium, chest, abdomen, or pelvic cavity) or inpatient hospital stay within the past 3 months prior to Screening.
16. Dependence on alcohol or drugs of abuse.
17. Administration or ingestion of an investigational drug within 30 days or 5 half-lives, whichever is longer, prior to the Screening Visit, or current enrollment in an investigational study.
18. Screening laboratory parameters (e.g., chemistry panel, hematology, coagulation) and ECG outside of the normal limits based on standard ranges, or as defined in Table 10, below, or as judged to be clinically significant by the Investigator. A single repeat evaluation is acceptable.

TABLE 10

| Laboratory Parameter | Acceptable Range |
|---|---|
| White blood cells (WBCs) | 3.0-14.0 × 10⁹/L |
| Platelets | >100 × 10⁹/L |
| Hemoglobin | >10 g/dL |
| Estimated glomerular filtration rate | >60 mL/min/1.73 m² |
| (eGFR) by the Chronic Kidney Disease | |
| Epidemiology Collaboration equation | |
| Asparate aminotransferase (AST) | ≤2× upper limit of normal |
| | (ULN) |
| Alanine aminotransferase (ALT) | ≤2× ULN |
| Bilirubin | <ULN, unless diagnosed with |
| | Gilbert's syndrome |

### Treatment of Subjects

SYNB1353 or placebo will be administered orally immediately following meals according to the study schedule. Within this protocol, IMP refers to SYNB 1353 or placebo. Detailed instructions for the storage, handling, and administration of IMP will be provided in the Pharmacy Manual.

SYNB1353 IMP is formulated as a nonsterile solution intended for oral administration. After growth and purification of SYNB1353, the material is concentrated into 50 mM Tris, pH 7.5 buffer containing 10% w/v trehalose. SYNB1353 is subsequently lyophilized to form the bulk drug product. The lyophilized product is sieved into powder form and filled into high-density polyethylene (HDPE) bottles. Placebo will be manufactured using an inactive powder that is color matched to the SYNB1353 drug product.

The manufactured SYNB1353 drug product will have a prespecified mass of powder in HDPE bottles. The powder will be resuspended in a diluent containing sodium bicarbonate and masking agents, with vitamin B₆ (<100 mg/day) or without vitamin B₆ depending on the cohort, prior to dosing. The placebo will be resuspended in the same diluent.

L-Methionine is supplied as dry powder and will be suspended in diluent prior to use.

### Proton Pump Inhibitor

In this study, subjects will take a PPI (esomeprazole) at a dose of 20 mg QD administered 60 to 90 minutes prior to breakfast starting 2 days before the first dose of IMP, continuing until the last day of dosing. Subjects who are not yet admitted to the CRU on Day −2 will be instructed to begin taking the PPI prior to breakfast on Day −2 at home. The PPI should be taken at approximately the same time each day, even if no meal is consumed.

### Study Procedures and Assessments

The study Schedule of Events is presented in Table 11 and the Methionine loading study details are provided in Table 12.

TABLE 11

| | Screening | CRU | Treatment | | | Safety Follow-up |
|---|---|---|---|---|---|---|
| | Period | Admission | Period | | | Visit/End of Study ^{c} |
| | Study Day(s) → | | | | | |
| | | | Treatment | Treatment | | |
| | Days −30 | | Day | Day | Day | 28 (+3) days after |
| Study Procedure ↓ | to −2 | Day −1 ^{a} | 1-6 | 7 | 8 ^{b} | last dose |
| Informed consent | □ | | | | | |
| Medical history | □ | □ | | | | |
| Height and weight | □^{d} | □^{e} | | | | |
| Screening for drugs of abuse | □ | | | | | |
| (urine drug screen) | | | | | | |
| Screening for acute | | □ | | | | |
| respiratory infections (e.g., | | | | | | |
| COVID-19) ^{f} | | | | | | |
| Serology/Screening | □ | | | | | |
| infectious disease | | | | | | |
| (HIV/hepatitis B, C) | | | | | | |
| Serum pregnancy test | □ | | | | | |
| (WOCBP only) | | | | | | |
| Urine pregnancy test | | □ | | | | |
| (WOCBP only) | | | | | | |
| FSH test (for women 12+ | □ | | | | | |
| months amenorrhea | | | | | | |
| and/or >45 years of | | | | | | |
| age if applicable at | | | | | | |
| Investigator discretion) | | | | | | |
| Record prior/concomitant | □ | □ | □ | □ | □ | □ |
| medications | | | | | | |
| Adverse event reporting ^{g} | □ | | | | | |
| Admit to inpatient facility | | □ | | | | |
| Required inpatient stay | | □ | □ | □ | | |
| Controlled standardized diet ^{h} | | □ | □ | □ | □ | |
| Physical examination | □ | □ | | | | |
| (complete or symptom- | | | | | | |
| directed) ⁱ | | | | | | |
| Vital signs | □ | □ | □ | □ | □ | |
| (systolic/diastolic | | | | | | |
| blood pressure, pulse, | | | | | | |
| body temperature) ^{j} | | | | | | |
| Electrocardiogram (semi- | □ | □^{k} | | | □ | |
| supine for 5 minutes) | | | | | | |
| Coagulation (PT/aPTT/INR) | □ | | | | | |
| Safety laboratory tests | □ | □ | •^{l} | | □ | |
| (hematology/CBC with | | | | | | |
| differential, | | | | | | |
| serum/plasma chemistry, | | | | | | |
| CRP, urinalysis) (>4 | | | | | | |
| hours after meal) | | | | | | |
| Subject randomization | | | □^{m} | | | |
| Administer assigned IMP | | | □ | □ | | |
| immediately following | | | | | | |
| meals (SYNB1353 or | | | | | | |
| placebo) ⁿ | | | | | | |
| Administer PPI ^{o} | | □ | □ | □ | | |
| Fasting amino acid panel | □ ^{p} | | □^{q} | | | |
| Methionine loading study ^{r} | | □ | | □ | | |
| Feces for SYNB1353 qPCR ^{s} | | □ | | | | □ ^{r} |
| Plasma^{u} and urine^{v} for | | □ | | □ | | |
| future exploratory analyses | | | | | | |

Abbreviations: AE = adverse event; aPTT = activated partial thromboplastin time; CBC = complete blood count; CRP = C-reactive protein; CRU = clinical research unit; eCRF = electronic case report form; FSH = follicle-stimulating hormone; HIV = human immunodeficiency virus; ICF = informed consent form; IMP = investigational product; INR = international normalized ratio; PPI = proton pump inhibitor; PT = prothrombin time; qPCR = quantitative polymerase chain reaction; SAE = serious adverse event; TID = 3 times per day; WOCBP = women of childbearing potential
^{a} Subjects may be admitted to the CRU on Day −2, if preferred. The activities in this column should be performed on Day −1, regardless of the day of admission, except for medical history, weight, screening for acute respiratory infections, urine pregnancy test, and physical examination, which may be conducted on Day −2.
^{b} Discharge following assessments on Day 8.
^{c} May be performed either in the clinic or by telemedicine.
^{d}Body mass index will be calculated.
^{e}Weight only.
^{f} Screening for acute respiratory infections will be conducted according to current local guidelines. May be repeated as necessary.
^{g} All AEs occurring from the start of the PPI through study discharge will be recorded, regardless of causal assessment to IMP. All SAEs occurring from the signing of the ICF through study discharge will be recorded, regardless of causal assessment to IMP.
^{h} Details provided in the Diet Manual.
ⁱ Physical examination at Screening and baseline. Symptom-directed physical examinations may be performed at the Investigator's discretion at nonscheduled times if warranted. Any abnormal findings observed after the Screening physical examination should be recorded as AEs.
^{j} Subjects are required to remain in the semi-supine position for at least 5 minutes prior to obtaining vital signs. Vital signs will be obtained within 1 hour prior to breakfast and then every 8 hours (±1 hour) during the inpatient period.
^{k}Triplicate electrocardiograms to be obtained on Day −1.
^{l}Safety labs during screening, at baseline (Day−1), Day 4, and Day 8. Additionally, unscheduled clinical laboratory tests may be obtained at any time during the study at the Investigator's discretion. The diagnosis corresponding to any clinically significant abnormality must be recorded as an AE
^{m}Randomization will occur only on the first day of IMP dosing.
ⁿ IMP administered immediately following meals, once daily on Day 1, twice daily (BID) on Day 2 and 3, and 3 times daily (TID) on Treatment Days 4 to 7.
^{o} Administered 60-90 minutes before breakfast. The PPI should be taken for 2 days prior to the first dose of IMP. Subjects will be instructed to begin taking the PPI with breakfast on Day −2 (i.e., at home, if necessary).
^{p} Day −2
^{q}Days 5 and 6
^{r} Refer to Table 12 for timing of assessments during the Methionine loading study.
^{s} Subjects should have 1 baseline fecal sample prior to the first dose of IMP; however, lack of baseline stool sample does not prevent participation. The baseline fecal sample collection must be recorded in the eCRF with collection date, time, and weight; other fecal samples will be recorded in the eCRF with the collection date and time only. Fecal samples will be collected using a fecal collection kit. During follow-up, fecal samples will be collected weekly for the first 4 weeks after last dose; thereafter monthly until 3 months of last dose, as needed.
t Baseline collection can occur any time before dosing on Day 1.
^{u}Subjects will be followed in the study for at least 28 days after the last dose, Fecal samples will be collected weekly, beginning 1 week after the last dose, until 28 days after the last dose or until two documented, negative samples as analyzed by qPCR are obtained, whichever occurs later. If still colonized with SYNB1353 at 4 weeks post last dose, fecal samples should be collected at 8 and 12 weeks post last dose of IMP. Subjects who remain colonized 12 weeks following the last dose of IMP will be treated with a 3-day course of oral ciprofloxacin 500 mg by mouth (or another antibiotic as per the Investigator's Brochure). Clearance will be defined in a separate study document. An additional fecal sample for qPCR assessment will be required at least 7 days after completion of antibiotic treatment to confirm clearance. If clearance is confirmed, subjects will not require any further follow-up and will be considered to have completed the study requirements. If clearance is not demonstrated at that time, the Safety Review Committee, inclusive of the Sponsor Medical Monitor, the Study Principal Investigator, and the CRO Physician will meet and come to a unanimous decision on the appropriate next steps to ensure the safety of the subject, including consideration of an additional course of antibiotics.
^{v}Overnight fasting sample (fast starting at 10 PM the previous day).
w Spot urine samples.

TABLE 12
Schedule of Events: Methionine Loading Study on Day −1 and Treatment Day 7

| Time Relative to Methionine | | | | | | | | | | |
|---|---|---|---|---|---|---|---|---|---|---|
| Loading Study or IMP | 0.25 | | | | | | | | | |
| Administration (hours)^{a} | to 0 | 0 | 0.25 | 0.5 | 1 | 2 | 4 | 8 | 16 | 24 |
| Blood for Met and | □ | | □ | □ | □ | □ | □ | □ | □ | □ |
| metabolites, tHcy, | | | | | | | | | | |
| cystathionine and | | | | | | | | | | |
| total cysteine | | | | | | | | | | |
| Spot urine collection | □ | | | | | | | | | |
| Standard breakfast | | □^{b} | | | | | | | | |
| meal replacement | | | | | | | | | | |
| shake | | | | | | | | | | |
| Administer Methionine | | □^{b} | | | | | | | | |
| (up to 100 mg/kg in | | | | | | | | | | |
| 100 mL diluent) | | | | | | | | | | |
| Administer assigned | | □^{b} | | | | | | | | |
| IMP (SYNB 1353 or | | | | | | | | | | |
| placebo)^{c} | | | | | | | | | | |
| Urine intervals for | | 0 to 4 | | | | | 4 to 8 | 8 to 24 | | |
| Met and metabolites, | | hours | | | | | hours | hours | | |
| tHcy, and 3-MTP^{d} | | | | | | | | | | |

Abbreviations: 3-MTP = 3-methylthiolpropylamine; eCRF = electronic case report form; IMP = investigational product; Met = methionine; tHcy = total Homocysteine
^{a}Activities will be conducted in order from top to bottom of the table. All times are ±5 minutes.
^{b}Methionine must be administered immediately after the standard breakfast meal replacement shake, and the IMP must be administered immediately after the methionine. The timing to complete all must not exceed 15 minutes.
^{c}IMP will be administered only on Treatment Day 7. Subjects will fast until the 4-hour sample is taken after which they may resume the study diet.
^{d}After the spot urine sample is collected, urine will be collected as a continuous collection during the intervals of 0 to 4 hours, 4 to 8 hours, and 8 to 24 hours after administration of IMP. At the end of each urine interval a forced void will be requested to end the collection.
Note:
The volume of each urine interval and the urine spot measurement will be recorded in the eCRF.

### Screening

During the Screening Period, assessments as listed in the Schedule of Events (Table 11) will be conducted within 30 days prior to enrollment. A unique number will be assigned to each subject who signs an informed consent form (ICF). Any ongoing condition as well as signs and symptoms observed prior to the subject signing the ICF should be recorded as medical history. A single repeat evaluation of eligibility laboratory assessments is acceptable.

### Safety Assessments

### Adverse Events

Adverse events will be assessed continuously by direct observation and subject event recording and interviews. The severity of AEs will be evaluated using the NCI CTCAE22. All AEs occurring from the start of the PPI through study discharge will be recorded, regardless of causal assessment to IMP.

### Vital Signs and Physical Examination

Resting vital signs (systolic blood pressure, diastolic blood pressure, pulse, and body temperature) will be collected as specified in Table 11. Subjects are required to remain in the semi-supine position for at least 5 minutes prior to obtaining vital signs.

Complete and symptom-directed physical examinations will be performed by trained medical personnel as specified in Table 11. Symptom-directed physical examinations may be performed at the Investigator's discretion at nonscheduled times if warranted. Any abnormal findings observed after the Screening physical examination should be recorded as AEs.

### Clinical Laboratory Measurements

The clinical laboratory tests listed in Table 11 will be performed as specified. Screening results will be assessed by the Investigator for inclusion of subjects in the study. Additionally, unscheduled clinical laboratory tests may be obtained at any time during the study at the Investigator's discretion. The diagnosis corresponding to any clinically significant abnormality must be recorded as an AE.

TABLE 13
Clinical Laboratory Tests

| | | | |
|---|---|---|---|
| Hematology | Basophils % | Serology^{a} | HIV1 and HIV2 |
| (CBC with | Basophils | | antibody Hepatitis B |
| differential) | Eosinophils % | | surface antigen |
| | Eosinophils | | Hepatitis B core |
| | Hematocrit | | antibody Hepatitis C |
| | Hemoglobin | | antibody |
| | Lymphocytes % | | Hepatitis C RNA (only if |
| | Lymphocytes | | hepatitis C antibody is positive) |
| | Mean corpuscular hemoglobin | Coagulation^{a} | Prothrombin time |
| | Mean corpuscular volume | | aPTT |
| | Monocytes % | | INR |
| | Monocytes | Serum | Glucose |
| | Neutrophils % | Chemistry | BUN |
| | Neutrophils | | Creatinine with eGFR |
| | Platelet count | | Sodium |
| | Red blood cells | | Potassium |
| | White blood cells | | Chloride |
| Urinalysis | Specific | | Carbon |
| | gravity pH | | dioxide |
| | Glucose | | Calcium |
| | Bilirubin | | Total |
| | Ketones | | protein |
| | Occult | | Albumin |
| | blood | | Fractionated bilirubin (total |
| | Protein | | direct and indirect) |
| | Nitrite | | Alkaline |
| | Leukocyte esterase | | phosphatase |
| | | | ALT |
| | | | AST |
| | | | Phosphorus |
| | | Plasma | 3-MTP, Met, tHcy, and other |
| | | Amino | amino acids |
| | | Acids and | |
| | | Met | |
| | | metabolites | |
| | | Urine | 3-MTP, Met, tHcy, and other |
| | | Amino | amino acids |
| | | Acids and | |
| | | Met | |
| | | metabolites | |
| | | CRP | C-reactive protein |
| | | Pregnancy | Serum follicle-stimulating |
| | | Related | hormone (for postmenopausal |
| | | Laboratory | women only)^{a} Serum pregnancy |
| | | Tests | (for WOCBP only)^{b} |
| | | | Urine pregnancy (for WOCBP |
| | | | only)^{b} |

Abbreviations: ALT = alanine aminotransferase; aPTT = activated partial thromboplastin time; AST = aspartate aminotransferase; BUN = blood urea nitrogen; CBC = complete blood count; CRP = C-reactive protein; eGFR = estimated glomerular filtration rate; HIV = human immunodeficiency virus; INR = international normalized ratio; WOCBP = women of childbearing potential.
^{a}Performed at Screening only.
^{b}Serum pregnancy at Screening; urine pregnancy at all other time points.

### Electrocardiograms

Semi-supine single 12-lead ECGs will be performed as part of Screening and as specified in Table 3, with triplicate ECGs performed at Day −1. ECG parameters to be evaluated include the RR, QT, QRS, and PR intervals. In addition, Fridericia's formula should be used to calculate the QT interval corrected for heart rate using Fridericia's formula (QTcF). Subjects are required to remain in the semi-supine position for at least 5 minutes prior to obtaining ECGs.

### Investigational Medicinal Product Clearance

Fecal samples will be collected at Day-1 and during the Safety Follow-up Period as specified in Table 11 and as described herein to evaluate the clearance of SYNB1353.

### Pharmacodynamic Assessments

The Methionine loading study will be conducted as detailed in Table 12, and as described herein. Met and metabolites, including 3-MTP and tHcy will be collected at the time points specified in Table 11. Fecal samples will be collected and analyzed by qPCR at baseline (Day-1) and during the Safety Follow-up Period (as described herein) to evaluate SYNB1353 clearance after completion of dosing. Plasma and urine samples will also be collected at the time points outlined in Table 11, for future exploratory analyses.

### Adverse Event Reporting

An AE is any untoward medical occurrence, including the exacerbation of a preexisting condition, in a subject administered a pharmaceutical product, regardless of causality. The severity rating of an AE refers to its intensity. The severity of each AE will be categorized using the NCI CTCAE, version 5.0.24. For any term that is not specifically listed in the CTCAE scale, intensity should be assigned a grade of 1 through 5 using the following CTCAE guidelines:
Grade 1: Mild; asymptomatic or mild symptoms; clinical or diagnostic observations only; intervention not indicated.
Grade 2: Moderate; minimal, local, or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to AE.

### Serious Adverse Events

An SAE is any untoward medical occurrence that meets any of the following criteria: 1) results in death; 2) is immediately life threatening (refers to an event in which the subject is at risk of death at the time of the event; it does not refer to an event, which hypothetically might have caused death if it were more severe); 3) requires inpatient hospitalization or prolongation of existing hospitalization; 4) results in a persistent or significant incapacity or substantial disruption of the ability to conduct normal life functions; 5) is a congenital anomaly/birth defect; and/or 6) based on appropriate medical judgment, represents an important medical event that may jeopardize the subject or may require intervention to prevent one of the other outcomes described above.

Death is an outcome of an SAE and not an SAE in itself. When death is an outcome, the event(s) resulting in death should be reported (e.g., “pulmonary embolism” with a fatal outcome). The appropriate diagnosis or term should be recorded and assigned severity Grade 5.

In instances of death due ultimately to the underlying disease, the cause of death should be indicated as the specific event or condition resulting in death to the extent possible. If no appropriate term with a Grade 5 severity in the CTCAE can be identified, then a term should be selected from the CTCAE category “death.”

“Life threatening” means that the subject was at immediate risk of death from the event as it occurred. This does not include an event that might have led to death if it had occurred with greater severity. Grade 4 events (e.g., thrombocytopenia) are not always serious unless they have life-threatening consequences or result in hospitalization.

Preplanned or elective hospitalizations, including social and/or convenience situations (e.g., respite care), are excluded from SAE reporting.

Overdose of either IMP or concomitant medication, without any overdose signs or symptoms unless the event meets SAE criteria (e.g., hospitalization), are excluded from SAE reporting; however, such events should still be reported on the appropriate eCRF page. All overdoses of IMP or other concomitant medication provided by the Sponsor will be recorded as major protocol deviations in the clinical study report.

All AEs that meet seriousness criteria are recorded as such, regardless of causality. The Investigator must immediately report to the Sponsor all serious adverse events, regardless of whether the Investigator believes that they are related to the IMP.

In accordance with regulatory requirements, the Sponsor or designee will immediately notify regulatory authorities and the investigators, who will in turn notify their Institutional Review Board (IRB)/Independent Ethics Committee (IEC) as necessary, of any AE associated with IMP administration or study procedures that is a serious, unexpected, suspected adverse reaction or any finding from tests in laboratory animals that suggests a significant risk for human subjects, including reports of mutagenicity, teratogenicity, or carcinogenicity. An AE or suspected adverse reaction is considered “unexpected” if it is not listed in the Investigator's Brochure or is not listed at the specificity or severity that has been previously observed.

### Statistical Methods and Determination of Sample Size

### Definition of Baseline

Baseline will be defined as the last scheduled measurement prior to the first IMP administration, unless specifically described below. If a sequence of baseline measurements is taken predose on the same day, time-matched baseline will be used. If multiple measurements are included within a baseline measurement (e.g., repeated measures at the same nominal time), the arithmetic mean of the multiple samples will be considered the baseline. If a parameter is calculated (e.g., AUC), the final predose calculated value will be considered the baseline. If the study includes multiple periods, then the baseline will be included as the baseline for the current period; if missed for the current period, it will be the average of other periods.

### Populations for Analysis

The following populations will be defined:
Efficacy/PD: all subjects in the safety population who have at least one baseline and post-baseline PD measurement and have not missed >50% of the IMP doses during the Treatment Period and all planned doses on the Methionine loading study days are fully administered
Per protocol: all subjects in the safety population who complete the Treatment Period and do not have any major protocol deviations
Safety: all subjects who receive at least 1 dose of IMP

### Safety Analysis

Safety will be evaluated by continuous monitoring of AEs, vital signs, clinical laboratory measurements, ECGs, and physical examinations. Safety parameters will be summarized descriptively by the treatment regimen that the subject was on at the time of the safety measurement. By-subject listings of all measurements and parameters will be presented in tabular format, including absolute values and changes from baseline (if applicable), by study day.

Adverse events will be coded using the Medical Dictionary for Regulatory Activities (MedDRA), and severity of AEs and laboratory abnormalities will be graded using the NCI CTCAE.22 Adverse events will be tabulated by system organ class and preferred term. Incidence tables of subjects with AEs will be presented for all AEs by maximum severity, SAEs, AEs assessed as related to IMP or the Met Load, and AEs resulting in discontinuation of study dosing.

Feces will be collected for analysis by qPCR of SYNB 1353 clearance.

### Pharmacodynamic Analysis

Urine, blood, and fecal samples will be collected during the Screening Period and on study. The following laboratory measurements will be performed to evaluate the preliminary PD of SYNB1353: 1) Plasma Met and metabolites, Total Homocysteine, Cystathionine and Total Cysteine, and 2) Urine Met and metabolites, Urine Total Homocysteine, Urine 3-MTP, and other urine amino acids.

AUC will be calculated for plasma Met and metabolites, and Aet will be calculated for urine 3-MTP and Met metabolites.

The exploratory endpoint of 3-MTP Aet change from baseline will be analyzed on the log scale by a mixed model with repeated measures with fixed effects for treatment, time (baseline or on-treatment), treatment by time, and (if applicable) period and a random effect by subject. Reporting will convert the log-scale estimate of change from baseline and change from baseline, change from placebo to the percent scale.

### Determination of Sample Size

### Multiple-Ascending Dose Cohorts

The sample size for the MAD cohorts is primarily designed for empirical evaluation of safety and tolerability in HVs.

### Randomization and Blinding

Subjects will be randomized on the first day of IMP dosing in a 3:1 ratio with a block size of 4 subjects to receive either SYNB1353 (in escalating dose cohorts) or placebo in a single treatment period.

### Changes in the Conduct of the Study or Planned Analysis

Only the Sponsor may modify the protocol. Amendments to the protocol will be made only after consultation and agreement between the Sponsor and the Investigator. The only exception is when a subject's safety would be compromised unless there is immediate action. In these circumstances, the Investigator should inform the Sponsor and the full IRB/IEC within 1 working day after the emergency occurred. All amendments that have an impact on subject risk or the study objectives or require revision of the ICF must receive approval from the IRB/IEC prior to implementation.

### Example 3. In Vitro Effects of Addition of Pydidoxal Phosphate or Pyridoxine to Methionine Consumption by SYNB1353

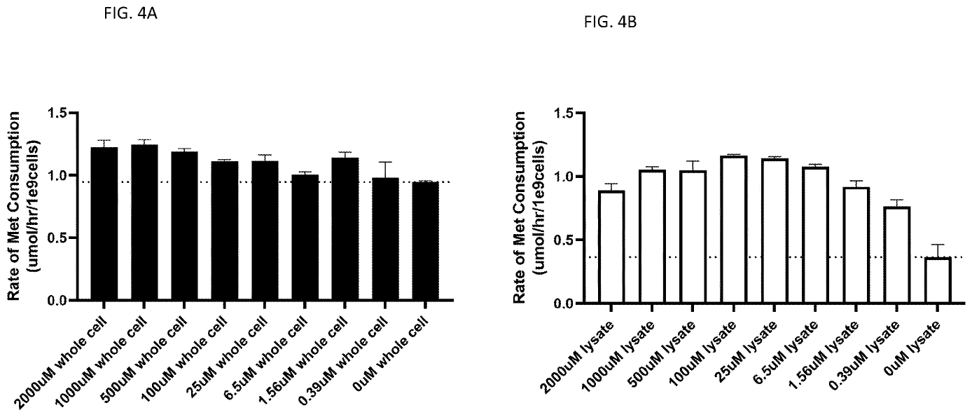

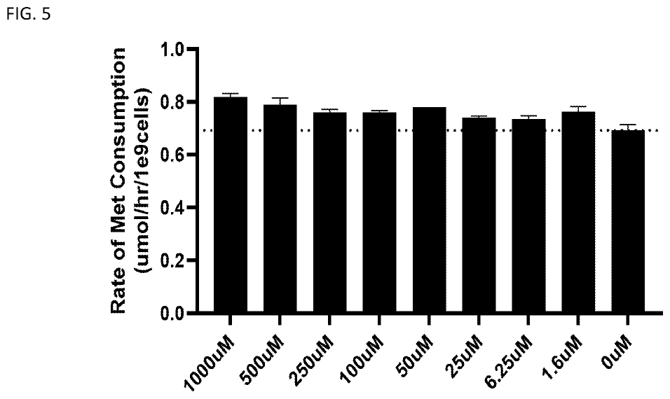

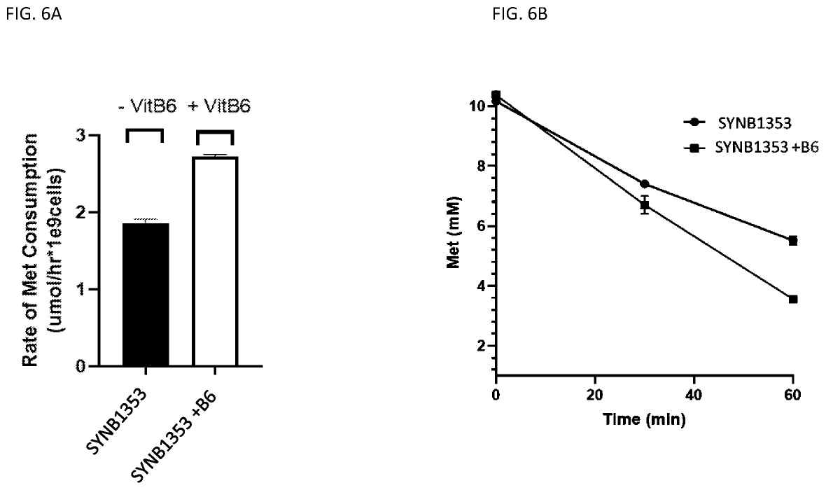

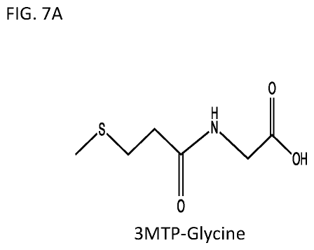

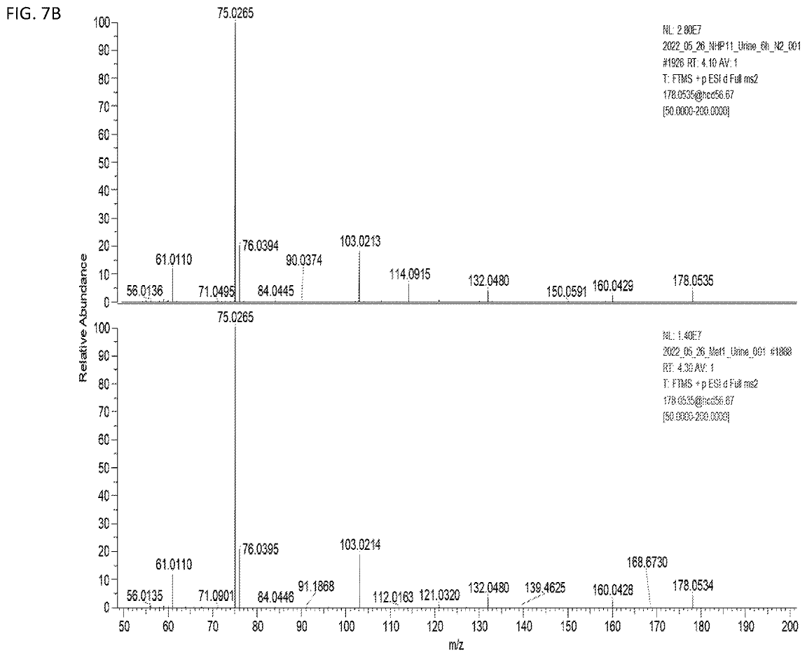

For the methionine consumption assay, cells were thawed on ice and OD600 was measured. The volume of cells equivalent to an OD of 1 were added as whole cells or lysed cells to 1 mL of M9 minimal media containing 0.5% glucose. Pyridoxal 5′ phosphate (PLP) or pyidoxine (PN) (both of which were dissolved in M9) were added to final concentrations as shown in FIG. 4A and FIG. 4B, or FIG. 5. The tube was vortexed briefly to evenly distribute the cells, and the tubes were placed at 37° C. with no shaking. Next, 150 μL of cell/media suspension was removed at 30 and 60 minute time points, spun at high speed for about 1 minute to pellet cells, and 100 μL was added to the well in a 96-well plate. The amount of L-methionine was measured using HPLC. Results shown in FIG. 4A, FIG. 4B, and FIG. 5 indicate that addition of PLP increases activity of SYNB1353 by ~30%. The addition of PN (the PLP precursor) results in a ~20% increase in rate of Met consumption by SYNB1353.

### Example 4. Detection of 3-MTP Glycine in Plasma and Urine Upon Oral 3-MTP Exposure

For this study, 12 male cynomolgus monkeys (2-5 years-old) were used. Animals were fasted the night prior to the study and a clean urine/feces pan was placed at the bottom of each cage for baseline collection (for approximately 16-18 hours). On the morning of the experiment, urine and feces were collected and each monkey was removed from its cage to orally receive 3-MTP at 10, 30, or 100 mg/kg. Animals were then returned to their cages, and a clean urine/feces collection pan was placed at the bottom of each cage. Blood was collected in heparin sodium tubes at 0, 0.5, 1, 2, 4, 6, and 24 hours post-3-MTP administration, processed for plasma extraction (2000 g at 4° C. for 10 minutes) and stored at −80° C. until analysis. Urine and feces were collected at baseline, 0 to 6, and 6 to 24 hours post dosing with total urine volume and fecal weight recorded, and samples stored at −80° C. until analysis. Study design is shown in Table 14.

TABLE 14
Study Design

| | | | | Dose | | |
|---|---|---|---|---|---|---|
| | N/ | | Dosing | concentration | | Collection |
| Group | gender | Treatment | route | (per dose) | Collection | time points |
| 1 | 3/M | Water | PO | N/A | Blood, | Blood: 0, 0.5, 1, 2, 4, 6, |
| | | | | | urine and | and 24 hours post 3-MTP |
| 2 | 3/M | 3-MTP | | 10 mg/kg | feces | Urine/feces: pre-dose, |
| 3 | 3/M | | | 30 mg/kg | | 0-6, and 6-24-hours post |
| 4 | 3/M | | | 100 mg/kg | | 3-MTP |

M: males, 3-MTP: 3-methylthiopropylamine, PO: per os, N/A: not applicable.

Metabolomics assessment of urine and fecal samples from NHPs dosed with 100 mg/kg 3-MTP was performed to identify possible 3-MTP related metabolites and to understand its metabolic fate in vivo. Three possible 3-MTP-related metabolites were identified: an oxidized metabolite (+O), a product of oxidative deamination (+O, —NH3), and an oxidative deamination >glycine conjugate (+C2 O₃) that has a tandem mass spec fragment suggesting the presence of a glycine residue. This data indicates that 3-MTP can undergo both Phase 1 and Phase 2 metabolism in NHPs.

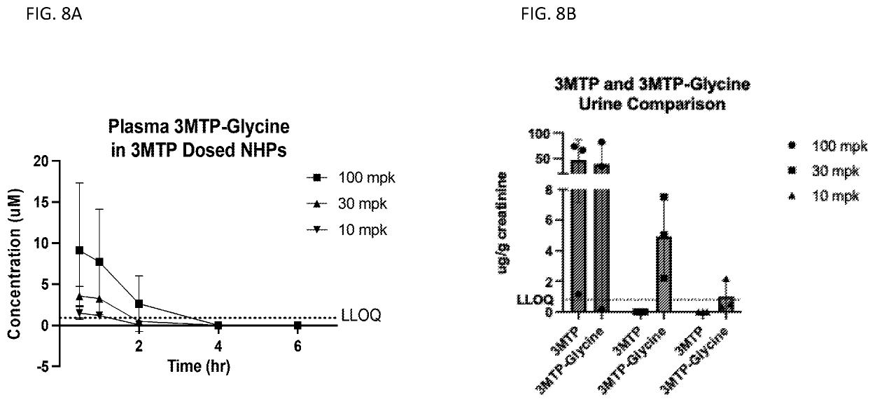

Of those three metabolites, 3-MTP glycine was validated against a synthetic standard and found in SYNB1353 dosed NHPs. Samples analyzed for 3-MTP and 3-MTP glycine levels by LC-MS/MS are shown in FIG. 8A and FIG. 8B and demonstrate that 3-MTP glycine detectable in NHP plasma in a dose dependent manner.

### Example 5. Detection of 3-MTP Glycine in Plasma and Urine Upon Administration of SYNB1353

In this study, animals were fasted the night prior to the study for approximately 16-18 hours. On the morning of the experiment, each monkey was removed from its cage and orally administered methionine, sodium bicarbonate and SYNB1353v1 or vehicle. Animals were then returned to their cages, and a clean urine collection pan was placed at the bottom of each cage. Blood was collected at 0-, 0.5-1-, 2-, 4-, 6-, and 24-hours and urine at 6-hours post-dosing. Total urine volume was recorded. The study design is shown in Table 15.

TABLE 15
Activity of SYNB1353v1 in 100 mg/kg Methionine Load

| | | | | Dose | | | |
|---|---|---|---|---|---|---|---|
| | | | Dose | volume | Stock | | Collection |
| Group | N | Treatment | Route | (mL) | concentration | Collection | time point |
| 1 | 12 | Methionine | PO | 15 | 20 mg/mL | Blood | 0, 0.5, 1, 2, 4 |
| | | Sodium | | 5 | 0.36M | | and 6 hours |
| | | bicarbonate | | | | | |
| | | Formulation | | 15 | N/A | Urine | 6 hours |
| | | buffer | | | | | |
| 2 | 12 | Methionine | PO | 15 | 20 mg/mL | Blood | 0, 0.5, 1, 2, 4 |
| | | Sodium | | 5 | 0.36M | | and 6 hours |
| | | bicarbonate | | | | | |
| | | SYNB1353v1 | | 15 | 1.36 × 10¹¹ | Urine | 6 hours |
| | | | | | live cells/mL | | |

Methionine, homocysteine, 3MTP, and 3MTP glycine were quantitated in cynomolgus monkey and human plasma and urine using LC-MS/MS. Samples were first reduced with DL-dithiothreitol, then extracted with acetonitrile containing heavy isotope methionine and homocysteine internal. Supernatants were diluted with water and analytes separated using reversed phase liquid chromatography and detected using selected reaction monitoring of compound specific ions. Peaks were integrated and analyte/internal standard ratios were used to calculate unknown concentrations relative to a standard curve.

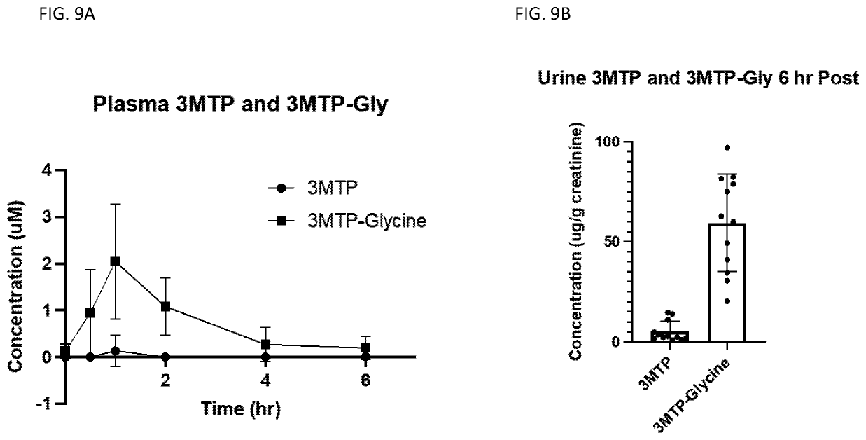

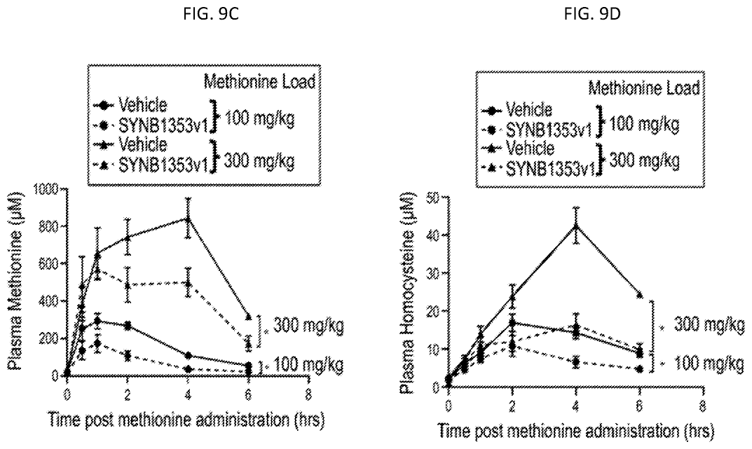

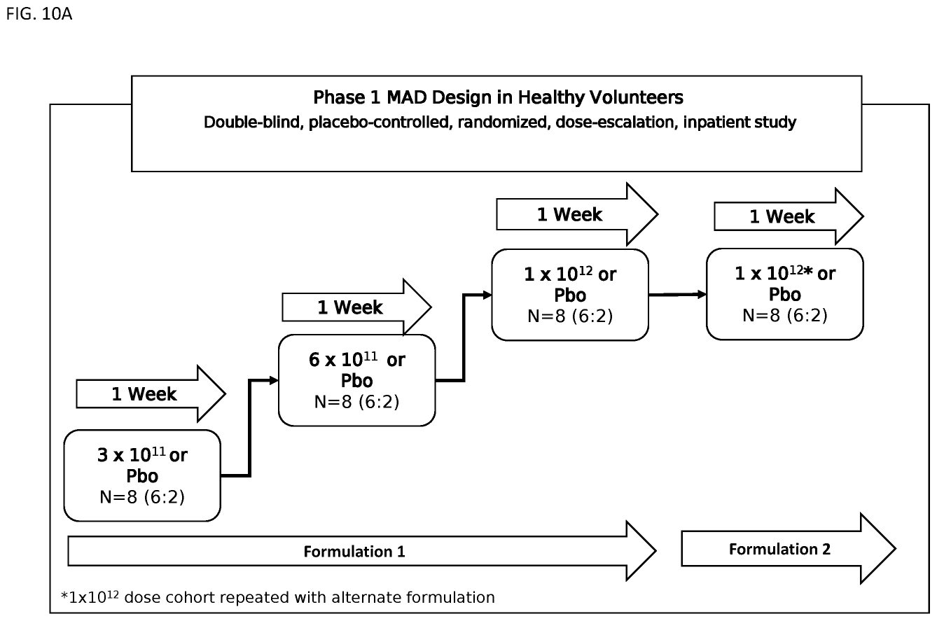

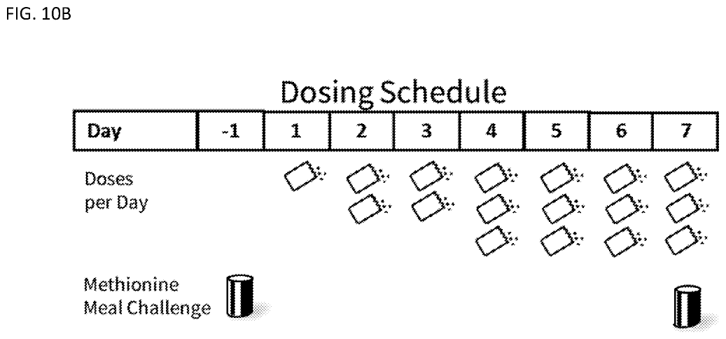

Results show that 3-MTP glycine is detectable in NHP plasma from 0.5 to 6 hours post dose (FIG. 9A). Moreover, 3-MTP glycine is detected in NHP urine at levels approximately 12-fold higher than 3-MTP on average (FIG. 9A).

In conclusion, these results suggest that 3-MTP glycine exhibits a dose response in both plasma and urine. 3-MTP glycine is detected in plasma at multiple time points and higher concentrations after strain administration. 3-MTP glycine is detected in urine at levels 12-fold higher than 3-MTP on average after strain administration to NHPs. Accordingly, 3-MTP glycine is a sensitive marker of in vivo strain activity.

### Example 6. SYNB1353 Phase 1 Study Results

The phase 1 study included a double-blind, dose-escalation, randomized, placebo-controlled, multiple-ascending dose (MAD) design in healthy volunteers in an inpatient setting. The objectives were to evaluate the safety and tolerability, assess clearance measured with quantitative polymerase chain reaction following dosing, and evaluate the pharmacodynamic effects on plasma methionine and methionine metabolites following a methionine loading study, providing a dietary model of HCU. Four cohorts of 8 healthy volunteers were enrolled, using dose levels, 3×10¹¹, 6×10¹¹ and 1×10¹² SYNB 1353 live cells, were evaluated for safety, tolerability, and capacity to metabolize methionine in was challenged with 30 mg/kg methionine before and after exposure to SYNB1353.

The bacteria was lyophilized and resuspended in formulation buffer prior to administration. Within each cohort, HVs were randomly assigned to receive SYNB1353 (6 HVs) or placebo (2 HVs). HVs reported to the clinical research unit on Day −2. On Day −1, baseline evaluations were performed, and patients were started on a controlled diet and esomeprazole 20 mg daily, both maintained until Day 7 and discharged from the clinical research unit.

Methionine loading studies were performed on Day −1 and Day 7 after an overnight fast. Subjects received a standard meal replacement shake followed by an oral methionine dose of 30 mg/kg. Blood and urine samples were collected over the following 24 hours. Fasting amino acid samples were obtained on Day −2 and Days 5 and 6.

SYNB1353 or matched placebo (taken with meals) was administered daily on Day 1, twice daily on Days 2 and 3, and three times daily on Days 4-7. Following the completion of dosing, subjects were followed for at least 28 days or until fecal samples were negative for SYNB1353 by qPCR.

The study demographics are shown in Table 16.

TABLE 16
Study demographics

| | Placebo | Dose 3E+11 | Dose 6E+11 | Dose 1E+12 | Dose 1E+12* |
|---|---|---|---|---|---|
| Parameter | (N = 8) | (n = 6) | (n = 5) | (n = 6) | (n = 5) |
| Age (years) | | | | | |
| Mean (SD) | 37.9 | 39.7 | 32.8 | 33.2 | 47.4 |
| | (8.41) | (12.75) | (4.66) | (11.27) | (13.97) |
| Min, Max | 26, 47 | 25, 56 | 25, 36 | 23, 54 | 31, 63 |
| Sex [F, M] | 0, 8 | 3, 3 | 1, 4 | 2, 4 | 2, 3 |
| Race | | | | | |
| African | 4 | 4 | 2 | 0 | 5 |
| American | | | | | |
| White | 4 | 2 | 3 | 6 | 0 |
| Other | 0 | 0 | 1 | 0 | 0 |
| Ethnicity | | | | | |
| Hispanic | 1 | 1 | 1 | 2 | 2 |
| Not Hispanic | 7 | 5 | 4 | 4 | 3 |
| BMI (kg/m²) | | | | | |
| Mean (SD) | 26.929 | 29.035 | 26.808 | 25.907 | 27.500 |
| | (3.655) | (4.162) | (4.608) | (3.369) | (3.381) |
| Min, Max | 22.16, 32.70 | 24.56, 34.80 | 21.82, 33.65 | 21.07, 30.72 | 23.95, 31.83 |
| Completed | 8 | 5 | 5 | 6 | 5 |
| Dosing | | | | | |
| Discontinued | 0 | 1 | 0 | 0 | 0 |

A total of 30 subjects over four cohorts were dosed, which evaluated three different dose levels (3×10¹¹, 6×10¹¹ and 1×10¹² live cells) and two different formulations (with or without added B₆) at the 1×10¹² dose. There were 2 placebo patients in each cohort. Subjects had normal plasma homocysteine levels at baseline. In each cohort, the subjects were randomly assigned to receive either SYNB 1353 or a placebo (6 active/2 placebo per cohort).

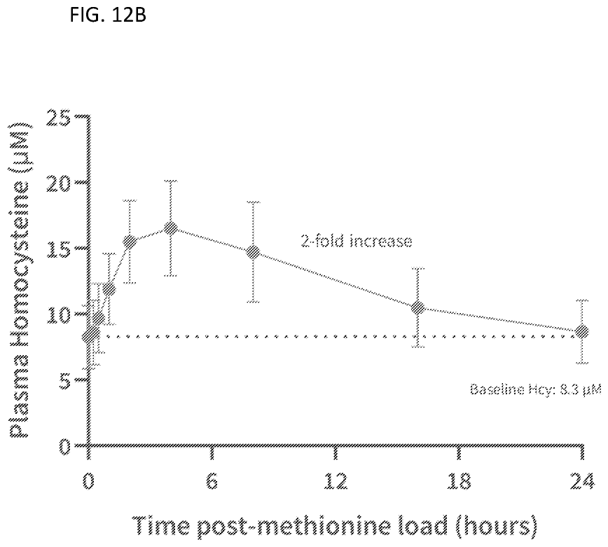

A methionine loading study was performed on day −1 and day 7 after an overnight fast, followed by a 24 hour collection period for the AUC assessments. Subjects were followed in the study for at least 28 days after the last dose. Increased plasma methionine and total Hcy following methionine challenge was observed through 24 h after receiving a 30 mg/kg methionine load challenge. (FIGS. 12A and 12B). Methionine challenge resulted in an 11-fold increase in plasma methionine (Cmax) and a 2-fold increase in plasma total Hcy (Cmax) compared to baseline values.

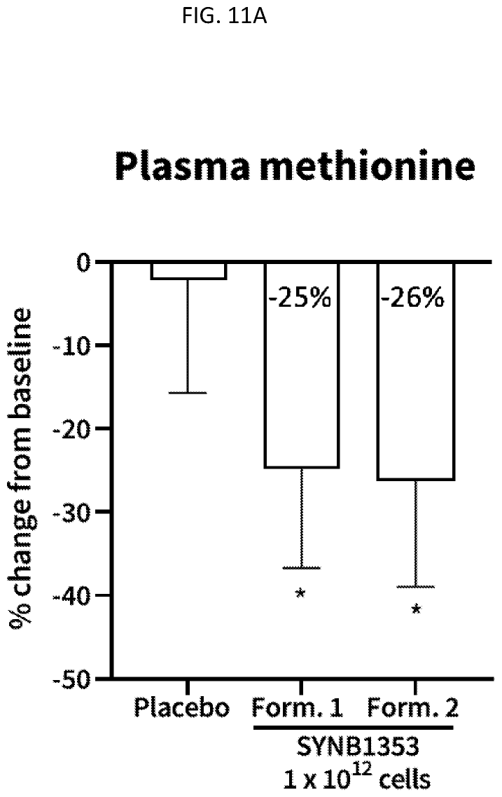

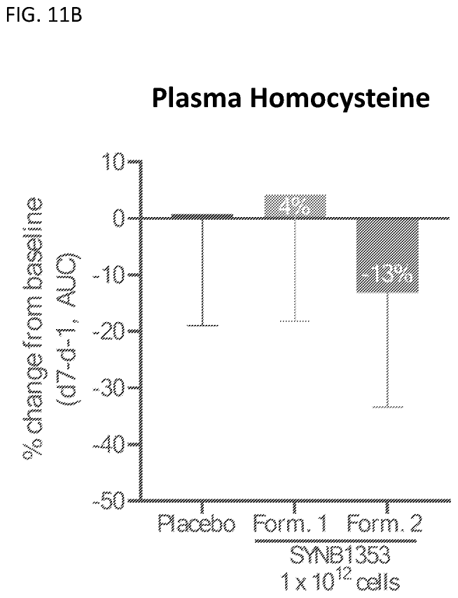

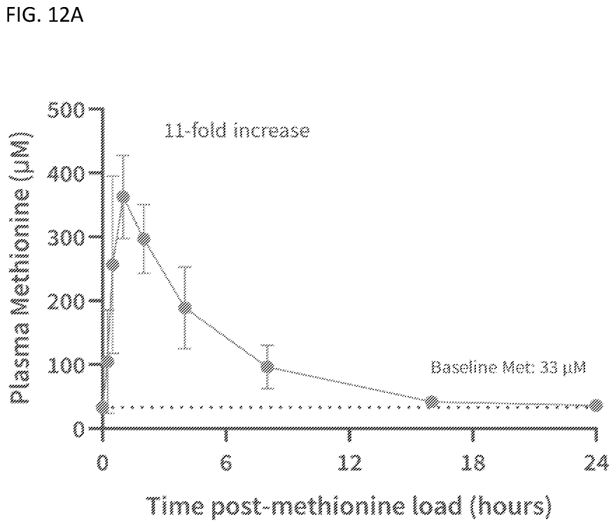

At the 1×10¹² dose, SYNB 1353 decreased plasma methionine levels, as measured by the change in AUC from baseline, by −24.8% (95% CI −36.7, −10.6) and −26.2% (95% CI −39.0, −10.9) for the two different SYNB1353 formulations, compared to −2.1% (95% CI −15.7, 13.7) in the placebo group (FIG. 11A). Plasma homocysteine levels were also tested, and Form. 2 showed a trend towards decreasing levels of homocysteine from baseline (FIG. 11B).

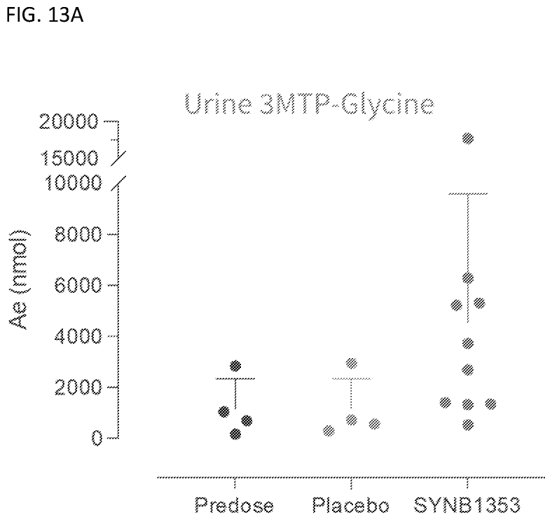

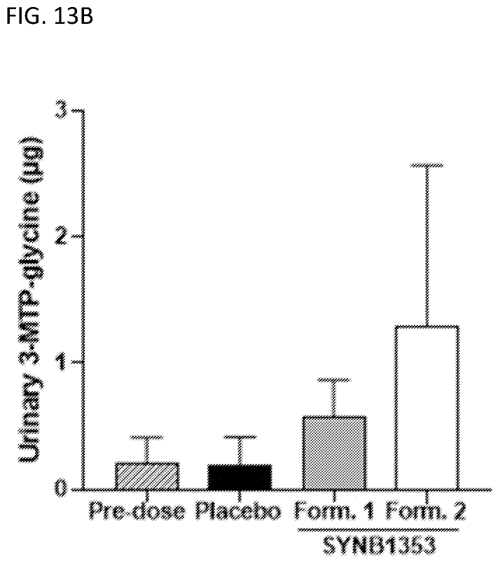

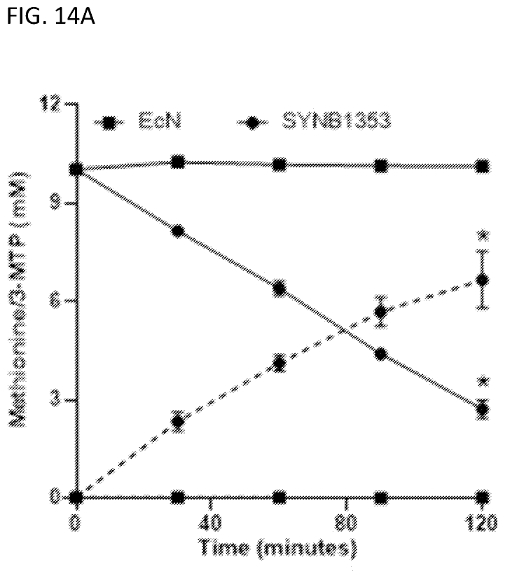

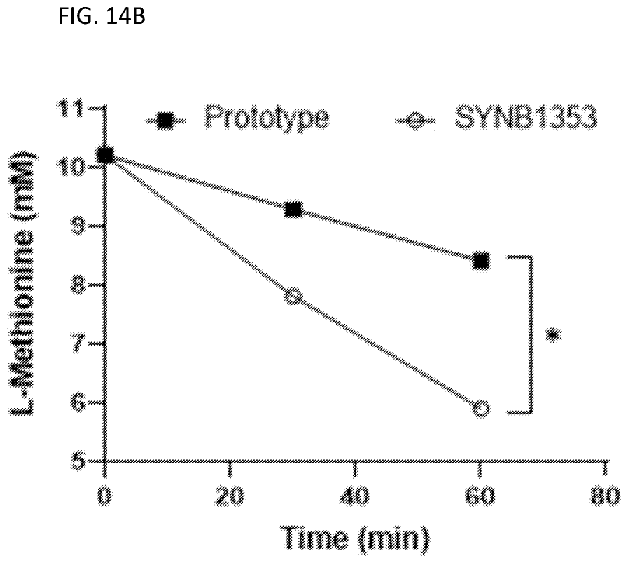

3MTP-glycine is increased in SYNB1353 dosed subjects (FIG. 13). Total amount of 3-MTP-glycine in urine was determined for all subjects pre-dose on day −1 (n=30), all placebo dosed subjects (n=8) and all subjects dosed with SYNB1353 (n=22) on day 7. Urine was collected for 24 h after methionine challenge on either day −1 (pre-dose) or on day 7 for placebo and SYNB1353 dosed subjects. Table 17 shows the number and percentage of positive samples in each cohort and the corresponding mean.

TABLE 17
Number and percentage of positive samples

| | SYNB1353 | % Positive | Mean Ae | Mean ae |
|---|---|---|---|---|
| Cohort | Dose | Samples | (nmol) | (mg) |
| Placebo | — | 50% (4/8) | 1139 | 0.20 |
| SYNB1353 | 3 × 10¹¹ | 17% (1/6) | 1330 | 0.24 |
| | 6 × 10¹¹ | 20% (1/5) | 5303 | 0.94 |
| | 1 × 10¹²* | 73% (8/11) | 4865 | |
| | 1 × 10¹²** | 67% (4/6) | | 0.58 |
| | 1 × 10¹²*** | 80% (4/5) | | 1.29 |

*pooled data from both cohorts receiving 1 × 10¹² live cells.
**Formulation 1
***Formulation 2

These data provide proof of mechanism by demonstrating the effects of SYNB1353's GI-based metabolism of methionine on both plasma methionine and homocysteine, in healthy volunteers. Given the presence of functioning CBS enzyme in healthy individuals, more moderate effects on homocysteine levels as compared to methionine levels were observed. In a patient having a defective CBS enzyme, the level of methionine lowering would more directly reflect the level of homocysteine lowering.

SYNB 1353 has demonstrated methionine metabolism in the GI tract of healthy volunteers, resulting in a lowering of plasma methionine and production of 3MTP-glycine, assessed following a meal challenge to elevate methionine levels. SYNB 1353 was well tolerated in healthy volunteers with GI adverse event rates and severity similar between active and placebo groups. There were no SAEs. One subject discontinued dosing due to an adverse event. Adverse events (AEs) were mild to moderate, transient and predominantly GI in nature. Frequency and severity of GI-related AEs were similar in the SYNB 1353 and placebo groups (7 of 22 SYNB1353 (31.8%) compared to 3 of 8 placebo (37.5%) subjects had at least 1 GI-related AE). All subjects completing the 28 day analysis cleared SYNB 1353 in feces.

TABLE 18
Adverse events in healthy subjects receiving SYNB1353 or placebo

| | Dose | Dose | Dose | Dose | Total | |
|---|---|---|---|---|---|---|
| | 3E+11 | 6E+11 | 1E+12 | 1E+12B | SYNB1353 | Placebo |
| Cohort | (n = 6) | (n = 5)* | (n = 6) | (n = 5)* | (n = 22) | (n = 8) |
| Subjects with at | 3 | 1 | 4 | 3 | 11 | 3 |
| least one TEAE | | | | | | |
| Maximum Grade 1 | 0 | 1 | 0 | 3 | 4 | 0 |
| Maximum Grade 2 | 3 | 0 | 4 | 0 | 7 | 3 |
| Total Number of TEAE | 16 | 2 | 17 | 3 | 38 | 9 |
| Subjects with at | 3 | 0 | 5 | 3 | 11 | 1 |
| least one IMP- | | | | | | |
| related TEAE | | | | | | |
| Maximum Grade 1 | 1 | 0 | 0 | 1 | 3 | 1 |
| Maximum Grade 2 | 2 | 0 | 5 | 2 | 9 | 0 |
| Any SAE | 0 | 0 | 0 | 0 | 0 | 0 |
| TEAE causing | 0 | 0 | 0 | 0 | 0 | 0 |
| withdrawal | | | | | | |

*in both cohorts 6E+11 and 1E+12 B, one volunteer randomized to SYNB1353 withdrew due to AE prior to receiving IMP

TABLE 19
Additional E. coli Strains

| | | Antibiotic |
|---|---|---|
| Strain No. | Background/genotype | resistance |
| SYN094 | wt Nissle, strepR | Strep |
| SYN7349 | ΔyjeH containing Logic2279, which is a medium-copy p15a | cam, carb, kan |
| | plasmid (pTET, atc induction) expressing a methionine | |
| | decarboxylase from Streptomyces sp. 590 to convert methionine | |
| | into 3-methylthiopropylamine and Logic2375, which is a low- | |
| | copy pSC101 plasmid (pTET, atc induction) expressing an | |
| | endogenous methionine importer (metNIQ) | |
| SYN7815 | ΔyjeH containing Logic2279, which is a medium-copy p15a | Cam, kan, spec |
| | plasmid expressing a methionine decarboxylase from | |
| | Streptomyces sp. 590. to convert methionine into 3- | |
| | methylthiopropylamine, (pTET, atc induction). | |
| | Also containing logic2501 with an engineered MetNIQ importer | |
| | (P281S); (pLacI, IPTG induction) | |
| SYN7816 | ΔyjeH containing Logic2279, which is a medium-copy p15a | Cam, kan, spec |
| | plasmid expressing a methionine decarboxylase from | |
| | Streptomyces sp. 590. to convert methionine into 3- | |
| | methylthiopropylamine; (pTET, atc induction). | |
| | Also containing logic2502 with an engineered MetNIQ importer | |
| | (P281G); (pLacI, IPTG induction) | |
| SYN7817 | ΔyjeH containing Logic2279, which is a medium-copy p15a | Cam, kan, spec |
| | plasmid expressing a methionine decarboxylase from | |
| | Streptomyces sp. 590. to convert methionine into 3- | |
| | methylthiopropylamine; (pTET, atc induction). Also containing | |
| | logic2503 with a recoded MetNIQ importer; (pLacI, IPTG | |
| | induction) | |
| SYN7818 | ΔyjeH containing Logic2279, which is a medium-copy p15a | Cam, kan, spec |
| | plasmid expressing a methionine decarboxylase from | |
| | Streptomyces sp. 590. to convert methionine into 3- | |
| | methylthiopropylamine; (pTET, atc induction). Also containing | |
| | logic2534 with the MetP importer (F. frigoris); (pLacI, IPTG | |
| | induction) | |
| SYN7819 | ΔyjeH containing Logic2279, which is a medium-copy p15a | Cam, kan, spec |
| | plasmid expressing a methionine decarboxylase from | |
| | Streptomyces sp. 590. to convert methionine into 3- | |
| | methylthiopropylamine; (pTET, atc induction). Also containing | |
| | logic2535 with the MetP importer (F. segetis); (pLacI, IPTG | |
| | induction) | |
| SYN7346 | ΔyjeH containing Logic2279, which is a medium-copy p15a | cam, kan |
| | plasmid (pTET, atc induction) expressing a methionine | |
| | decarboxylase from Streptomyces sp. 590. to convert methionine | |
| | into 3-methylthiopropylamine. | |
| SYN7640 | ΔyjeH, recoded Streptomyces MetDC, pSC101 (pTET, atc | Cam, kan |
| | induction) | |
| SYN7641 | ΔyjeH, protein engineered MetDC (V491L A500P) with high | Cam, kan |
| | 3MTP production, pSC101 (pTET, atc induction) | |
| SYN7642 | ΔyjeH, protein engineered MetDC (Q70D N82H) with high | Cam, kan |
| | 3MTP production, pSC101 (pTET, atc induction) | |
| SYN7643 | ΔyjeH, protein engineered MetDC (R41Q Q70D) with high | Cam, kan |
| | 3MTP production, pSC101 (pTET, atc induction) | |
| SYN7644 | ΔyjeH, Stanieria sp. NIES-3757 Methionine decarboxylase from | Cam kan |
| | MetDC metagenomic library screen with high 3MTP production, | |
| | pSC101 (pTET, atc induction) | |
| SYN7689 | SYN7345 ΔyjeH, containing Logic2491, which expresses an | Cam kan |
| | engineered MetDC from Mus musculus. pSC101 (pTET, atc | |
| | induction) | |
| SYN7690 | SYN7345 ΔyjeH, containing Logic2492, which expresses an | Cam kan |
| | engineered LeuDC from Mus musculus. pSC101 (pTET, atc | |
| | induction) | |
| SYN7691 | SYN7345 ΔyjeH, containing Logic2493, which expresses a | Cam kan |
| | MetDC from Entamoeba histolytica. pSC101 (pTET, atc | |
| | induction) | |
| SYN7345 | EcN with endogenous methionine and branched-chain a.a. | cam |
| | exporter (yjeH) knocked out. | |
| SYN7970 | (lacI-Ptac, IPTG) 2x MetDC (Q70D N82H); 1x MetP (F. segetis); | none |
| | Δdap; ΔyjeH, Δprophage | |
| SYN8002 | (lacI-Ptac, IPTG) 3x MetDC (Q70D N82H); 1x MetP (F. segetis); | None |
| | Δdap; ΔyjeH; Δ_{Φ} | |
| SYN8003 | (lacI-Ptac, IPTG) 3x MetDC (Q70D N82H); 1x MetP (F. segetis); | none |
| | Δdap; ΔyjeH; Δ_{Φ} (stronger RBS than SYN8002) | |
| SYN8070 | (lacI-Ptac, IPTG) 3x MetDC (Q70D N82H); 1x MetP (F. segetis); | None |
| | Δprophage; Δdap; ΔyjeH; Δclb | |
| | (integration: thiC/rsd::attB2-lacI-P_{tac}-metDC; glmS/pstS::attB5- | |
| | lacI-P_{tac}-metP-P_{tac}-metDC; hypothetical protein/yfjJ::attB7-P_{tac}- | |
| | metDC) | |
| SYNB1353 | (lacI-Ptac, IPTG) 3x MetDC (Q70D N82H); 1x MetP (F. segetis); | None |
| | Δprophage; Δdap; ΔyjeH; Δclb | |
| | (integration: thiC/rsd::attB2-lacI-P_{tac}-metDC; glmS/pstS::attB5- | |
| | lacI-P_{tac}-metP-P_{tac}-metDC; hypothetical protein/yfjJ::attB7-P_{tac}- | |
| | metDC) | |

TABLE 20
Exemplary Methionine Decarboxylase Nucleotide Sequences

| Description | |
|---|---|
| SEQ ID NO: | Nucleotide Sequence |
| MetDC Q70D | ATGtccccgacggcgtttccagcggccgaaacagc |
| N82H | tactgcccctgcaactgccgtcgatcctgggccag |
| SEQ ID NO: | aactggacggcggagatttcgcccttccagagggc |
| 627 | gggctggatgacgatcgtcgcttacgtgcattgga |
| | cgcagttgacgagtatttgacccgcaagcgcaagc |
| | atttggttgggtaccaagctacccaggatatggac |
| | ggaacggccttggatttagcccgtttcatgcccca |
| | caacatcaacaacctgggagatcctttccagtcgg |
| | ggggtataaaccaaatacgaaagtcgttgagcgtg |
| | ccgtactggactactatgcaaaattgtggcacgca |
| | gaacgtccacacgacccagctgacccagaaagcta |
| | ctggggttacatgttatcgatgggctcaactgagg |
| | gcaacatgtacgccctgtggaatgcacgtgactac |
| | ctgtcgggtaaggctttgattcagcctcccacggc |
| | accatttgacgctgttcgctacgtgaaggctgacc |
| | ccgatcgccgcaatcctaacgcacaccacccagtc |
| | gcattctactcggaggatacccactattcttttgc |
| | taaagccgttgcggtgctgggtgtcgaaactttcc |
| | acgctgtgggtctggagaaatacgctgacgagtgc |
| | cccttggtggatccagtaaccggccttcgtacctg |
| | gccgaccgaagttccatcgcgcccggggccgtcgg |
| | gtttaagctgggacggccctggtgagattgatgtt |
| | gatgcgcttgcagtactggtcgagttcttcgcagc |
| | gaagggtcaccccgtcttcgtcaaccttaacttgg |
| | ggtctacatttaaaggagcacatgatgacgtacgt |
| | gcggtatgtgaacgcttattaccaatcttcgagcg |
| | ccatggcttagtacaacgtgaagttgtatatggga |
| | gctgtccccaaaccggccgccctttagtggatgta |
| | cgtcgcggattttggatccacgtagatggggcact |
| | tggggggggtatgccccttttctgcgtcttgccgc |
| | cgaagacccggaaggttatggttggacccctgagg |
| | cagaattacctgagttcgacttcggcttacgtttg |
| | ccgacggcggggcatggagaagttgatatggttag |
| | cagcatcgccatgagtggacataagtgggcaggcg |
| | cgccgtggccatgcggcatctatatgacgaaagtg |
| | aaatatcagattagtccaccgtcacagcccgatta |
| | tattggtgctcctgacacaacatttgccggttccc |
| | gtaacggcttttcgccgttaattttgtgggatcat |
| | ttatcgcgctactcgtaccgcgaccaggtagagcg |
| | catccgcgaagcacaggagcttgcagcatatttgg |
| | aacgccgccttaccgctatggagcgcgagctggga |
| | gtggaactttggccagcccgcacaccgggtgctgt |
| | aaccgtacgttttcgcaaaccctctgctgagctgg |
| | ttgcgaagtggtccttgtcgtcgcaggatgtttta |
| | atggtgccgggtgatgaaactacgcgtcgtagtta |
| | cgttcatgtgttcgtgatgccttctgttgatcgtg |
| | caaagttagatgcgttgctggcagaattggccgaa |
| | gatcccgtcatcttgggtgcgccttaa |
| MetDC | ATGTCCCCGACGGCGTTTCCAGCGGCCGAAACAGC |
| (Streptomyces) | TACTGCCCCTGCAACTGCCGTCGATCCTGGGCCAG |
| SEQ ID NO: | AACTGGACGGCGGAGATTTCGCCCTTCCAGAGGGC |
| 596 | GGGCTGGATGACGATCGTCGCTTACGTGCATTGGA |
| | CGCAGTTGACGAGTATTTGACCCGCAAGCGCAAGC |
| | ATTTGGTTGGGTACCAAGCTACCCAGGATATGCAG |
| | GGAACGGCCTTGGATTTAGCCCGTTTCATGCCCAA |
| | CAACATCAACAACCTGGGAGATCCTTTCCAGTCGG |
| | GTGGGTATAAACCAAATACGAAAGTCGTTGAGCGT |
| | GCCGTACTGGACTACTATGCAAAATTGTGGCACGC |
| | AGAACGTCCACACGACCCAGCTGACCCAGAAAGCT |
| | ACTGGGGTTACATGTTATCGATGGGCTCAACTGAG |
| | GGCAACATGTACGCCCTGTGGAATGCACGTGACTA |
| | CCTGTCGGGTAAGGCTTTGATTCAGCCTCCCACGG |
| | CACCATTTGACGCTGTTCGCTACGTGAAGGCTGAC |
| | CCCGATCGCCGCAATCCTAACGCACACCACCCAGT |
| | CGCATTCTACTCGGAGGATACCCACTATTCTTTTG |
| | CTAAAGCCGTTGCGGTGCTGGGTGTCGAAACTTTC |
| | CACGCTGTGGGTCTGGAGAAATACGCTGACGAGTG |
| | CCCCTTGGTGGATCCAGTAACCGGCCTTCGTACCT |
| | GGCCGACCGAAGTTCCATCGCGCCCGGGGCCGTCG |
| | GGTTTAAGCTGGGACGGCCCTGGTGAGATTGATGT |
| | TGATGCGCTTGCAGTACTGGTCGAGTTCTTCGCAG |
| | CGAAGGGTCACCCCGTCTTCGTCAACCTTAACTTG |
| | GGGTCTACATTTAAAGGAGCACATGATGACGTACG |
| | TGCGGTATGTGAACGCTTATTACCAATCTTCGAGC |
| | GCCATGGCTTAGTACAACGTGAAGTTGTATATGGG |
| | AGCTGTCCCCAAACCGGCCGCCCTTTAGTGGATGT |
| | ACGTCGCGGATTTTGGATCCACGTAGATGGGGCAC |
| | TTGGGGCGGGGTATGCCCCTTTTCTGCGTCTTGCC |
| | GCCGAAGACCCGGAAGGTTATGGTTGGACCCCTGA |
| | GGCAGAATTACCTGAGTTCGACTTCGGCTTACGTT |
| | TGCCGACGGCGGGGCATGGAGAAGTTGATATGGTT |
| | AGCAGCATCGCCATGAGTGGACATAAGTGGGCAGG |
| | CGCGCCGTGGCCATGCGGCATCTATATGACGAAAG |
| | TGAAATATCAGATTAGTCCACCGTCACAGCCCGAT |
| | TATATTGGTGCTCCTGACACAACATTTGCCGGTTC |
| | CCGTAACGGCTTTTCGCCGTTAATTTTGTGGGATC |
| | ATTTATCGCGCTACTCGTACCGCGACCAGGTAGAG |
| | CGCATCCGCGAAGCACAGGAGCTTGCAGCATATTT |
| | GGAACGCCGCCTTACCGCTATGGAGCGCGAGCTGG |
| | GAGTGGAACTTTGGCCAGCCCGCACACCGGGTGCT |
| | GTAACCGTACGTTTTCGCAAACCCTCTGCTGAGCT |
| | GGTTGCGAAGTGGTCCTTGTCGTCGCAGGATGTTT |
| | TAATGGTGCCGGGTGATGAAACTACGCGTCGTAGT |
| | TACGTTCATGTGTTCGTGATGCCTTCTGTTGATCG |
| | TGCAAAGTTAGATGCGTTGCTGGCAGAATTGGCCG |
| | AAGATCCCGTCATCTTGGGTGCGCCTtaa |
| MetDC (V491L | atgtccccgacggcgtttccagcggccgaaacagc |
| A500P) | tactgcccctgcaactgccgtcgatcctgggccag |
| SEQ ID NO: | aactggacggcggagatttcgcccttccagagggc |
| 628 | gggctggatgacgatcgtcgcttacgtgcattgga |
| | cgcagttgacgagtatttgacccgcaagcgcaagc |
| | atttggttgggtaccaagctacccaggatatgcag |
| | ggaacggccttggatttagcccgtttcatgcccaa |
| | caacatcaacaacctgggagatcctttccagtcgg |
| | ggggtataaaccaaatacgaaagtcgttgagcgtg |
| | ccgtactggactactatgcaaaattgtggcacgca |
| | gaacgtccacacgacccagctgacccagaaagcta |
| | ctggggttacatgttatcgatgggctcaactgagg |
| | gcaacatgtacgccctgtggaatgcacgtgactac |
| | ctgtcgggtaaggctttgattcagcctcccacggc |
| | accatttgacgctgttcgctacgtgaaggctgacc |
| | ccgatcgccgcaatcctaacgcacaccacccagtc |
| | gcattctactcggaggatacccactattcttttgc |
| | taaagccgttgcggtgctgggtgtcgaaactttcc |
| | acgctgtgggtctggagaaatacgctgacgagtgc |
| | cccttggtggatccagtaaccggccttcgtacctg |
| | gccgaccgaagttccatcgcgcccggggccgtcgg |
| | gtttaagctgggacggccctggtgagattgatgtt |
| | gatgcgcttgcagtactggtcgagttcttcgcagc |
| | gaagggtcaccccgtcttcgtcaaccttaacttgg |
| | ggtctacatttaaaggagcacatgatgacgtacgt |
| | gcggtatgtgaacgcttattaccaatcttcgagcg |
| | ccatggcttagtacaacgtgaagttgtatatggga |
| | gctgtccccaaaccggccgccctttagtggatgta |
| | cgtcgcggattttggatccacgtagatggggcact |
| | tggggggggtatgccccttttctgcgtcttgccgc |
| | cgaagacccggaaggttatggttggacccctgagg |
| | cagaattacctgagttcgacttcggcttacgtttg |
| | ccgacggcggggcatggagaagttgatatggttag |
| | cagcatcgccatgagtggacataagtgggcaggcg |
| | cgccgtggccatgcggcatctatatgacgaaagtg |
| | aaatatcagattagtccaccgtcacagcccgatta |
| | tattggtgctcctgacacaacatttgccggttccc |
| | gtaacggcttttcgccgttaattttgtgggatcat |
| | ttatcgcgctactcgtaccgcgaccaggtagagcg |
| | catccgcgaagcacaggagcttgcagcatatttgg |
| | aacgccgccttaccgctatggagcgcgagctggga |
| | gtggaactttggccagcccgcacaccgggtgctct |
| | gaccgtacgttttcgcaaaccctctccggagctgg |
| | ttgcgaagtggtccttgtcgtcgcaggatgtttta |
| | atggtgccgggtgatgaaactacgcgtcgtagtta |
| | cgttcatgtgttcgtgatgccttctgttgatcgtg |
| | caaagttagatgcgttgctggcagaattggccgaa |
| | gatcccgtcatcttgggtgcgccttaa |
| MetDC (R41Q | atgtccccgacggcgtttccagcggccgaaacagc |
| Q70D) | tactgcccctgcaactgccgtcgatcctgggccag |
| SEQ ID NO: | aactggacggcggagatttcgcccttccagagggc |
| 629 | gggctggatgacgatcagcgcttacgtgcattgga |
| | cgcagttgacgagtatttgacccgcaagcgcaagc |
| | atttggttgggtaccaagctacccaggatatggac |
| | ggaacggccttggatttagcccgtttcatgcccaa |
| | caacatcaacaacctgggagatcctttccagtcgg |
| | ggggtataaaccaaatacgaaagtcgttgagcgtg |
| | ccgtactggactactatgcaaaattgtggcacgca |
| | gaacgtccacacgacccagctgacccagaaagcta |
| | ctggggttacatgttatcgatgggctcaactgagg |
| | gcaacatgtacgccctgtggaatgcacgtgactac |
| | ctgtcgggtaaggctttgattcagcctcccacggc |
| | accatttgacgctgttcgctacgtgaaggctgacc |
| | ccgatcgccgcaatcctaacgcacaccacccagtc |
| | gcattctactcggaggatacccactattcttttgc |
| | taaagccgttgcggtgctgggtgtcgaaactttcc |
| | acgctgtgggtctggagaaatacgctgacgagtgc |
| | cccttggtggatccagtaaccggccttcgtacctg |
| | gccgaccgaagttccatcgcgcccggggccgtcgg |
| | gtttaagctgggacggccctggtgagattgatgtt |
| | gatgcgcttgcagtactggtcgagttcttcgcagc |
| | gaagggtcaccccgtcttcgtcaaccttaacttgg |
| | ggtctacatttaaaggagcacatgatgacgtacgt |
| | gcggtatgtgaacgcttattaccaatcttcgagcg |
| | ccatggcttagtacaacgtgaagttgtatatggga |
| | gctgtccccaaaccggccgccctttagtggatgta |
| | cgtcgcggattttggatccacgtagatggggcact |
| | tggggggggtatgccccttttctgcgtcttgccgc |
| | cgaagacccggaaggttatggttggacccctgagg |
| | cagaattacctgagttcgacttcggcttacgtttg |
| | ccgacggcggggcatggagaagttgatatggttag |
| | cagcatcgccatgagtggacataagtgggcaggcg |
| | cgccgtggccatgcggcatctatatgacgaaagtg |
| | aaatatcagattagtccaccgtcacagcccgatta |
| | tattggtgctcctgacacaacatttgccggttccc |
| | gtaacggcttttcgccgttaattttgtgggatcat |
| | ttatcgcgctactcgtaccgcgaccaggtagagcg |
| | catccgcgaagcacaggagcttgcagcatatttgg |
| | aacgccgccttaccgctatggagcgcgagctggga |
| | gtggaactttggccagcccgcacaccgggtgctgt |
| | aaccgtacgttttcgcaaaccctctgctgagctgg |
| | ttgcgaagtggtccttgtcgtcgcaggatgtttta |
| | atggtgccgggtgatgaaactacgcgtcgtagtta |
| | cgttcatgtgttcgtgatgccttctgttgatcgtg |
| | caaagttagatgcgttgctggcagaattggccgaa |
| | gatcccgtcatcttgggtgcgccttaa |
| MetDC | atggggttccagttactgtctaaacataagctgtc |
| (Stanieria sp. | agccgaggatcaacagaaacttgaccgcttttatc |
| NIES-3757) | gtgatattcagacagaagcagaacgattcctgggt |
| SEQ ID NO: | tacccatgtaacgaactctttgactactccccctt |
| 630 | gttccggttcctgcaatatccgctgaataacgtcg |
| | gcgacccgtacctgccgagtaactaccacctgaac |
| | acgcacaactttgagtgcgaagtactggaaatctt |
| | ccgtaccctgaccgaggctactgaaggttcgactt |
| | ggggctacgtgaccaacggcggtacggaaggtaat |
| | cattatggtcttttcctggcgagagagctgctgcc |
| | tgaaggccttgtttactattctcaggatgcgcact |
| | actcgatcgataaaatcctgaggtgcctcaacctc |
| | cgtagcataatgattcgcagccacgacgacggacg |
| | catggacctggatgatctgcgtgaaactctgcgta |
| | tccatcgcgacttgccgccgatcgtttgcgctacc |
| | attggtactacaatgaagggcgctgtagatgacat |
| | cgcaggcattaaaaagatcttcaaagatctggcaa |
| | tacaccgtcactatatccatgctgacgcggcccta |
| | ggtggcatgattttaccgttcctggataactcccc |
| | accgtggaattttaaagctggaatcgactctatcg |
| | ctatctccggtcacaaaatggtgggcagtcctatc |
| | ccgtgtggggttgtcctggctaaaaagtcgaacgt |
| | tgaacgtattgcacagagcgtggaatacattggta |
| | ctctggataccaccctgtctggctcccgtaacgcc |
| | ttgactccgttatttctgtggtacgcgttccacac |
| | cgttggtatcgaaggtttcaaacgtatcatcccgg |
| | catgcttaaaaatggcggactatgccatcgctcag |
| | ctgaacaaaattaaccgcaatgcgtggcgctaccc |
| | ttacagcaacacggtagtcttcgatcgcccaagcc |
| | ccgaagtgactcgttattggcagctggcttgtcag |
| | ggcaacctgagccacctaatcaccatgccacacgt |
| | tacatctactcaaattgatcatctggttgctgaca |
| | tcatcgcttctgagccgataccgccgctgccgacc |
| | ctgtcagttactccggcatgcgaactgctgacttc |
| | taccccggaccaggatattacgctgatcggcaccg |
| | ctaatcataatctgctctccgaagtatctaccgcc |
| | ctggctgccgagggtctgtcaattgaaaacctggc |
| | tgctgtggcggtagaaagcgaggacgttgaagttg |
| | taaggctccgcgttaacaaccgtgagcgtgcactg |
| | caaatcctgaaccagaacctggatatcggtcgttg |
| | ctacggtcaggctcgaccctttggcaacgaagaag |
| | cgacgcaggtactgtcccagctggaatatcaaagc |
| | gtgggggaggatgcactactggtccagcttgacga |
| | ttgccctggcagcctggcggagctgttgaaggatt |
| | gccgcaacgaagcggtaaaaatccgtaatatccga |
| | ctgctttggcgtgggcacggtaagggcgtcgtagc |
| | aattgctaccacttctccagatgcgctgaaaacgc |
| | tgctgaaagaccgtattcttttgagctaa |
| MetDC | ATGtccacacctagtgaagtaaagaaggatttgct |
| (Mus musculus) | gggtgcagcagggtcattatggccgtcggagccca |
| SEQ ID NO: | ttacgctgggtccaggtgaaagtgcttggcagctg |
| 631 | gtattgaagaagatccaagagttgagtgacagcgg |
| | tcatcaagacccgttcatggttgcagaccttgatg |
| | tccttgtgtctcgtcatcagacgttctgtcaagca |
| | ctgcctagagtacaacccttctatgcagtaaagtg |
| | caatagtaacccatgggtgttacgggtgttggcag |
| | ctcttggcacgggatttgattgtgcttctcaggga |
| | gaattggagcaagttttgggcttgggtgtagcgcc |
| | gtcacggataatcttcgcaaatccctgtaaagcag |
| | tcagccacattcagtttgcagctcggtgcggtgtg |
| | caattgttgacattcgacagcgaagaggagttaat |
| | caaggttgcgcagtaccatccaggcgcacggttgg |
| | tgcttcggattcaaacccaggactcacaatcaacg |
| | ttcccactttccaccaagttcggtgcttctttaga |
| | agcatgtggacaccttctgcaggttgccagagagc |
| | tgggtcttgccgtggtaggtgctagctttcatgta |
| | ggaagcgactgccacacacctcagagttttcgtca |
| | ggccatcgcagattgtcatcgtgtgttcgagatgg |
| | gccgtaaggcaggtcatgatatgtcgcttcttgat |
| | ttgggtggagggttcccaggtgtggaaggttccga |
| | ggcgaagtttgaggagatggcaagagtaatcaatg |
| | ccgctcttgctcagtactttccggaagagactggc |
| | atcgaggtgatcgcggaacctggtcgtttctacgc |
| | tgggtcggtgtgcactgcagctgtgaacatcatcg |
| | cgaagaagtctgtcttggaaccaggtggtcatcgt |
| | aagcttatgtactaccttaatgaaggacattacgg |
| | ttctttcagattgttcttgcgtgatccagtgcctc |
| | gtattcccatcgtggtgaaagagttcccatccgaa |
| | ccaccactgtttccttgcactttgtacggtcccac |
| | atgtgacgcctatgatcggttgttttccgaagagg |
| | tacaattgccagagctggatgttggagattggttg |
| | atcttcccagatatgggtgcctatacctcctcaat |
| | gtcctcgaccttcaacggatttccaccggccaccg |
| | tgtattgcgcaatgtcaccgcagttacgctccctg |
| | ttggagactgtaccataa |
| LeuDC | atgaacacacctagtgaagtaaagaaggatttgct |
| (Mus musculus) | gggtgttgcagaacatttacgtccgtcggagccca |
| SEQ ID NO: | ttacgctgggtccaggtgcgagtgcttggcagctg |
| 632 | gtattgaagaagatcaaggagttgagtattagcgg |
| | tcgtcaagacgctttcatggttgcagaccttgatg |
| | tccttgtgtctcgtcatcggacgttcttacaagca |
| | ctgcctagagtacaacccttctatgcagtaaagtg |
| | caatagtaacccatgggtgttacttgtgttggcag |
| | ctcttggcacgggatttgattgtgcttctcaggga |
| | gaattggagcaagttttgggcttgggtgtagcgcc |
| | gtcacggataatcttcgcaaatccctgtaaagcag |
| | tcagccacattcagtttgcagctcggtgcggtgtg |
| | caattgttgacattcgacaatgaagaggagttaat |
| | caagttagcgcgttaccatccacgtgcacggttgg |
| | tgcttcggattcaaaccctggactcacaatcaacg |
| | ttcccacttagcaccaagttcggtgctcacttaga |
| | agcatgtggacaccttctgcaggttgccagagagc |
| | tgggtcttgccgtggtaggtgctagctttcatgta |
| | ggaagcgactgccacacacctgagagttaccgtca |
| | ggccatcgcagattgtcatcgtgtgttcgagatgg |
| | gctgtaaggcaggtcatcacatgtcgcttcttgat |
| | ttgggtggagggttcccaggtgtgaaaggttccga |
| | ggcgaagtttgaggaggttgcaagagtaatcaata |
| | ccgctcttgctcagtactttccggaagagactggc |
| | atcgaggtgatcgcggaacctggtcgtttctacgc |
| | tgggtcggtgtgcactgcagctgtgaacatcatcg |
| | ccaagaagtctagtttggacccaggtggtcatcgt |
| | aagcttgcttactaccttaatgaaggacattacgg |
| | tgtattcagattgttcttgcgtgatccagtgcctc |
| | gtattcccatcgtggtgaaagagttcccatccgaa |
| | ccaccactgtttccttgcactttgtacggtcccac |
| | atgtgacgcctatgatcggttgttttccaccgagg |
| | tacaattgccagagctggatgttggagattggttg |
| | atcttcccagatatgggtgcctattcgtcctcaat |
| | gtcctcgaccttcaacggatttccaatagccaccg |
| | tgtatgatgcaatgtcaccgcagttacgctccctg |
| | ttggagactgtaccataa |
| MetDC | atgaaacaaacgtcccttgaggtgaaggaatttgc |
| (Entamoeba | cttgaatctcatttctcagttcgaaccagaaaacc |
| histolytica) | agcctctgggtttctggatattcgacaccgaaggc |
| SEQ ID NO: | gttgagaaagcggtagaacgctggaaaaagaacat |
| 633 | gccgactgtccgtccctgttttgcagttaaatgca |
| | acccggagccgcacctggtgaaattactgggggaa |
| | ctgggttgcggcttcgattgcgctagcctgaacga |
| | aatcaaagaggtactggacttgggttttaatccgg |
| | aagatatcacttatagtcagaccttcaaaccgtac |
| | aaccagttaattgaagcttcgcatctgggcatcaa |
| | ccacacgatcgttgattcaatcgacgaagttcaaa |
| | aaattgctaaatacgcgcctaagatgggtatcatg |
| | attcggatcatggaaaatgacacaagcgcaggcca |
| | cgtctttggagagaaattcggtctgcatgatgatg |
| | aagttgagatcgtactgaaggaaattaaagacaaa |
| | ggtctgaacctggacggcgttcatttccacgttgg |
| | ctctgattcccacaacagcgaagtgtttactaagg |
| | cactgaccaaagctcgtaacactgtaaccctggcc |
| | gaacagttcggcatgaaaccgtacctgatcgacat |
| | tggtggcgggttctctcaggttgcgccgttcgaag |
| | aatttgctgctaccatcgaaaaaactataaaggaa |
| | ctggaatttccagagcgaactcgtttcattgcaga |
| | gccgggtcgctatatggcatcaaatgcctttcacc |
| | ttgtctcttcgctgcatggtaaaagggtgcgcatc |
| | cagaacggtaagaaacagatcgaatacaccagcgg |
| | cgatgggctgcacggctccttcggctgttgcatct |
| | ggttcgaaaaacagaagtcttgcgaatgtataaca |
| | caaaaagtaaacgagaacaccaaaatgtatgaaag |
| | catcatctacggcccatcttgcaacggttcggaca |
| | aagtggccacgcaggagttgccggaaatggagccg |
| | ggtaaagattggctgctgttccccaatatgggtgc |
| | ttacactatttccatggcgaccaactttaacggct |
| | tcgaagaacgtaaccatgtaatctatacgttacca |
| | ctcaaaagtactaaaataattcagatccctaaaag |
| | cattgaatgcaactccgttccgtctttaaacggaa |
| | tcccacactacgcgtaa |

TABLE 21
Exemplary Transporter Nucleotide Sequences

| Description | |
|---|---|
| SEQ ID NO: | Nucleotide Sequence |
| MetP | atggggaccattaacacgaagatctataaatacatgagca |
| (F. segetis) | tctggaaaacaaaacctctgtccgtgctcttgtctgaagc |
| SEQ ID NO: | aactgaggatgaaaaaggcctgaagcgcactctgtcggcc |
| 634 | cgttcacttgttgcgctgggtgtcggtgctattatcggcg |
| | ctggtttattctctctgaccggcatagctgcggcagacaa |
| | tgctggaccggcagtaaccctgagctttatcctggcctcc |
| | gttggttgcgcgttcgctggcctgtgttacgcagaatttg |
| | cttctatgattccagttgcgggtagcgcctacacttatag |
| | ttatgctaccatgggcgagttcgtggcgtggatcatcggt |
| | tgggatctggtactcgaatacgcattgggcgcagctactg |
| | ttgccgttagctggtcccagtacgtggacaaattcttgca |
| | aaactacggcatccatattccgaactctatcctccacggg |
| | ccgtgggataccacccccggtattatcaatttaccgtcga |
| | tatttatcatctgcctgctgagcgtgctgctgattcgtgg |
| | tactaaagaatctgctctgatcaacaacattctggtaatc |
| | ctgaaagtcacggttgtcatcgtgttcattggcctgggct |
| | gggggttcatgaactccgcaaaccacacgccctttatccc |
| | ggttaacgaaggtgaggctctactgtcttctggtgaaatg |
| | agtttcctcaactttttcagcagtgactactttggacact |
| | acggatggtccggtattcttcgcggcgctggtgtagtatt |
| | cttcgcatttatcggcttcgacgcggtgagcactgcggca |
| | caggaggccaaggatccgcagaaaggcatgccaatcggta |
| | ttctgggctcactgatcatttgcaccgttctgtacgtgct |
| | tttcgctttcgttctgaccggtctggaaaactatctaaac |
| | ttcaaaggtgacgcttctcctgtcaccactgcatttgcca |
| | aaacaggctatactttcctgaatagcggtctgacgatcgc |
| | tatcatagcgggctacacatccgttatgctggtaatgttg |
| | atgggtcagtcccgtgtcttttatagtatgtctgtggatg |
| | gcctgcttccgaagtttttctcgaccctgcataccaaaaa |
| | caggactccgtacaaaactaatttgctgttcatggttttc |
| | gtaagcctgttcgctggctttgttccggtcagcgacctgg |
| | gccatatggtatccatcggtaccctcttcgctttctgcct |
| | ggtgtgtatcggcgttatcgttatgcgaaaaaccaaccca |
| | gacgccgttcgcggttttcgtgttccttttgtaccggttt |
| | tcccgattatcggtgtagttatttgtctggttctaatggc |
| | gggcctgccgattgaatcttgggaacgtctggcgatctgg |
| | atgattctgggtgtcgtgatctacttcttctactctaaaa |
| | agaactctaaactgaataaccccgaataa |
| MetP | atggggacgatcaatactaagaccaacaaatatatgagca |
| (F. frigoris) | tttggaaaaccaaaccgttgtctgtactgttaaacgaggc |
| SEQ ID NO: | ctcagaagatgaaaagggcctgaaaaggactctgtcctct |
| 635 | cgttccctcgtggctctgggtgtcggtgcgatcattggcg |
| | caggtctgtttagcctaacaggcatcgcagctgcggaaca |
| | tgctggtccagcggttactctgagtttcatactggccgct |
| | gttggttgtgctttcgcaggcctgtgctacgcggagtttg |
| | cgtcgatgatccctgtggctgggtctgcttacacctatag |
| | ctacgcaaccatgggcgaatttatggcgtggatcattggc |
| | tgggaccttgtactggaatacgctctgggtgcagcgactg |
| | ttggtgtatcctggtcccgttacttactggaattgctgaa |
| | caaatatggtgttcacctgaacccgaaattcatctgctct |
| | ccgtgggagacacttaccctgggcgacggcactattatcg |
| | atgggggtacatcaatctgccggcaattctgatcgtgagc |
| | gccctcagcttgctgctgattagaggtacccaggaatctg |
| | cttctattaacaacatcctggttgtgctgaaagtaatagt |
| | cgtgatcatgttcatcgttttaggatgggactatatcgat |
| | cccgcaaattactcaccttacatcccggaaaacaccggcg |
| | taaagggccaattcggttggtcgggtatcgctgcgggtgc |
| | tggtacggttttctttgccttcattggtttcgacgccgtt |
| | tccactgcggctcaggaggctaaaaacccgcagaaaggca |
| | tgccaattggcatcctggggtctttggtaatttgtacgat |
| | cctgtacgtcctttttgcccacgttatgacgggcctggtg |
| | ccgtattataagttcgctggagatgctaaacccgctgcga |
| | cagcattcgcagtcaccggttacagttttctgcaaactgg |
| | actgattgttgcgatcctggctggctatactagcgttatg |
| | ctggtcatgctgatggggcagagtcgtgttttctacacca |
| | tgagcaaagacggtctgctaccaccgctgttcggtcagat |
| | ccattcgaaatttcgcactccgtacaagactaacctgttc |
| | tttatggtattcgtttctttattcgcgggtttcgtgccgg |
| | ttagcgacctcggccacatggtcagcatcggtaccctcct |
| | ggcgtttgttcttgtgtgcataggtgtgctggtgatgcga |
| | aaaaagatgccagatgctccgcgttctttcaaaaccccgt |
| | tcgttccgtatgtacccatcgcaggcgtcctggtgtgcac |
| | ttacctgatgtactccctcccttacgaatcctggattcgc |
| | ttagtgctttggatggctatcggcgtagccctgtacttcg |
| | tgtatggaaaaaagcactcaaaactgaacaatccggataa |
| MetNIQ | atgataaaactttcgaatatcaccaaagtgttccaccagg |
| SEQ ID NO: | gcacccgcaccatccaggcgttgaacaacgtcagcctgca |
| 636 | tgtgccagctggacaaatttatggcgttatcggtgcctca |
| | ggcgcgggtaagagtacgcttatacgttgtgtaaacctgc |
| | tggagcgcccaaccgagggtagcgtgctggtcgatggcca |
| | ggaactgaccacgctgtcagaatccgagttgaccaaagct |
| | cgtcgccagattggtatgattttccagcattttaacctgc |
| | tctcttcgcgtactgtttttggcaacgtggctctgccgct |
| | ggagctggacaacacaccgaaagacgagatcaaacgtcgc |
| | gtgacggaattgctgtcattggttggtcttggcgataagc |
| | atgatagctacccgtcgaatctttccggtgggcagaaaca |
| | acgtgtggcgattgcccgtgcattagccagcaatcccaaa |
| | gtattgctgtgtgatgaagccaccagcgcgctggacccgg |
| | caacgacacgttctattctcgaactgctgaaagacatcaa |
| | ccgccgtctgggtttgacgattctgttgatcactcacgaa |
| | atggacgttgtgaagcgcatttgtgattgcgtggcggtca |
| | tcagcaatggcgaactgatcgagcaggacacggtaagtga |
| | agtgttctcgcatccgaaaacgccgctggcgcagaagttt |
| | attcagtcgaccctgcatctggatatcccggaagattacc |
| | aggaacgtctgcaagcggagccatttactgactgcgtgcc |
| | gatgctgcgtctggagtttaccggtcaatcggtcgatgcc |
| | ccactgctttctgaaaccgcgcgtcgtttcaacgtcaaca |
| | acaacattattagcgcgcagatggattacgccggtggcgt |
| | taagttcggcatcatgctgactgaaatgcacggcacacaa |
| | caagatacgcaagccgccattgcctggctgcaggaacacc |
| | atgtaaaagtagaggtactgggttatgtctgagccgatga |
| | tgtggctgctggttcgtggcgtatgggaaacgctggcaat |
| | gaccttcgtatccggtttttttggctttgtgattggtctg |
| | ccggttggcgttctgctttatgtcacgcgtccggggcaaa |
| | ttattgctaacgcgaagttgtatcgtaccatttctgcgat |
| | tgtgaacattttccgttccatcccgttcattatcttgctg |
| | gtatggatgattccgtttacccgcgttattgtcggtacat |
| | cgattggattgcaggcagcgattgttccgttaaccgttgg |
| | tgcagcaccgtttattgcccgtatggtcgagaacgctctg |
| | ctggagatcccaaccgggttaattgaagcttcccgcgcaa |
| | tgggggccacgccaatgcagatcgtccgtaaagtgctgtt |
| | accggaagcgttgccgggtctggtgaatgcggcaactatc |
| | accctgattaccctggttggttattccgcgatgggtggtg |
| | cagtcggtgccggtggtttaggtcagattggctatcagta |
| | tggctacatcggctacaacgcgacggtgatgaatacggta |
| | ctggtattgctggtcattctggtttatttaattcagttcg |
| | caggcgaccgcatcgtccgggctgtcactcgcaagtaacg |
| | ttcaacacaacataaataattgaagaaggaataaggtatg |
| | gcgttcaaattcaaaacctttgcggcagtgggagccctga |
| | ttggatcactggcactggtaggctgcggtcaggatgaaaa |
| | agatccaaaccacattaaagtcggcgtgattgttggtgcc |
| | gaacagcaggttgcagaagtcgcgcagaaagttgcgaaag |
| | acaaatatggcctggacgttgagctggtaaccttcaacga |
| | ctatgttctgccaaacgaagcattgagcaaaggcgatatc |
| | gacgccaacgccttccagcataaaccgtaccttgatcagc |
| | aactgaaagatcgtggctacaaactggtcgcagtaggcaa |
| | cacatttgtttatccgattgctggttactccaagaaaatc |
| | aaatcactggatgaactgcaggatggttcgcaggttgccg |
| | tgccaaacgacccaactaaccttggtcgttcactgctgct |
| | gctgcaaaaagtgggcttgatcaaactgaaagatggcgtt |
| | ggcctgctgccgaccgttcttgatgttgttgagaacccaa |
| | aaaatctgaaaattgttgaactggaagcaccgcagctacc |
| | gcgctctctggacgacgcgcaaatcgctctggcagttatc |
| | aataccacctatgccagccagattggcctgactccagcga |
| | aagacggtatctttgtcgaagataaagagtccccgtacgt |
| | aaacctgatcgtaacgcgtgaagacaacaaagacgccgaa |
| | aacgtgaagaaattcgttcaggcttatcagtctgacgaag |
| | tttacgaagcagcaaacaaagtgtttaacggcggcgctgt |
| | taaaggctggtaa |
| Methionine | atgtttgagaagtattttccaaatgttgacttgaccgagt |
| import system | tatggaatgccacatatgaaactctgtatatgacattgat |
| permease | ttccttactgtttgccttcgtaatcggcgtcatcctggga |
| protein MetP | ttgctgttattcttaacatctaaggggtctctttggcaaa |
| (Bacillus | ataaagcagtaaattccgttatcgcagccgttgtcaacat |
| subtilis) | ctttcgttcaattcccttccttattttaatcatcctgctt |
| SEQ ID NO: | cttggtttcactaaattcttagtgggaacaattttgggac |
| 637 | caaatgcggctcttcccgcgttagtcatcggtagtgctcc |
| | cttttatgctcgtctggtcgaaatcgcacttcgtgaagtg |
| | gacaaaggagtgattgaggcggcgaaatcgatgggggcta |
| | agacgagcactattatttttaaggttcttatccccgagtc |
| | catgcccgcgctgatttccggaattacagtgactgcgatt |
| | gcattgatcgggtcaaccgccatcgcaggagctattggtt |
| | ctggtggattgggaaacttagcatacgttgaaggctatca |
| | atcgaataatgcggatgtgaccttcgtggccacagttttc |
| | atcctgattattgttttcatcattcagatcattggtgacc |
| | ttattaccaacatcatcgataaacgc |
| MetNIQ | atgataaaactttcgaatatcaccaaagtgttccaccagg |
| (P281G) | gcacccgcaccatccaggcgttgaacaacgtcagcctgca |
| SEQ ID NO: | tgtgccagctggacaaatttatggcgttatcggtgcctca |
| 638 | ggcgcgggtaagagtacgcttatacgttgtgtaaacctgc |
| | tggagcgcccaaccgagggtagcgtgctggtcgatggcca |
| | ggaactgaccacgctgtcagaatccgagttgaccaaagct |
| | cgtcgccagattggtatgattttccagcattttaacctgc |
| | tctcttcgcgtactgtttttggcaacgtggctctgccgct |
| | ggagctggacaacacaccgaaagacgagatcaaacgtcgc |
| | gtgacggaattgctgtcattggttggtcttggcgataagc |
| | atgatagctacccgtcgaatctttccggtgggcagaaaca |
| | acgtgtggcgattgcccgtgcattagccagcaatcccaaa |
| | gtattgctgtgtgatgaagccaccagcgcgctggacccgg |
| | caacgacacgttctattctcgaactgctgaaagacatcaa |
| | ccgccgtctgggtttgacgattctgttgatcactcacgaa |
| | atggacgttgtgaagcgcatttgtgattgcgtggcggtca |
| | tcagcaatggcgaactgatcgagcaggacacggtaagtga |
| | agtgttctcgcatccgaaaacgccgctggcgcagaagttt |
| | attcagtcgaccctgcatctggatatcccggaagattacc |
| | aggaacgtctgcaagcggagccatttactgactgcgtgcc |
| | gatgctgcgtctggagtttaccggtcaatcggtcgatgcc |
| | ggcctgctttctgaaaccgcgcgtcgtttcaacgtcaaca |
| | acaacattattagcgcgcagatggattacgccggtggcgt |
| | taagttcggcatcatgctgactgaaatgcacggcacacaa |
| | caagatacgcaagccgccattgcctggctgcaagaacacc |
| | atgtaaaagtagaggtactgggttatgtctgagccgatga |
| | tgtggctgctggttcgtggcgtatgggaaacgctggcaat |
| | gaccttcgtatccggtttttttggctttgtgattggtctg |
| | ccggttggcgttctgctttatgtcacgcgtccggggcaaa |
| | ttattgctaacgcgaagttgtatcgtaccatttctgcgat |
| | tgtgaacattttccgttccatcccgttcattatcttgctg |
| | gtatggatgattccgtttacccgcgttattgtcggtacat |
| | cgattggattgcaggcagcgattgttccgttaaccgttgg |
| | tgcagcaccgtttattgcccgtatggtcgagaacgctctg |
| | ctggagatcccaaccgggttaattgaagcttcccgcgcaa |
| | tgggggccacgccaatgcagatcgtccgtaaagtgctgtt |
| | accggaagcgttgccgggtctggtgaatgcggcaactatc |
| | accctgattaccctggttggttattccgcgatgggtggtg |
| | cagtcggtgccggtggtttaggtcagattggctatcagta |
| | tggctacatcggctacaacgcgacggtgatgaatacggta |
| | ctggtattgctggtcattctggtttatttaattcagttcg |
| | caggcgaccgcatcgtccgggctgtcactcgcaagtaacg |
| | ttcaacacaacataaataattgaagaaggaataaggtatg |
| | gcgttcaaattcaaaacctttgcggcagtgggagccctga |
| | ttggatcactggcactggtaggctgcggtcaggatgaaaa |
| | agatccaaaccacattaaagtcggcgtgattgttggtgcc |
| | gaacagcaggttgcagaagtcgcgcagaaagttgcgaaag |
| | acaaatatggcctggacgttgagctggtaaccttcaacga |
| | ctatgttctgccaaacgaagcattgagcaaaggcgatatc |
| | gacgccaacgccttccagcataaaccgtaccttgatcagc |
| | aactgaaagatcgtggctacaaactggtcgcagtaggcaa |
| | cacatttgtttatccgattgctggttactccaagaaaatc |
| | aaatcactggatgaactgcaagatggttcgcaggttgccg |
| | tgccaaacgacccaactaaccttggtcgttcactgctgct |
| | gctgcaaaaagtgggcttgatcaaactgaaagatggcgtt |
| | ggcctgctgccgaccgttcttgatgttgttgagaacccaa |
| | aaaatctgaaaattgttgaactggaagcaccgcagctacc |
| | gcgctctctggacgacgcgcaaatcgctctggcagttatc |
| | aataccacctatgccagccagattggcctgactccagcga |
| | aagacggtatctttgtcgaagataaagagtccccgtacgt |
| | aaacctgatcgtaacgcgtgaagacaacaaagacgccgaa |
| | aacgtgaagaaattcgttcaggcttatcagtctgacgaag |
| | tttacgaagcagcaaacaaagtgtttaacggcggcgctgt |
| | taaaggctggtaa |
| Recoded | atgataaaactttcgaatatcaccaaagtgttccaccagg |
| MetNIQ | gcacccgcaccatccaggcgttgaacaacgtcagcctgca |
| SEQ ID NO: | tgtgccagctggacaaatttatggcgttatcggtgcctca |
| 639 | ggcgcgggtaagagtacgcttatacgttgtgtaaacctgc |
| | tggagcgcccaaccgagggtagcgtgctggtcgatggcca |
| | ggaactgaccacgctgtcagaatccgagttgaccaaagct |
| | cgtcgccagattggtatgattttccagcattttaacctgc |
| | tctcttcgcgtactgtttttggcaacgtggctctgccgct |
| | ggagctggacaacacaccgaaagacgagatcaaacgtcgc |
| | gtgacggaattgctgtcattggttggtcttggcgataagc |
| | atgatagctacccgtcgaatctttccggtgggcagaaaca |
| | acgtgtggcgattgcccgtgcattagccagcaatcccaaa |
| | gtattgctgtgtgatgaagccaccagcgcgctggacccgg |
| | caacgacacgttctattctcgaactgctgaaagacatcaa |
| | ccgccgtctgggtttgacgattctgttgatcactcacgaa |
| | atggacgttgtgaagcgcatttgtgattgcgtggcggtca |
| | tcagcaatggcgaactgatcgagcaggacacggtaagtga |
| | agtgttctcgcatccgaaaacgccgctggcgcagaagttt |
| | attcagtcgaccctgcatctggatatcccggaagattacc |
| | aggaacgtctgcaagcggagccatttactgactgcgtgcc |
| | gatgctgcgtctggagtttaccggtcaatcggtcgatgcc |
| | ccactgctttctgaaaccgcgcgtcgtttcaacgtcaaca |
| | acaacattattagcgcgcagatggattacgccggtggcgt |
| | taagttcggcatcatgctgactgaaatgcacggcacacaa |
| | caagatacgcaagccgccattgcctggctgcaagaacacc |
| | atgtaaaagtagaggtactgggttatgtctgagccgatga |
| | tgtggctgctggttcgtggcgtatgggaaacgctggcaat |
| | gaccttcgtatccggtttttttggctttgtgattggtctg |
| | ccggttggcgttctgctttatgtcacgcgtccggggcaaa |
| | ttattgctaacgcgaagttgtatcgtaccatttctgcgat |
| | tgtgaacattttccgttccatcccgttcattatcttgctg |
| | gtatggatgattccgtttacccgcgttattgtcggtacat |
| | cgattggattgcaggcagcgattgttccgttaaccgttgg |
| | tgcagcaccgtttattgcccgtatggtcgagaacgctctg |
| | ctggagatcccaaccgggttaattgaagcttcccgcgcaa |
| | tgggggccacgccaatgcagatcgtccgtaaagtgctgtt |
| | accggaagcgttgccgggtctggtgaatgcggcaactatc |
| | accctgattaccctggttggttattccgcgatgggtggtg |
| | cagtcggtgccggtggtttaggtcagattggctatcagta |
| | tggctacatcggctacaacgcgacggtgatgaatacggta |
| | ctggtattgctggtcattctggtttatttaattcagttcg |
| | caggcgaccgcatcgtccgggctgtcactcgcaagtaacg |
| | ttcaacacaacataaataattgaagaaggaataaggtatg |
| | gcgttcaaattcaaaacctttgcggcagtgggagccctga |
| | ttggatcactggcactggtaggctgcggtcaggatgaaaa |
| | agatccaaaccacattaaagtcggcgtgattgttggtgcc |
| | gaacagcaggttgcagaagtcgcgcagaaagttgcgaaag |
| | acaaatatggcctggacgttgagctggtaaccttcaacga |
| | ctatgttctgccaaacgaagcattgagcaaaggcgatatc |
| | gacgccaacgccttccagcataaaccgtaccttgatcagc |
| | aactgaaagatcgtggctacaaactggtcgcagtaggcaa |
| | cacatttgtttatccgattgctggttactccaagaaaatc |
| | aaatcactggatgaactgcaagatggttcgcaggttgccg |
| | tgccaaacgacccaactaaccttggtcgttcactgctgct |
| | gctgcaaaaagtgggcttgatcaaactgaaagatggcgtt |
| | ggcctgctgccgaccgttcttgatgttgttgagaacccaa |
| | aaaatctgaaaattgttgaactggaagcaccgcagctacc |
| | gcgctctctggacgacgcgcaaatcgctctggcagttatc |
| | aataccacctatgccagccagattggcctgactccagcga |
| | aagacggtatctttgtcgaagataaagagtccccgtacgt |
| | aaacctgatcgtaacgcgtgaagacaacaaagacgccgaa |
| | aacgtgaagaaattcgttcaggcttatcagtctgacgaag |
| | tttacgaagcagcaaacaaagtgtttaacggcggcgctgt |
| | taaaggctggtaa |
| MetNIQ | atgataaaactttcgaatatcaccaaagtgttccaccagg |
| (P281S) | gcacccgcaccatccaggcgttgaacaacgtcagcctgca |
| SEQ ID NO: | tgtgccagctggacaaatttatggcgttatcggtgcctca |
| 640 | ggcgcgggtaagagtacgcttatacgttgtgtaaacctgc |
| | tggagcgcccaaccgagggtagcgtgctggtcgatggcca |
| | ggaactgaccacgctgtcagaatccgagttgaccaaagct |
| | cgtcgccagattggtatgattttccagcattttaacctgc |
| | tctcttcgcgtactgtttttggcaacgtggctctgccgct |
| | ggagctggacaacacaccgaaagacgagatcaaacgtcgc |
| | gtgacggaattgctgtcattggttggtcttggcgataagc |
| | atgatagctacccgtcgaatctttccggtgggcagaaaca |
| | acgtgtggcgattgcccgtgcattagccagcaatcccaaa |
| | gtattgctgtgtgatgaagccaccagcgcgctggacccgg |
| | caacgacacgttctattctcgaactgctgaaagacatcaa |
| | ccgccgtctgggtttgacgattctgttgatcactcacgaa |
| | atggacgttgtgaagcgcatttgtgattgcgtggcggtca |
| | tcagcaatggcgaactgatcgagcaggacacggtaagtga |
| | agtgttctcgcatccgaaaacgccgctggcgcagaagttt |
| | attcagtcgaccctgcatctggatatcccggaagattacc |
| | aggaacgtctgcaagcggagccatttactgactgcgtgcc |
| | gatgctgcgtctggagtttaccggtcaatcggtcgatgcc |
| | tccctgctttctgaaaccgcgcgtcgtttcaacgtcaaca |
| | acaacattattagcgcgcagatggattacgccggtggcgt |
| | taagttcggcatcatgctgactgaaatgcacggcacacaa |
| | caagatacgcaagccgccattgcctggctgcaagaacacc |
| | atgtaaaagtagaggtactgggttatgtctgagccgatga |
| | tgtggctgctggttcgtggcgtatgggaaacgctggcaat |
| | gaccttcgtatccggtttttttggctttgtgattggtctg |
| | ccggttggcgttctgctttatgtcacgcgtccggggcaaa |
| | ttattgctaacgcgaagttgtatcgtaccatttctgcgat |
| | tgtgaacattttccgttccatcccgttcattatcttgctg |
| | gtatggatgattccgtttacccgcgttattgtcggtacat |
| | cgattggattgcaggcagcgattgttccgttaaccgttgg |
| | tgcagcaccgtttattgcccgtatggtcgagaacgctctg |
| | ctggagatcccaaccgggttaattgaagcttcccgcgcaa |
| | tgggggccacgccaatgcagatcgtccgtaaagtgctgtt |
| | accggaagcgttgccgggtctggtgaatgcggcaactatc |
| | accctgattaccctggttggttattccgcgatgggggtgc |
| | agtcggtgccggtggtttaggtcagattggctatcagtat |
| | ggctacatcggctacaacgcgacggtgatgaatacggtac |
| | tggtattgctggtcattctggtttatttaattcagttcgc |
| | aggcgaccgcatcgtccgggctgtcactcgcaagtaacgt |
| | tcaacacaacataaataattgaagaaggaataaggtatgg |
| | cgttcaaattcaaaacctttgcggcagtgggagccctgat |
| | tggatcactggcactggtaggctgcggtcaggatgaaaaa |
| | gatccaaaccacattaaagtcggcgtgattgttggtgccg |
| | aacagcaggttgcagaagtcgcgcagaaagttgcgaaaga |
| | caaatatggcctggacgttgagctggtaaccttcaacgac |
| | tatgttctgccaaacgaagcattgagcaaaggcgatatcg |
| | acgccaacgccttccagcataaaccgtaccttgatcagca |
| | actgaaagatcgtggctacaaactggtcgcagtaggcaac |
| | acatttgtttatccgattgctggttactccaagaaaatca |
| | aatcactggatgaactgcaagatggttcgcaggttgccgt |
| | gccaaacgacccaactaaccttggtcgttcactgctgctg |
| | ctgcaaaaagtgggcttgatcaaactgaaagatggcgttg |
| | gcctgctgccgaccgttcttgatgttgttgagaacccaaa |
| | aaatctgaaaattgttgaactggaagcaccgcagctaccg |
| | cgctctctggacgacgcgcaaatcgctctggcagttatca |
| | ataccacctatgccagccagattggcctgactccagcgaa |
| | agacggtatctttgtcgaagataaagagtccccgtacgta |
| | aacctgatcgtaacgcgtgaagacaacaaagacgccgaaa |
| | acgtgaagaaattcgttcaggcttatcagtctgacgaagt |
| | ttacgaagcagcaaacaaagtgtttaacggcggcgctgtt |
| | aaaggctggtaa |

TABLE 22
Exemplary Methionine Decarboxylase Amino Acid Sequences

| Description | |
|---|---|
| SEQ ID NO: | Amino Acid Sequence |
| MetDC Q70D | MSPTAFPAAETATAPATAVDPGPELDGGDFALPEGGLDDD |
| N82H | RRLRALDAVDEYLTRKRKHLVGYQATQDMDGTALDLARFM |
| SEQ ID NO: | PHNINNLGDPFQSGGYKPNTKVVERAVLDYYAKLWHAERP |
| 641 | HDPADPESYWGYMLSMGSTEGNMYALWNARDYLSGKALIQ |
| | PPTAPFDAVRYVKADPDRRNPNAHHPVAFYSEDTHYSFAK |
| | AVAVLGVETFHAVGLEKYADECPLVDPVTGLRTWPTEVPS |
| | RPGPSGLSWDGPGEIDVDALAVLVEFFAAKGHPVFVNLNL |
| | GSTFKGAHDDVRAVCERLLPIFERHGLVQREVVYGSCPQT |
| | GRPLVDVRRGFWIHVDGALGAGYAPFLRLAAEDPEGYGWT |
| | PEAELPEFDFGLRLPTAGHGEVDMVSSIAMSGHKWAGAPW |
| | PCGIYMTKVKYQISPPSQPDYIGAPDTTFAGSRNGFSPLI |
| | LWDHLSRYSYRDQVERIREAQELAAYLERRLTAMERELGV |
| | ELWPARTPGAVTVRFRKPSAELVAKWSLSSQDVLMVPGDE |
| | TTRRSYVHVFVMPSVDRAKLDALLAELAEDPVILGAP |
| MetDC | MSPTAFPAAETATAPATAVDPGPELDGGDFALPEGGLDDD |
| (Streptomyces) | RRLRALDAVDEYLTRKRKHLVGYQATQDMQGTALDLARFM |
| SEQ ID NO: | PNNINNLGDPFQSGGYKPNTKVVERAVLDYYAKLWHAERP |
| 642 | HDPADPESYWGYMLSMGSTEGNMYALWNARDYLSGKALIQ |
| | PPTAPFDAVRYVKADPDRRNPNAHHPVAFYSEDTHYSFAK |
| | AVAVLGVETFHAVGLEKYADECPLVDPVTGLRTWPTEVPS |
| | RPGPSGLSWDGPGEIDVDALAVLVEFFAAKGHPVFVNLNL |
| | GSTFKGAHDDVRAVCERLLPIFERHGLVQREVVYGSCPQT |
| | GRPLVDVRRGFWIHVDGALGAGYAPFLRLAAEDPEGYGWT |
| | PEAELPEFDFGLRLPTAGHGEVDMVSSIAMSGHKWAGAPW |
| | PCGIYMTKVKYQISPPSQPDYIGAPDTTFAGSRNGFSPLI |
| | LWDHLSRYSYRDQVERIREAQELAAYLERRLTAMERELGV |
| | ELWPARTPGAVTVRFRKPSAELVAKWSLSSQDVLMVPGDE |
| | TTRRSYVHVFVMPSVDRAKLDALLAELAEDPVILGAP |
| MetDC (V491L | MSPTAFPAAETATAPATAVDPGPELDGGDFALPEGGLDDD |
| A500P) | RRLRALDAVDEYLTRKRKHLVGYQATQDMQGTALDLARFM |
| SEQ ID NO: | PNNINNLGDPFQSGGYKPNTKVVERAVLDYYAKLWHAERP |
| 643 | HDPADPESYWGYMLSMGSTEGNMYALWNARDYLSGKALIQ |
| | PPTAPFDAVRYVKADPDRRNPNAHHPVAFYSEDTHYSFAK |
| | AVAVLGVETFHAVGLEKYADECPLVDPVTGLRTWPTEVPS |
| | RPGPSGLSWDGPGEIDVDALAVLVEFFAAKGHPVFVNLNL |
| | GSTFKGAHDDVRAVCERLLPIFERHGLVQREVVYGSCPQT |
| | GRPLVDVRRGFWIHVDGALGAGYAPFLRLAAEDPEGYGWT |
| | PEAELPEFDFGLRLPTAGHGEVDMVSSIAMSGHKWAGAPW |
| | PCGIYMTKVKYQISPPSQPDYIGAPDTTFAGSRNGFSPLI |
| | LWDHLSRYSYRDQVERIREAQELAAYLERRLTAMERELGV |
| | ELWPARTPGALTVRFRKPSPELVAKWSLSSQDVLMVPGDE |
| | TTRRSYVHVFVMPSVDRAKLDALLAELAEDPVILGAP |
| MetDC (R41Q | SPTAFPAAETATAPATAVDPGPELDGGDFALPEGGLDDDQ |
| Q70D) | RLRALDAVDEYLTRKRKHLVGYQATQDMDGTALDLARFMP |
| SEQ ID NO: | NNINNLGDPFQSGGYKPNTKVVERAVLDYYAKLWHAERPH |
| 644 | DPADPESYWGYMLSMGSTEGNMYALWNARDYLSGKALIQP |
| | PTAPFDAVRYVKADPDRRNPNAHHPVAFYSEDTHYSFAKA |
| | VAVLGVETFHAVGLEKYADECPLVDPVTGLRTWPTEVPSR |
| | PGPSGLSWDGPGEIDVDALAVLVEFFAAKGHPVFVNLNLG |
| | STFKGAHDDVRAVCERLLPIFERHGLVQREVVYGSCPQTG |
| | RPLVDVRRGFWIHVDGALGAGYAPFLRLAAEDPEGYGWTP |
| | EAELPEFDFGLRLPTAGHGEVDMVSSIAMSGHKWAGAPWP |
| | CGIYMTKVKYQISPPSQPDYIGAPDTTFAGSRNGFSPLIL |
| | WDHLSRYSYRDQVERIREAQELAAYLERRLTAMERELGVE |
| | LWPARTPGAVTVRFRKPSAELVAKWSLSSQDVLMVPGDET |
| | TRRSYVHVFVMPSVDRAKLDALLAELAEDPVILGAP |
| MetDC | MGFQLLSKHKLSAEDQQKLDRFYRDIQTEAERFLGYPCNE |
| (Stanieria sp. | LFDYSPLFRFLQYPLNNVGDPYLPSNYHLNTHNFECEVLE |
| NIES-3757) | IFRTLTEATEGSTWGYVTNGGTEGNHYGLFLARELLPEGL |
| SEQ ID NO: | VYYSQDAHYSIDKILRCLNLRSIMIRSHDDGRMDLDDLRE |
| 645 | TLRIHRDLPPIVCATIGTTMKGAVDDIAGIKKIFKDLAIH |
| | RHYIHADAALGGMILPFLDNSPPWNFKAGIDSIAISGHKM |
| | VGSPIPCGVVLAKKSNVERIAQSVEYIGTLDTTLSGSRNA |
| | LTPLFLWYAFHTVGIEGFKRIIPACLKMADYAIAQLNKIN |
| | RNAWRYPYSNTVVFDRPSPEVTRYWQLACQGNLSHLITMP |
| | HVTSTQIDHLVADIIASEPIPPLPTLSVTPACELLTSTPD |
| | QDITLIGTANHNLLSEVSTALAAEGLSIENLAAVAVESED |
| | VEVVRLRVNNRERALQILNQNLDIGRCYGQARPFGNEEAT |
| | QVLSQLEYQSVGEDALLVQLDDCPGSLAELLKDCRNEAVK |
| | IRNIRLLWRGHGKGVVAIATTSPDALKTLLKDRILLS |
| MetDC | MSTPSEVKKDLLGAAGSLWPSEPITLGPGESAWQLVLKKI |
| (Mus musculus) | QELSDSGHQDPFMVADLDVLVSRHQTFCQALPRVQPFYAV |
| SEQ ID NO: | KCNSNPWVLRVLAALGTGFDCASQGELEQVLGLGVAPSRI |
| 646 | IFANPCKAVSHIQFAARCGVQLLTFDSEEELIKVAQYHPG |
| | ARLVLRIQTQDSQSTFPLSTKFGASLEACGHLLQVARELG |
| | LAVVGASFHVGSDCHTPQSFRQAIADCHRVFEMGRKAGHD |
| | MSLLDLGGGFPGVEGSEAKFEEMARVINAALAQYFPEETG |
| | IEVIAEPGRFYAGSVCTAAVNIIAKKSVLEPGGHRKLMYY |
| | LNEGHYGSFRLFLRDPVPRIPIVVKEFPSEPPLFPCTLYG |
| | PTCDAYDRLFSEEVQLPELDVGDWLIFPDMGAYTSSMSST |
| | FNGFPPATVYCAMSPQLRSLLETVP |
| LeuDC | MNTPSEVKKDLLGVAEHLRPSEPITLGPGASAWQLVLKKI |
| (Mus musculus) | KELSISGRQDAFMVADLDVLVSRHRTFLQALPRVQPFYAV |
| SEQ ID NO: | KCNSNPWVLLVLAALGTGFDCASQGELEQVLGLGVAPSRI |
| 647 | IFANPCKAVSHIQFAARCGVQLLTFDNEEELIKLARYHPR |
| | ARLVLRIQTLDSQSTFPLSTKFGAHLEACGHLLQVARELG |
| | LAVVGASFHVGSDCHTPESYRQAIADCHRVFEMGCKAGHH |
| | MSLLDLGGGFPGVKGSEAKFEEVARVINTALAQYFPEETG |
| | IEVIAEPGRFYAGSVCTAAVNIIAKKSSLDPGGHRKLAYY |
| | LNEGHYGVFRLFLRDPVPRIPIVVKEFPSEPPLFPCTLYG |
| | PTCDAYDRLFSTEVQLPELDVGDWLIFPDMGAYSSSMSST |
| | FNGFPIATVYDAMSPQLRSLLETVP |
| MetDC | MKQTSLEVKEFALNLISQFEPENQPLGFWIFDTEGVEKAV |
| (Entamoeba | ERWKKNMPTVRPCFAVKCNPEPHLVKLLGELGCGFDCASL |
| histolytica) | NEIKEVLDLGENPEDITYSQTFKPYNQLIEASHLGINHTI |
| SEQ ID NO: | VDSIDEVQKIAKYAPKMGIMIRIMENDTSAGHVFGEKFGL |
| 648 | HDDEVEIVLKEIKDKGLNLDGVHFHVGSDSHNSEVFTKAL |
| | TKARNTVTLAEQFGMKPYLIDIGGGFSQVAPFEEFAATIE |
| | KTIKELEFPERTRFIAEPGRYMASNAFHLVSSLHGKRVRI |
| | QNGKKQIEYTSGDGLHGSFGCCIWFEKQKSCECITQKVNE |
| | NTKMYESIIYGPSCNGSDKVATQELPEMEPGKDWLLFPNM |
| | GAYTISMATNFNGFEERNHVIYTLPLKSTKIIQIPKSIEC |
| | NSVPSLNGIPHYA |

TABLE 23
Exemplary Transporter Amino Acid Sequences

| Description | |
|---|---|
| SEQ ID NO: | Nucleotide Sequence |
| MetP | MGTINTKIYKYMSIWKTKPLSVLLSEATEDEKGLKRTLSA |
| (F. segetis) | RSLVALGVGAIIGAGLFSLTGIAAADNAGPAVTLSFILAS |
| SEQ ID NO: | VGCAFAGLCYAEFASMIPVAGSAYTYSYATMGEFVAWIIG |
| 649 | WDLVLEYALGAATVAVSWSQYVDKFLQNYGIHIPNSILHG |
| | PWDTTPGIINLPSIFIICLLSVLLIRGTKESALINNILVI |
| | LKVTVVIVFIGLGWGFMNSANHTPFIPVNEGEALLSSGEM |
| | SFLNFFSSDYFGHYGWSGILRGAGVVFFAFIGFDAVSTAA |
| | QEAKDPQKGMPIGILGSLIICTVLYVLFAFVLTGLENYLN |
| | FKGDASPVTTAFAKTGYTFLNSGLTIAIIAGYTSVMLVML |
| | MGQSRVFYSMSVDGLLPKFFSTLHTKNRTPYKTNLLFMVF |
| | VSLFAGFVPVSDLGHMVSIGTLFAFCLVCIGVIVMRKTNP |
| | DAVRGFRVPFVPVFPIIGVVICLVLMAGLPIESWERLAIW |
| | MILGVVIYFFYSKKNSKLNNPE |
| MetP | MGTINTKTNKYMSIWKTKPLSVLLNEASEDEKGLKRTLSS |
| (F. frigoris) | RSLVALGVGAIIGAGLFSLTGIAAAEHAGPAVTLSFILAA |
| SEQ ID NO: | VGCAFAGLCYAEFASMIPVAGSAYTYSYATMGEFMAWIIG |
| 650 | WDLVLEYALGAATVGVSWSRYLLELLNKYGVHLNPKFICS |
| | PWETLTLGDGTIIDGGYINLPAILIVSALSLLLIRGTQES |
| | ASINNILVVLKVIVVIMFIVLGWDYIDPANYSPYIPENTG |
| | VKGQFGWSGIAAGAGTVFFAFIGFDAVSTAAQEAKNPQKG |
| | MPIGILGSLVICTILYVLFAHVMTGLVPYYKFAGDAKPAA |
| | TAFAVTGYSFLQTGLIVAILAGYTSVMLVMLMGQSRVFYT |
| | MSKDGLLPPLFGQIHSKFRTPYKTNLFFMVFVSLFAGFVP |
| | VSDLGHMVSIGTLLAFVLVCIGVLVMRKKMPDAPRSFKTP |
| | FVPYVPIAGVLVCTYLMYSLPYESWIRLVLWMAIGVALYF |
| | VYGKKHSKLNNPD |
| MetN | MIKLSNITKVFHQGTRTIQALNNVSLHVPAGQIYGVIGAS |
| SEQ ID NO: | GAGKSTLIRCVNLLERPTEGSVLVDGQELTTLSESELTKA |
| 651 | RRQIGMIFQHFNLLSSRTVFGNVALPLELDNTPKDEIKRR |
| | VTELLSLVGLGDKHDSYPSNLSGGQKQRVAIARALASNPK |
| | VLLCDEATSALDPATTRSILELLKDINRRLGLTILLITHE |
| | MDVVKRICDCVAVISNGELIEQDTVSEVFSHPKTPLAQKF |
| | IQSTLHLDIPEDYQERLQAEPFTDCVPMLRLEFTGQSVDA |
| | PLLSETARRFNVNNNIISAQMDYAGGVKFGIMLTEMHGTQ |
| | QDTQAAIAWLQEHHVKVEVLGYV |
| MetI | MSEPMMWLLVRGVWETLAMTFVSGFFGFVIGLPVGVLLYV |
| SEQ ID NO: | TRPGQIIANAKLYRTISAIVNIFRSIPFIILLVWMIPFTR |
| 652 | VIVGTSIGLQAAIVPLTVGAAPFIARMVENALLEIPTGLI |
| | EASRAMGATPMQIVRKVLLPEALPGLVNAATITLITLVGY |
| | SAMGGAVGAGGLGQIGYQYGYIGYNATVMNTVLVLLVILV |
| | YLIQFAGDRIVRAVTRK |
| MetQ | MAFKFKTFAAVGALIGSLALVGCGQDEKDPNHIKVGVIVG |
| SEQ ID NO: | AEQQVAEVAQKVAKDKYGLDVELVTFNDYVLPNEALSKGD |
| 653 | IDANAFQHKPYLDQQLKDRGYKLVAVGNTFVYPIAGYSKK |
| | IKSLDELQDGSQVAVPNDPTNLGRSLLLLQKVGLIKLKDG |
| | VGLLPTVLDVVENPKNLKIVELEAPQLPRSLDDAQIALAV |
| | INTTYASQIGLTPAKDGIFVEDKESPYVNLIVTREDNKDA |
| | ENVKKFVQAYQSDEVYEAANKVFNGGAVKGW |
| Methionine | MFEKYFPNVDLTELWNATYETLYMTLISLLFAFVIGVILG |
| import system | LLLFLTSKGSLWQNKAVNSVIAAVVNIFRSIPFLILIILL |
| permease | LGFTKFLVGTILGPNAALPALVIGSAPFYARLVEIALREV |
| protein MetP | DKGVIEAAKSMGAKTSTIIFKVLIPESMPALISGITVTAI |
| (Bacillus | ALIGSTAIAGAIGSGGLGNLAYVEGYQSNNADVTFVATVF |
| subtilis) | ILIIVFIIQIIGDLITNIIDKR |
| SEQ ID NO: | |
| 654 | |
| MetN (P281G) | MIKLSNITKVFHQGTRTIQALNNVSLHVPAGQIYGVIGAS |
| SEQ ID NO: | GAGKSTLIRCVNLLERPTEGSVLVDGQELTTLSESELTKA |
| 655 | RRQIGMIFQHFNLLSSRTVFGNVALPLELDNTPKDEIKRR |
| | VTELLSLVGLGDKHDSYPSNLSGGQKQRVAIARALASNPK |
| | VLLCDEATSALDPATTRSILELLKDINRRLGLTILLITHE |
| | MDVVKRICDCVAVISNGELIEQDTVSEVFSHPKTPLAQKF |
| | IQSTLHLDIPEDYQERLQAEPFTDCVPMLRLEFTGQSVDA |
| | GLLSETARRFNVNNNIISAQMDYAGGVKFGIMLTEMHGTQ |
| | QDTQAAIAWLQEHHVKVEVLGYV |
| MetN (P281S) | MIKLSNITKVFHQGTRTIQALNNVSLHVPAGQIYGVIGAS |
| SEQ ID NO: | GAGKSTLIRCVNLLERPTEGSVLVDGQELTTLSESELTKA |
| 656 | RRQIGMIFQHFNLLSSRTVFGNVALPLELDNTPKDEIKRR |
| | VTELLSLVGLGDKHDSYPSNLSGGQKQRVAIARALASNPK |
| | VLLCDEATSALDPATTRSILELLKDINRRLGLTILLITHE |
| | MDVVKRICDCVAVISNGELIEQDTVSEVFSHPKTPLAQKF |
| | IQSTLHLDIPEDYQERLQAEPFTDCVPMLRLEFTGQSVDA |
| | SLLSETARRFNVNNNIISAQMDYAGGVKFGIMLTEMHGTQ |
| | QDTQAAIAWLQEHHVKVEVLGYV |

TABLE 24
Exemplary Sequences

| Description | Sequence |
|---|---|
| DL- | atgattaaactgagcaacattactaaggtgttccaccaaggtaca |
| methionine | cgtacgatccaggctcttaataatgtgtcactgcacgttcctgct |
| transporter | ggtcagatttatggggttatcggtgccagtggggctgggaagagc |
| subunit | actctgatccgctgcgtcaatctgttagagcgccctacagagggc |
| metN | tcggtactggtggacggtcaagagttgactactctgtcggagtcc |
| (Escherichia | gagttgacaaaagcacgccgccagattggcatgattttccaacat |
| coli | ttcaatttgttatcgagccgtacagttttcgggaacgtggcctta |
| K-12]) | ccactggagttggacaatactcccaaagacgaagtcaaacgtcgt |
| SEQ ID | gtgaccgaattattgtccttggtgggtcttggtgacaaacacgac |
| NO: 657 | agttatcccagtaatttgagtggcgggcaaaaacagcgtgttgcc |
| | atcgcacgcgcattagcttcgaatcccaaggtgctgttatgtgat |
| | gaagcgaccagcgcccttgacccagccacaactcgtagcatcctg |
| | gagcttttgaaagatatcaatcgtcgcctgggtttgaccatctta |
| | ttgattacgcacgagatggacgttgtaaagcgtatctgtgactgt |
| | gtagcggtgatctccaacggtgaattaatcgaacaggacaccgta |
| | tcggaggtcttctcacatcctaagacaccccttgcacaaaaattc |
| | atccaaagcacgctgcatttagatattcctgaagattatcaggaa |
| | cgcctgcaggctgaaccgtttactgattgcgttccaatgcttcgc |
| | ttagagttcacagggcaatcggttgacgctcccttattgagtgaa |
| | accgcccgccgtttcaatgttaataacaacatcatttccgcgcaa |
| | atggactacgcggggggtgttaaatttggaatcatgttaaccgaa |
| | atgcacggcacacagcaggatacacaggcggcgatcgcatggctg |
| | caggaacatcatgttaaagtagaagtccttgggtatgtg |
| metI | atgtctgagccgatgatgtggctgctggttcgtggcgtatgggaa |
| (Escherichia | acgctggcaatgaccttcgtatccggtttttttggctttgtgatt |
| coli) | ggtctgccggttggcgttctgctttatgtcacgcgtccggggcaa |
| SEQ ID | attattgctaacgcgaagctgtatcgtaccgtttctgcgattgtg |
| NO: 658 | aacattttccgttccatcccgttcattatcttgcttgtatggatg |
| | attccgtttacccgcgttattgtcggtacatcgattggtttgcag |
| | gcagcgattgttccgttaaccgttggtgcagcaccgtttattgcc |
| | cgtatggtcgagaacgctctgctggagatcccaaccgggttaatt |
| | gaagcttcccgcgcaatgggtgccacgccgatgcagatcgtccgt |
| | aaggtgctgttaccggaagcgctgccgggtctggtgaatgcggca |
| | actatcaccctgattaccctggtcggttattccgcgatgggtggt |
| | gcagtcggtgccggtggtttaggtcagattggctatcagtatggc |
| | tacatcggctataacgcgacggtgatgaatacggtactggtattg |
| | ctggtcattctggtttatttaattcagttcgcaggcgaccgcatc |
| | gtccgggctgtcactcgcaagtaa |
| metQ | atggcgttcaaattcaaaacctttgcggcagtgggagccctgatc |
| (Escherichia | ggatcactggcactggtaggctgcggtcaggatgaaaaagatcca |
| coli) | aaccacattaaagtcggcgtgattgttggtgccgaacagcaggtt |
| SEQ ID | gcagaagtcgcgcagaaagttgcgaaagacaaatatggcctggac |
| NO: 659 | gttgagctggtaaccttcaacgactatgttctgccaaacgaagca |
| | ttgagcaaaggcgatatcgacgccaacgccttccagcataaaccg |
| | taccttgatcagcaactgaaagatcgtggctacaaactggtcgca |
| | gtaggcaacacttttgtttatccgattgctggttactccaagaaa |
| | atcaaatcactggatgaactgcaggatggttcgcaggttgccgtg |
| | ccaaacgacccaactaaccttggtcgttcactgctgctgctgcaa |
| | aaagtgggcttgatcaaactgaaagatggcgttggcctgctgccg |
| | accgttcttgatgttgttgagaaccccaaaaatctgaaaattgtt |
| | gaactggaagcaccgcaactgccgcgttctctggacgacgcgcaa |
| | atcgctctggcagttatcaataccacctatgccagccagattggc |
| | ctgactccggcgaaagacggtatctttgttgaagataaagagtcc |
| | ccgtacgtaaacctgatcgtgacgcgtgaagataacaaagacgcc |
| | gagaacgtgaagaaattcgtccaggcttatcagtctgacgaagtt |
| | tacgaagcagcaaacaaagtgtttaacggcggagctgttaaaggc |
| | tggtaa |
| lacI-Ptac | TCACTGCCCGCTTTCCAGTCGGGAAACCTGTCGTGCCAGCTGCAT |
| SEQ ID | TAATGAATCGGCCAACGCGCGGGGAGAGGCGGTTTGCGTATTGGG |
| NO: 660 | CGCCAGGGTGGTTTTTCTTTTCACCAGTGAGACTGGCAACAGCTG |
| | ATTGCCCTTCACCGCCTGGCCCTGAGAGAGTTGCAGCAAGCGGTC |
| | CACGCTGGTTTGCCCCAGCAGGCGAAAATCCTGTTTGATGGTGGT |
| | TAACGGCGGGATATAACATGAGCTATCTTCGGTATCGTCGTATCC |
| | CACTACCGAGATATCCGCACCAACGCGCAGCCCGGACTCGGTAAT |
| | GGCGCGCATTGCGCCCAGCGCCATCTGATCGTTGGCAACCAGCAT |
| | CGCAGTGGGAACGATGCCCTCATTCAGCATTTGCATGGTTTGTTG |
| | AAAACCGGACATGGCACTCCAGTCGCCTTCCCGTTCCGCTATCGG |
| | CTGAATTTGATTGCGAGTGAGATATTTATGCCAGCCAGCCAGACG |
| | CAGACGCGCCGAGACAGAACTTAATGGGCCCGCTAACAGCGCGAT |
| | TTGCTGGTGACCCAATGCGACCAGATGCTCCACGCCCAGTCGCGT |
| | ACCGTCCTCATGGGAGAAAATAATACTGTTGATGGGTGTCTGGTC |
| | AGAGACATCAAGAAATAACGCCGGAACATTAGTGCAGGCAGCTTC |
| | CACAGCAATGGCATCCTGGTCATCCAGCGGATAGTTAATGATCAG |
| | CCCACTGACGCGTTGCGCGAGAAGATTGTGCACCGCCGCTTTACA |
| | GGCTTCGACGCCGCTTCGTTCTACCATCGACACCACCACGCTGGC |
| | ACCCAGTTGATCGGCGCGAGATTTAATCGCCGCGACAATTTGCGA |
| | CGGCGCGTGCAGGGCCAGACTGGAGGTGGCAACGCCAATCAGCAA |
| | CGACTGTTTGCCCGCCAGTTGTTGTGCCACGCGGTTGGGAATGTA |
| | ATTCAGCTCCGCCATCGCCGCTTCCACTTTTTCCCGCGTTTTCGC |
| | AGAAACGTGGCTGGCCTGGTTCACCACGCGGGAAACGGTCTGATA |
| | AGAGACACCGGCATACTCTGCGACATCGTATAACGTTACTGGTTT |
| | CATATTCACCACCCTGAATTGACTCTCTTCCGGGCGCTATCATGC |
| | CATACCGCGAAAGGTTTTGCGCCATTCGATGGCGCGCCGCTTCGT |
| | CAGGCCACATAGCTTTCTTGTTCTGATCGGAACGATCGTTGGCTG |
| | tgttgacaattaatcatcggctcgtataatgtgtggaattgtgag |
| | cgctcacaatt |
| Ptac-metP | ttgacaattaatcatcggctcgtataatgtgtggaattgtgagcg |
| SEQ ID | ctcacaattagctgtgaccagaggtaaggaggtaacaaccatgcg |
| NO: 661 | agtgttgaagaaacatcttaatcatgctgcggagggtttctaatg |
| | gggaccattaacacgaagatctataaatacatgagcatctggaaa |
| | acaaaacctctgtccgtgctcttgtctgaagcaactgaggatgaa |
| | aaaggcctgaagcgcactctgtcggcccgttcacttgttgcgctg |
| | ggtgtcggtgctattatcggcgctggtttattctctctgaccggc |
| | atagctgcggcagacaatgctggaccggcagtaaccctgagcttt |
| | atcctggcctccgttggttgcgcgttcgctggcctgtgttacgca |
| | gaatttgcttctatgattccagttgcgggtagcgcctacacttat |
| | agttatgctaccatgggcgagttcgtggcgtggatcatcggttgg |
| | gatctggtactcgaatacgcattgggcgcagctactgttgccgtt |
| | agctggtcccagtacgtggacaaattcttgcaaaactacggcatc |
| | catattccgaactctatcctccacgggccgtgggataccaccccc |
| | ggtattatcaatttaccgtcgatatttatcatctgcctgctgagc |
| | gtgctgctgattcgtggtactaaagaatctgctctgatcaacaac |
| | attctggtaatcctgaaagtcacggttgtcatcgtgttcattggc |
| | ctgggctgggggttcatgaactccgcaaaccacacgccctttatc |
| | ccggttaacgaaggtgaggctctactgtcttctggtgaaatgagt |
| | ttcctcaactttttcagcagtgactactttggacactacggatgg |
| | tccggtattcttcgcggcgctggtgtagtattcttcgcatttatc |
| | ggcttcgacgcggtgagcactgcggcacaggaggccaaggatccg |
| | cagaaaggcatgccaatcggtattctgggctcactgatcatttgc |
| | accgttctgtacgtgcttttcgctttcgttctgaccggtctggaa |
| | aactatctaaacttcaaaggtgacgcttctcctgtcaccactgca |
| | tttgccaaaacaggctatactttcctgaatagcggtctgacgatc |
| | gctatcatagcgggctacacatccgttatgctggtaatgttgatg |
| | ggtcagtcccgtgtcttttatagtatgtctgtggatggcctgctt |
| | ccgaagtttttctcgaccctgcataccaaaaacaggactccgtac |
| | aaaactaatttgctgttcatggttttcgtaagcctgttcgctggc |
| | tttgttccggtcagcgacctgggccatatggtatccatcggtacc |
| | ctcttcgctttctgcctggtgtgtatcggcgttatcgttatgcga |
| | aaaaccaacccagacgccgttcgcggttttcgtgttccttttgta |
| | ccggttttcccgattatcggtgtagttatttgtctggttctaatg |
| | ggggcctgccgattgaatcttgggaacgtctggcgatctggatga |
| | ttctgggtgtcgtgatctacttcttctactctaaaaagaactcta |
| | aactgaataaccccgaataa |
| Ptac-metDC | ttgacaattaatcatcggctcgtataatgtgtggaattgtgagcg |
| SEQ ID | ctcacaattaagtgaATTGCCAATAACAATTACTAAGGAGGTTTT |
| NO: 662 | TTATGtccccgacggcgtttccagcggccgaaacagctactgccc |
| | ctgcaactgccgtcgatcctgggccagaactggacggcggagatt |
| | tcgcccttccagaggggggctggatgacgatcgtcgcttacgtgc |
| | attggacgcagttgacgagtatttgacccgcaagcgcaagcattt |
| | ggttgggtaccaagctacccaggatatggacggaacggccttgga |
| | tttagcccgtttcatgccccacaacatcaacaacctgggagatcc |
| | tttccagtcggggggtataaaccaaatacgaaagtcgttgagcgt |
| | gccgtactggactactatgcaaaattgtggcacgcagaacgtcca |
| | cacgacccagctgacccagaaagctactggggttacatgttatcg |
| | atgggctcaactgagggcaacatgtacgccctgtggaatgcacgt |
| | gactacctgtcgggtaaggctttgattcagcctcccacggcacca |
| | tttgacgctgttcgctacgtgaaggctgaccccgatcgccgcaat |
| | cctaacgcacaccacccagtcgcattctactcggaggatacccac |
| | tattcttttgctaaagccgttgcggtgctgggtgtcgaaactttc |
| | cacgctgtgggtctggagaaatacgctgacgagtgccccttggtg |
| | gatccagtaaccggccttcgtacctggccgaccgaagttccatcg |
| | cgcccggggccgtcgggtttaagctgggacggccctggtgagatt |
| | gatgttgatgcgcttgcagtactggtcgagttcttcgcagcgaag |
| | ggtcaccccgtcttcgtcaaccttaacttggggtctacatttaaa |
| | ggagcacatgatgacgtacgtgcggtatgtgaacgcttattacca |
| | atcttcgagcgccatggcttagtacaacgtgaagttgtatatggg |
| | agctgtccccaaaccggccgccctttagtggatgtacgtcgcgga |
| | ttttggatccacgtagatggggcacttggggcggggtatgcccct |
| | tttctgcgtcttgccgccgaagacccggaaggttatggttggacc |
| | cctgaggcagaattacctgagttcgacttcggcttacgtttgccg |
| | acggcggggcatggagaagttgatatggttagcagcatcgccatg |
| | agtggacataagtgggcaggcgcgccgtggccatgcggcatctat |
| | atgacgaaagtgaaatatcagattagtccaccgtcacagcccgat |
| | tatattggtgctcctgacacaacatttgccggttcccgtaacggc |
| | ttttcgccgttaattttgtgggatcatttatcgcgctactcgtac |
| | cgcgaccaggtagagcgcatccgcgaagcacaggagcttgcagca |
| | tatttggaacgccgccttaccgctatggagcgcgagctgggagtg |
| | gaactttggccagcccgcacaccgggtgctgtaaccgtacgtttt |
| | cgcaaaccctctgctgagctggttgcgaagtggtccttgtcgtcg |
| | caggatgttttaatggtgccgggtgatgaaactacgcgtcgtagt |
| | tacgttcatgtgttcgtgatgccttctgttgatcgtgcaaagtta |
| | gatgcgttgctggcagaattggccgaagatcccgtcatcttgggt |
| | gcgccttaa |
| yjeH | atgagtggactcaaacaagaactggggctggcccagggcatcggc |
| SEQ ID | ctactatcgacgtcattattaggcactggcgtgtttgccgttcct |
| NO: 607 | gcgttagctgcgctagtagcaggcaataacagcctgtgggcgtgg |
| | cccgttttgattatcttagtgttcccgattgcgattgtgtttgcg |
| | attctgggtcgccactatcccagcgcaggcggcgtcgcacacttc |
| | gtcggtatggcgtttggttcgcggcttgagcgagtcaccggctgg |
| | ttgtttttatcggtcattcccgtgggtttgcctgccgcgctacaa |
| | attgctgccggattcggccaggcaatgtttggctggcatagcggg |
| | caactgttgttggcagaactcggtacgctggcgctggtgtggtat |
| | atcggtactcgaggtgccagttccagtgctaatctacaaacagtt |
| | attgccgggcttatcgtcgcactgattgtcgctatctggtgggcg |
| | ggcgatatcaaacctgcgaatatccccttccctgcgccaggaaat |
| | atcgaacttaccgggttattcgctgcgttatcagtgatgttctgg |
| | tgttttgtcggtctggaagcatttgcccatcttgcctcggaattt |
| | aaaaatccagagcgtgattttcctcgtgctttgatgattggcctg |
| | ctgctggcaggattagtctattggggctgtacggtagtcgtctta |
| | cacttcgacgcctatggtgaacaaatggcggcggcagcatcgctt |
| | cccaaaattgtagtgcagttattcggtgtaggagcgttatggatt |
| | gcctgcgtaattggctatctggcctgctttgccagtctcaacatt |
| | tatatacagagcttcgcccgcctggtctggtcgcaggcgcaacat |
| | aatcctgaccattacctggcacgcctctcttctcgccatattccg |
| | aataatgccctcaatgcggtgctcggctgctgcgtggtgagcacg |
| | ttggtgattcatgctttagagatcaatctggacgctcttattatt |
| | tatgccaatggcatctttattatgatttatctgttatgcatgctg |
| | gcaggctgtaaattattgcaaggacgttatcgactactggcagtg |
| | gttggcgggctattatgcgttctgttactggcaatggtcggctgg |
| | aaaagtctctacgcgctgatcatgctggcggggttatggctgttt |
| | ctgccaaaacgaaaaacgccggaaaatggcataaccacataa |
| yjeH K/O | gtgaaaaccctgattcacctcacatttcatcgctatttcttcatc |
| (100 bp up | accgtcataagcgaatctgattgtgctaccatcgaaaatctacgc |
| and | agttgcccaaCTGACATGGGAATTAGCCATGGTCCATATGAATAT |
| downstream | CCTCCttAGTTCCTATTCCGAAGTTCCTATTCTCTAGAAAGTATA |
| and the scar | GGAACTTCGAAGCAGCTCCAGCCTACACAATCGCTCAAGACGTGT |
| site in | AATtccggcgtttcgacattaatcctggcgatcgtctttatgatc |
| between) | aaggcggtcgcggtcatcatcctttcgctggtactcaccatcaaa |
| SEQ ID | agtattaccgccac |
| NO: 663 | |
| YjeH | MSGLKQELGLAQGIGLLSTSLLGTGVFAVPALAALVAGNNSLWAW |
| Amino acid | PVLIILVFPIAIVFAILGRHYPSAGGVAHFVGMAFGSRLERVTGW |
| sequence | LFLSVIPVGLPAALQIAAGFGQAMFGWHSGQLLLAELGTLALVWY |
| SEQ ID | IGTRGASSSANLQTVIAGLIVALIVAIWWAGDIKPANIPFPAPGN |
| NO: 664 | IELTGLFAALSVMFWCFVGLEAFAHLASEFKNPERDFPRALMIGL |
| | LLAGLVYWGCTVVVLHFDAYGEQMAAAASLPKIVVQLFGVGALWI |
| | ACVIGYLACFASLNIYIQSFARLVWSQAQHNPDHYLARLSSRHIP |
| | NNALNAVLGCCVVSTLVIHALEINLDALIIYANGIFIMIYLLCML |
| | AGCKLLQGRYRLLAVVGGLLCVLLLAMVGWKSLYALIMLAGLWLF |
| | LPKRKTPENGITT |

1. A method of treating a disease associated with methionine metabolism in a human subject, the method comprising orally administering to the subject a pharmaceutical composition comprising a pharmaceutically acceptable carrier and a recombinant bacterium, wherein the recombinant bacterium is an E. coli Nissle bacterium comprising:
two copies of a metDC gene from Streptomyces sp. 590, wherein the metDC gene encodes a MetDC polypeptide, wherein the MetDC polypeptide comprises two modifications (Q70D and N82H), and wherein each copy of the metDC gene is operably linked to an IPTG inducible promoter;
a metP-metDC gene cassette comprising one copy of a metP gene from Flavobacterium segetis and a third copy of the metDC gene, wherein the metP-metDC gene cassette is operably linked to an IPTG inducible promoter, and wherein the metP gene encodes a MetP polypeptide;
a deletion in yjeH gene;
a deletion of the dapA gene;
a deletion in the pks island; and
an endogenous E. coli Nissle prophage gene deletion; and
wherein the recombinant bacterium is administered to the subject at a dose of about 3×10¹¹, about 6×10¹¹, about 1×10¹², or about 2×10¹² live cells;
thereby treating the disease associated with methionine metabolism in the subject.

2. (canceled)

3. The method of claim 1,
wherein each copy of the metDC gene encodes a MetDC polypeptide comprising a sequence that has at least 90%, 91%, 92%, 93%, 94%, 95%, 96%, 97%, 98%, or 99% identity to SEQ ID NO: 641;
wherein each copy of the metDC gene comprises a sequence that has at least 90%, 91%, 92%, 93%, 94%, 95%, 96%, 97%, 98%, or 99% identity to, comprises, or consists of SEQ ID NO: 627;
wherein the metP gene encodes a MetP polypeptide comprising a sequence having at least 90%, 91%, 92%, 93%, 94%, 95%, 96%, 97%, 98%, or 99% identity to SEQ ID NO: 649;
wherein the yjeH gene comprises a sequence having at least 90%, 91%, 92%, 93%, 94%, 95%, 96%, 97%, 98%, or 99% identity to, comprises, or consists of SEQ ID NO: 607; and/or
wherein the endogenous E. coli Nissle prophage gene deletion is a deletion of the endogenous phage gene comprising a sequence of SEQ ID NO: 292.

4.-12. (canceled)

13. The method of claim 1, wherein the recombinant bacterium is SYNB1353.

14.-15. (canceled)

16. The method of claim 1, wherein the pharmaceutical composition further comprises 25 mM to 100 mM Tris;
wherein the pH is between 6.0-8.0; and/or
wherein the pharmaceutical composition further comprises 5%-15% w/v trehalose.

17.-21. (canceled)

22. The method of claim 1, wherein the pharmaceutical composition is resuspended in bicarbonate;
wherein the pharmaceutical composition further comprises flavoring.

23.-24. (canceled)

25. The method of claim 1, wherein the subject has homocystinuria, cystinuria, or a metabolic disease.

26. The method of claim 1, wherein the pharmaceutical composition is administered to the subject once per day, twice per day, or three times per day;
wherein the subject is fed a meal within one hour of administering the pharmaceutical composition; and/or
wherein the subject is fed a meal concurrently with administering the pharmaceutical composition.

27.-28. (canceled)

29. The method of claim 1, further comprising administering a proton pump inhibitor (PPI) to the subject.

30.-37. (canceled)

38. The method of claim 1, wherein the pharmaceutical composition further comprises vitamin B₆, pyridoxal 5-phosphate (PLP), and/or pyridoxine.

39.-48. (canceled)

49. A pharmaceutical composition comprising a pharmaceutically acceptable carrier, 25 mM to 100 mM Tris, 5%-15% trehalose, pH 6.0-8.0, and a recombinant bacterium,
wherein the recombinant bacterium is an E. coli Nissle bacterium comprising:
two copies of a metDC gene, wherein each copy of the metDC gene is operably linked to an IPTG inducible promoter;
a metP-metDC gene cassette comprising one copy of a metP gene and a third copy of the metDC gene, wherein the metP-metDC gene cassette is operably linked to an IPTG inducible promoter;
a deletion in yjeH gene;
a deletion of the dapA gene;
a deletion in the pks island; and
an endogenous E. coli Nissle prophage gene deletion.

50. The pharmaceutical composition of claim 49, wherein the recombinant bacterium is SYNB1353.

51. A pharmaceutical composition comprising a pharmaceutically acceptable carrier, a recombinant bacterium, and vitamin B₆, pyridoxal 5 phosphate, and/or pyridoxine,
wherein the recombinant bacterium is an E. coli Nissle bacterium comprising:
one or more metDC gene(s), wherein the metDC gene(s) is operably linked to an non-native promoter;
one or more metP gene(s) operably linked to a non-native promoter;
a deletion in yjeH gene;
a deletion of the dapA gene;
a deletion in the pks island; and
an endogenous E. coli Nissle prophage gene deletion.

52. The pharmaceutical composition of claim 51, wherein the non-native promoters operably linked to the metDC gene(s) and the metP gene(s) is an inducible promoter.

53. The pharmaceutical composition of claim 51, wherein the non-native promoters operably linked to the metDC gene(s) and the metP gene(s) is a promoter inducible by IPTG.

54. (canceled)

55. The pharmaceutical composition of claim 51, comprising vitamin B₆, pyridoxal 5-phosphate (PLP), and/or pyridoxine (PN) in an amount of about 1 mg to about 5 mg, about 5 mg to about 10 mg, about 10 mg to about 15 mg, about 15 mg to about 20 mg, about 20 mg to about 25 mg, or about 25 mg to about 30 mg.

56. The pharmaceutical composition of claim 51, comprising vitamin B₆, pyridoxal 5-phosphate (PLP), and/or pyridoxine (PN) in an amount of about 50 mg to about 75 mg, about 75 mg to about 100 mg, about 100 mg to about 125 mg, about 125 mg to about 150 mg, about 150 mg to about 175 mg, or about 175 mg to about 200 mg.

57. The pharmaceutical composition of claim 51, comprising vitamin B₆, pyridoxal 5-phosphate (PLP), and/or pyridoxine (PN) in an amount about 25 mg per dose.

58. The pharmaceutical composition of claim 51, comprising vitamin B₆, pyridoxal 5-phosphate (PLP), and/or pyridoxine (PN) in an amount equal to or less than about 100 mg.

59. A method for monitoring the effectiveness of a treatment of a subject, the method comprising administering to the subject a pharmaceutical composition of claim 51, and measuring a level of 3-MTP in urine or 3-MTP glycine in plasma or urine of the subject.

60. A method for monitoring activity of the pharmaceutical composition of claim 51, the method comprising administering to the subject the pharmaceutical composition and measuring a level of 3-MTP in urine or 3-MTP glycine in plasma or urine of the subject.
